(19) United States
(12) Patent Application Publication
(10) Pub. No.: US 2004/0204417 A1
(43) Pub. Date: Oct. 14, 2004

(54) Novel benzothienyl or indole derivatives, preparation and use thereof as inhibitors of prenyl transferase proteins

(76) Inventors: Michel Perez, Castres (FR); Marie Lamothe, Castres (FR); Bridget Hill, London (GB); Chantal Etievant, Castres (FR)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/480,098

(22) PCT Filed: Jun. 5, 2002
(86) PCT No.: PCT/FR02/01905

(30) Foreign Application Priority Data
Jun. 6, 2001 (FR) 01 07384

(51) Int. Cl. C 07D 4 9/02; C 07D 4 3/02; C07D 413/02

(52) U.S. Cl. 514/249; 514/266.2; 514/266.24; 514/339; 514/314; 514/362; 514/364; 514/394; 514/397; 514/381; 514/378; 514/414; 544/284; 544/350; 546/167; 546/277.4; 546/281.1; 548/305.1; 548/311.4; 548/125; 548/240; 548/465

(57) ABSTRACT

The invention concerns compounds of general formula (1), wherein, in particular; W represents H, SO₂R₅. CO(CH₂)ₙR₅, (CH₂)ₙR₆, CS(CH₂)ₙR₅; X represents S or NH; Y represents (CH₂)ₚ, CO, (CH₂)ₚCO, CH═CH—CO; Z represents a hetcrocycle, imidazole, benzimidazole, isoxazole, tetrazole, oxadiazole, thiadazole, pyridine, quinazoline, quinoxaline, quinoline, thiophene; R₁ represents COOR₆, CONR₆R₇, CO—NH—CH(R₆)—COOR₇, CH₂NR₆R₇, CH₂OR₆, (CH₂)ₚR₆, CH═CHR₆; R₂ represents in particular hydrogen, C₁-C₁₀ alkyl, a substituted or unsubstituted phenyl; R₅ and R₆ represents hydrogen, C₁—C₆ alkyl; R₅ represents a substituted or unsubstituted phenyl or naphthyl; R₆ and R₇, identical or different, represent hydrogen, C₁—C₁₅ alkyl, a hetcrocycle. an aryl; n represents 0 to 10; p represents 1 to 6.

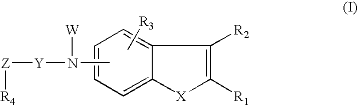

The present invention relates to novel benzothienyl or indole derivatives, to a process for manufacturing them, to pharmaceutical compositions containing them and to their use as medicinal products, in particular as protein prenyl transferase inhibitors.

The ras oncogenes (Ha-ras, Ki4a-ras, Ki4b-ras and N-ras) are present in many human cancers, for instance cancer of the pancreas and of the colon, and also in certain types of leukemia (Barbacid M. Ann. Rev. Biochem., 1987, 56:779-827; Bos J.-L. Cancer Res., 1989, 49: 4682-4689). The Ras proteins are involved in the signaling process that links the growth factors, of the cell surface, to cell proliferation.

In normal cells, biochemical studies have shown that the Ras proteins in inactive form are linked to GDP. After activation of the growth factor receptors, the Ras proteins exchange the GDP for GTP and undergo a conformational change. This activated form of the Ras protein propagates the growth signal until the Ras protein returns to its inactive form by hydrolysis of the GTP to GDP. Mutated Ras proteins, derived from the ras oncogenes, remain in the activated form and as a result transmit a permanent growth signal (Polakis P. and McCormick F. J. Biol. Chem, 1993, 268:13, 9157-9160; Glomset J. A. and Farnsworth CC. Annu. Rev. Cell. Biol., 1994, 10:181-205).

In all cases, the Ras proteins must be associated with the cell membrane in order to be active. This process especially involves the addition of an isoprenoid unit (C15 or C20) to the cysteine of the terminal tetrapeptide of the Ras proteins known as the “CAAX box” (in which C represents a cysteine, A an aliphatic amino acid, and X any amino acid).

This alkylation is catalyzed, depending on the nature of the sequence, by the enzyme Protein Farnesyl Transferase (PFTase) or by the enzyme Protein Geranyl Geranyl Transferase (PGGTase I) which respectively transfer a farnesyl (C 15) or geranyl geranyl (C20) group.

Blockage of the function of the Ras proteins should result in inhibition of the growth of the tumoral cells which depend on the activation of Ras or which express mutated Ras proteins (Perrin D., Halazy S. and Hill B. T. J Enzyme Inhi., 1996; 11:77-95; Levy R. Presse Med., 1995, 24:725-729; Sebolt-Leopold J. S. Emerging Drugs, 1996, 1:219-239; Hamilton A. D. and Sebti S. M. Drugs News Perspect, 1995, 8:138-145; Der C. J., Cox A. D., Sebti S. M. and Hamilton A. D. Anti-Cancer Drugs, 1996, 7:165-172; Halazy S., Gotteland J.-P., Lamothe M., Perrin D. and Hill B. T. Drugs of the Future, 1997, 22:1133-1146; Rowinsky E. K., Windle J. J, Von Hoff D. D. J. Clin. Oncol., 1999, 17:3631-3652, Lamothe M. and Perez M. IDrugs, 2000, 3:11, 1336-1345).

The inhibition of PFTase and/or of PGGTase I and thus of the prenylation of the Ras proteins makes it possible to control the proliferation of the ras-mutated cancer cells. This has been demonstrated using PFTase inhibitors such as BZA-5B (James G. L., Goldstein J.-L., Brown M. S. et al Science, 1993, 260:1937-1942) or L-731,734 (Kohl N. E., Mosser S. D., De Solms S. J. et al. Science, 1993, 260:1934-1937) on cell proliferation, and also with ras-dependent grafted tumors in mice (Kohl N. E., Wilson F. R., Mosser S. D. et al. Proc. Natl. Acad. Sci. USA, 1994, 91:9141-9145; Kohl N. E., Omer C. A., Conner M. W. et al. Nature Med., 1995, 1:792-797). This has also been demonstrated using PGGTase I inhibitors on cell differentiation and proliferation (Lemer E. C. Hamilton A. D. and Sebti S. M. Anti-Cancer Drug Design, 1997, 12:229-238; Sun J. et al Cancer Research, 1999, 59:4919-4926). PFTase and/or PGGTase I inhibitors may thus be useful as anticancer agents since they can serve to control cell proliferation in tumors in which the farnesylation of proteins plays a determining role. These inhibitors may also be useful in controlling the proliferation of smooth muscle cells (Indolfi et al. Nature Med, 1995, 1:541-545) and are therefore potentially useful for treating or preventing atherosclerosis and restenosis (JP H7-112930, Cohen, L. H. et al. Biochem. Pharm., 2000, 60, 1061-1068).

One subject of the present invention is a novel class of protein prenylation inhibitors and more particularly of PFTase and/or PGGTase I inhibitors, which are distinguished from the prior art by their different chemical structure and their noteworthy biological property.

A subject of the present invention is benzothienyl or indole derivatives, which have the capacity of inhibiting PFTase and/or PGGTase I not only at the enzymatic level but also at the cellular level.

The prior art in this field is illustrated especially by:

imidazole derivatives that may contain a benzothienyl or an indole, and which are described as prenyl transferase inhibitors (WO 99/65898);

pyrazole derivatives that may contain an indole as substituent of an amino acid (tryptophan), and which are described as PFTase and PGGTase inhibitors (WO 00/39083);

peptide derivatives that may contain an indole as substituent of an amino acid (tryptophan), and which are described as PFTase inhibitors (WO 96/10037, WO 95/11917, WO 96/17861).

The compounds of the present invention are of general formula (I):

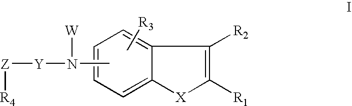

in which:

W represents:

hydrogen, SO₂R₅, CO(CH₂)ₙR₅, (CH₂)ₙR₆, CS(CH₂)ₙR₅

X represents:

S or NH

Y represents:

(CH₂)ₚ, CO, (CH₂)ₚCO, CH═CH—CO

when Y═CO, (CH₂)ₚCO or CH═CH—CO, then W represents only

hydrogen or (CH₂)ₙR₆

When Y═CO, then X represents only S.

Z represents:

imidazole, benzimidazole, isoxazole, tetrazole, oxadiazole, thiadiazole, pyridine, quinazoline, quinoxaline, quinoline, thiophene. These heterocycles may be unsubstituted or substituted with one or more groups chosen from C₁-C₁₅ alkyl, halogen, OMe, CN, NO₂, OH, CF₃, OCF₃, OCH₂Ph, SMe, COOMe, COOEt, COOH, CONHOH, SO₂NH2, CONH₂.

When Z=pyridine, then X represents only S.

R₁ represents:

COOR₆, CONR₆R₇, CO—NH—CH(R₆)—COOR₇, CH₂NR₆R₇, CH₂OR₆, (CH₂)ₚR₆, CH═CHR₆.

R₂ represents:

a) hydrogen,

b) C₁-C₁₀ alkyl, cycloalkyl, C₃-C₃₀ alkenyl, C₃-C₂₀ alkynyl

c) a phenyl, which is unsubstituted or substituted with one or more residues chosen from C₁-C₆ alkyl, halogen, phenyl, naphthyl, NO₂, CN, CF₃, OR₆, SR₆, NR₆R₇, COOR₆, CONR₆R₇, COR₆.

R₃ represents:

hydrogen, C₁-C₆ alkyl, halogen, OMe, CN, NO₂, OH, CF₃, OCF₃, OCH₂Ph, SMe, COOMe, COOEt, COOH, CONHOH, SO₂NH2, CONH₂.

R₄ represents:

a) hydrogen,

b) C₁-C₆ alkyl, which is unsubstituted or substituted with one or more residues chosen from aryl, cyanophenyl, nitrophenyl, aminophenyl, methoxyphenyl, hydroxyphenyl, heterocycle, halogen, CN, NO₂, OR₂, SR₂, NR₂R₃, COOR₂;

c) an aryl,

d) a heterocycle.

R₅ represents:

a) a phenyl or naphthyl, which is unsubstituted or substituted with one or more residues chosen from C₁-C₆ alkyl, halogen, phenyl, naphthyl, NO₂, CN, CF₃, OR₆, SR₆, NR₆R₇, COOR₆, CONR₆R₇, COR₆;

b) C₁-C₁₅ alkyl, C₃-C₃₀ alkenyl or C₃-C₂₀ alkynyl, which is unsubstituted or substituted with one or more residues chosen from halogen, COOMe, COOH, OR₂, CF₃, CN, SR₂; a cycloalkyl, which is unsubstituted or substituted with a halogen, OR₂, CF₃, CN, SR₂; an alkylcycloalkyl, which is unsubstituted or substituted with a halogen, OR₂, CF₃, CN, SR₂;

c) a heterocycle,

d) NR₆R₇

R₆ and R₇, which may be identical or different, represent:

a) hydrogen; C₁-C₁₅ alkyl, C₃-C₃₀ alkenyl or C₃-C₂₀ alkynyl, which is unsubstituted or substituted with one or more residues chosen from halogen, COOMe, COOH, OR₂, CF₃, CN, SR₂; a cycloalkyl, which is unsubstituted or substituted with a halogen, OR₂, CF₃, CN, SR₂; an alkylcycloalkyl, which is unsubstituted or substituted with a halogen, OMe, OH, CF₃, CN or SMe,

b) a heterocycle or an alkylheterocycle,

c) an aryl, an alkylaryl or an alkyldiaryl,

d) R₆ and R₇, when they are adjacent, taken together, may form a 4- to 6-membered ring with the nitrogen atom to which they are attached, which may contain one or more hetero atoms chosen from N, S and O and which may be unsubstituted or substituted with one or more groups chosen from C₁-C₁₅ alkyl, aryl and alkylaryl.

n represents:

0 to 10

p represents:

1 to 6

and the therapeutically acceptable salts and solvates thereof.

In the preceding definitions and also in the claims:

All the combinations of substituents or of variables are possible provided that they lead to stable compounds.

The term “alkyl” represents linear or branched, saturated aliphatic hydrocarbon-based chains, which are unsubstituted or substituted with one or more groups chosen from halogen, NH₂, OH and phenyl, and which comprise the specified number of carbon atoms.

The term “cycloalkyl” represents cyclic hydrocarbon-based chains containing from 3 to 10 carbon atoms.

The term “alkenyl” represents linear or branched hydrocarbon-based chains comprising 1 to 6 double bonds, which may be unsubstituted or substituted with one or more groups chosen from halogen, NH₂, OH and phenyl, and comprising the specified number of carbon atoms. Examples that may be mentioned include a residue chosen from farnesyl, geranyl, geranylgeranyl, allyl and vinyl.

The term “alkynyl” represents linear or branched hydrocarbon-based chains comprising 1 to 4 triple bonds, which may be unsubstituted or substituted with one or more groups chosen from halogen, NH2, OH and phenyl, and comprising the specified number of carbon atoms.

The term “halogen” represents a fluorine, chlorine, bromine or iodine.

The term “aryl” represents any monocyclic or bicyclic carbon-based ring possibly containing up to 7 atoms per ring and in which at least one of the rings is aromatic. Examples that may be mentioned include a phenyl, biphenyl, naphthyl, tetrahydronaphthyl or indanyl. These aromatic nuclei may be unsubstituted or substituted with one or more groups chosen from C₁-C₁₅ alkyl, halogen, OMe, CN, NO₂, OH, CF₃, OCF₃, OCH₂Ph, SMe, COOMe, COOEt, COOH.

The term “heterocycle” represents either a stable monocycle containing from 5 to 7 atoms or a stable bicycle containing from 8 to 11 atoms, which may be either saturated or unsaturated, and may consist of carbon atoms and of one to four hetero atoms chosen from N, O and S. Monocyclic heterocycles fused to a benzene nucleus are also included in the definition of bicycles. Examples that may be mentioned include a residue chosen from fuiran, pyrrole, thiophene, thiazole, isothiazole, oxadiazole, imidazole, oxazole, isoxazole, pyridine, pyrimidine, quinazoline, quinoline, quinoxaline, tetrahydroquinoline, benzofuran, benzothiophene, indole, indoline, benzothiazole, benzothienyl, benzopyran, benzoxazole, benzo[1,3]dioxole, benzisoxazole, benzimidazole, chroman, dihydrobenzofuiran, dihydrobenzothienyl, dihydroisoxazole, isoquinoline, morpholine, thiomorpholine, piperazine and piperidine. These heterocycles may be unsubstituted or substituted with one or more groups chosen from C₁-C₁₅ alkyl, halogen, OMe, CN, NO₂, OH, CF₃, OCF₃, OCH₂Ph, SMe, COOMe, COOEt and COOH.

In the terms “alkylcycloalkyl”, “alkylaryl”, “alkyldiaryl” and “alkylheterocycle” the prefix “alkyl” represents linear or branched, saturated or unsaturated aliphatic hydrocarbon-based chains containing from 1 to 15 carbon atoms and preceding the groups mentioned, the definition of which has been given previously.

The therapeutically acceptable salts of the compounds of the present invention comprise the conventional nontoxic salts of the compounds of the invention, such as those formed from organic or mineral acids. Examples that may be mentioned include the salts derived from mineral acids, for instance hydrochloric acid, hydrobromic acid, phosphoric acid or sulfuric acid, and those derived from organic acids, for instance acetic acid, trifluoroacetic acid, propionic acid, succinic acid, fumaric acid, malic acid, tartaric acid, citric acid, ascorbic acid, maleic acid, glutamic acid, benzoic acid, salicylic acid, toluenesulfonic acid, methanesulfonic acid, stearic acid or lactic acid.

These salts may be synthesized from the compounds according to the invention containing a basic portion and the corresponding acids according to the conventional chemical methods.

The therapeutically acceptable solvates of the compounds of the present invention comprise conventional solvates such as those formed during the final step of preparation of the compounds of the invention due to the presence of solvents. Examples that may be mentioned include the solvates due to the presence of water or ethanol.

All the stereoisomers, including all the optical isomers, of the compounds of general formula (I) also form part of the present invention, as does the mixture thereof in racemic form.

Among the compounds of general formula (I) forming part of the present invention, one category of compounds that is particularly suitable corresponds to the compounds of general formula (I) in which R₂, R₃ and R₄ each represent a hydrogen and Y represents a methylene (CH₂).

Another category of compounds forming part of the present invention that is particularly satisfactory corresponds to the compounds of general formula (I) in which Z represents an imidazolyl or pyridyl residue.

A third category of compounds forming part of the present invention that is particularly satisfactory corresponds to the compounds of general formula (I) in which Z represents an imidazolyl residue and R₄ represents a methyl or benzyl group, which is unsubstituted or substituted with a nitrile, nitro or methoxy group in position 4.

A fourth category of compounds forming part of the present invention that is particularly satisfactory corresponds to the compounds of general formula (I) in which X represents a sulfur atom.

A fifth category of compounds forming part of the present invention that is particularly satisfactory corresponds to the compounds of general formula (I) in which X represents an NH and R₂ represents a phenyl.

The present invention also relates to the preparation of the compounds of general formula (I) by the general processes described in the synthetic schemes below, completed, where appropriate, by any standard manipulation described in the literature or well known to those skilled in the art, or else given as an example in the experimental section.

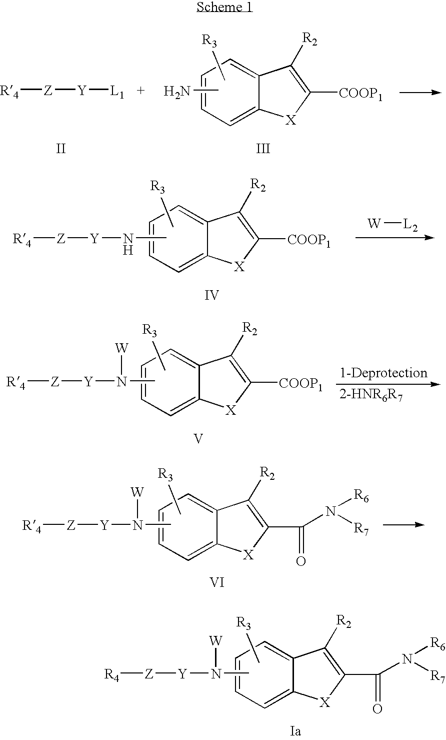

Scheme 1 illustrates the first general process that may be used for preparing the compounds of general formula (Ia). In the above general formulae, Z, Y, X, W, R₂, R₃, R₄, R₆ and R₇ are defined as in the description preceding the general formula (I). R′₄ corresponds either to R₄ (defined above) or to a precursor of R₄, or to a protecting group of Z, or alternatively to a resin in the case of a synthesis on a solid support. This group R′₄ may be removed or converted at the end of the synthesis to allow the introduction of R₄. P₁ represents either a protecting group or the species COOP, may represent an ester. L₁ may represent a leaving group such as, for example, Cl, Br, I, OSO₂CH₃, OSO₂CF₃ or O-tosyl. In this case, the reaction with the amine of general formula (III) will be performed in the presence of an organic or mineral base, such as, for example, Et₃N, iPr₂NEt, pyridine, NaH, Cs₂CO₃ or K₂CO₃, in a polar anhydrous solvent such as THF, DMF, DMSO or CH₂Cl₂ at a temperature of between −20° C. and 100° C. In the case where Y represents CO, (CH₂)ₚCO or CH═CHCO, L₁ may also represent a hydroxyl. In this case, the reaction with the amine of general formula (III) amounts to the formation of an amide by condensation between this amine and a carboxylic acid derivative. This reaction may be performed by the methods and techniques that are well known to those skilled in the art. One method that is particularly satisfactory consists in condensing a carboxylic acid of general formula (II) with an amine of general formula (III) in the presence of 1-(3-dimethylaminopropyl)-3-ethylcarbodiimide (EDC), 3-hydroxy-1,2,3-benzotriazin-4(3H)-one or a tertiary amine such as diisopropylethylamine, in a polar aprotic solvent such as dichloromethane, at a temperature of between −15° C. and 40° C. In the particular case of the intermediates of formula (IV) in which Y represents (CH₂)ₚ, one preparation method consists in performing a reductive amination using an aldehyde of formula R′₄-Z-(CH₂)ₙ₋₁—CHO in which R′₄ and Z are defined as above, an amine of general formula (III) and a reducing agent such as NaBH₄, NaBH₃CN or NaBH(OAc)₃ in a polar solvent such as 1,2-dichloroethane, THF, DMF or MeOH, at a pH that may be controlled by the addition of an acid, for instance acetic acid, at a temperature of between −20° C. and 100° C.

The intermediate of general formula (IV) is converted into an intermediate of general formula (V) by reaction with W-L₂ in which L₂ may represent a leaving group such as, for example, Cl, Br, I, OSO₂CH₃, OSO₂CF₃ or O-tosyl. In this case, the reaction with the amine of general formula (IV) will be performed in the presence of an organic or mineral base such as, for example, Et₃N, iPr₂NEt, NaH, pyridine, Cs₂CO₃ or K₂CO₃, in a polar anhydrous solvent such as THF, DMF, DMSO or CH₂Cl₂ at a temperature of between −20° C. and 100° C. L₂ may also represent a hydroxyl. In this case, the reaction with the amine of general formula (IV) amounts to the formation of an amide by condensation between this amine and a carboxylic acid derivative. This reaction may be performed by methods and techniques that are well known to those skilled in the art. One method that is particularly satisfactory consists in condensing a carboxylic acid of general formula W-L₂ with an amine of general formula (IV) in the presence of 1-(3-dimethylaminopropyl)-3-ethylcarbodiimide (EDC), 3-hydroxy-1,2,3-benzotriazin-4(3H)-one, and a tertiary amine such as diisopropylethylamine, in a polar aprotic solvent such as dichloromethane, at a temperature of between −15° C. and 40° C. In the particular case of the intermediates of general formula (V) in which W represents CO(CH₂)ₙR₅ or CS(CH₂)ₙR₅ with n=0 and R₅═NR₆R₇, one preparation method consists in performing a condensation between an isocyanate or an isothiocyanate of formula R₆NCO or R₆NCS, respectively, in which R is defined as above and R₇ represents a hydrogen, with an amine of general formula (IV). In this case, the reaction with the amine of general formula (IV) will be performed in an apolar solvent such as toluene or benzene at a temperature of between 400 and 100° C. In the particular case of the intermediates of general formula (V) in which W represents (CH₂)ₙR₆, one preparation method consists in performing a reductive amination using an aldehyde of formula R₆-(CH₂)ₙ₋₁—CHO in which R₆is defined as above, an amine of general formula (IV) and a reducing agent such as NaBH, NaBH₃CN or NaBH(OAc)₃, in a polar solvent such as 1,2-dichloroethane, THF, DMF or MeOH, at a pH that may be controlled by the addition of an acid, such as acetic acid, at a temperature of between −20° C. and 100° C. After deprotection of the species COOP₁ of the intermediate (V) via methods and techniques that are well known to those skilled in the art (“Protective Groups in Organic Synthesis”, T. W. Greene, John Wiley & Sons, 1981, and “Protecting Groups”, P. J. Kocienski, Thieme Verlag, 1994) or alternatively via saponification in basic medium in the case where the species COOP₁ represents an ester, the carboxylic acid obtained may react with the amine of general formula HNR₆R₇. This reaction may be performed via the methods and techniques that are well known to those skilled in the art. One method that is particularly satisfactory consists in condensing these 2 species in the presence of 1,3-diisopropylcarbodiimide (DIC), 3-hydroxy-1,2,3-benzotriazin-4(3H)-one and a tertiary amine such as diisopropylethylamine, in a polar aprotic solvent such as dichloromethane, at a temperature of between −15° C. and 40° C., or alternatively, by way of example, using benzotriazol-1-yloxytris(dimethylamino)phosphonium hexafluorophosphate (BOP) in the presence of 1-hydroxybenzotriazole and a tertiary amine such as diisopropylethylamine, in a polar solvent (DMF, CH₂Cl₂ or DMSO), at a temperature of between 100 and 40° C., or alternatively, by way of example, using PS-carbodiimide in the presence of 1-hydroxybenzotriazole, in a polar solvent (DMF, CH₂Cl₂ or DMSO) at a temperature of between −10° and 35° C. The conversion of R′₄ of the intermediate (VI) into R₄ of the compounds of general formula (Ia) will be dependent on the nature of R′₄. In the case where R′₄ represents a protecting group, the methods and techniques that are well known to those skilled in the art will be used (“Protective Groups in Organic Synthesis”, T. W. Greene, John Wiley & Sons, 1981 and “Protecting Groups”, P. J. Kocienski, Thieme Verlag, 1994). In the case where R′₄ represents a solid support such as, for example, a trityl resin, cleavage from this solid support may be performed so as to recover the final product. One cleavage method that is particularly suitable consists in treating the intermediate (VI) with trifluoroacetic acid (TFA) in a polar solvent such as dichloromethane, in the presence of triethylsilane, at a temperature of between 0° and 40° C. In the case where R′₄ is equal to R₄, the last step is omitted.

Scheme 2 illustrates the second general process that may be used to prepare the compounds of general formula (Ia). In the general formulae below, Z, Y, X, W, R₂, R₃, R₄, R₆, R₇, L₁ and L₂ are defined as in the above description. R′₆ corresponds either to R₆ or to a precursor of R₆ or to a resin in the case of a synthesis on a solid support. The reaction between the intermediate of general formula (VII) and the amine R′₆R₇NH may be performed according to the same procedures as those described in the first process above. The conversion of the intermediate of formula (VIII) into intermediates of formulae (IX) and (X) may be performed according to the procedures described in the first process above. In the case where the compounds of general formula (Ia) containing a group W equal to hydrogen are desired, then the step for conversion of the intermediate of formula (IX) into an intermediate of formula (X) is omitted. The conversion of the intermediate of general formula (X) into a compound of general formula (Ia) will depend on the nature of R′₆. In the case where R′₆ represents a resin such as a Wang resin presubstituted with an amino acid such as methionine or leucine, cleavage from this solid support may be performed in order to recover the final product. One cleavage method that is particularly satisfactory consists in treating the intermediate (X) with trifluoroacetic acid (TFA) in a polar solvent such as dichloromethane in the presence of triethylsilane at a temperature of between 0° and 40° C. A second cleavage method consists in treating the intermediate (X) with a base such as LiOH or NaOH in polar solvents such as methanol, THF and water, at a temperature of between 20° and 60° C. One cleavage method that is particularly satisfactory consists in treating the resin with a THF/MeOH/LiOH (1M/water) mixture in 5/2/1 proportions, at 55° C. In the case where R′₆ is equal to R₆, the last step is omitted.

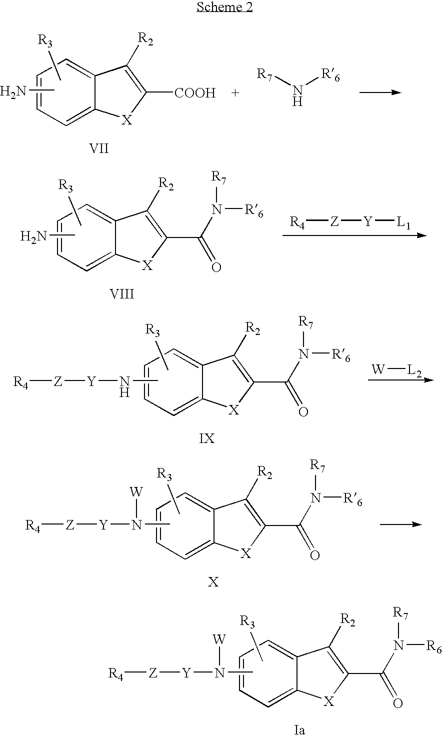

A third cleavage method making it possible this time to obtain a terminal methyl ester consists in performing a transesterification by treatment of the intermediate (X) with an organic base such as triethylamine (Et₃N) in a polar solvent such as methanol or THF, at a temperature of between 20° and 60° C. One cleavage method that is particularly satisfactory consists in treating the resin with a THF/MeOH/Et₃N mixture in 1/2/2 proportions at 55° C. In the case where R′₆ represents a protecting group, the methods and techniques that are well known to those skilled in the art will be used (“Protective Groups in Organic Synthesis”, T. W. Greene, John Wiley & Sons, 1981 and “Protecting Groups”, P. J. Kocienski, Thieme Verlag, 1994).

Scheme 3 illustrates the first general process that may be used for the preparation of the compounds of general formula (Ib). In the general formulae below, Z, Y, X, W, R₂, R₃ and R′₄ are defined as in the above descriptions, except that these groups will be carefully selected so as to be compatible with the reduction step and P₁ will preferably be a methyl or an ethyl. R′₆ corresponds either to R₆ (defined above) or to a precursor of R6.

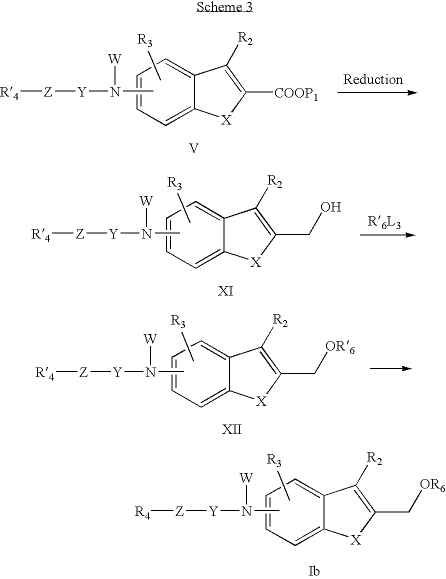

The intermediate of general formula (V) is converted into an intermediate of general formula (XI) by reduction using a reducing agent such as the BH₃·THF complex or AlH₃ or alternatively LiAlH4 in the case where the other functions present on the molecule allow it, in an anhydrous polar solvent such as THF or ethyl ether, at a temperature of between −20 and 40° C. The intermediate (XI) obtained may then be treated with the species R′₆L₃ in which L₃ may represent a leaving group, for instance Cl, Br, I, OSO₂CH₃, OSO₂CF₃ or O-tosyl. In this case, the reaction with the alcohol of general formula (XI) will be performed in the presence of an organic or mineral base, for instance Et₃N, iPr₂NEt, pyridine, NaH, Cs₂CO₃, K₂CO₃ or a base supported on a solid support, for instance PS-carbonate resin, in a polar anhydrous solvent such as THF, DMF, CH₂Cl₂ or DMSO, at a temperature of between −20° and 100° C. L₃ may also represent a hydroxyl. In this case, the reaction with the alcohol of general formula (XI) amounts to the Mitsunobu reaction and may be performed in the presence of diethyl azodicarboxylate (DEAD) and triphenylphosphine in a polar anhydrous solvent such as THF, at a temperature of between 0 and 60° C. The conversion of R′₄ of the intermediate (XII) into R₄ of the compounds of general formula (Ib) will be performed, depending on the nature of R′₄, under the conditions described in the first general process.

Scheme 4 illustrates the second general process that may be used for the preparation of the compounds of general formula (Ib). In the general formulae below, Z, Y, X, W, R₂, R₃, R₄, R₆, P₁, L₁, L₂ and L₃ are defined as in the above descriptions. R′₆ corresponds either to R′₆ or to a precursor of R′₆, or to a resin in the case of a synthesis on a solid support. The reduction reaction of the intermediate of general formula (XIII) may be performed according to the same procedures as those described in the first process above. The conversion of the intermediate of formula (XIV) into intermediates of formula (XV) may be performed according to the procedures described in the first process above. The conversion of the intermediate of general formula (XV) into the intermediate of general formula (XVI) will be performed in 3 steps. The first consists in reducing the nitro group via methods and techniques that are well known to those skilled in the art. One method that is particularly satisfactory consists in treating the nitro compound with hydrogen gas in a polar solvent such as methanol, ethanol or THF, at room temperature, in the presence of a catalyst such as Pd/C or Pd(OH)₂/C. When the reaction is performed on a solid support, one method that is particularly satisfactory consists in treating the nitro compound with tin chloride dihydrate in a polar solvent such as ethanol at a temperature of between 25 and 90° C. The second and third steps may be performed according to the procedures described in the above processes. Finally, the conversion of the intermediate of general formula (XVI) into a compound of general formula (Ib) will depend on the nature of R″₆. In the case where R″₆ represents a resin such as a Wang resin presubstituted with a group R₆, cleavage from this solid support may be performed in order to recover the final product. One cleavage method that is particularly satisfactory consists in treating the intermediate (XVI) with trifluoroacetic acid (TFA) in a polar solvent such as dichloromethane in the presence of triethylsilane at a temperature of between 0° and 40° C. Other methods of cleavage in basic medium may also be used as described above. In the case where R″₆ represents a protecting group, the deprotection methods and techniques that are well known to those skilled in the art will be used.

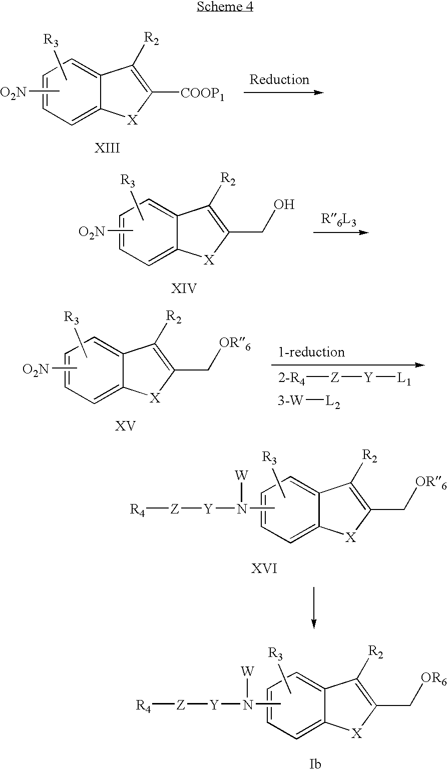

Scheme 5 illustrates the general process that may be used for the preparation of the compounds of general formulae (Ic) and (Id). In the general formulae below, Z, Y, X, W, R₂, R₃, R′₄ and R₆ are defined as in the above descriptions. The conversion of the intermediate of general formula (XI) into an intermediate of general formula (XVII) will be performed via oxidation of the alcohol into an aldehyde via methods and techniques that are well known to those skilled in the art. One method that is particularly satisfactory consists in treating the intermediate (XI) with oxalyl chloride and DMSO in a polar aprotic solvent such as dichloromethane at a temperature of between 78 and −40° C. The conversion of the intermediate of general formula (XVII) into an intermediate of general formula (XVIII) may be performed by reacting a phosphonium salt of general formula Ph₃P⁺CH₂R₆ V⁻in which R₆ is defined as above and V represents a halogen, in an anhydrous solvent such as THF, in the presence of a base such as butyllithium or potassium tert-butoxide, at a temperature of between −78 and 25° C. The next step consists in reducing the double bond of the intermediate of general formula (XVIII) via methods and techniques that are well known to those skilled in the art. One method that is particularly satisfactory consists in hydrogenating the compound in the presence of an insoluble catalyst such as palladium-on-charcoal, in a polar solvent such as methanol or ethyl acetate. The conversion of R′₄ of the intermediate (XVIII) and (XIX) into R₄ of the compounds of general formulae (Ic) at (Id) will be performed, depending on the nature of R′₄, under the conditions described in the first general process.

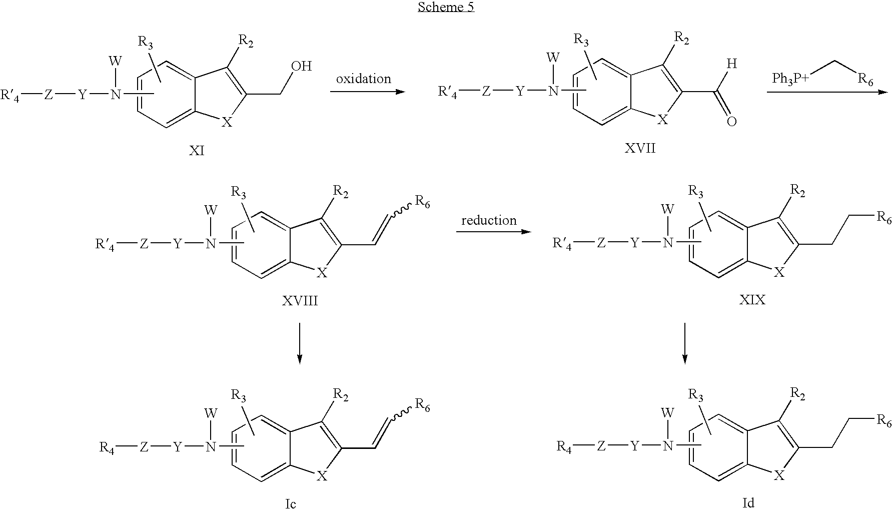

Any method for preparing a compound of general formula (I) starting with another derivative of general formula (I) in which at least one of the substituents is different should also be considered as forming part of the present invention. Thus, for example, a compound of general formula (I) in which Z represents an imidazole and R₄ represents H may be converted into a compound of general formula (I) in which Z represents an imidazole and R₄ represents a benzyl, by selective protection of the imidazole by reaction with trityl chloride followed by a reaction with a benzyl halide according to a method that is well known to those skilled in the art.

It will be understood that in certain chemical reactions or sequences of chemical reactions leading to the preparation of compounds of general formula (I), it is necessary or desirable to protect any sensitive groups in the synthetic intermediates so as to avoid undesirable side reactions. This may be performed by using (introducing and deprotecting) conventional protecting groups such as those described in “Protective Groups in Organic Synthesis”, T. W. Greene, John Wiley & Sons, 1981 and “Protecting Groups”, P. J. Kocienski, Thieme Verlag, 1994. The suitable protecting groups will thus be introduced and removed during the step that is most appropriate to do so and using the methods and techniques described in the references mentioned previously.

When it is desired to isolate a compound of general formula (I) containing at least one basic function in salt form by addition with an acid, this may be achieved by treating the free base of general formula (I) with a suitable acid, preferably in equivalent amount.

When the processes described above for preparing the compounds of the invention give mixtures of diastereoisomers, these isomers may be separated by conventional methods such as preparative chromatography.

When the novel compounds of general formula (I) contain one or more asymmetric centers, they may be prepared in the form of a racemic mixture or in the form of enantiomers, whether by enantioselective synthesis or by resolution.

The examples that follow illustrate the invention without, however, limiting its scope.

### EXAMPLE 1

(2S)-2-({4-[(3H-Imidazol-4-ylmethyl)amino]benzo[b]thiophen-2-carbonyl}amino)-4-(methylsulfanyl)butyric acid trifluoroacetate(1)

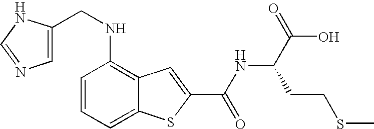

### EXAMPLE 1A

### 2-(1,3-dioxo-1,3-dihydroisoindol-2-yl)-6-fluorobenzaldehyde

2,6-Difluorobenzaldehyde (79.7 g; 350 mmol) and potassium phthalimide (77.8 g; 420 mmol) are dissolved in DMF (900 ml) under a nitrogen atmosphere. The reaction mixture is heated at 150° C. for 1.5 hours and then concentrated. The oily residue is dissolved in water (700 ml) and then extracted with dichloromethane (800 ml). The organic phase is washed successively with water and with saturated aqueous sodium chloride solution, and then dried over magnesium sulfate, filtered and concentrated. This crude reaction product is then purified by flash chromatography (petroleum ether/CH₂Cl₂ gradient: 50/50 to 20/80) to give the desired product (37.5 g, 40%).

¹H NMR, DMSO-d₆ (ppm): 7.48 (d, 1H, 7.9 Hz); 7.61 (t, 1H, 9.5 Hz); 7.85-8.1 (m, 5H); 10.19 (s, 1H).

Elemental analysis (C₁₅H₈FNO₃) % calculated: C 66.92; H 3.00; N 5.20% found: C 66.59; H 3.15; N 5.33

### EXAMPLE 1B

### ethyl 4-(1,3-dioxo-1,3-dihydroisoindol-2-yl)benzo[b]thiophene-2-carboxylate

Compound 1A (35.8 g, 133 mmol) and ethyl 2-mercaptoacetate (14.6 ml, 200 mmol) are dissolved in acetonitrile (900 ml) under a nitrogen atmosphere and in the presence of potassium carbonate (27.6 g, 200 mmol). The reaction mixture is refluxed for 18 hours and then concentrated. The solid obtained is dissolved in water (600 ml). This solution is extracted with ethyl acetate (2×700 ml). The organic phases are combined, washed with water (600 ml), dried over magnesium sulfate, filtered and concentrated. This crude reaction product is then purified by flash chromatography (petroleum ether/CH₂Cl₂ gradient: 50/50 to 20/80) to give the desired product (13.9 g, 30%).

All the aqueous phases are combined, filtered and then acidified to pH 1 with 1N HCl solution. The precipitate formed is filtered off, rinsed with acetonitrile and dried. It is then dissolved under a nitrogen atmosphere in dichloromethane (250 ml) in the presence of benzotriazol-1-yloxytris(dimethylamino)phosphonium hexafluorophosphate (BOP) (35 g, 108 mmol) and diisopropylethylamine (DIPEA) (18.9 g, 108 mmol). The reaction mixture is stirred at room temperature for 2.5 hours and then washed with water (2×100 ml), dried over magnesium sulfate, filtered and concentrated. The residue obtained is purified by flash chromatography (20/80 petroleum ether/CH₂Cl₂) to give a second batch of desired product (1.5 g, 25%).

¹H NMR, DMSO-d₆ (ppm): 1.30 (t, 3H, 7.2 Hz); 4.34 (q, 2H, 7.2 Hz); 7.55 (d, 1H, 7.5 Hz); 7.70 (t, 1H, 7.8 Hz); 7.85-7.95 (m, 2H); 7.95-8.05 (m, 2H); 8.20 (s, 1H); 8.21 (d, 1H, 9.4 Hz).

Elemental analysis (C₁₉H₁₃NO₄S·0.2H₂O) % calculated: C 64.29; H 3.80; N 3.95% found: C 64.17; H 3.90; N 3.87

### Example 1C

### ethyl 4-aminobenzo[b]thiophene-2-carboxylate

Compound 1B (17.1 g, 49 mmol) is dissolved, under a nitrogen atmosphere, in ethanol (950 ml). Hydrazine (17 ml) is added and the reaction is heated at 76° C. for 24 hours. The medium becomes heterogeneous. It is cooled to room temperature and then filtered. The solid is rinsed twice with dichloromethane. The filtrates are combined and then evaporated to dryness. The solid obtained is co-evaporated twice with ethanol. It is finally purified by flash chromatography (20/80 petroleum ether/CH₂Cl₂) to give the desired product (9.7 g, 89%).

¹H NMR, DMSO-d₆ (ppm): 1.35 (t, 3H); 4.35 (q, 2H); 6.10 (s, 2H, NH2); 6.56 (d, 1H); 7.10 (d, 1H); 7.20 (t, 1H); 8.48 (s, 1H).

Elemental analysis (C₁₁H₁₁NO₂S) % calculated: C 59.71; H 5.01; N 6.33% found: C 59.62; H 5.10; N 6.32

### Example 1D

### b 4-aminobenzo[b]thiophene-2-carboxylic acid

Compound 1C (5 g; 22 mmol) is dissolved in THF (77 ml) and water (26 ml). Sodium hydroxide (30% in water; 3.4 ml; 34 mmol) is added and the reaction mixture is heated at 80° C. for 3.5 hours. The reaction mixture is concentrated. The resulting aqueous solution is neutralized with 1N HCl to pH 5.3. The desired product precipitates out. It is filtered off, rinsed with acetonitrile and dried (3.5 g, 88%).

¹HNMR, DMSO-d₆ (ppm): 5.0-7.0 (se, 2H); 6.52 (d, 1H); 7.07 (d, 1H); 7.17 (t, 1H); 8.35 (s, 1H); 11.5-13.5 (se, 1H).

Elemental analysis (C₉H₇NO₂S) % calculated: C 55.94; H 3.65; N 7.25% found: C 55.54; H 3.45; N 7.11

### Example 1E

### b 1-tritylresin-1H-imidazole-4-carboxaldehyde (Resin)

Trityl chloride resin (2.1 mmol/g) (30 g; 63 mmol) is swollen with CH₂Cl₂ (2×80 ml) and a solution of 4(5)-imidazolecarboxaldehyde (18.2 g; 189 mmol) in DMF (134 ml) is added, followed by addition of DIPEA (134 ml). The mixture is stirred for 36 hours at room temperature and the resin is then filtered off and washed successively with DMF (2×), CH₂Cl₂ (2×), H₂O (2×), MeOH (1×), CH₂Cl₂ (2×), MeOH (2×).

A sample of this resin (80 g) is cleaved by treatment with ¼ TFA/CH₂Cl₂ solution (2 ml) for 10 minutes. After evaporating off the solvents, the product obtained is monitored by HPLC (C18, λ 230 nM, 100% H₂O to 100% CH₃CN (+0.1% TFA) over 25 minutes) and has a purity of 99%.

### Example 1F

### b 4-[(3-tritylresin-3H-imidazol-4-ylmethyl)amino]benzo[b]thio-phene-2-carboxylic acid (Resin)

Resin 1E (4 g; 5.6 mmol) is swollen with CH₂Cl₂ (2×80 ml) and a solution of aniline 1D (2.18 g; 11 mmol) in 1,2-dichloroethane (DCE) (30 ml) and methanol (5 ml) is added, along with acetic acid (1.3 ml). The mixture is stirred for 1 minute at room temperature, and sodium triacetoxyborohydride (4.78 g; 22 mmol) is then added. The reaction mixture is stirred for 24 hours. The resin is then filtered off, washed successively with MeOH (2×), H₂O (2×), MeOH (2×), CH₂Cl₂ (2×) and finally dried (4.5 g; 90%).

A sample of this resin (50 mg) is cleaved by treatment with a 50/50/10 TFA/CH₂Cl₂/Et₃SiH solution (2 ml) for 1 hour. After evaporating off the solvents, the product obtained is monitored by HPLC (C18, λ 220 nM, 100% H₂O to 100% CH₃CN (+0.1% TFA) over 25 minutes) and has a purity of 70%.

### Example 1

### (2S)-2-({4-[(3H-Imidazol-4-ylmethyl)amino]benzo[b]thiophen-2-carbonyl}amino)₄-(methylsulfanyl)butyric acid trifluoroacetate

Resin 1F (500 mg; 2.24 mmol) is swollen with CH₂Cl₂ (2×80 ml) and H-Met-O-tert-Bu hydrochloride (540 mg; 2.24 mmol), dichloromethane (11 ml), DIPEA (0.39 ml; 2.2 mmol), 3-hydroxy-1,2,3-benzotriazin-4-(3H)-one (HOOBT; 360 mg; 2.24 mmol) and 1,3-diisopropylcarbodiimide (DIC) (0.35 ml; 2.4 mmol) are then added. The mixture is stirred for 18 hours at room temperature. The resin is then filtered off, washed successively with DMF (2×), CH₂Cl₂ (2×), MeOH (2×), CH₂Cl₂ (2×) and finally dried (572 mg; 94%).

A sample of this resin (100 mg) is cleaved by treatment with a 50/50/10 TFA/CH₂Cl₂/Et₃SiH solution (3 ml) for 2.5 hours. The suspension is filtered and the resin is rinsed with CH₂Cl₂ (2×). The filtrates are combined and concentrated to give the desired product 1 (22 mg; 37%).

HPLC (C18, λ 220 nM), 100% H₂O to 100% CH₂CN (+0.1% TFA) over 8 minutes): purity: 87%.

Mass spectrum (ESI): m/z 405 (MH+).

### EXAMPLE 2

### N-(Thiophen-2-ylmethyl)-4-[(3H-imidazol-4-ylmethyl)amino]benzo[b]thiophene-2-carboxamide trifluoroacetate (2)

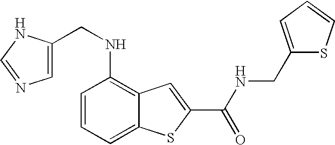

Compound 2 is prepared from resin 1F (100 mg; 0.112 mmol) and thiophen-2-ylmethylamine according to the conditions used for the preparation of 1 and abiding by the proportions of the various reagents. Amount obtained: 22 mg (43%).

HPLC (C18, λ 220 nM, 100% H₂O to 100% CH₂CN (+0.1% TFA) over 8 minutes): purity: 77%.

Mass spectrum (ESI): m/z 369 (MH+).

### Examples 3 to 9

Compounds 3 to 2 were synthesized according to the following general procedure:

Resin 1F (100 mg; 1.12 mmol/g; 0.112 mmol) is swollen with CH₂Cl₂ (2×). Next, 1.1 ml of a solution of DIC (0.4 M) and HOOBT (0.4 M) in dichloromethane, and 1.1 ml of a solution of amine (0.4 M) in dichloromethane are added. In the case of amines of amino acid type, the tert-butyl ester of the amino acid is used. The mixture is stirred for 18 hours at room temperature under a nitrogen atmosphere. The resin is then filtered off and rinsed successively with DMF (2×), CH₂Cl₂ (2×), MeOH (2×), H₂O (2×), MeOH (2×) and CH₂Cl₂ (2×).

A solution of acid chloride (0.36 M in pyridine; 2.5 ml) or of sulfonyl chloride (0.36 M in 50/50 CH₂Cl₂/pyridine; 2.5 ml) is added. The mixture is stirred for 5.5 hours at room temperature and under a nitrogen atmosphere. The resin is filtered off and then washed successively with DMF (2×), CH₂Cl₂ (2×), MeOH (2×), H₂O (2×), MeOH (2×) and CH₂Cl₂ (2×). The resin is then cleaved by treatment with a 5/5/1 TFA/CH₂Cl₂/Et₃SiH mixture (3 ml) for 2.5 hours to give, after evaporation of the filtrate, the expected product in the form of the trifluoroacetate salt.

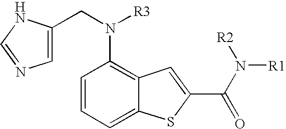

| | | | | Mass | HPLC |
|---|---|---|---|---|---|
| Example | NR1R2 | R3 | Compound name | (M + H)⁺ | purity* |
| 3 | 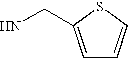 | SO₂Ph | N-(thiophen-2-ylmethyl)-4-[benzene- sulfonyl(3H-imidazol-4-ylmethyl)- amino]benzo[b]thiophene-2- carboxamide | 509 | 79 |
| 4 | 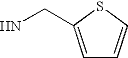 | 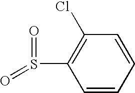 | N-(thiophen-2-ylmethyl)-4-[2-chloro- benzenesulfonyl(3H-imidazol-4- ylmethyl)amino]benzo[b]thiophene-2- carboxamide | 543 | 81 |
| 5 | 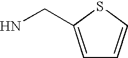 | COPh | N-(thiophen-2-ylmethyl)-4-[benzene- carbonyl(3H-imidazol-4- ylmethyl)amino]benzo[b]thiophene-2- carboxamide | 473 | 72 |
| 6 | Met-OH | SO₂Ph | (2S)-2-({4-[benzenesulfonyl(3H- imidazol-4-ylmethyl)amino]benzo- [b]thiophene-2-carbonyl}amino)-4- (methylsulfanyl)butyric acid | 545 | 82 |
| 7 | Met-OH | 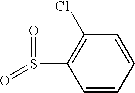 | (2S)-2-({4-[2-chlorobenzene- sulfonyl(3H-imidazol-4-ylmethyl)- amino]benzo[b]thiophene-2- carbonyl}amino)-4- (methylsulfanyl)butyric acid | 579 | 81 |
| 8 | Leu-OH | SO₂Ph | (2S)-2-({4-[benzenesulfonyl(3H- imidazol-4-ylmethyl)amino]benzo- [b]thiophene-2-carbonyl}amino)-4- methylpentanoic acid | 527 | 86 |
| 9 | Leu-OH | 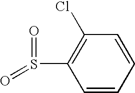 | (2S)-2-({4-[2-chlorobenzene- sulfonyl(3H-imidazol-4-ylmethyl)- amino]benzo[b]thiophene-2- carbonyl}amino)-4-methylpentanoic acid | 561 | 84 |

HPLC conditions [C18 symmetry, 4.6×50 mm, 50 μm; λ=220 nM; gradient 100% H₂O (+0.05% TFA) to 100% CH₂CN (+0.05% TFA) over 8 minutes]

### EXAMPLE 10

(2S)-2-{[5-(2-3H-Imidazol-4-ylacetylamino)benzo[b]thiophene-2-carbonyl]amino}4-methylpentanoic acid trifluoroacetate (10)

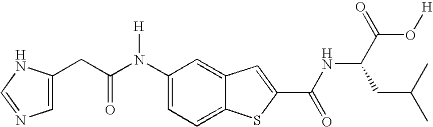

### EXAMPLE 10A

### Ethyl 5-nitrobenzo[b]thiophene-2-carboxylate

Compound 10A is prepared from 2-fluoro-5-nitrobenzaldehyde (15 g; 89 mmol) according to the conditions used for the preparation of 1B and abiding by the proportions of the various reagents. The crude reaction product is then purfied by flash chromatography (30/70 petroleum ether/CH₂Cl₂) to give the desired product (19 g, 85%).

¹HNMR, DMSO-d₆ (Ppm): 1.36 (t, 3H); 4.35 (q, 2H); 8.30 (dd, 1H); 8.35 (d, 1H); 8.41 (s, 1H); 8.98 (d, 1H).

Elemental analysis (C₁₁H₉NO₄S) % calculated: C 52.58; H 3.61; N 5.57% found: C 52.59; H 3.85; N 5.57

### Example 10B

### Ethyl 5-aminobenzo[b]thiophene-2-carboxylate

Compound 10A (18.9 g; 75 mmol) is dissolved in ethanol (600 ml) under a nitrogen atmosphere. Tin chloride dehydrate (84.9 g; 376 mmol) is added and the reaction mixture is heated at 90° C. for 18 hours. The reaction mixture is cooled to room temperature and then poured onto ice (800 g) and brought to pH 7-8 by adding saturated sodium bicarbonate solution. The solution is extracted with ethyl acetate (2×2 1). The organic phases are combined, dried over magnesium sulfate, filtered and concentrated. The crude reaction product is then purified by flash chromatography (20/80 petroleum ether/CH₂Cl₂ and then 100% CH₂Cl₂) to give the desired product (13.6 g, 82%).

¹H NMR, DMSO-d₆ (Ppm): 1.32 (t, 3H); 4.32 (q, 2H); 5.29 (s, 2H, NH2); 6.91 (dd, 1H); 7.08 (d, 1H); 7.65 (d, 1H); 7.92 (s, 1H).

Elemental analysis (Cl H₉NO₄S) % calculated: C 59.71; H 5.01; N 6.33% found: C 59.61; H 4.98; N 6.31

### Example 10C

### b 5-Aminobenzo[b]thiophene-2-carboxylic acid

Compound 10C is prepared from compound 10B (6.8 g; 31 mmol) according to the conditions used for the preparation of 1D and abiding by the proportions of the various reagents. Amount obtained: 4.99 g (83%).

¹H NMR, DMSO-d₆ (ppm): 6.87 (dd, 1H); 7.05 (d, 1H); 7.63 (d, 1H); 7.83 (s, 1H); 6.0-10.0 (bs).

Elemental analysis (C₉H₇NO₂S·0.3H₂O) % calculated: C 54.42; H 3.86; N 7.05% found: C 54.43; H 3.67; N 7.07

### Example 10

### (2S)-2-{[5-(2-3H-imidazol-4-ylacetylamino)benzo[b]thiophene-2-carbonyl]amino}-4-methylpentanoic acid trifluoroacetate

The fmoc-Leu-Wang resin (2.3 g; 0.6 mmol/g; 1.3 mmol) is suspended in piperidine (20% in DMF; 35 ml) and stirred at room temperature for 1.5 hours. It is then filtered off and rinsed successively with DMF (2×), CH₂Cl₂ (2×), MeOH (2×) and CH₂Cl₂ (2×). BOP (1.78 g; 5.52 mmol), N-methylpyrrolidone (NMP) (25 ml), DIPEA (0.96 ml; 5.5 mmol) and derivative 10C (400 mg; 2.07 mmol) are added. The mixture is stirred at room temperature for 18 hours. The resin is then filtered off, rinsed successively with DMF (2×), CH₂Cl₂ (2×), MeOH (2×), CH₂Cl₂ (2×) and MeOH (2×) and dried. 2.23 g of new resin are obtained. A fraction of this resin (200 mg) is treated at room temperature with 1-hydroxybenzotriazole (HOBT) (67 mg; 0.5 mmol), DIC (44 III; 0.5 mmol) and 2-(1-trityl-3H-imidazol-4-yl)acetic acid (Polushin, N. N.; Chen, B.-C.; Anderson, L. W.; Cohen, J. S. J. Org. Chem. 1993, 58(17), 4606) (68 mg; 0.19 mmol) in DMF (4 ml), for 20 hours. The resin is then filtered off and rinsed successively with DMF (2×), CH₂Cl₂ (2×), MeOH (2×), CH₂Cl₂ (2×), MeOH (2×) and CH₂Cl₂ (2×). It is then cleaved off by treatment with a 5/5/1 TFA/CH₂Cl₂/Et₃SiH mixture (3 ml) for 2.5 hours. The oily residue obtained after evaporation is purified by preparative HPLC (Waters Prep 4000) on a Prep Nova-Pak HR C-18 column (Waters; 25×100 mm; 6 μm) using a total gradient of from 100% water (0.1% TFA) to 100% acetonitrile (0.1% TFA) over 15 minutes, to give the expected product (30 mg; 45%).

¹H NMR, DMSO-d₆ (ppm): 0.91 (d, 3H); 0.91 (d, 3H); 1.5-1.8 (m, 3H); 3.92 (s, 2H); 4.35-4.5 (m, 1H); 7.57 (s, 1H); 7.59 (dd, 1H); 7.97 (d, 1H); 8.19 (s, 1H); 8.28 (d, 1H); 8.86 (d, 1H); 9.02 (s, 1H); 10.52 (s, 1H); 12.72 (bs, 1H); 14.29 (bs, 1H).

HPLC [C 18 symmetry, 4.6×50 mm, 5 μm; λ=220 nM; gradient 100% H₂O (+0.05% TFA) to 100% CH₂CN (+0.05% TFA) over 8 minutes]: purity: 99%.

Mass spectrum (ESI): m/z 415 (MH+).

### Examples 11 to 26

Compounds 11 to 26 are prepared from the fmoc-Leu-Wang or Fmoc-Met-Wang resins, and from the derivatives 1D or 10C, according to the conditions used for the preparation of 10 and abiding by the proportions of the various reagents. The desired products are obtained in the form of trifluoroacetate salts. The carboxylic acids used in these syntheses are known:

| RCOOH | Reference |
|---|---|
| 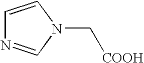 | Kamijo, T.; Yamamoto, R.; Harada, H.; Iizuka, K. Chem. Pharm. Bull. 1983, 31(4), 1213 |
| 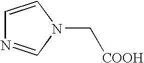 | Jones; Young Can. J. Chem. 1970, 48, 1566 |
| 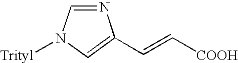 | Hunt, J.T.; Lee, V.G.; Leftheris, K.; Seizinger, B.; Carboni, J. et al. J. Med. Chem. 1996, 39(2), 353 |
| 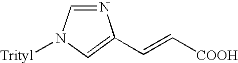 | Jung, G.L.; Anderson, P.C.; et al. Bioorg. Med. Chem. 1998, 6(12), 2317 |
| 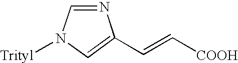 | Cloninger, M.J.; Frey, P.A. Bioorg. Chem. 1998, 26(6), 323 |
| 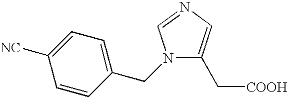 | Anthony, N.J.; Gomez, R.P.; Schaber, M.D.; et al. J. Med. Chem. 1999, 42(17), 3356 |

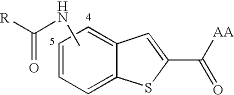

| | RCONH | | | | Mass | HPLC |
|---|---|---|---|---|---|---|
| Example | position | RCO | AA | Compound name | (M + H)⁺ | purity* |
| 11 | 5 | 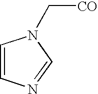 | Leu-OH | (2S)-2-({5-[2-imidazol-1- yl)acetylamino]benzo[b]- thiophene-2-carbonyl}- amino)-4-methylpentanoic acid | 415 | 90 |
| 12 | 5 | 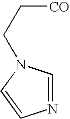 | Leu-OH | (2S)-2-({5-[3-imidazol-1- yl)propionylamino]benzo- [b]thiophene-2-carbonyl}- amino)-4-methylpentanoic acid | 429 | 99 |
| 13 | 5 | 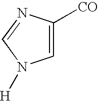 | Leu-OH | (2S)-2-({5-[3H-imidazol- 4-yl)carbonylamino]- benzo[b]thiophene-2- carbonyl}amino)-4- methylpentanoic acid | 401 | 97 |
| 14 | 5 | 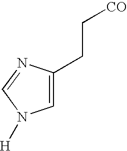 | Leu-OH | (2S)-2-({5-[3-(3H- imidazol-4-yl)propionyl- amino]benzo[b]thiophene- 2-carbonyl}amino)-4- methylpentanoic acid | 429 | 99 |
| 15 | 5 | 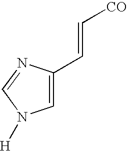 | Leu-OH | (2S)-2-({5-[3-(3H- imidazol-4-yl)acryloyl- amino]benzo[b]thiophene- 2-carbonyl}amino)-4- methylpentanoic acid | 427 | 99 |
| 16 | 5 | 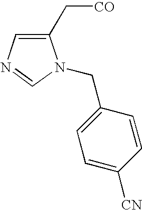 | Leu-OH | (2S)-2-[(5-{2-[3-(4- cyanobenzyl)-3H- imidazol-4-yl]acetyl- amino}benzo[b]thiophene- 2-carbonyl)amino]-4- methylpentanoic acid | 530 | 88 |
| 17 | 4 | 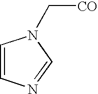 | Leu-OH | (2S)-2-({4-[2-imidazol-1- yl)acetylamino]benzo[b]- thiophene-2-carbonyl}- amino)-4-methylpentanoic acid | 415 | 98 |
| 18 | 4 | 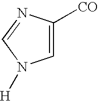 | Leu-OH | (2S)-2-{[4-(3H-imidazol- 4-ylcarbonylamino)- benzo[b]thiophene-2- carbonyl]amino}-4- methylpentanoic acid | 401 | 85 |
| 19 | 4 | 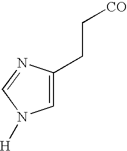 | Leu-OH | (2S)-2-({4-[3-(3H- imidazol-4-yl)propionyl- amino]benzo[b]thiophene- 2-carbonyl}amino)-4- methylpentanoic acid | 429 | 92 |
| 20 | 4 | 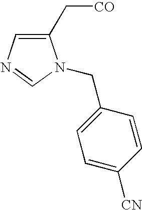 | Leu-OH | (2S)-2-[(4-{2-[3-(4-cyano- benzyl)-3H-imidazol-4- yl)acetylamino}benzo[b]- thiophene-2-carbonyl)- amino]-4-methylpentanoic acid | 530 | 92 |
| 21 | 4 | 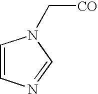 | Met-OH | (2S)-2-({4-[2-(imidazol-1- yl)acetylamino]- benzo[b]thiophene-2- carbonyl}amino)-4- (methylsulfanyl)butyric acid | 433 | 99 |
| 22 | 4 | 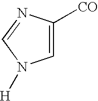 | Met-OH | (2S)-2-{[4-(3H-imidazol- 4-ylcarbonylamino)- benzo[b]thiophene-2- carbonyl]amino}-4- (methylsulfanyl)butyric acid | 419 | 79 |
| 23 | 4 | 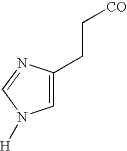 | Met-OH | (2S)-2-({4-[3-(3H- imidazol-4-yl)propionyl- amino]benzo[b]thiophene- 2-carbonyl}amino)-4- (methylsulfanyl)butyric acid | 447 | 70 |
| 24 | 4 | 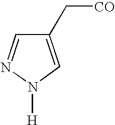 | Met-OH | (2S)-2-{[4-(2-3H- imidazol-4-ylacetyl- amino)benzo[b]thiophene- 2-carbonyl]amino}-4- (methylsulfanyl)butyric acid | 433 | 91 |
| 25 | 4 | 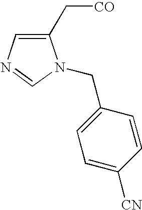 | Met-OH | (2S)-2-[(4-{2-[3-(4- cyanobenzyl)-3H- imidazol-4-yl]acetyl- amino}benzo[b]thiophene- 2-carbonyl)amino]-4- (methylsulfanyl)butyric acid | 548 | 81 |
| 26 | 4 | 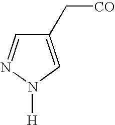 | Leu-OH | (2S)-2-{[4-(2-3H- imidazol-4-ylacetyl- amino)benzo[b]thiophene- 2-carbonyl]amino}-4- methylpentanoic_acid | 415 | 93 |

*HPLC conditions [C18 symmetry, 4.6 × 50 mm, 5 μm; λ = 220 nM; gradient 100% H₂O (+0.05% TFA) to 100% CH₂CN (+0.05% TFA) over 8 minutes]

### EXAMPLE 27

### (2S)-2-[(5-{[3-(4-Cyanobenzyl)-3H-imidazol-4-ylmethyl]amino}benzo[b]-thiophene-2-carbonyl)amino]-4-methylpentanoic acid trifluoroacetate (27)

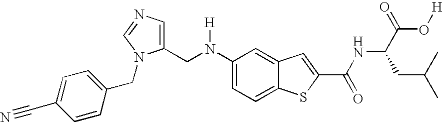

### Example 27A

### b 4-(5-Formylimidazol-1-ylmethyl)benzonitrile

1-Trityl-1H-imidazole-4-carboxaldehyde (Daminos-Zeghal S. et al., Tetrahedron, 1997, 53(22), 7605-14) (25 g; 74.0 mmol) dissolved in dichloromethane (125 ml) in the presence of sodium iodide (16.6 g; 111.0 mmol) is treated with 4-cyanobenzyl bromide (21.74 ml; 111.0 mmol) at room temperature. The medium is then refluxed under nitrogen for 24 hours and then diluted with dichloromethane and washed with saturated NaHCO₃ solution and with water. The organic phase is dried over sodium sulfate, filtered and then evaporated to dryness. The syrup obtained is purified by chromatography on a column of silica eluted with a 9/1 and then 1/1 CH₂Cl₂/acetone mixture to give the pure product in the form of a yellow solid (4.8 g; 27%).

¹HNMR, DMSO-d₆ (ppm): 5.62 s, 2H; 7.32 d, 2H; 7.82 d, 2H; 8.01 s, 1H; 8.31 s, 1H; 9.70 s, 1H

### Example 27

### (2S)-2-[(4-{[3-(4-cyanobenzyl)-3H-imidazol-4-ylmethyl]amino}-benzo[b]thiophene-2-carbonyl)amino]4-methylpentanoic acid trifluoroacetate

The fmoc-Leu-Wang resin (700 mg; 0.6 mmol/g; 0.42 mmol) is suspended in piperidine (20% in DMF; 15 ml) and stirred at room temperature for 1.5 hours. It is then filtered off and rinsed successively with DMF (2×), CH₂Cl₂ (2×), MeOH (2×) and CH₂Cl₂ (2×). BOP (0.54 g; 1.7 mmol), NMP (10 ml), DIPEA (0.29 ml; 1.7 mmol) and derivative 1D (121 mg; 0.63 mmol) are added. The mixture is stirred at room temperature for 18 hours. The resin is then filtered off and rinsed successively with DMF (2×), CH₂Cl₂ (2×), MeOH (2×) and CH₂Cl₂ (2×). This resin is then treated at room temperature with derivative 27A (250 mg; 1.19 mmol) in DCE (10 ml) and acetic acid (91 μl; 1.6 mmol) for a few minutes, sodium triacetoxyborohydride (340 mg; 1.6 mmol) is then added and the mixture is stirred for 18 hours. The resin is then filtered off and rinsed successively with MeOH (2×), H₂O (2×), MeOH (2×) and CH₂Cl₂ (2×). This resin is again treated at room temperature with derivative 27A (250 mg; 1.19 mmol) in DCE (10 ml) and acetic acid (91 μl; 1.6 mmol) for a few minutes, sodium triacetoxyborohydride (340 mg; 1.6 mmol) is then added and the mixture is stirred for 18 hours. The resin is then filtered off and rinsed successively with MeOH (2×), H₂O (2×), MeOH (2×) and CH₂Cl₂ (2×). A portion of this resin (100 mg) is then cleaved by treatment with a 1/2/5 LiOH(1M/H₂O)/MeOH/THF mixture (3 ml) for 15 minutes at 50° C. The oily residue obtained after evaporation is purified by preparative HPLC (Waters Prep 4000) on a Prep Nova-Pak HR C-18 column (Waters; 25×100 mm; 6 μm) using a total gradient of from 100% water (0.1% TFA) to 100% acetonitrile (0.1% TFA) over 15 minutes, to give the expected product (9 mg; 33%).

HPLC [C18, λ 220 nM, 100% H₂O to 100% CH₂CN (+0.1% TFA) over 8 minutes]: purity: 99%.

Mass spectrum (ESI): m/z 502 (MH+).

### EXAMPLES 28 AND 29

Compounds 28 and 29 are prepared from the fmoc-Leu-Wang or Fmoc-Met-Wang resins and from derivative 10C according to the conditions described for the preparation of 22.7 and abiding by the proportions of the various reagents. The desired products are obtained in the form of trifluoroacetate salts.

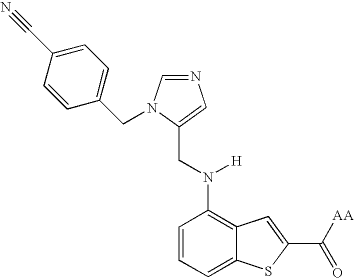

| | | | Mass | |
|---|---|---|---|---|
| Exam- | | | (M + | Purity |
| ple | AA | Compound name | H)⁺ | HPLC* |
| 28 | Leu-OH | (2S)-2-[(4-{[3-(4-cyanobenzyl)-3H- | 502 | 91 |
| | | imidazol-4-ylmethyl]amino}benzo- | | |
| | | [b]thiophene-2-carbonyl)amino]-4- | | |
| | | methylpentanoic acid | | |
| 29 | Met-OH | (2S)-2-[(4-{[3-(4-cyanobenzyl)-3H- | 520 | 78 |
| | | imidazol-4-ylmethyl]amino}benzo- | | |
| | | [b]thiophene-2-carbonyl)amino]-4- | | |
| | | methylsulfanyl)butyric acid | | |

*HPLC conditions [C18 symmetry, 4.6 × 50 mm, 5 μm; λ = 220 nM; gradient 100% H₂O (+0.05% TFA) to 100% CH₂CN (+0.05% TFA) over 8 minutes]

### Examples 30 to 33

Compounds 30 to 33 are prepared from the fmoc-Leu-Wang or Fmoc-Met-Wang resins, from derivatives 1D or 10C and from benzenesulfonyl chloride or from 2-chlorobenzenesulfonyl chloride, according to the conditions described for the preparation of 30, and abiding by the proportions of the various reagents.

The fmoc-Leu-Wang resin (700 mg; 0.6 mmol/g; 0.42 mmol) is suspended in piperidine (20% in DMF; 15 ml) and stirred at room temperature for 1.5 hours. It is then filtered off and rinsed successively with DMF (2×), CH₂Cl₂ (2×), MeOH (2×) and CH₂Cl₂ (2×). BOP (0.54 g; 1.7 mmol), NMP (10 ml), DIPEA (0.29 ml; 1.7 mmol) and derivative 1D (121 mg; 0.63 mmol) are added. The mixture is stirred at room temperature for 18 hours. The resin is then filtered off and rinsed successively with DMF (2×), CH₂Cl₂ (2×), MeOH (2×) and CH₂Cl₂ (2×). This resin is then treated at room temperature with derivative 27A (250 mg; 1.19 mmol) in DCE (10 ml) and acetic acid (91 μl; 1.6 mmol) for a few minutes, sodium triacetoxyborohydride (340 mg; 1.6 mmol) is then added and the mixture is stirred for 18 hours. The resin is then filtered off and rinsed successively with MeOH (2×), H₂O (2×), MeOH (2×) and CH₂Cl₂ (2×). This resin is again treated at room temperature with derivative 27A (250 mg; 1.19 mmol) in DCE (10 ml) and acetic acid (91 μl; 1.6 mmol) for a few minutes, sodium triacetoxyborohydride (340 mg; 1.6 mmol) is then added and the mixture is stirred for 18 hours. The resin is then filtered off and rinsed successively with MeOH (2×), H₂O (2×), MeOH (2×) and CH₂Cl₂ (2×). A portion of this resin (150 mg) is then treated with benzylsulfonyl chloride (99 μl; 0.78 mmol) in dichloromethane (1.25 ml) and pyridine (1.25 ml) at room temperature for 18 hours. The resin is then filtered off, rinsed successively with DMF (2×), CH₂Cl₂ (2×), H₂O (2×), MeOH (2×) and CH₂Cl₂ (2×), and cleaved by treatment with a 1/2/5 LiOH(1M/H₂O)/MeOH/THF mixture (3 ml) for 15 minutes at 55° C. The oily residue obtained after evaporation is purified by filtration on silica (10/90 MeOH/CH₂Cl₂) to give the expected product (25 mg; 39%).

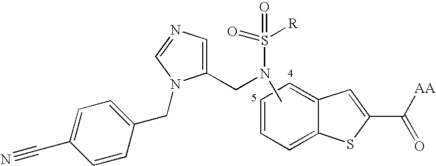

| | Position | | | | Mass | Purity |
|---|---|---|---|---|---|---|
| Example | R₁R₂N | RSO₂ | AA | Compound name | (M + H)⁺ | HPLC* |
| 30 | 4 | PhSO₂ | Met-OH | (2S)-2-[(4-{benzene- | 660 | 88 |
| | | | | sulfonyl-[3-(4-cyano- | | |
| | | | | benzyl)-3H-imidazol-4- | | |
| | | | | ylmethyl]amino}benzo- | | |
| | | | | [b]thiophene-2-carbonyl)- | | |
| | | | | amino]-4-(methyl- | | |
| | | | | sulfanyl)butyric acid | | |
| 31 | 4 | PhSO₂ | Leu-OH | (2S)-2-[(4-{benzene- | 642 | 82 |
| | | | | sulfonyl-[3-(4-cyano- | | |
| | | | | benzyl)-3H-imidazol-4- | | |
| | | | | ylmethyl]amino}benzo- | | |
| | | | | [b]thiophene-2-carbonyl)- | | |
| | | | | amino]-4-methyl- | | |
| | | | | pentanoic acid | | |
| 32 | 4 | 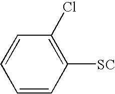 | Leu-OH | (2S)-2-[(4-{(2-chloro- benzenesulfonyl)-[3-(4- cyanobenzyl)-3H- imidazol-4-ylmethyl]- amino}benzo[b]- thiophene-2-carbonyl)- amino]-4-methyl- pentanoic acid | 676 | 99 |
| 33 | 5 | PhSO₂ | Leu-OH | (2S)-2-[(5-{benzene- | 642 | 99 |
| | | | | sulfonyl-[3-(4-cyano- | | |
| | | | | benzyl)-3H-imidazol-4- | | |
| | | | | ylmethyl]amino}benzo- | | |
| | | | | [b]thiophene-2-carbonyl)- | | |
| | | | | amino]-4-methyl- | | |
| | | | | pentanoic acid | | |

*HPLC conditions [C18 symmetry, 4.6 × 50 mm, 5 μm; λ = 220 nM; gradient 100% H₂O (+0.05% TFA) to 100% CH₂CN (+0.05% TFA) over 8 minutes]

### EXAMPLE 34

### 5 Ethyl 5-{[3-(4-cyanobenzyl)-3H-imidazol-4-ylmethyl]amino}benzo[b]thiophene-2-carboxylate (34)

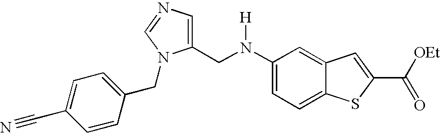

Compound 10B (2.5 g; 11 mmol) is dissolved in DCE (39 ml) in the presence of derivative 27A (2.5 g; 12 mmol) and acetic acid (2.9 ml; 56 mmol) at room temperature and under a nitrogen atmosphere, for a few minutes, and sodium triacetoxyborohydride (2.5 g; 12 mmol) is then added. After stirring for 18 hours, a little more sodium triacetoxyborohydride (0.7 g; 3 mmol) is added. After stirring for 2 hours, dichloromethane (100 ml) is added and the reaction mixture is washed twice with saturated aqueous sodium carbonate solution (100 ml) and then dried over magnesium sulfate, filtered and concentrated. This crude reaction product is then purified by flash chromatography (70/30 CH₂Cl₂/acetone; and then 95/5 to 90/10 CH₂Cl₂/MeOH gradient) to give the desired product (3.15 g; 69%).

HPLC [C18, λ 220 nM, 100% H₂O to 100% CH₂CN (+0.1% TFA) over 8 minutes]: purity: 95%.

Mass spectrum (ESI): m/z 417 (MH+).

¹HNMR, DMSO-d₆ (ppm): 1.32 (t, 3H); 4.11 (d, 2H); 4.32 (q, 2H); 5.40 (s, 2H); 6.17 (t, 1H); 6.87 (dd, 1H); 6.97 (d, 1H); 7.00 (s, 1H); 7.25 (d, 2H); 7.66 (d, 1H); 7.77 (s, 1H); 7.79 (d, 2H); 7.92 (s, 1H).

Elemental analysis (C₂₃H₂₀N₄O₂S) % calculated: C 66.33; H 4.84; N 13.45% found: C 66.18; H 4.84; N 13.41

### EXAMPLE 35

### Ethyl 4-{[3-(4-cyanobenzyl)-3H-imidazol-4-ylmethyl]amino)}-benzo[b]thiophene-2-carboxylate (35)

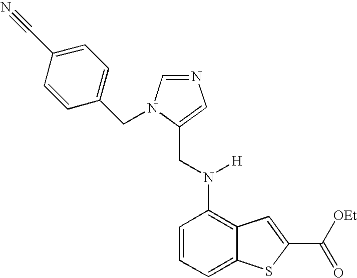

Compound 35 is prepared from derivative 1C according to the conditions described for the preparation of 34, and abiding by the proportions of the various reagents.

HPLC [C18, λ 220 nM, 100% H₂O to 100% CH₂CN (+0.1% TFA) over 8 minutes]: purity: 92%.

Mass spectrum (ESI): m/z 417 (MH+).

¹H NMR, DMSO-d₆ (ppm): 1.34 (t, 3H); 4.2-4.4 (m, 4H); 5.42 (s, 2H); 6.44 (d, 1H); 6.74 (t, 1H); 7.03 (s, 1H); 7.04 (d, 2H); 7.11 (d, 1H); 7.22 (t, 1H); 7.54 (d, 2H); 7.53 (s, 1H); 8.08 (s, 1H).

Elemental analysis (C₂₃H₂₀N₄O₂S·0.5H₂O) % calculated: C 64.92; H 4.97; N 13.17% found: C 64.59; H 4.70; N 12.94

### EXAMPLE 36

### Methyl 5-{(benzenesulfonyl)-[3-(4-cyanobenzyl)-3H-imidazol-4-ylmethyl]amino}benzo[b]thiophene-2-carboxylate (36)

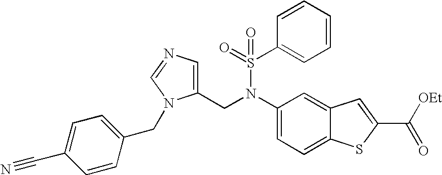

Compound 34 (3.15 g; 7.6 mmol) is dissolved in pyridine (39 ml) at room temperature and under a nitrogen atmosphere, and benzenesulfonyl chloride (1.9 ml; 15 mmol) is then added. The solution is stirred for 18 hours and then co-evaporated twice with toluene (2×100 ml). The residue is taken up in water (80 ml) and extracted with dichloromethane (6×100 ml). The organic phases are successively combined, washed with saturated aqueous NaCl solution, dried over magnesium sulfate, filtered and concentrated. This crude reaction product is then purified by flash chromatography (98/2 to 85/15 CH₂Cl₂/MeOH gradient) to give two fractions containing the desired product. The first (2.22 g) corresponds to the free base and the second (2.15 g) to the benzenesulfonate salt. This second fraction is desalified under cold conditions (0° C.) using sodium hydroxide (1N/water) to give a second batch of free base (1.43 g). 3.66 g (86%) of derivative 36 were recovered in total.

HPLC [C18, λ 220 nM, 100% H₂O to 100% CH₂CN (+0.1% TFA) over 8 minutes]: purity: 99%.

Mass spectrum (ESI): m/z 557 (MH+).

¹H NMR, DMSO-d₆ (Ppm): 1.34 (t, 3H); 4.2-4.4 (m, 4H); 5.42 (s, 2H); 6.44 (d, 1H); 6.74 (t, 1H); 7.03 (s, 1H); 7.04 (d, 2H); 7.11 (d, 1H); 7.22 (t, 1H); 7.54 (d, 2H); 7.53 (s, 1H); 8.08 (s, 1H).

Elemental analysis (C₂₉H₂₄N₄O₄S₂) % calculated: C 61.97; H 4.41; N 9.97% found: C 62.18; H 4.67; N 9.59

### EXAMPLE 37

### Methyl 4-{(benzenesulfonyl)-[3-(4-cyanobenzyl)-3H-imidazol-4-ylmethyl]amino}benzo[b]thiophene-2-carboxylate (37)

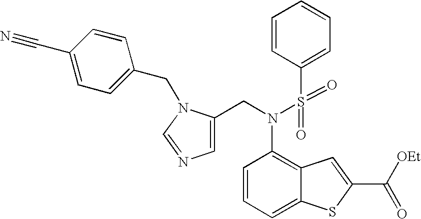

Compound 37 is prepared from derivative 35, according to the conditions described for the preparation of 36, and abiding by the proportions of the various reagents.

HPLC [C18, λ 220 nM, 100% H₂O to 100% CH₂CN (+0.1% TFA) over 25 minutes]: purity: 99%.

Mass spectrum (ESI): m/z 557 (MH+).

### EXAMPLE 38

### 5-{(Benzenesulfonyl)-[3-(4-cyanobenzyl)-3H-imidazol-4-ylmethyl]amino}benzo[b]thiophene-2-carboxylic acid (38)

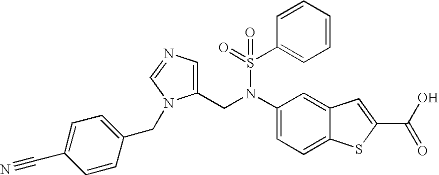

Compound 36 (3.6 g; 6.6 mmol) is dissolved in THF (23 ml) at room temperature and sodium hydroxide (30% in water; 1 ml; 9.9 mmol) is then added. The solution is stirred for 1.5 hours at 80° C. and then cooled to room temperature. Aqueous hydrochloric acid solution (1N; 9.9 ml) is added and the mixture is then concentrated. The white solid is washed with water and dried (2.45 g; 70%).

HPLC [C18, λ 220 nM, 100% H₂O to 100% CH₂CN (+0.1% TFA) over 8 minutes]: purity: 91%.

Mass spectrum (ESI): m/z 529 (MH+).

¹H NMR, DMSO-d₆ (ppm): 4.76 (s, 2H); 5.43 (s, 2H); 6.65 (s, 1H); 6.91 (dd, 1H); 7.26 (d, 2H); 7.44 (s, 1H); 7.5-8.0 (m, 10H); 12-14 (m, 1H).

### EXAMPLE 39

### 4-{(Benzenesulfonyl)-[3-(4-cyanobenzyl)-3H-imidazol-4-ylmethyl]amino}benzo[b]thiophene-2-carboxylic acid (39)

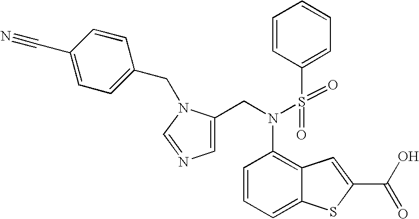

Compound 39 is prepared from derivative 37, according to the conditions described for the preparation of 38 and abiding by the proportions of the various reagents.

HPLC [C18, λ 220 nM, 100% H₂O to 100% CH₂CN (+0.1% TFA) over 8 minutes]: purity: 85%.

Mass spectrum (ESI): m/z 529 (MH+).

### Examples 40 to 86

Compounds 40 to 86 are prepared from derivatives 38 or 39, and from commercial amines, according to the conditions described for the preparation of 40, and abiding by the proportions of the various reagents.

Compound 38 (120 mg; 0.23 mmol) is dissolved in a mixture of DMF and CH₂Cl₂ (3 ml; 50/50 v/v) in the presence of H-Met-OMe (25 mg; 0.15 mmol), HOBT (34 mg; 0.26 mmol) and PS-Carbodiimide (Argonaut Technologies; 288 mg; 0.31 mmol). The mixture is stirred at room temperature for 24 hours and MP-Carbonate (Argonaut Technologies; 276 mg; 0.76 mmol) is added and the reaction mixture is stirred for a further 18 hours. The mixture is filtered and concentrated to give derivative 40.

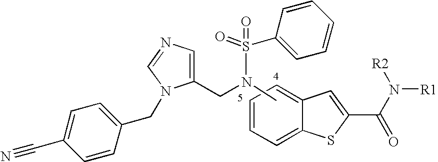

| | Substituent | | | Mass | HPLC |
|---|---|---|---|---|---|
| Ex. | position | NR₁R₂ | Compound name | (M + H)⁺ | purity* |
| 40 | 5 | Met-OMe | Methyl (2S)-2-[(5-benzene- | 674 | 99** |
| | | | sulfonyl-[3-(4-cyano- | | |
| | | | benzyl)-3H-imidazol-4- | | |
| | | | ylmethyl]amino}benzo[b]- | | |
| | | | thiophene-2-carbonyl)- | | |
| | | | amino]-4-(methylsulfanyl)- | | |
| | | | butyrate | | |
| 41 | 4 | Met-OMe | Methyl (2S)-2-[(4-benzene- | 674 | 90 |
| | | | sulfonyl-[3-(4-cyano- | | |
| | | | benzyl)-3H-imidazol-4- | | |
| | | | ylmethyl]amino}benzo[b]- | | |
| | | | thiophene-2-carbonyl)- | | |
| | | | amino]-4-(methylsulfanyl)- | | |
| | | | butyrate | | |
| 42 | 4 | 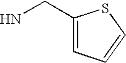 | N-(Thiophen-2-ylmethyl)- 4-{benzenesulfonyl-[3-(4- cyanobenzyl)-3H-imidazol- 4-ylmethyl]amino}benzo- [b]thiophene-2- carboxamide | 624 | 82 |
| 43 | 4 | 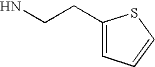 | N-(2-Thiophen-2-ylethyl)- 4-{benzenesulfonyl-[3-(4- cyanobenzyl)-3H-imidazol- 4-ylmethyl]amino}benzo- [b]thiophene-2- carboxamide | 638 | 79 |
| 44 | 4 | HN(CH₂)₂SMe | N-(2-Methylsulfanylethyl)- | 602 | 84 |
| | | | 4-{benzenesulfonyl-[3-(4- | | |
| | | | cyanobenzyl)-3H-imidazol- | | |
| | | | 4-ylmethyl]amino}benzo- | | |
| | | | [b]thiophene-2- | | |
| | | | carboxamide | | |
| 45 | 4 | 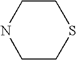 | N-[3-(4-Cyanobenzyl)-3H- imidazol-4-ylmethyl]-N-[2- (thiomorpholine-4- carbonyl)benzo[b]thiophen- 4-yl]benzenesulfonamide | 614 | 81 |
| 46 | 5 | 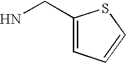 | N-(Thiophen-2-ylmethyl)- 5-{benzenesulfonyl-[3-(4- cyanobenzyl)-3H-imidazol- 4-ylmethyl]amino}benzo- [b]thiophene-2- carboxamide | 624 | 85 |
| 47 | 5 | 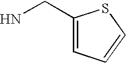 | N-(2-Thiophen-2-ylethyl)- 5-{benzenesulfonyl-[3-(4- cyanobenzyl)-3H-imidazol- 4-ylmethyl]amino}benzo- [b]thiophene-2- carboxamide | 638 | 84 |
| 48 | 5 | HN(CH₂)₂SMe | N-(2-Methylsulfanylethyl)- | 602 | 89 |
| | | | 5-{benzenesulfonyl-[3-(4- | | |
| | | | cyanobenzyl)-3H-imidazol- | | |
| | | | 4-ylmethyl]amino}benzo- | | |
| | | | [b]thiophene-2- | | |
| | | | carboxamide | | |
| 49 | 5 | 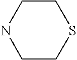 | N-[3-(4-Cyanobenzyl)-3H- imidazol-4-ylmethyl]-N-[2- (thiomorpholine-4- carbonyl)benzo[b]thiophen- 5-yl]benzenesulfonamide | 614 | 88 |
| 50 | 4 | Leu-OMe | Methyl (2S)-2-[(4- | 656 | 98** |
| | | | {benzenesulfonyl-[3-(4- | | |
| | | | cyanobenzyl)-3H-imidazol- | | |
| | | | 4-ylmethyl]amino}benzo- | | |
| | | | [b]thiophene-2-carbonyl)- | | |
| | | | amino]-4-methylpentanoate | | |
| 51 | 5 | Leu-OMe | Methyl (2S)-2-[(5- | 656 | 91** |
| | | | {benzenesulfonyl-[3-(4- | | |
| | | | cyanobenzyl)-3H-imidazol- | | |
| | | | 4-ylmethyl]amino}benzo- | | |
| | | | [b]thiophene-2-carbonyl)- | | |
| | | | amino]-4-methylpentanoate | | |
| 52 | 4 | Gly-OMe | Methyl (2S)-2-[(4- | 600 | 90** |
| | | | {benzenesulfonyl-[3-(4- | | |
| | | | cyanobenzyl)-3H-imidazol- | | |
| | | | 4-ylmethyl]amino}benzo- | | |
| | | | [b]thiophene-2-carbonyl)- | | |
| | | | amino]acetate | | |
| 53 | 5 | Gly-OMe | Methyl (2S)-2-[(5- | 600 | 82 |
| | | | {benzenesulfonyl-[3-(4- | | |
| | | | cyanobenzyl)-3H-imidazol- | | |
| | | | 4-ylmethyl]amino}benzo- | | |
| | | | [b]thiophene-2-carbonyl)- | | |
| | | | amino]acetate | | |
| 54 | 4 | 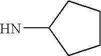 | N-Cyclopentyl-4-{benzene- sulfonyl-[3-(4-cyano- benzyl)-3H-imidazol-4- ylmethyl]amino}benzo[b]- thiophene-2-carboxamide | 596 | 98** |
| 55 | 5 | 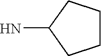 | N-Cyclopentyl-5-{benzene- sulfonyl-[3-(4-cyano- benzyl)-3H-imidazol-4- ylmethyl]amino}benzo[b]- thiophene-2-carboxamide | 596 | 98** |
| 56 | 4 | HNnBu | N-Butyl-4-{benzene- | 584 | 98** |
| | | | sulfonyl-[3-(4-cyano- | | |
| | | | benzyl)-3H-imidazol-4- | | |
| | | | ylmethyl]amino}benzo[b]- | | |
| | | | thiophene-2-carboxamide | | |
| 57 | 5 | HNnBu | N-Butyl-5-{benzene- | 584 | 98** |
| | | | sulfonyl-[3-(4-cyano- | | |
| | | | benzyl)-3H-imidazol-4- | | |
| | | | ylmethyl]amino}benzo[b]- | | |
| | | | thiophene-2-carboxamide | | |
| 58 | 4 | HN(CH₂)₃SMe | N-[3-(Methylsulfanyl)- | 616 | 77 |
| | | | propyl]-4-{benzene- | | |
| | | | sulfonyl-[3-(4-cyano- | | |
| | | | benzyl)-3H-imidazol-4- | | |
| | | | ylmethyl]amino}benzo[b]- | | |
| | | | thiophene-2-carboxamide | | |
| 59 | 5 | HN(CH₂)₃SMe | N-[3-(Methylsulfanyl)- | 616 | 99** |
| | | | propyl]-5-{benzene- | | |
| | | | sulfonyl-[3-(4-cyano- | | |
| | | | benzyl)-3H-imidazol-4- | | |
| | | | ylmethyl]amino}benzo[b]- | | |
| | | | thiophene-2-carboxamide | | |
| 60 | 4 | HN(CH₂)₃OiPr | N-[3-(Isopropoxy)propyl]- | 628 | 99** |
| | | | 4-{benzenesulfonyl-[3-(4- | | |
| | | | cyanobenzyl)-3H-imidazol- | | |
| | | | 4-ylmethyl]amino}benzo- | | |
| | | | [b]thiophene-2- | | |
| | | | carboxamide | | |
| 61 | 5 | HN(CH₂)₃OiPr | N-[3-(Isopropoxy)propyl]- | 628 | 99** |
| | | | 5-{benzenesulfonyl-[3-(4- | | |
| | | | cyanobenzyl)-3H-imidazol- | | |
| | | | 4-ylmethyl]amino}benzo- | | |
| | | | [b]thiophene-2- | | |
| | | | carboxamide | | |
| 62 | 4 | HN-geranyl | N-[3,7-Dimethylocta-2,6- | 664 | 91** |
| | | | dienyl]-4-{benzene- | | |
| | | | sulfonyl-[3-(4-cyano- | | |
| | | | benzyl)-3H-imidazol-4- | | |
| | | | ylmethyl]amino}benzo[b]- | | |
| | | | thiophene-2-carboxamide | | |
| 63 | 5 | HN-geranyl | N-[3,7-Dimethylocta-2,6- | 664 | 96** |
| | | | dienyl]-5-{benzene- | | |
| | | | sulfonyl-[3-(4-cyano- | | |
| | | | benzyl)-3H-imidazol-4- | | |
| | | | ylmethyl]amino}benzo[b] | | |
| | | | thiophene-2-carboxamide | | |
| 64 | 5 | 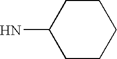 | N-Cyclohexyl-5-{benzene- sulfonyl-[3-(4-cyano- benzyl)-3H-imidazol-4- ylmethyl]amino}benzo[b]- thiophene-2-carboxamide | 610 | 96** |
| 65 | 5 | HNCH₂CHMe₂ | N-(2-Methylpropyl)-5- | 584 | 82 |
| | | | {benzenesulfonyl-[3-(4- | | |
| | | | cyanobenzyl)-3H-imidazol- | | |
| | | | 4-ylmethyl]amino}benzo- | | |
| | | | [b]thiophene-2- | | |
| | | | carboxamide | | |
| 66 | 5 | HNMe | N-Methyl-5-{benzene- | 542 | 80 |
| | | | sulfonyl-[3-(4-cyano- | | |
| | | | benzyl)-3H-imidazol-4- | | |
| | | | ylmethyl]amino}benzo[b]- | | |
| | | | thiophene-2-carboxamide | | |
| 67 | 5 | HNEt | N-Ethyl-5-{benzene- | 556 | 77 |
| | | | sulfonyl-[3-(4-cyano- | | |
| | | | benzyl)-3H-imidazol-4- | | |
| | | | ylmethyl]amino}benzo[b]- | | |
| | | | thiophene-2-carboxamide | | |
| 68 | 5 | NEt₂ | N,N-Diethyl-5-{benzene- | 584 | 80 |
| | | | sulfonyl-[3-(4-cyano- | | |
| | | | benzyl)-3H-imidazol-4- | | |
| | | | ylmethyl]amino}benzo[b] | | |
| | | | thiophene-2-carboxamide | | |
| 69 | 5 | HNCH₂tBu | N-(2,2-Dimethylpropyl)-5- | 598 | 80 |
| | | | {benzenesulfonyl-[3-(4- | | |
| | | | cyanobenzyl)-3H-imidazol- | | |
| | | | 4-ylmethyl]amino}benzo- | | |
| | | | [b]thiophene-2- | | |
| | | | carboxamide | | |
| 70 | 5 | HNnPr | N-Propyl-5-{benzene- | 570 | 79 |
| | | | sulfonyl-[3-(4-cyano- | | |
| | | | benzyl)-3H-imidazol-4- | | |
| | | | ylmethyl]amino}benzo[b]- | | |
| | | | thiophene-2-carboxamide | | |
| 71 | 5 | HN-allyl | N-Allyl-5-{benzene- | 568 | 72 |
| | | | sulfonyl-[3-(4-cyano- | | |
| | | | benzyl)-3H-imidazol-4- | | |
| | | | ylmethyl]amino}benzo[b]- | | |
| | | | thiophene-2-carboxamide | | |
| 72 | 5 | HN(CH₂)₂OMe | N-(2-Methoxyethyl)-5- | 586 | 77 |
| | | | {benzenesulfonyl-[3-(4- | | |
| | | | cyanobenzyl)-3H-imidazol- | | |
| | | | 4-ylmethyl]amino}benzo- | | |
| | | | [b]thiophene-2- | | |
| | | | carboxamide | | |
| 73 | 5 | 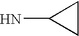 | N-Cyclopropyl-5-{benzene- sulfonyl-[3-(4-cyano- benzyl)-3H-imidazol-4- ylmethyl]amino}benzo[b]- thiophene-2-carboxamide | 568 | 99** |
| 74 | 5 | 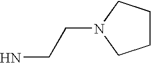 | N-(2-Pyrrolidin-1-ylethyl)- 5-{benzenesulfonyl-[3-(4- cyanobenzyl)-3H-imidazol- 4-ylmethyl]amino}benzo- [b]thiophene-2- carboxamide | 625 | 96** |
| 75 | 5 | 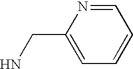 | N-(Pyrid-2-ylmethyl)-5- {benzenesulfonyl-[3-(4- cyanobenzyl)-3H-imidazol- 4-ylmethyl]amino}benzo- [b]thiophene-2- carboxamide | 619 | 99** |
| 76 | 5 | 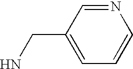 | N-(Pyrid-3-ylmethyl)-5- {benzenesulfonyl-[3-(4- cyanobenzyl)-3H-imidazol- 4-ylmethyl]amino}benzo- [b]thiophene-2- carboxamide | 619 | 92** |
| 77 | 5 | 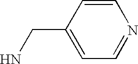 | N-(Pyrid-4-ylmethyl)-5- {benzenesulfonyl-[3-(4- cyanobenzyl)-3H-imidazol- 4-ylmethyl]amino}benzo- [b]thiophene-2- carboxamide | 619 | 99** |
| 78 | 5 | 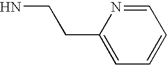 | N-(Pyrid-2-ylethyl)-5- {benzenesulfonyl-[3-(4- cyanobenzyl)-3H-imidazol- 4-ylmethyl]amino}benzo- [b]thiophene-2- carboxamide | 633 | 92** |
| 79 | 5 | 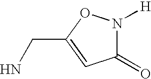 | N-(3-Oxo-2,3-dihydro- isoxazol-5-ylmethyl)-5- {benzenesulfonyl-[3-(4- cyanobenzyl)-3H-imidazol- 4-ylmethyl]amino}benzo- [b]thiophene-2- carboxamide | 625 | 84 |
| 80 | 5 | 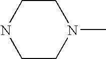 | N-[3-(4-Cyanobenzyl)-3H- imidazol-4-ylmethyl]-N-[2- (4-methylpiperazine-4- carbonyl)benzo[b]thiophen- 5-yl]benzenesulfonamide | 611 | 97** |
| 81 | 5 | 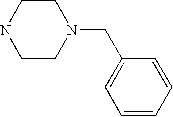 | N-[3-(4-Cyanobenzyl)-3H- imidazol-4-ylmethyl]-N-[2- (4-benzylpiperazine-4- carbonyl)benzo[b]thiophen- 5-yl]benzenesulfonamide | 687 | 91** |
| 82 | 5 | 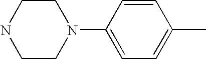 | N-[3-(4-Cyanobenzyl)-3H- imidazol-4-ylmethyl]-N-{2- [4-(4-fluorophenyl)- piperazine-4-carbonyl]- benzo[b]thiophen-5- yl}benzenesulfonamide | 691 | 72 |
| 83 | 5 | 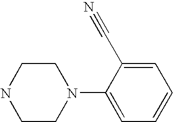 | N-[3-(4-Cyanobenzyl)-3H- imidazol-4-ylmethyl]-N-{2- [4-(2-cyanophenyl)- piperazine-4-carbonyl]- benzo[b]thiophen-5- yl}benzenesulfonamide | 698 | 99** |
| 84 | 5 | 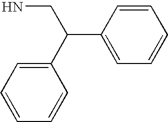 | N-(2,2-Diphenylethyl)-5- {benzenesulfonyl-[3-(4- cyanobenzyl)-3H-imidazol- 4-ylmethyl]amino}benzo- [b]thiophene-2- carboxamide | 708 | 97** |
| 85 | 5 | 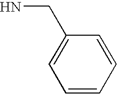 | N-(Benzyl)-5- {benzenesulfonyl-[3-(4- cyanobenzyl)-3H-imidazol- 4-ylmethyl]amino}benzo- [b]thiophene-2- carboxamide | 618 | 83** |
| 86 | 5 | 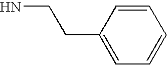 | N-(2-Phenylethyl)-5- {benzenesulfonyl-[3-(4- cyanobenzyl)-3H-imidazol- 4-ylmethyl]amino}benzo- [b]thiophene-2- carboxamide | 932 | 98** |

*HPLC conditions [C18 symmetry, 4.6 × 50 mm, 5 μm; λ = 220 nM; gradient 100% H₂O (+0.05% TFA) to 100% CH₂CN (+0.05% TFA) over 8 minutes]
**The compound was purified by preparative HPLC (Waters Prep 4000) on a Prep Nova-Pak HR C-18 column (Waters; 25 × 100 mm; 6 μm) using a total gradient of from 100% water (0.1% TFA) to 100% acetonitrile (0.1% TFA) over 15 minutes.

### EXAMPLE 87

### (2S)-2-[(4-{Benzenesulfonyl-[3-(4-cyanobenzyl)-3H-imidazol-4-ylmethyl]amino}benzo[b]thiophene-2-carbonyl)amino] acetic acid (87)

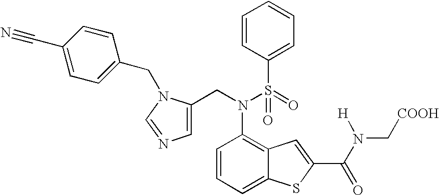

Compound 52 (0.09 mmol) is dissolved in THF (1 ml) in the presence of lithium hydroxide (1M/water; 2 equivalents). The reaction mixture is stirred for 18 hours at room temperature and then concentrated. The residue is purified by preparative HPLC (Waters Prep 4000) on a Prep Nova-Pak HR C-18 column (Waters; 25×100 mm; 6 μm) using a total gradient of from 100% water (0.1% TFA) to 100% acetonitrile (0.1% TFA) over 15 minutes, to give the desired product.

HPLC [C18, λ 220 nM, 100% H₂O to 100% CH₂CN (+0.1% TFA) over 8 minutes]: purity: 93%.

Mass spectrum (ESI): m/z 586 (MH+).

### EXAMPLE 88

4-{[3-(4-Cyanobenzyl)-3H-imidazol-4-ylmethyl]amino}benzo[b]thiophene-2-carboxylic acid (8)

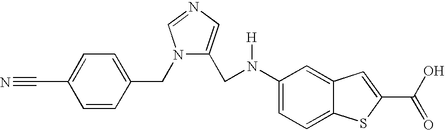

Compound 10C (50 mg; 0.26 mmol) is dissolved in methanol (2 ml) under a nitrogen atmosphere, in the presence of derivative 27A (55 mg; 0.26 mmol). After stirring for a few minutes, sodium cyanoborohydride (18 mg; 0.18 mmol) is added and the reaction mixture is stirred at room temperature for 18 hours. The reaction mixture is concentrated and the solid residue is washed with 6 ml of water and then dried to give the desired product (14 mg; 14%).

HPLC [C18 λ 220 nM, 100% H₂O to 100% CH₂CN (+0.1% TFA) over 8 minutes]: purity: 99%.

Mass spectrum (ESI): m/z 389 (MH+).

¹H NMR, DMSO-d₆ (ppm): 3-5 (m, H₂O+COOH); 4.09 (s, 2H); 5.40 (s, 2H); 5.90 (s, 1H, NH); 6.71 (d, 1H); 6.88 (s, 1H); 6.97 (s, 1H); 7.27 (d, 2H); 7.43 (s, 1H); 7.50 (d, 1H); 7.72-7.89 (m, 3H).

### EXAMPLE 89

### 4-(5-{[2-(Thiomorpholine-4-carbonyl)benzo[b]thiophen-5-ylamino]methyl}imidazol-1-ylmethyl)benzonitrile (89)

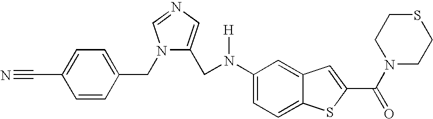

### Example 89A

### (5-Aminobenzo[b]thiophen-2-yl)thiomorpholin-4-ylmethanone

Compound 10C (2.0 g; 10 mmol) is dissolved in dichloromethane (40 ml) and DMF (80 ml) under a nitrogen atmosphere. DIPEA (3.3 ml; 29 mmol), 1-(3-dimethylaminopropyl)-3-ethylcarbodiimide hydrochloride (EDC) (1.89 g; 10 mmol), HOOBT (1.69 g; 10 mmol) and thiomorpholine (0.89 ml; 9 mmol) are added and the reaction is stirred for 18 hours at room temperature. It is then concentrated. The residue is subsequently taken up in dichloromethane (250 ml), washed with water (80 ml), dried over magnesium sulfate, filtered and concentrated. The residual oil is then purified by flash chromatography (80/20 CH₂Cl₂/acetone) to give the desired product (1.66 g; 66%).

¹HNMR, DMSO-d₆ (ppm): 2.69 (bs, 4H); 3.86 (bs, 4H); 5.19 (s, 2H); 6.79 (dd, 1H); 6.99 (d, 1H); 7.42 (s, 1H); 7.59 (d, 1H).

### Example 89

### 4-(5-{1[2-Thiomorpholine-4-carbonyl)benzo[b]thiophen-5-ylamino]methyl}imidazol-1-ylmethyl)benzonitrile

Compound 89 is prepared from derivative 89B (1.57 g) according to the conditions used for the preparation of 34 and abiding by the proportions of the various reagents. The crude reaction product is then purified by flash chromatography (20/80 acetone/CH₂Cl₂ and then 5/95 MeOH/CH₂Cl₂) to give the desired product (1.92 g; 72%).

HPLC [C18, λ 220 nM, 100% H₂O to 100% CH₂CN (+0.1% TFA) over 8 minutes]: purity: 93%.

Mass spectrum (ESI): m/z 474 (MH+).

¹HNMR, DMSO-d₆ (ppm): 2.70 (bs, 4H); 3.88 (bs, 4H); 4.10 (d, 2H); 5.39 (s, 2H); 6.07 (t, 1H); 6.78 (dd, 1H); 6.87 (d, 1H); 6.96 (s, 1H); 7.26 (d, 2H); 7.42 (s, 1H); 7.62 (d, 1H); 7.77 (s, 1H); 7.80 (d, 2H).

### EXAMPLE 90

### 4-(5-{1[2-(Thiomorpholine-4-carbonyl)benzo[b]thiophen-4-ylamino]methyl}imidazol-1-ylmethyl)benzonitrile (20)

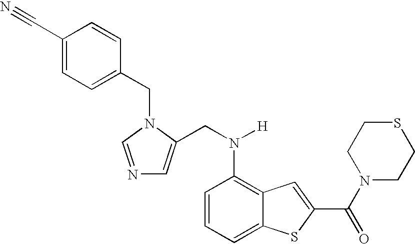

### Example 90A

### (4-Aminobenzo[b]thiophen-2-yl)thiomorpholin-4-ylmethanone

Compound 90A is prepared from derivative 1D (2.27 g) according to the conditions used for the preparation of 89A, and abiding by the proportions of the various reagents. The residual oil is purified by flash chromatography (CH₂Cl₂ and then 80/20 CH₂Cl₂/acetone) to give the desired product (3.3 g).

¹H NMR, DMSO-d₆ (ppm): 2.72 (bs, 4H); 3.93 (bs, 4H); 5.88 (s, 2H); 6.59 (d, 1H); 7.05-7.20 (m, 2H); 7.86 (s, 1H).

### Example 90

### 4-(5-{[2-(Thiomorpholine-4-carbonyl)benzo[b]thiophen-4-yl-amino]methyl}imidazol-1-ylmethyl)benzonitrile

Compound 90 is prepared from derivative 90A (2.32 g) according to the conditions used for the preparation of 89, and abiding by the proportions of the various reagents. The crude reaction product is then purified by flash chromatography (CH₂Cl₂ and then acetone/CH₂Cl₂ gradient: 20/80 to 50/50, and then MeOH/CH₂Cl₂ gradient: 5/95 to 10/90) to give the desired product (1.43 g; 25%) and the nonreduced intermediate imine (2.62 g; 46%).

HPLC [C18, λ 220 nM, 100% H₂O to 100% CH₂CN (+0.1% TFA) over 8 minutes]: purity: 98%.

Mass spectrum (ESI): m/z 474 (MH+).

¹H NMR, DMSO-dr (ppm): 2.71 (bs, 4H); 3.89 (bs, 4H); 4.28 (d, 2H); 5.41 (s, 2H); 6.41 (dd, 1H); 6.56 (t, 1H); 6.99 (s, 1H); 7.1-7.2 (m, 4H); 7.65 (d, 2H); 7.72 (s, 1H); 7.75 (s, 1H).

### Examples 91 to 107

Compounds 91 to 107 are prepared from derivatives 89 or 90, and from commercial acid chlorides, according to the conditions described for the preparation of 9.1 and abiding by the proportions of the various reagents.

Compound 89 (30 mg; 0.06 mmol) is dissolved in dichloromethane (2.5 ml) in the presence of 2-thiophenecarbonyl chloride (24 mg; 0.16 mmol) and PS-DIEA (Argonaut Technologies; 52 mg; 0.19 mmol). The mixture is stirred at room temperature for 6 hours, PS-trisamine (Argonaut Technologies; 66 mg; 0.25 mmol) is then added and the reaction mixture is stirred for a further 18 hours. The mixture is filtered and concentrated to give derivative 91.

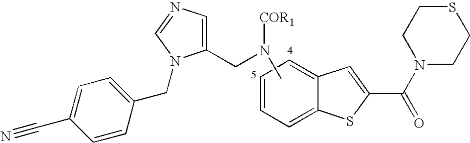

| | Substituent | | | Mass | HPLC |
|---|---|---|---|---|---|
| Ex. | position | R₁CO | Compound name | (M + H)⁺ | purity* |
| 91 | 5 | 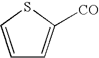 | N-[3-(4-Cyanobenzyl)-3H- imidazol-4-ylmethyl]-N-[2- (thiomorpholine-4- carbonyl)benzo[b]thiophen- 5-yl]thiophene-2- carboxamide | 584 | 95 |
| 92 | 5 | CH₃(CH₂)₂CO | N-[3-(4-Cyanobenzyl)-3H- | 544 | 95 |
| | | | imidazol-4-ylmethyl]-N-[2- | | |
| | | | (thiomorpholine-4- | | |
| | | | carbonyl)benzo[b]thiophen- | | |
| | | | 5-yl]butyramide | | |
| 93 | 5 | PhCO | N-[3-(4-Cyanobenzyl)-3H- | 578 | 99** |
| | | | imidazol-4-ylmethyl]-N-[2- | | |
| | | | (thiomorpholine-4- | | |
| | | | carbonyl)benzo[b]thiophen- | | |
| | | | 5-yl]benzamide | | |
| 94 | 5 | 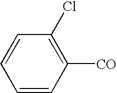 | N-[3-(4-Cyanobenzyl)-3H- imidazol-4-ylmethyl]-N-[2- (thiomorpholine-4- carbonyl)benzo[b]thiophen- 5-yl]-2-chlorobenzamide | 612 | 99** |
| 95 | 5 | 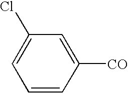 | N-[3-(4-Cyanobenzyl)-3H- imidazol-4-ylmethyl]-N-[2- (thiomorpholine-4- carbonyl)benzo[b]thiophen- 5-yl]-3-chlorobenzamide | 612 | 86 |
| 96 | 5 | 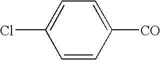 | N-[3-(4-Cyanobenzyl)-3H- imidazol-4-ylmethyl]-N-[2- (thiomorpholine-4- carbonyl)benzo[b]thiophen- 5-yl]-4-chlorobenzamide | 612 | 90 |
| 97 | 5 | 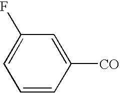 | N-[3-(4-Cyanobenzyl)-3H- imidazol-4-ylmethyl]-N-[2- (thiomorpholine-4- carbonyl)benzo[b]thiophen- 5-yl]-3-fluorobenzamide | 596 | 86 |
| 98 | 5 | 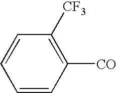 | N-[3-(4-Cyanobenzyl)-3H- imidazol-4-ylmethyl]-N-[2- (thiomorpholine-4- carbonyl)benzo[b]thiophen- 5-yl]-2-trifluoromethyl- benzamide | 646 | 85 |
| 99 | 5 | 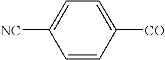 | N-[3-(4-Cyanobenzyl)-3H- imidazol-4-ylmethyl]-N-[2- (thiomorpholine-4- carbonyl)benzo[b]thiophen- 5-yl]-4-cyanobenzamide | 603 | 85 |
| 100 | 5 | 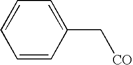 | N-[3-(4-Cyanobenzyl)-3H- imidazol-4-ylmethyl]-N-[2- (thiomorpholine-4- carbonyl)benzo[b]thiophen- 5-yl]-2-phenylacetamide | 592 | 97 |
| 101 | 5 | 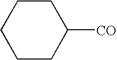 | N-[3-(4-Cyanobenzyl)-3H- imidazol-4-ylmethyl]-N-[2- (thiomorpholine-4- carbonyl)benzo[b]thiophen- 5-yl]cyclohexane- carboxamide | 584 | 97 |
| 102 | 4 | CH₃(CH₂)₂CO | N-[3-(4-Cyanobenzyl)-3H- | 544 | 92 |
| | | | imidazol-4-ylmethyl]-N-[2- | | |
| | | | (thiomorpholine-4- | | |
| | | | carbonyl)benzo[b]thiophen- | | |
| | | | 4-yl]butyramide | | |
| 103 | 4 | 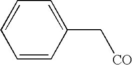 | N-[3-(4-Cyanobenzyl)-3H- imidazol-4-ylmethyl]-N-[2- (thiomorpholine-4- carbonyl)benzo[b]thiophen- 4-yl]-2-phenylacetamide | 592 | 99** |
| 104 | 4 | 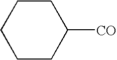 | N-[3-(4-Cyanobenzyl)-3H- imidazol-4-ylmethyl]-N-[2- (thiomorpholine-4- carbonyl)benzo[b]thiophen- 4-yl]cyclohexane- carboxamide | 584 | 95** |
| 105 | 4 | 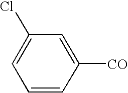 | N-[3-(4-Cyanobenzyl)-3H- imidazol-4-ylmethyl]-N-[2- (thiomorpholine-4- carbonyl)benzo[b]thiophen- 4-yl]-3-chlorobenzamide | 612 | 99** |
| 106 | 4 | 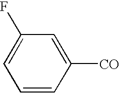 | N-[3-(4-Cyanobenzyl)-3H- imidazol-4-ylmethyl]-N-[2- (thiomorpholine-4- carbonyl)benzo[b]thiophen- 4-yl]-3-fluorobenzamide | 596 | 98** |
| 107 | 4 | 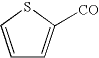 | N-[3-(4-Cyanobenzyl)-3H- imidazol-4-ylmethyl]-N-[2- (thiomorpholine-4- carbonyl)benzo[b]thiophen- 4-yl]thiophene-2- carboxamide | 584 | 93** |

*HPLC conditions [C18 symmetry, 4.6 × 50 mm, 5 μm; λ = 220 nM; gradient 100% H₂O (+0.05% TFA) to 100% CH₂CN (+0.05% TFA) over 8 minutes]
**The compound was purified by preparative HPLC (Waters Prep 4000) on a Prep Nova-Pak HR C-18 column (Waters; 25 × 100 mm; 6 μm) using a total gradient of from 100% water (0.1% TFA) to 100% acetonitrile (0.1% TFA) over 15 minutes.

### EXAMPLES 108 AND 109

Compounds 108 and 109 are prepared from derivatives 89 or 90 according to the conditions described for the preparation of 108, and abiding by the proportions of the various reagents.

Compound 89 (50 mg; 0.10 mmol) is dissolved in dichloromethane (0.5 ml) and toluene (2 ml) in the presence of 2-(methylthio)phenyl isocyanate (0.20 mmol). The mixture is stirred at 60° C. for 6 hours, PS-trisamine (Argonaut Technologies; 137 mg; 0.52 mmol) is then added and the reaction is stirred at room temperature for 18 hours. The mixture is filtered and concentrated. The residue is purified by preparative HPLC (Waters Prep 4000), on a Prep Nova-Pak HR C-18 column (Waters; 25×100 mm; 6 μm) using a total gradient of from 100% water (0.1% TFA) to 100% acetonitrile (0.1% TFA) over 15 minutes, to give the desired product.

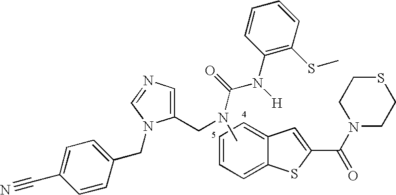

| | Substituent | | Mass | HPLC |
|---|---|---|---|---|
| Ex. | position | Compound name | (M + H)⁺ | purity* |
| 108 | 5 | 1-[3-(4-Cyanobenzyl)-3H-imi- | 639 | 99 |
| | | dazol-4-yl]-3-[2-(methylsulfa- | | |
| | | nyl)phenyl]-1-[2-(thiomorpho- | | |
| | | line-4-carbonyl)benzo[b]-thio- | | |
| | | phen-5-yl]urea | | |
| 109 | 4 | 1-[3-(4-Cyanobenzyl)-3H-imi- | 639 | 100 |
| | | dazol-4-yl]-3-[2-(methylsulfa- | | |
| | | nyl)phenyl]-1-[2-(thiomorpho- | | |
| | | line-4-carbonyl)benzo[b]-thio- | | |
| | | phen-4-yl]urea | | |

*HPLC conditions [C18 symmetry, 4.6 × 50 mm, 5 μm; λ = 220 nM; gradient 100% H₂O (+0.05% TFA) to 100% CH₂CN (+0.05% TFA) over 8 minutes]

### Examples 110 to 118

Compounds 110 to 118 are prepared from derivatives 89 or 90, and from the corresponding aldehydes, according to the conditions described for the preparation of 110, and abiding by the proportions of the various reagents.

Compound 89 (50 mg; 0.10 mmol) is dissolved in DCE (3 ml) in the presence of phenylacetaldehyde (62 μl; 0.53 mmol) and acetic acid (55 μl; 1 mmol). Sodium triacetoxyborohydride (110 mg; 0.53 mmol) is added and the mixture is stirred at room temperature (or at 50° C. in the case of !0) until product 89 has completely disappeared. Ethyl acetate (15 ml) is added. The solution is washed twice with saturated aqueous sodium bicarbonate solution and then dried over magnesium sulfate, filtered and concentrated. The residue is purified by preparative HPLC (Waters Prep 4000) on a Prep Nova-Pak HR C-18 column (Waters; 25×100 mm; 6 μm) using a total gradient of from 100% water (0.1% TFA) to 100% acetonitrile (0.1% TFA) over 15 minutes, to give the desired product.

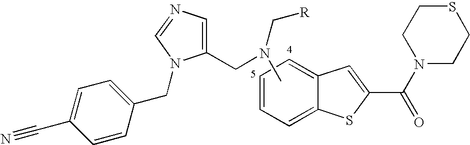

| | Substituent | | | Mass | HPLC |
|---|---|---|---|---|---|
| Ex. | position | CH₂R | Compound name | (M + H)⁺ | purity* |
| 110 | 5 | CH₂—CH₂Ph | 4-[5-({(2-Phenylethyl)[2- | 578 | 99 |
| | | | (thiomorpholine-4-carbonyl)- | | |
| | | | benzo[b]thiophen-5-yl]- | | |
| | | | amino}methyl)imidazol-1- | | |
| | | | ylmethyl]benzonitrile | | |
| 111 | 5 | Et | 4-[5-({Ethyl[2-(thio- | 502 | 95 |
| | | | morpholine-4-carbonyl)- | | |
| | | | benzo[b]thiophen-5-yl]- | | |
| | | | amino}methyl)imidazol-1- | | |
| | | | ylmethyl]benzonitrile | | |
| 112 | 5 | nPr | 4-[5-({Propyl[2-(thio- | 516 | 99 |
| | | | morpholine-4-carbonyl)- | | |
| | | | benzo[b]thiophen-5-yl]- | | |
| | | | amino}methyl)imidazol-1- | | |
| | | | ylmethyl]benzonitrile | | |
| 113 | 5 | nBu | 4-[5-({Butyl[2-(thio- | 530 | 98 |
| | | | morpholine-4-carbonyl)- | | |
| | | | benzo[b]thiophen-5-yl]- | | |
| | | | amino}methyl)imidazol-1- | | |
| | | | ylmethyl]benzonitrile | | |
| 114 | 5 | 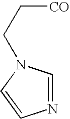 | 4-[5-({Cyclohexylmethyl[2- (thiomorpholine-4-carbonyl)- benzo[b]thiophen-5-yl]- amino}methyl)imidazol-1- ylmethyl]benzonitrile | 570 | 97 |
| 115 | 4 | CH₂—CH₂Ph | 4-[5-({(2-Phenylethyl)[2- | 578 | 98 |
| | | | (thiomorpholine-4-carbonyl)- | | |
| | | | benzo[b]thiophen-4-yl]- | | |
| | | | amino}methyl)imidazol-1- | | |
| | | | ylmethyl]benzonitrile | | |
| 116 | 4 | nPr | 4-[5-({Propyl[2-(thio- | 516 | 99 |
| | | | morpholine-4-carbonyl)- | | |
| | | | benzo[b]thiophen-4-yl]- | | |
| | | | amino}methyl)imidazol-1- | | |
| | | | ylmethyl]benzonitrile | | |
| 117 | 4 | nBu | 4-[5-({Butyl[2-(thio- | 530 | 93 |
| | | | morpholine-4-carbonyl)- | | |
| | | | benzo[b]thiophen-4-yl]- | | |
| | | | amino}methyl)imidazol-1- | | |
| | | | ylmethyl]benzonitrile | | |
| 118 | 4 | 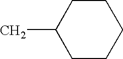 | 4-[5-({Cyclohexylmethyl[2- (thiomorpholine-4-carbonyl)- benzo[b]thiophen-4-yl]- amino}methyl)imidazol-1- ylmethyl]benzonitrile | 570 | 96 |

*HPLC conditions [C18 symmetry, 4.6 × 50 mm, 5 μm; λ = 220 nM; gradient 100% H₂O (+0.05% TFA) to 100% CH₂CN (+0.05% TFA) over 8 minutes]

### EXAMPLE 119

5 Ethyl 3-butyl-7-{1[3-(4-cyanobenzyl)-3H-imidazol-4-ylmethyl]amino}benzo[b]thiophene-2-carboxylate (119)

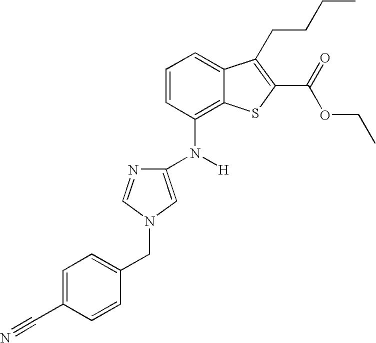

### Example 119A

### 1-(2-Fluorophenol)pentan-1-ol

2-Fluorobenzaldehyde (10 g; 80 mmol) is dissolved in THF (400 ml). The solution is cooled to −78° C. and nBuLi (1.6 M/THF; 50 ml; 80 mmol) is then added. The cold bath is removed. When the reaction mixture has returned to room temperature, saturated aqueous ammonium chloride solution (40 ml) is added and the mixture is concentrated. Ethyl acetate (300 ml) and water (100 ml) are added. The organic phase is recovered, dried over magnesium sulfate, filtered and concentrated. The residual oil is purified by flash chromatography (90/10 EDP/EtOAc) to give the desired product (1.5 g; 79%).

¹H NMR, DMSO-d₆ (ppm): 7.47 (dt, 1H); 7.3-7.05 (m, 3H); 5.23 (d, 1H); 4.80 (dd, 1H); 1.75-1.5 (m, 2H); 1.4-1.15 (m, 4H); 0.84 (t, 3H).

HPLC (C18, λ 220 nM, 100% H₂O to 100% CH₂CN (+0.1% TFA) over 8 minutes): purity: 100%.

### Example 119B

### 1-(2-Fluorophenyl)pentan-1-one

Pyridinium chlorochromate (19.25 g; 59.5 mmol) is introduced into a suspension of Celite (22 g) in dichloromethane (300 ml) in a IL three-necked round-bottomed flask equipped with mechanical stirring. Derivative 119A (11.2 g; 59.5 mmol) predissolved in dichloromethane (30 ml) is introduced and the reaction medium is then stirred for 17 hours at room temperature. It is then filtered through a mixture of ⅔ of silica and 1/3 of Celite. The filtrate is concentrated under reduced pressure to give the desired product (10.6 g; 98%).

HPLC (C18, λ 220 nM, 100% H₂O to 100% CH₂CN (+0.1% TFA) over 8 minutes): purity: 100%.

¹H NMR, DMSO-d₆ (ppm): 7.67 (dt, 1H); 7.66-7.62 (m, 1H); 7.37-7.31 (m, 2H); 2.93 (dt, 2H); 1.58 (p, 2H); 1.33 (sext., 2H); 0.89 (t, 3H).

### Example 119C

### Ethyl 3-(butan-1-yl)benzo[b]thiophene-2-carboxylate

Derivative 119B (10 g; 35.5 mmol) is dissolved under a nitrogen atmosphere in acetonitrile (400 ml) in the presence of potassium carbonate (19.2 g; 138 mmol) and ethyl 2-mercaptoacetate (11.7 ml; 111 mmol). The reaction mixture is stirred for 16 hours at 85° C. The acetonitrile is then evaporated off and the residual solid obtained is recovered in 300 ml of water. This aqueous phase is extracted twice with 300 ml of ethyl acetate. The organic phases are combined and then washed with 300 ml of water. The organic phase is dried over magnesium sulfate, filtered and concentrated under reduced pressure. The residual oil is purified by flash chromatography (80/20 to 50/50 EDP/CH₂Cl₂ gradient) to give the desired product (3.05 g; 20%).

HPLC (C18, λ 220 nM, 100% H₂O to 100% CH₂CN (+0.1% TFA) over 8 minutes): purity: 100%.

Mass spectrum (ESI): m/z 263 (MH+).

¹H NMR, DMSO-d₆ (ppm): 7.99 (d, 1H); 8.02 (d, 1H); 7.52 (t, 1H); 7.49 (t, 1H); 4.34 (q, 2H); 3.26 (t, 2H); 1.25-1.7 (m, 7H); 0.92 (t, 3H).

Elemental analysis (C₁₅H₁₈O₂S) % calculated: C 68.67; H 6.92% found: C 68.24; H 6.86

### Example 119D

### Ethyl 3-(butan-1-yl)-7-nitrobenzo[b]thiophene-2-carboxylate

Compound 119C (3 g; 11 mmol) is dissolved in 40 ml of trifluoroacetic acid under a nitrogen atmosphere. The reaction medium is cooled to 0° C. and sodium nitrate (3.3 g; 34 mmol) is then added. The reaction medium is maintained at 0° C. for 4 hours and is then poured into 150 ml of water, and saturated aqueous sodium bicarbonate solution is added until a pH of 8 is obtained. The aqueous phase is then extracted twice with chloroform. The organic phase is dried over magnesium sulfate, filtered and concentrated under reduced pressure. The residual oil is purified by flash chromatography (80/20 to 70/30 EDP/CH₂Cl₂ gradient) to give the desired product (744 mg; 21%).

HPLC (C18, λ 220 nM, 100% H₂O to 100% CH₂CN (+0.1% TFA) over 8 minutes): purity: 100%.

Mass spectrum (ESI): m/z 263 (MH+).

¹H NMR, DMSO-d₆ (ppm): 8.40 (d, 1H); 7.94 (d, 1H); 7.71 (t, 1H); 4.37 (q, 2H); 3.05 (dd, 2H); 1.6-1.15 (m, 7H); 0.86 (t, 3H).

Elemental analysis (C₁₅H₁₇NO₄S) % calculated: C 58.61; H 5.57; N 4.56% found: C 58.57; H 5.66; N 4.34

### Example 119E

### Ethyl 7-amino-3-(butan-1-yl)benzo[b]thiophene-2-carboxylate

Compound 119E is prepared from compound 119F (1.56 g; 5 mmol) according to the conditions used for the preparation of 10B, and abiding by the proportions of the various reagents. Amount obtained: 1.26 g (85%).

HPLC (C18, λ 220 nM, 100% H₂O to 100% CH₂CN (+0.1% TFA) over 8 minutes): purity: 100%.

¹HNMR, DMSO-d₆ (ppm): 7.18 (t, 1H); 7.12 (d, 1H); 6.66 (d, 1H); 5.45 (s, 2H, NH2); 4.29 (q, 2H); 3.44 (dd, 2H); 1.65-1.55 (m, 2H); 1.46-1.35 (m, 2H); 1.30 (t, 3H); 0.91 (t, 3H).

Mass spectrum (ESI): m/z 278 (MH+)

### EXAMPLE 119

### Ethyl 3-butyl-7-{[3-(4-cyanobenzyl)-3H-imidazol-4-ylmethyl]-amino}benzo[b]thiophene-2-carboxylate

Derivative 119E (1.26 g; 4.5 mmol) and compound 27A (1.15 g; 4.5 mmol) are dissolved under a nitrogen atmosphere in DCE (32 ml) in the presence of acetic acid (1.2 ml; 23 mmol). The mixture is stirred for 48 hours at room temperature and is then neutralized with saturated aqueous sodium bicarbonate solution. The two phases are separated and the aqueous phase is washed twice with dichloromethane. The organic phases are combined, dried over magnesium sulfate, filtered and concentrated. The residual oil is purified by flash chromatography (gradient: 0/100 to 50/50 acetone/CH₂Cl₂) to give the intermediate imine (1.19 g; 55%). This imine is dissolved in THF (30 ml) under a nitrogen atmosphere, and sodium borohydride (190 mg) is added. After stirring for 18 hours at room temperature, methanol (100 μl) is added. After stirring a further 5 hours, 0.5 equivalent of reducing agent is added. After stirring for a further 3 hours, 0.5 equivalent of reducing agent is added. After stirring for 18 hours, methanol (10 ml) is added. After stirring for 1 hour, the reaction is finally complete. The reaction mixture is then concentrated. The residual solid is purified by flash chromatography (30/70 acetone/CH₂Cl₂ and then 5/95 MeOH/CH₂Cl₂) to give the desired compound (1 g; 68%).

HPLC (C18, λ 220 nM, 100% H₂O to 100% CH₂CN (+0.1% TFA) over 8 minutes): purity: 77%.

¹HNMR, DMSO-d₆ (ppm): 0.78 (t, 3H); 1.21 (hex, 2H); 1.30 (t, 3H); 1.4-1.5 (m, 2H); 3.17-3.24 (m, 2H); 4.25-4.32 (m, 4H); 5.23 (brs, 1H, NH); 5.45 (s, 2H); 6.55 (d, 1H); 7.00 (s, 1H); 7.16-7.25 (m, 4H); 7.67 (d, 2H); 7.82 (s, 1H).

Mass spectrum (ESI): m/z 473 (MH+)

### EXAMPLE 120

### 3-Butyl-7-{[3-(4-cyanobenzyl)-3H-imidazol-4-ylmethyl]amino}benzo[b]thiophene-2-carboxylic acid (120)

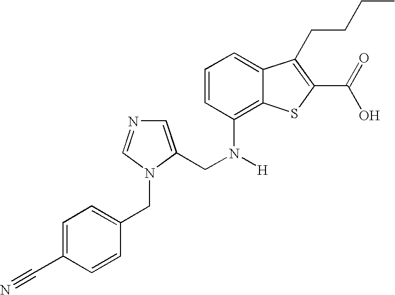

Compound 120 is prepared from compound 119 (750 mg; 1.6 mmol) according to the conditions used for the preparation of 1D, and abiding by the proportions of the various reagents. Amount obtained: 277 mg (50%).

HPLC (C18, λ 220 nM, 100% H₂O to 100% CH₂CN (+0.1% TFA) over 8 minutes): purity: 94%.

¹HNMR, DMSO-d₆ (ppm): 0.77 (t, 3H); 1.10-1.24 (m, 2H); 1.42-1.50 (m, 2H); 3.1-3.4 (m, 2H+H₂O); 4.26 (brs, 2H); 5.20 (brs, 1H, NH); 4.45 (s, 2H); 6.50 (dd, 1H); 7.17-7.19 (m, 2H); 7.22 (d, 2H); 7.73 (d, 2H); 7.82 (s, 2H).

Mass spectrum (ESI): m/z 445 (MH+)

### Examples 121 to 126

Compounds 121 to 126 are prepared from compound 120 (50 mg; 0.11 mmol) and commercial amines, according to the conditions used for the preparation of 40, and abiding by the proportions of the various reagents.

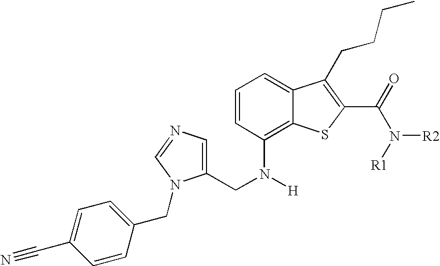

| | | | Mass | HPLC |
|---|---|---|---|---|
| Ex. | NR₁R₂ | Compound name | (M + H)⁺ | purity* |
| 121 | 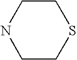 | 4-(5-{[3-Butyl-2-(thio- morpholine-4-carbonyl) benzo[b]thiophen-7-yla- mino]-methyl}imidazol- 1-ylmethyl)benzo-nitrile | 530 | 83 |
| 122 | HN-geranyl | N-(3,7-Dimethylocta-2,6- | 580 | 77 |
| | | dienyl)-3-butyl-7-{[ | | |
| | | 3-(4-cyanobenzyl)-3H-imi- | | |
| | | dazol-4-ylmethyl]a- | | |
| | | mino}-benzo[b]thio- | | |
| | | phene-2-carboxamide | | |
| 123 | 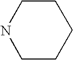 | 4-(5-{[3-Butyl-2-(piperi- dine-1-carbonyl)benzo[b]thiophen-7-ylamino]-me- thyl}imidazol-1-ylme- thyl)benzo-nitrile | 512 | 89 |
| 124 | NHBu | N-Butyl-3-butyl-7-{[3- | 500 | 86 |
| | | (4-cyanobenzyl)-3H-imida- | | |
| | | zol-4-ylmethyl]amino}- | | |
| | | benzo[b]thiophene-2-car- | | |
| | | boxamide | | |
| 125 | HN(CH₂)₂SMe | N-[2-(Methylsulfanyl)e- | 518 | 96** |
| | | thyl]-3-butyl-7-{[ | | |
| | | 3-(4-cyanobenzyl)-3H-imi- | | |
| | | dazol-4-ylmethyl]amino} | | |
| | | benzo[b]thiophene-2- | | |
| | | carboxamide | | |
| 126 | HN(CH₂)₂OiPr | N-[3-(Isopropoxy)propyl]- | 544 | 98** |
| | | 3-butyl-7-{[3-(4-cyano- | | |
| | | benzyl)-3H-imidazol-4-yl- | | |
| | | methyl]amino}benzo[b] | | |
| | | thiophene-2-carboxamide | | |

*HPLC conditions [C18 symmetry, 4.6 × 50 mm, 5 μm; λ = 220 nM; gradient 100% H₂O (+0.05% TFA) to 100% CH₂CN (+0.05% TFA) over 8 minutes]
**The compound was purified by preparative HPLC (Waters Prep 4000), on a Prep Nova-Pak HR C-18 column (Waters; 25 × 10 mm; 6 μm) using a total gradient of from 100% water (0.1% TFA) to 100% acetonitrile (0.1% TFA) over 15 minutes.

### EXAMPLE 127

### N-(2-Thiophen-2-ylethyl)-5-{[3-(4-cyanobenzyl)-3H-imidazol-4-ylmethyl]amino}benzo[b]thiophene-2-carboxamide (127)

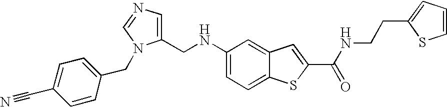

### Example 127A

### N-(2-Thiophen-2-ylethyl)-5-aminobenzo[b]thiophene-2-carboxamide

Compound 127A is prepared from the derivative 10C (1.5 g) and thiophene-2-ethylamine (1.36 ml; 11 mmol) according to the conditions used for the preparation of 89A, and abiding by the proportions of the various reagents. The residual oil is purified by flash chromatography (CH₂Cl₂ and then 80/20 CH₂Cl₂/acetone and then 70/30 CH₂Cl₂/EtOAc) to give the desired product (1.87 g; 79%).

¹H NMR, DMSO-d₆ (ppm): 3.07 (t, 2H); 3.49 (q, 2H); 5.17 (s, 2H); 6.80 (dd, 1H); 6.9-7.0 (m, 3H); 7.34 (dd, 1H); 7.59 (d, 1H); 7.76 (s, 1H); 8.74 (t, 1H).

Elemental analysis (C₁₅H₁₄N₂OS₂) % calculated: C 59.57; H 4.67; N 9.26% found: C 59.50; H 4.81; N 9.05

Mass spectrum (ESI): m/z 303 (MH+).

### EXAMPLE 127

### N-(2-Thiophen-2-ylethyl)-5-{[3-(4-cyanobenzyl)-3H-imidazol-4-ylmethyl]amino}benzo[b]thiophene-2-carboxamide

Compound 127 is prepared from derivative 127A (908 mg) according to the conditions used for the preparation of 89, and abiding by the proportions of the various reagents. The crude reaction product is then purified by flash chromatography (CH₂Cl₂, then 50/50 acetone/CH₂Cl₂ and then 10/90 MeOH/CH₂Cl₂) to give the desired product (1.06 g; 71%).

HPLC (C18, λ 220 nM, 100% H₂O to 100% CH₂CN (+0.1% TFA) over 8 minutes): purity: 96%.

Mass spectrum (ESI): m/z 498 (MH+)

¹H NMR, DMSO-d₆ (ppm): 3.07 (t, 2H); 3.49 (q, 2H); 4.12 (d, 2H); 5.39 (s, 2H); 6.06 (t, 1H); 6.78 (dd, 1H); 6.9-6.98 (m, 4H); 7.26 (d, 2H); 7.34 (d, 1H); 7.61 (d, 1H); 7.75-7.82 (m, 4H); 8.76 (t, 1H).

### Examples 128 to 136

Compounds 128 to 136 are prepared from compound 10C and from commercial amines, according to the conditions used for the preparation of 127, and abiding by the proportions of the various reagents. Certain aldehydes used are not commercial, and were prepared in the following manner:

1-Methyl-1H-imidazole-5-carboxaldehyde

1-Trityl-1H-imidazole-4-carboxaldehyde (Daminos-Zeghal S. et al., Tetrahedron, 1997, 53(22), 7605-14) (5 g; 14.8 mmol) is dissolved in dichloromethane (35 ml) under a nitrogen atmosphere and then cooled to −78° C. Methyl trifluoromethanesulfonate (1.7 ml; 14.8 mmol) is added dropwise and the reaction mixture is allowed to warm slowly to room temperature (over 2 hours). Phosphate buffer solution (pH 7; 50 ml) is added and the two-phase mixture is stirred vigorously for 15 minutes. The two phases are then separated and the aqueous phase is extracted three times with dichloromethane. The organic phases are combined, dried over magnesium sulfate, filtered and concentrated. The orange-colored solid obtained is purified by flash chromatography (CH₂Cl₂, then 10/90 acetone/CH₂Cl₂ and then 5/95 MeOH/CH₂Cl₂) to give the desired product (1.39 g; 85%).

¹H NMR, DMSO-d₆ (ppm): 3.87 (s, 3H); 7.88 (s, 1H); 8.00 (s, 1H); 9.75 (s, 1H).

1-Benzyl-1H-imidazole-5-carboxaldehyde

1-Trityl-1H-imidazole-4-carboxaldehyde (Daminos-Zeghal S. et al., Tetrahedron, 1997, 53(22), 7605-14) (2 g; 5.9 mmol) is dissolved in dichloromethane (14 ml) under a nitrogen atmosphere, and is then cooled to −78° C. Trifluoromethanesulfonic anhydride (0.99 ml; 5.9 mmol) is diluted in dichloromethane (22 ml) in another round-bottomed flask and under a nitrogen atmosphere, and the mixture is then cooled to −78° C. and treated with a solution of benzyl alcohol (0.61 ml; 5.9 mmol) and 2,6-diisopropylpyridine (1.34 ml; 6.0 mmol) in dichloromethane (9 ml). The benzyl trifluoromethanesulfonate thus formed is cannulated into the first solution and the reaction is allowed to warm slowly to room temperature (over 2 hours). Phosphate buffer solution (pH 7; 20 ml) is added and the two-phase mixture is stirred vigorously for 15 minutes. The two phases are then separated and the aqueous phase is extracted three times with dichloromethane. The organic phases are combined, dried over magnesium sulfate, filtered and concentrated. The residual oil is purified by flash chromatography (CH₂Cl₂, then 5/95 acetone/CH₂Cl₂ and then 5/95 MeOH/CH₂Cl₂) to give the desired product (615 mg; 56%).

¹H NMR, DMSO-d₆ (ppm): 5.52 (s, 2H); 7.18 (d, 2H); 7.2-7.4 (m, 3H); 7.93 (s, 1H); 8.25 (s, 1H); 9.71 (s, 1H).

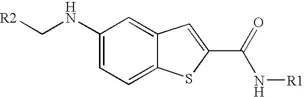

| | | | | Mass | HPLC |
|---|---|---|---|---|---|
| Ex. | NHR₁ | R₂ | Compound name | (M + H)⁺ | purity* |
| 128 | 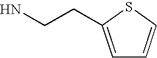 | 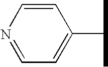 | N-(2-Thiophen-2-ylethyl)-5-{[py- rid-4-ylmethyl]-a- mino}benzo[b]thiophene-2-carbox- amide | 394 | 96** |
| 129 | 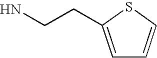 | 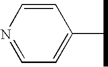 | N-(2-Thiophen-2-ylethyl)-5-{[py- rid-3-ylmethyl]-a- mino}benzo[b]thiophene-2-carbox- amide | 394 | 99** |
| 130 | 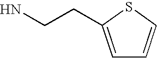 | 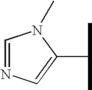 | N-(4-Thiophen-2-ylethyl)-5-{[3-meth- yl-3H-imidazol-4-ylmeth- yl]amino}benzo[b]-thio- phene-2-carboxamide | 397 | 99** |
| 131 | 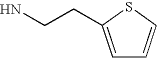 | 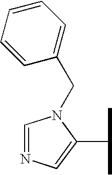 | N-(2-Thiophen-2-ylethyl)-5-{[3-ben- zyl-3H-imidazol-4-ylmeth- yl]amino}benzo[b]-thio- phene-2-carboxamide | 473 | 99*** |
| 132 | 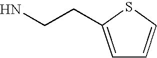 | 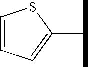 | N-(2-Thiophen-2-ylethyl)-5-{(thio- phen-2-ylmethyl]-a- mino}benzo[b]thiophene-2-carbox- amide | 399 | 94** |
| 133 | 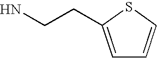 | 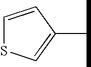 | N-(2-Thiophen-2-ylethyl)-5-{[thio- phen-3-ylmethyl]-a- mino}benzo[b]thiophene-2-carbox- amide | 399 | 98** |
| 134 | 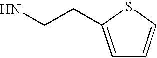 | 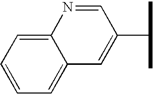 | N-(2-Thiophen-2-ylethyl)-5-{[quino- lin-3-ylmethyl]-a- mino}benzo[b]thiophene-2-carbox- amide | 444 | 99** |
| 135 | 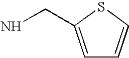 | 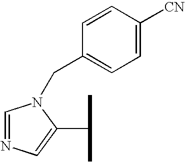 | N-(Thiophen-2-ylmethyl)-5-{[3-(4-cy- anobenzyl)-3H-imi- dazol-4-ylmethyl]amino}-ben- zo[b]thiophene-2-carbox- amide | 484 | 94** |
| 136 | NHnBu | 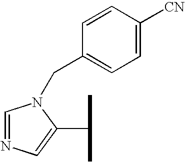 | N-Butyl-5-{[3-(4-cyano- benzyl)-3H-imidazol-4-ylmeth- yl]amino}benzo[b]-thio- phene-2-carboxamide | 444 | 99** |

*HPLC conditions [C18 symmetry, 4.6 × 50 mm, 5 μm; λ = 220 nM; gradient 100% H₂O (+0.05% TFA) to 100% CH₂CN (+0.05% TFA) over 8 minutes]
**The compound was purified by flash chromatography on silica.
***The compound was purified by preparative HPLC (Waters Prep 4000) on a Prep Nova-Pak HR C-18 column (Waters; 25 × 100 mm; 6 μm) with a total gradient of from 100% water (0.1% TFA) to 100% acetonitrile (0.1% TFA) over 15 minutes.

### EXAMPLE 137

4-[5-({2-[(2-Thiophen-2-ylethylamino)methyl]benzol[b]thiophen-5-ylamino}methyl)imidazol-1-ylmethyl]benzonitrile (7)

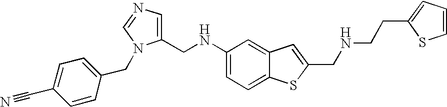

Compound 127A (200 mg; 0.66 mmol) is dissolved in anhydrous THF (8 ml) under a nitrogen atmosphere and treated with lithium aluminum hydride (LAH; 1.3 ml; 1.3 mmol). The reaction mixture is heated to 66° C. After 18 hours, LAH (0.65 ml) and THF are added. After stirring a further 24 hours, the reaction mixture is still incomplete. It is cooled to room temperature and neutralized by successive addition of water (100 μl), 10% sodium hydroxide (100 μl) and water (300 μl). The solid is filtered off and rinsed thoroughly with dichloromethane. The filtrate is concentrated and the solid residue is treated again with LAH using the same procedure, so as to complete the reaction. The final solid is then dissolved in DCE (7 ml) in the presence of derivative 27A (146 mg; 0.69 mmol) and acetic acid (170 μl; 3.3 mmol), at room temperature and under a nitrogen atmosphere for a few minutes, and sodium triacetoxyborohydride (153 mg; 0.72 mmol) is then added. After stirring for 24 hours, ethyl acetate (30 ml) is added and the reaction mixture is washed with saturated aqueous sodium bicarbonate solution (30 ml) and then dried over magnesium sulfate, filtered and concentrated. The residue is purified by preparative HPLC (Waters Prep 4000) on a Prep Nova-Pak HR C-18 column (Waters; 25×100 mm; 6 μm) using a total gradient of from 100% water (0.1% TFA) to 100% acetonitrile (0.1% TFA) over 15 minutes, to give the desired product (95 mg).

HPLC (C 18, λ 220 nM, 100% H₂O to 100% CH₂CN (+0.1% TFA) over 8 minutes): purity: 89%.

Mass spectrum (ESI): m/z 484 (MH+)

A fraction of product is desalified for the NMR spectrum.

¹H NMR, DMSO-d₆ (ppm): 2.78 (t, 2H); 2.94 (t, 2H); 3.94 (s, 2H); 4.08 (d, 2H); 5.38 (s, 2H); 5.86 (t, 1H, NH); 6.62 (dd, 1H); 6.74 (bs, 1H); 6.8-7.05 (m, 4H); 7.2-7.35 (m, 3H); 7.48 (d, 1H); 7.75-7.9 (m, 3H).

### Examples 138 to 155

Compounds 138 to 155 are prepared from derivatives 127, 135 or 136, and from commercial acid chlorides, according to the conditions described for the preparation of 91, and abiding by the proportions of the various reagents.

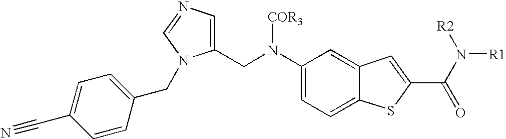

| | | | | Mass | HPLC |
|---|---|---|---|---|---|
| Ex. | NR₁R₂ | R₃CO | Compound name | (M + H)⁺ | purity* |
| 138 | 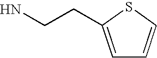 | NPrCO | N-(2-Thiophen-2-ylethyl)-5-{bu- tyryl-[3-(4-cyanobenzyl)-3H-imi- dazol-4-ylmethyl]-a- mino}benzo[b]thiophene-2-carbox- amide | 568 | 99 |
| 139 | 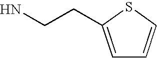 | NPentylCO | N-(2-Thiophen-2-ylethyl)-5-(hexa- noyl-[3-(4-cyano- benzyl)-3H-imidazol-4-ylmeth- yl]amino}benzo[b]-thio- phene-2-carboxamide | 596 | 99 |
| 140 | 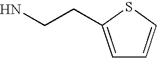 | PhCO | N-(2-Thiophen-2-ylethyl)-5-{ben- zoyl-[3-(4-cyanobenzyl)-3H-imi- dazo-4-ylmethyl]-a- mino}benzo[b]thiophene-2-carbox- amide | 602 | 98 |
| 141 | 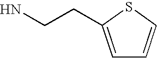 | 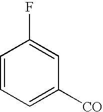 | N-(2-Thiophen-2-ylethyl)-5-{(3-fluoro- benzoyl)-[3-(4-cyano- benzyl)-3H-imidazol-4-ylmeth- yl]amino}benzo[b]-thio- phene-2-carboxamide | 620 | 91 |
| 142 | 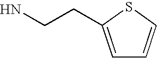 | 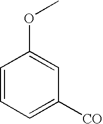 | N-(2-Thiophen-2-ylethyl)-5-{(3-meth- oxybenzoyl)-[3-(4-cyano- benzyl)-3H-imidazol-4-ylmeth- yl]amino}benzo[b]-thio- phene-2-carboxamide | 632 | 92 |
| 143 | 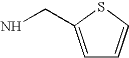 | 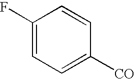 | N-(2-Thiophen-2-ylethyl)-5-{(4-fluoro- benzoyl)-[3-(4-cyano- benzyl)-3H-imidazol-4-ylmeth- yl]amino}benzo[b]-thio- phene-2-carboxamide | 620 | 95 |
| 144 | 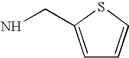 | NPrCO | N-(2-Thiophen-2-ylmethyl)-5-{bu- tyryl-[3-(4-cyano- benzyl)-3H-imidazol-4-ylmeth- yl]amino}benzo[b]-thio- phene-2-carboxamide | 554 | 95 |
| 145 | 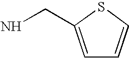 | NPentylCO | N-(2-Thiophen-2-ylmethyl)-5-{hex- anoyl-[3-(4-cyano- benzyl)-3H-imidazol-4-ylmeth- yl]amino}benzo[b]-thio- phene-2-carboxamide | 582 | 99 |
| 146 | 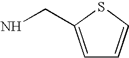 | PhCO | N-(2-Thiophen-2-ylmethyl)-5-{ben- zoyl-[3-(4-cyano- benzyl)-3H-imidazol-4-ylmeth- yl]amino}benzo[b]-thio- phene-2-carboxamide | 588 | 92 |
| 147 | 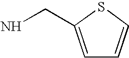 | 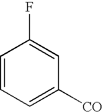 | N-(2-Thiophen-2-ylmethyl)-5-{(3-fluoro- benzoyl)-[3-(4-cyano- benzyl)-3H-imidazol-4-ylmeth- yl]amino}benzo[b]-thio- phene-2-carboxamide | 606 | 91 |
| 148 | 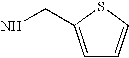 | 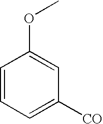 | N-(2-Thiophen-2-ylmethyl)-5-{(3-meth- oxybenzoyl)-[3-(4-cyano- benzyl)-3H-imi- dazol-4-ylmethyl]amino}-ben- zo[b]thiophene-2-carboxamide | 618 | 90 |
| 149 | 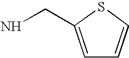 | 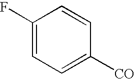 | N-(2-Thiophen-2-ylmethyl)-5-{(4-fluoro- benzoyl)-[3-(4-cyano- benzyl)-3H-imidazol-4-ylmeth- yl]amino}benzo[b]-thio- phene-2-carboxamide | 606 | 96 |
| 150 | NHnBu | NPrCO | N-Butyl-5-{butyryl-[3-(4-cyano- | 514 | 99 |
| | | | benzyl)-3H-imidazol-4-ylmeth- | | |
| | | | yl]amino}benzo[b]-thio- | | |
| | | | phene-2-carboxamide | | |
| 151 | NHnBu | NPentylCO | N-Butyl-5-{hexanoyl-[3-(4-cyano- | 542 | 99 |
| | | | benzyl)-3H-imidazol-4-ylmeth- | | |
| | | | yl]amino}benzo[b]-thio- | | |
| | | | phene-2-carboxamide | | |
| 152 | NHnBu | PhCO | N-Butyl-5-{benzoyl-[3-(4-cyano- | 548 | 96 |
| | | | benzyl)-3H-imidazol-4-ylmeth- | | |
| | | | yl]amino}benzo[b]-thio- | | |
| | | | phene-2-carboxamide | | |
| 153 | NHnBu | 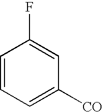 | N-Butyl-5-{(3-fluoro- benzoyl)-[3-(4-cyanobenzyl)-3H-imi- dazol-4-ylmethyl]-a- mino}benzo[b]thiophene-2-carbox- amide | 566 | 97 |
| 154 | NHnBu | 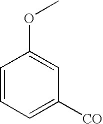 | N-Butyl-5-{(3-methoxy- benzoyl)-[3-(4-cyanobenzyl)-3H-imi- dazol-4-ylmethyl]-a- mino}benzoyl[b]thiophene-2-carbox- amide | 578 | 96 |
| 155 | NHnBu | 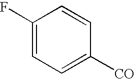 | N-Butyl-5-{(4-fluoro- benzoyl)-[3-(4-cyanobenzyl)-3H-imi- dazol-4-ylmethyl]-a- mino}benzo[b]thiophene-2-carbox- amide | 566 | 97 |

*HPLC conditions [C18 symmetry, 4.6 × 50 mm, 5 μm; λ = 220 nM; gradient 100% H₂O (+0.05% TFA) to 100% CH₂CN(+0.05% TFA) over 8 minutes]

### EXAMPLES 156 and 157

Compounds 156 and 157 are prepared from derivatives 38 or 39, according to the conditions described for the preparation of 40, and abiding by the proportions of the various reagents. The products were purified by preparative HPLC (Waters Prep 4000) on a Prep Nova-Pak HR C-18 column (Waters; 25×100 mm; 6 μm) using a total gradient of from 100% water (0.1% TFA) to 100% acetonitrile (0.1% TFA) over 15 minutes.

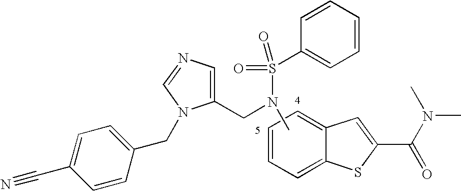

| Ex. | Substituent position | Compound name | Mass (M + H)⁺ | HPLC purity* |
|---|---|---|---|---|
| 156 | 4 | N,N-Dimethyl-4-{benzenesulfonyl-[3-(4-cyano- | 556 | 99 |
| | | benzyl)-3H-imidazol-4-ylmeth- | | |
| | | yl]amino}benzo[b]thiophene-2-carbox- | | |
| | | amide | | |
| 157 | 5 | N,N-Dimethyl-5-{benzenesulfonyl-[3-(4-cyano- | 556 | 96 |
| | | benzyl)-3H-imidazol-4-ylmeth- | | |
| | | yl]amino}benzo[b]thiophene-2-carbox- | | |
| | | amide | | |

*HPLC conditions [C18 symmetry, 4.6 × 5 mm, 5 μm; λ = 220 nM; gradient 100% H₂O (+0.05% TFA) to 100% CH₂CN(+0.05% TFA) over 8 minutes]

### EXAMPLE 158

### (2S)-2-({5-[(3H-Imidazol-4-ylmethyl)amino]-3-phenyl-1H-indole-2-carbonyl}amino)-4-(methylsulfanyl)butyric acid

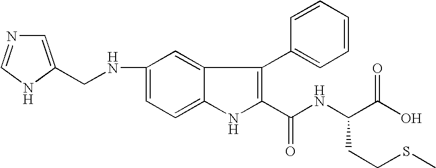

### Example 158A

### Methyl (2S)-4-methylsulfanyl-2-[(5-nitro-3-phenyl-1H-indole-2-carbonyl)amino]butyrate

5-Nitro-3-phenyl-1H-indole-2-carboxylic acid (1.5 g; 5.31 mmol) dissolved in dichloromethane (48 ml) is treated with HOOBT (953 mg; 5.84 mmol) and EDC (1.02 g; 5.84 mmol) and the mixture is then stirred for 1 hour at room temperature. A solution of H-Met-OMe hydrochloride (1.17 g; 5.84 mmol) and DIPEA (1.85 ml; 10.62 mmol) in dichloromethane (20 ml) is then cannulated into the first solution. The mixture is stirred for 18 hours at room temperature and then diluted with dichloromethane and washed successively with water and saturated aqueous sodium chloride solution. The organic phase is dried over magnesium sulfate, filtered and concentrated. This crude reaction product is then purified by flash chromatography (85/14/1 CH₂Cl₂/MeOH/NH₄OH) to give the desired product (1.86 g; 82%).

¹H NMR, DMSO-d₆ (ppm): 1.92 (m, 2H); 2.00 (s, 3H); 2.33 (m, 2H); 3.65 (s, 3H); 4.54 (m, 1H); 7.45 (t, 1H, 7.0 Hz); 7.55 (m, 4H); 7.65 (d, 1H, 9.0 Hz); 8.14 (dd, 1H, 1.7 and 9.0 Hz); 8.18 (d, 1H, 7.4 Hz); 8.40 (d, 1H, 1.6 Hz); 12.56 (s, 1H).

### Example 158B

### Methyl(2S)₄-methylsulfanyl-2-[(5-amino-3-phenyl-1H-indole-2-carbonyl)amino]butyrate

Compound 158A (1.67 g; 3.54 mmol) dissolved in a mixture of ethanol (50 ml) and methanol (20 ml) in the presence of a catalytic amount of palladium-on-charcoal (10%) is hydrogenated using a hydrogen balloon for 7 hours. The medium is filtered and the filtrate is evaporated to dryness. The syrup obtained is purified by flash chromatography (95/4.5/0.5 CH₂Cl₂/MeOH/NH₄OH) to give the desired product (999 mg; 71%).

¹H NMR, DMSO-dr (ppm): 1.91 (m, 2H); 2.00 (s, 3H); 2.33 (m, 2H); 3.63 (s, 3H); 4.48 (m, 1H); 4.64 (broad s, 2H); 6.62 (s, 1H); 6.66 (d, 1H, 8.7 Hz); 7.17 (d, 1H, 8.7 Hz); 7.35 (m, 1H); 7.44 (d, 4H, 7.4 Hz); 7.53 (d, 1H, 7.4 Hz); 11.29 (s, 1H).

### Example 158C

### Methyl(2S)-4-methylsulfanyl-2-({3-phenyl-5-[(1-trityl-1H-imidazol-4-ylmethyl)amino]-1H-indole-2-carbonyl}amino)butyrate

A mixture comprising compound 158B (350 mg; 0.88 mmol) and 1-trityl-1H-imidazole-4-carboxaldehyde (352 mg; 1.00 mmol) dissolved in 1,2-dichloroethane (DCE) (4.5 ml) in the presence of acetic acid (0.3 ml) is stirred for 5 minutes at room temperature and sodium triacetoxyborohydride (233 mg; 1.10 mmol) is then added. The mixture is stirred overnight at room temperature and is then diluted with ethyl acetate and washed successively with saturated NaHCO₃ solution, with water, and with saturated aqueous sodium chloride solution. The organic phase is dried over magnesium sulfate, filtered and concentrated. This crude reaction, product is then purified by flash chromatography (gradient: 4/1 and then 2/1 CH₂Cl₂/acetone) to give the desired product (304 mg; 48%).

¹H NMR, DMSO-d₆ (ppm): 1.88 (m, 2H); 2.00 (s, 3H); 2.33 (m, 2H); 3.63 (s, 3H); 4.03 (d, 2H, 5.2 Hz); 4.50 (m, 1H); 5.45 (t, 1H, 5.6 Hz); 6.55 (s, 1H); 6.70 (s, 1H); 6.76 (d, 1H, 8.7 Hz); 7.01 (m, 6H); 7.21 (d, 1H, 8.7 Hz); 7.25 (s, 1H); 7.31-7.41 (m, 13H); 7.55 (d, 1H, 7.4 Hz); 7.73 (d, 3H, 9.4 Hz); 11.35 (s, 1H).

### EXAMPLE 158

### -(2S)-2-({5-[(−3H-Imidazol-4-ylmethyl)amino]-3-phenyl-1H-indole-2-carbonyl}amino)-4-(methylsulfanyl)butyric acid

Compound 158C (200 mg; 0.298 mmol) dissolved in THF (3.5 ml) is treated with aqueous (1M) LiOH solution (0.6 ml; 0.596 mmol). After stirring overnight at room temperature, the medium is acidified with aqueous 1M HCl solution and then evaporated to dryness and coevaporated with toluene. This crude reaction product is then purified by flash chromatography (gradient: 5/1 CH₂Cl₂/MeOH) to give the pure carboxylic acid (190 mg; 97%). This intermediate (140 mg; 0.198 mmol) is taken up in dichloromethane and treated with TFA (0.412 ml; 5.34 mmol) for 1 hour 15 minutes and then with triethylsilyl hydride (63 μl; 0.382 mmol) for 30 minutes. The medium is evaporated to dryness and the crude reaction product is purified by preparative HPLC (C18, gradient: 100/0 to 50/50 water/CH₃CN over 25 minutes) to give the expected compound (16 mg; 14%).

¹H NMR, DMSO-d₆ (ppm): 1.95 (m, 2H); 2.00 (s, 3H); 2.30 (m, 2H); 4.28 (s, 2H); 4.42 (m, 1H); 6.61 (s, 1H); 6.81 (d, 1H, 8.8 Hz); 7.29 (d, 1H, 8.8 Hz); 7.33-7.46 (m, 6H); 7.50 (s, 1H); 8.98 (s, 1H); 11.45 (s, 1H); 14.10 (broad s, 1H).

Mass spectrum (ESI): m/z 464 (M+2H+).

### EXAMPLE 159

(2S)-2-[(5-{1[3-(4-Cyanobenzyl)-3H-imidazol-4-ylmethyl]amino}-3-phenyl-1H-indole-2-carbonyl)amino]-4-(methylsulfanyl)butyric acid

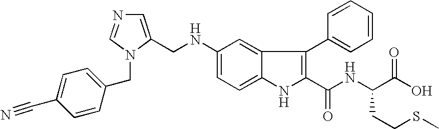

### Example 159A

### Methyl 4-methylsulfanyl-2-[(5-{[3-(4-cyanobenzyl)-3H-imidazol-4-ylmethyl]amino}-3-phenyl-1H-indole-2-carbonyl)amino]butyrate

A mixture comprising compound 158B (300 mg; 0.75 mmol) and compound 27A (159 mg; 0.75 mmol) dissolved in 1,2-dichloroethane (DCE) (3.5 ml) in the presence of acetic acid (1.3 ml) is stirred for 1 minute at room temperature and sodium triacetoxyborohydride (175 mg; 0.82 mmol) is then added. The mixture is stirred overnight at room temperature and is then diluted with ethyl acetate and washed successively with saturated NaHCO₃ solution, with water and with saturated aqueous sodium chloride solution. The organic phase is dried over magnesium sulfate, filtered and concentrated. This crude reaction product is then purified by flash chromatography (gradient: 2/1 and then 1/1 CH₂Cl₂/acetone) to give the desired product (321 mg; 72%).

¹H NMR, DMSO-d₆ (ppm): 1.88 (m, 2H); 2.00 (s, 3H); 2.33 (m, 2H); 3.63 (s, 3H); 3.98 (d, 2H, 5.2 Hz); 4.50 (m, 1H); 5.36 (s, 2H); 5.51 (t, 1H, 5.2 Hz); 6.53 (s, 1H); 6.67 (d, 1H, 8.9 Hz); 6.85 (s, 1H); 7.21 (t, 3H, 8.3 Hz); 7.35 (m, 1H); 7.45 (m, 4H, 7.4 Hz); 7.59 (d, 1H, 7.4 Hz); 7.73 (d, 3H, 9.4 Hz); 11.37 (s, 1H).

### Example 159

### (2S)-2-[(5-{[3-(4-Cyanobenzyl)-3H-imidazol-4-ylmethyl]amino}-3-phenyl-1H-indole-2-carbonyl)amino]-4-(methylsulfanyl)butyric acid

Compound 159A (120 mg; 0.20 mmol) dissolved in THF (2 ml) is treated with aqueous (1M) LiOH solution (0.4 ml; 0.4 mmol). After stirring overnight at room temperature, the medium is acidified with aqueous 1M HCl solution and then evaporated to dryness and coevaporated with toluene. This crude reaction mixture is then purified by flash chromatography (gradient: 10/1 CH₂Cl₂/MeOH) to give the pure product (104 mg; 89%).

¹H NMR, DMSO-d₆ (ppm): 1.94 (m, 2H); 2.01 (s, 3H); 2.36 (m, 2H); 4.02 (s, 2H); 4.43 (m, 1H); 5.44 (s, 2H); 6.50 (s, 1H); 6.68 (d, 1H, 7.5 Hz); 7.04 (s, 1H); 7.21 (d, 1H, 8.6 Hz); 7.29 (d, 1H, 8.0 Hz); 7.36 (d, 1H, 6.9 Hz); 7.43-7.48 (m, 2H); 7.75 (d, 1H, 7.8 Hz); 8.15 (s, 1H); 11.47 (s, 1H).

Mass spectrum (ESI): m/z 668 (M−H+).

### EXAMPLE 160

### (2S)-2-{(5-[Benzenesulfonyl-(3H-imidazol-4-ylmethyl)amino]-3-phenyl-1H-indole-2-carbonyl}amino)-4-(methylsulfanyl)butyric acid

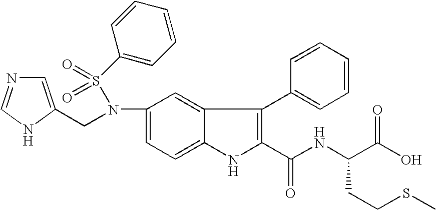

### Example 160A

### Methyl(2S)-2-({5-benzenesulfonyl-(1-trityl-1H-imidazol-4-yl-methyl)amino]-3-phenyl-1H-indole-2-carbonyl}amino)-4-(methylsulfanyl)-butyrate

Compound 158C (150 mg; 0.21 mmol) dissolved in pyridine (2.5 ml) is treated, at room temperature, with benzenesulfonyl chloride (53 II; 0.42 mmol). After stirring overnight at room temperature, the medium is evaporated to dryness. The residue is taken up in dichloromethane and washed successively with water and with saturated aqueous sodium chloride solution. The organic phase is dried over magnesium sulfate, filtered and concentrated. This crude reaction product is then purified by flash chromatography (gradient: 4/1 CH₂Cl₂/acetone) to give the desired product (137 mg; 77%).

### Example 160B

### (2S)-2-({5-[Benzenesulfonyl-(1-trityl-1H-imidazol-4-ylmethyl)-amino]-3-phenyl-1H-indole-2-carbonyl}amino)₄-methylsulfanylbutyric acid

Compound 160A (137 mg; 0.16 mmol) dissolved in THF (2 ml) is treated with 1M LiOH solution in water (320 μl; 0.32 mmol) at room temperature overnight. The solution is taken up in dichloromethane and washed successively with water and with saturated aqueous sodium chloride solution. The organic phase is dried over magnesium sulfate, filtered and concentrated. This crude reaction product is then purified by flash chromatography (gradient: 5/2 CH₂Cl₂/MeOH) to give the desired product (127 mg; 94%).

### Example 160

### 2S)-2-{(5-[Benzenesulfonyl-(3H-imidazol-4-ylmethyl)amino] 3-phenyl-1H-indole-2-carbonyl}amino)-4-(methylsulfanyl)butyric acid

Compound 160B (127 mg; 0.15 mmol) dissolved in dichloromethane (3 ml) is treated with TFA (354 μl; 4.05 mmol) at room temperature for 1 hour 15 minutes, Et₃SiH (54 μl; 0.30 mmol) is then added and the medium is stirred for a further 30 minutes. The mixture is evaporated to dryness and then triturated with ether to give a precipitate, which is isolated by filtration. This solid is purified by preparative HPLC (C18, gradient: 100% water (+0.1% TFA) to 100% CH₃CN (+0.1% TFA) over 25 minutes) to give the desired product after freeze-drying (27 mg; 30%).

¹H NMR, DMSO-d₆ (Ppm): 1.82 (m, 1H); 1.93 (m, 1H); 2.00 (s, 3H); 2.33 (m, 2H); 4.42 (m, 1H); 4.82 (d, 1H, 14.8 Hz); 4.91 (d, 1H, 14.8 Hz); 6.87 (dd, 1H, 8.7 and 1.7 Hz); 6.91 (s, 1H); 7.25 (d, 2H, 7.1 Hz); 7.32-7.42 (m, 6H); 7.66 (d, 4H, 4.2 Hz); 7.71 (d, 1H, 7.7 Hz); 7.83 (m, 1H); 8.89 (s, 1H); 11.82 (s, 1H); 14.30 (broad s, 1H).

### EXAMPLE 161

### (2S)-2-[(5-{Benzenesulfonyl-[3-(4-cyanobenzyl)-3H-imidazol-4-ylmethyl]-amino}-3-phenyl-1H-indole-2-carbonyl)amino]-4-(methylsulfanyl)butyric acid

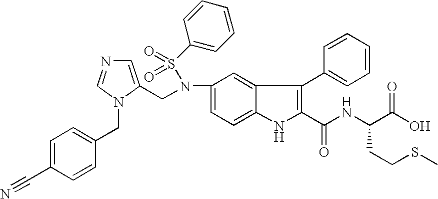

Compound 161 is prepared from compound 159A (200 mg; 0.34 mmol) according to the procedure described for the preparation of Example 160B starting with 158C. The pure compound is isolated in the form of a white foam (88 mg; 44%).

Mass spectrum (ESI): m/z 719 (M+H+).

### EXAMPLE 162

### N-(Thiophen-2-ylmethyl)-5-{[3-(4-cyanobenzyl)-3H-imidazol-4-ylmethyl]amino}-3-phenyl-1H-indole-2-carboxamide

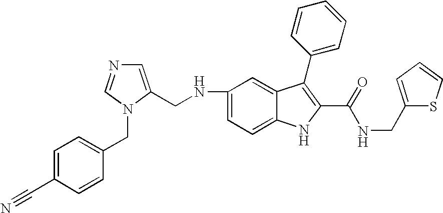

### Example 162A

### Methyl 5-{1[3-(4-cyanobenzyl)-3H-imidazol-4-ylmethyl]amino}-3-phenyl-1H-indole-2-carboxylate

A mixture comprising methyl 5-amino-3-phenyl-1H-indole-2-carboxylate (1.4 g; 5.26 mmol) and compound 27A (1.11 g; 5.26 mmol) dissolved in 1,2-dichloroethane (DCE) (24 ml) in the presence of acetic acid (1.7 ml) is stirred for 1 minute at room temperature and sodium triacetoxyborohydride (1.23 g; 5.78 mmol) is then added. The mixture is stirred overnight at room temperature and is then diluted with ethyl acetate and washed successively with saturated NaHCO₃ solution, with water and with saturated aqueous sodium chloride solution. The organic phase is dried over magnesium sulfate, filtered and concentrated. This crude reaction product is then purified by flash chromatography (gradient: 1/1 and then 1/2 CH₂Cl₂/acetone and then 9/1 CH₂Cl₂/MeOH) to give the desired product (1.22 g; 50%).

¹H NMR, DMSO-d₆ (ppm): 3.71 (s, 3H); 3.97 (d, 2H, 5.2 Hz); 5.35 (s, 2H); 5.61 (t, 1H, 5.2 Hz); 6.49 (s, 1H); 6.73 (d, 1H, 8.8 Hz); 6.84 (s, 1H); 7.21 (m, 3H); 7.35 (m, 1H); 7.42 (m, 4H); 7.72 (m, 3H); 11.58 (s, 1H).

Mass spectrum (ESI): m/z 462 (M+H+).

### Example 162B

### 5-{1[3-(4-Cyanobenzyl)-3H-imidazol-4-ylmethyl]amino}-3-phenyl-1H-indole-2-carboxylic acid

Compound 162A (500 mg; 1.08 mmol) dissolved in THF (4.3 ml) is treated with aqueous (1M) LiOH solution (4.3 ml; 4.3 mmol). After stirring for 32 hours at room temperature, the medium is acidified by addition of Dowex 50W resin for 2 hours and then filtered, washed with THF and with water and finally evaporated to dryness. The product 162A obtained (364 mg; 75%) is thus used for the following step.

Mass spectrum (ESI): m/z 448 (M+H+).

### EXAMPLE 162

### N-(Thiophen-2-ylmethyl)-5-{1[3-(4-cyanobenzyl)-3H-imidazol-4-ylmethyl]amino}-3-phenyl-1H-indole-2-carboxamide

The acid 162B (50 mg; 0.11 mmol) in DMF (0.5 ml) is treated with PS-carbodiimide resin (94 mg; 1.05 mmol/g; 0.11 mmol) and with a solution of HOBT (11.5 mg; 0.085 mmol) in DMF (3 ml) for 30 minutes, thiophen-2-ylmethylamine (8.4 mg; 0.0744 mmol) dissolved in DMF (1 ml) is then added and the mixture is stirred at room temperature for 24 hours. The medium is diluted with CH₂Cl₂ (3 ml) and then treated with MP-carbonate resin (77.5 mg; 3.2 mmol/g; 0.24 mmol). The medium is stirred at room temperature for 16 hours and is then filtered and the resin is washed with CH₂Cl₂ and with DMF. The solvents are evaporated off and the product is freeze-dried to give the expected product, which is purified by semipreparative HPLC (C18, gradient: 100% (+0.1% TFA) to 100% CH₃CN (+0.1% TFA) over 20 minutes) to give the desired product after freeze-drying (15 mg; 17%).

Mass spectrum (ESI): m/z 543 (M+H+).

### EXAMPLE 163

### N-(Thiophen-2-ylmethyl)-5-[[3-(4-cyanobenzyl)-3H-imidazol-4-ylmethyl]-(4-nitrobenzoyl)amino]-3-phenyl-1H-indole-2-carboxamide

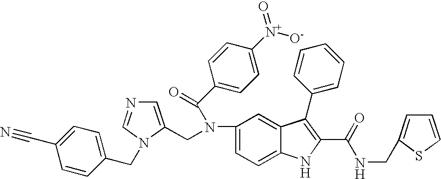

### Example 163A

### 5-[[3-(4-Cyanobenzyl)-3H-imidazol-4-ylmethyl]-(4-nitrobenzoyl)amino]-3-phenyl-1H-indole-2-carboxylic acid

Compound 162A (500 mg; 1.08 mmol) dissolved in dichloromethane (17 ml) in the presence of PS-DIEA (885 mg; 3.67 mmol/g; 3.24 mmol) is treated with 4-nitrobenzoyl chloride (260 mg; 1.40 mmol) at room temperature. The medium is stirred for 1 hour 30 minutes and the excess acid chloride is then trapped by addition of PS-trisamine (1.18 g; 3.66 mmol/g; 4.32 mmol) and stirred for 5 hours at room temperature. The medium is filtered, the resin is washed with dichloromethane and the filtrate is then evaporated to dryness. The crude product obtained is purified by flash chromatography (gradient: 2/1 CH₂Cl₂/acetone) to give the intermediate ester (640 mg). This intermediate is saponified according to the procedure described for the conversion of Example 162A into 162B, to give the desired acid (620 mg; 95%).

Mass spectrum (ESI): m/z 597 (M+H+).

### EXAMPLE 163

### N-(Thiophen-2-ylmethyl)-5-[[3-(4-cyanobenzyl)-3H-imidazol-4-ylmethyl]-(4-nitrobenzoyl)amino]-3-phenyl-1H-indole-2-carboxamide

Compound 163 is obtained from the acid 163A (50 mg; 0.083 mmol) and thiophen-2-ylmethylamine according to the procedure described for the conversion of Example 162B into 162 (30 mg; 45%).

Mass spectrum (ESI): m/z 692 (M+H+).

### Examples 164 to 169

Compounds 164 to 169 are prepared according to the conditions described for the preparation of examples 162 and 163, and abiding the proportions of the various reagents. The products were purified by chromatography on silica using a combiflash (gradient: CH₂Cl₂ to 8/2 CH₂Cl₂/MeOH over 12 minutes).

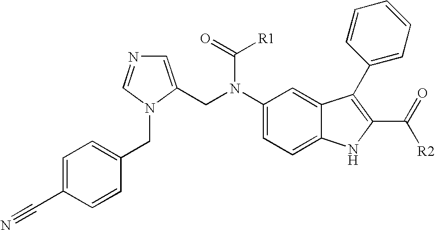

| | | | | Mass | HPLC |
|---|---|---|---|---|---|
| Example | R1 | R2 | Compound name | (M + H)⁺ | purity* |
| 164 | 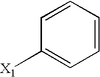 | 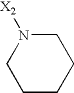 | N-[3-(4-Cyanobenzyl)-3H-imidazol-4-ylmeth- yl]-N-[3-phenyl-2-(piperidine-1-carbo- nyl)-1H-indol-5-yl]benzamide | 619 | 95 |
| 165 | 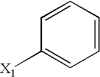 | 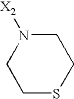 | N-[3-(4-Cyanobenzyl)-3H-imidazol-4-ylmeth- yl]-N-[3-phenyl-2-(thio- morpholine-4-carbonyl)-1H-indol-5-yl]benz- amide | 637 | 93 |
| 166 | 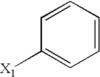 | 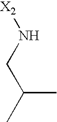 | N-(Isobutyl)-5-{benzoyl-[3-(4-cyano- benzyl)-3H-imidazol-4-ylmethyl]-a- mino}-3-phenyl-1H-indole-2-carbox- amide | 607 | 97 |
| 167 | 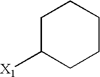 | 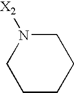 | Cyclohexanecarboxylic-[3-(4-cyano- benzyl)-3H-imidazol-4-ylmethyl]-[3-phe- nyl-2-(piperidine-1-carbonyl)-1H-in- dol-5-yl]amide acid | 625 | 96 |
| 168 | 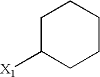 | 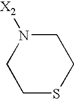 | Cyclohexanecarboxylic-[3-(4-cyano- benzyl)-3H-imidazol-4-ylmethyl]-[3-phe- nyl-2-(thiomorpholine-4-carbonyl)-1H-in- dol-5-yl]amide acid | 643 | 95 |
| 169 | 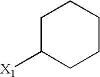 | 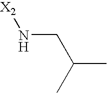 | N-(Isobutyl)-5-{[3-(4-cyanobenzyl)-3H-imi- dazol-4-ylmethyl]cyclo- hexanecarbonylamino}-3-phenyl-1H-in- dole-2-carboxamide | 613 | 98 |

*HPLC conditions [C18 symmetry, 4.6 × 50 mm, 5 μm; λ = 220 nM; gradient 100% H₂O (+0.05% TFA) to 100% CH₂CN (+0.05% TFA) over 8 minutes]

The derivatives of the present invention are inhibitors of protein prenylation and more particularly of the farnesylation of ras proteins, as shown by the studies of inhibition of protein farnesyl transferase and of protein geranylgeranyl transferase.

### Examples 170 to 186

Compounds 170 to 180 are prepared in the form of TFA salts from the derivatives 127, 135 or 136, and from the corresponding aldehydes, according to the conditions described for the preparation of 170, and abiding by the proportions of the various reagents.

Compound 127 (50 mg; 0.11 mmol) is dissolved in methanol (1.5 ml) in the presence of cyclohexanecarboxaldehyde (0.68 M/EtOH; 0.5 ml; 0.33 mmol) and acetic acid (0.9 M/EtOH; 0.5 ml; 0.45 mmol). Supported cyanoborohydride (Fluka; 2 mmol/g; 169 mg; 0.33 mmol) is added and the mixture is stirred at room temperature until product 127 has disappeared. The reaction mixture is filtered and the polymer is rinsed twice with methanol. The solution is concentrated. The residue is purified by preparative HPLC (Waters Prep 4000) on a Prep Nova-Pak HR C-18 column (Waters; 25×100 mm; 6 μm) using a total gradient of from 100% water (0.1% TFA) to 100% acetonitrile (0.1% TFA) over 15 minutes, and is then freeze-dried to give product 170 in the form of the TFA salt.

Compounds 181 to 185 are prepared in the form of the HCl salts from derivative 127, and from the corresponding aldehydes, according to the conditions described for the preparation of 159A, in the presence of a large excess of aldehyde. They are then purified by preparative HPLC (Waters Prep 4000) on a Prep Nova-Pak HR C-18 column (Waters; 25×100 mm; 6 μm) using a total gradient of from 100% water (0.1% HCl) to 100% acetonitrile (0.1% HCl) over 15 minutes, and then freeze-dried to give the corresponding HCl salts.

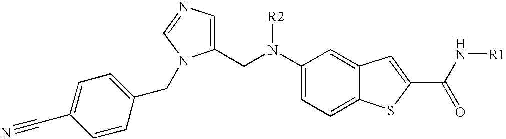

| | | | | Mass | HPLC |
|---|---|---|---|---|---|
| Ex. | R1 | R2 | Compound name | (M + H)⁺ | purity* |
| 170 | 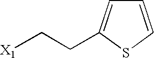 | 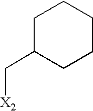 | N-(2-Thiophen-2-ylethyl)-5-{[3-(4-cyano- benzyl)-3H-imidazol-4-ylmeth- yl]cyclohexylmethylamino}-ben- zo[b]thiophene-2-carboxamide | 594 | 95 |
| 171 | 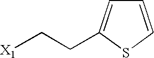 | 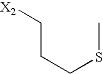 | N-(2-Thiophen-2-ylethyl)-5-{[3-(4-cyano- benzyl)-3H-imidazol-4-ylmeth- yl]-(3-methylsulfanylpropyl)-a- mino}benzo[b]thiophene-2-cabox- amide | 586 | 97 |
| 172 | 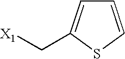 | 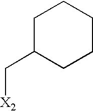 | N-(Thiophen-2-ylmethyl)-5-{[3-(4-cyano- benzyl)-3H-imidazol-4-ylmeth- yl]cyclohexylmethylamino}-ben- zo[b]thiophene-2-carboxamide | 580 | 99 |
| 173 | 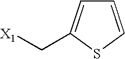 | 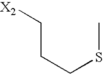 | N-(Thiophen-2-ylmethyl)-5-{[3-(4-cyano- benzyl)-3H-imidazol-4-ylmeth- yl]-(3-methylsulfanylpropyl)-a- mino}benzo[b]thiophene-2-carbox- amide | 572 | 96 |
| 174 | X₁nButyl | 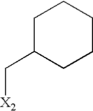 | N-Butyl-5-{[3-(4-cyanobenzyl)-3H-imi- dazol-4-ylmethyl]cyclohexyl-methyl- amino}benzo[b]thiophene-2-carbox- amide | 540 | 99 |
| 175 | X₁nButyl | 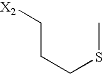 | N-Butyl-5-{[3-(4-cyanobenzyl)-3H-imi- dazol-4-ylmethyl]-(3-methyl- sulfanylpropyl)amino}benzo[b]-thio- phene-2-carboxamide | 532 | 96 |
| 176 | 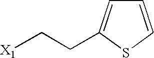 | X₂nHeptyl | N-(2-Thiophen-2-ylethyl)-5-{[3-(4-cyano- benzyl)-3H-imidazol-4-ylmeth- yl]heptylamino}benzo[b]-thio- phene-2-carboxamide | 596 | 97 |
| 177 | 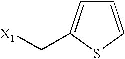 | X₂nHeptyl | N-(2-Thiophen-2-ylmethyl)-5-{[3-(4-cyano- benzyl)-3H-imidazol-4-ylmeth- yl]heptylamino}benzo[b]-thio- phene-2-carboxamide | 582 | 99 |
| 178 | X₁nButyl | X₂nHeptyl | N-Butyl-5-{[3-(4-cyanobenzyl)-3H-imi- | 542 | 99 |
| | | | dazol-4-ylmethyl]heptylamino}-ben- | | |
| | | | zo[b]thiophene-2-carboxamide | | |
| 179 | 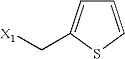 | X₂nButyl | N-(Thiophen-2-ylmethyl)-5-{[3-(4-cyano- benzyl)-3H-imidazol-4-ylmeth- yl]butylamino}benzo[b]-thio- phene-2-carboxamide | 540 | 99 |
| 180 | X₁nButyl | X₂uButyl | N-Butyl-5-{[3-(4-cyanobenzyl)-3H-imi- | 500 | 99 |
| | | | dazol-4-ylmethyl]butylamino}-ben- | | |
| | | | zo[b]thiophene-2-carboxamide | | |
| 181 | 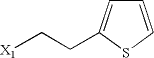 | 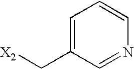 | N-(2-Thiophen-2-ylethyl)-5-{[3-(4-cyano- benzyl)-3H-imidazol-4-ylmeth- yl]pyridine-3-ylmethylamino}-ben- zo[b]thiophene-2-carboxamide | 589 | 94 |
| 182 | 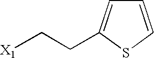 | 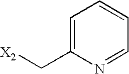 | N-(2-Thiophen-2-ylethyl)-5-{[3-(4-cyano- benzyl)-3H-imidazol-4-ylmeth- yl]pyridine-2-ylmethylamino}-ben- zo[b]thiphene-2-carboxamide | 589 | 96 |
| 183 | 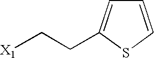 | 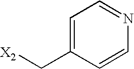 | N-(2-Thiophen-2-ylethyl)-5-{[3-(4-cyano- benzyl)-3H-imidazol-4-ylmeth- yl]pyridine-4-ylmethylamino}-ben- zo[b]thiophene-2-carboxamide | 589 | 93 |
| 184 | 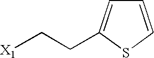 | 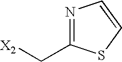 | N-(2-Thiophen-2-ylethyl)-5-{[3-(4-cyano- benzyl)-3H-imidazol-4-ylmeth- yl]thiazol-2-ylmethylamino}-ben- zo[b]thiophene-2-carboxamide | 595 | 94 |
| 185 | 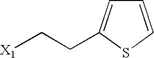 | 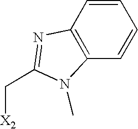 | N-(2-Thiophen-2-ylethyl)-5-{[3-(4-cyano- benzyl)-3H-imidazol-4-ylmeth- yl]-(1-methyl-1H-benzo- imidazol-2-ylmethyl)amino}-ben- zo[b]thiophene-2-carboxamide | 642 | 96 |

*HPLC conditions [C18 symmetry, 4.6 × 50 mm, 5 μm; λ = 220 nM; gradient 100% H₂O (+0.05% TFA) to 100% CH₂CN (+0.05% TFA) over 8 minutes]
¹H NMR, DMSO-d₆ (ppm).
Example 177: 9.30(t, 1H); 9.16(s, 1H); 7.90-7.86(m, 3H), 7.73(d, 1H); 7.50-7.40(m, 4H); 7.10-7.0(m, 2H); 6.97(dd, 1H); 6.84(dd, 1H); 5.60(s, 2H); 4.63(d, 2H); 4.45(s, 2H); 3.19(t, 2H); 1.38(bs, 2H); 1.3-1.2(m, 8H); 0.84(t, 3H).
Example 179: 9.30(t, 1H); 9.14(s, 1H); 7.9-7.86(m, 3H); 7.73(d, 1H); 7.5-7.4(m, 4H): 7.1-7.0(m, 2H); 6.98(dd, 1H); 6.83(dd, 1H); 5.59(s, 2H); 4.63(d, 2H); 4.46(s, 2H); 3.20(t, 2H); 1.38(quint., 2H); 1.21(sext., 2H); 0.83(t, 3H).
Example 181: 9.33(s, 1H); 8.93(t, 1H); 8.80(d, 1H); 8.76(s, 1H); 8.33(d, 1H); 7.96(t, 1H); 7.89(d, 2H); 7.83(s, 1H); 7.75(d, 1H); 7.60(s, 1H); 7.45(d, 2H); 7.33(d, 1H); 7.15-7.10(m, 2H); 7.00-6.90(m, 2H); 5.66(s, 2H); 4.79(s, 2H); 4.71(s, 2H); 3.48(q, 2H); 3.07(t, 2H).

### Examples 186 to 222

Compounds 186 to 222 are prepared from the derivative 127, 135 or 136, and from the corresponding acid chlorides, according to the conditions described for the preparation of 29, and abiding by the proportions of the various reagents. The products are then purified by filtration on silica using Combiflash Optix 10 (Isco) and using a gradient of methanol in dichloromethane (0 to 10%). Compounds 186 to 198 and 205 to 222 were taken up in a mixture of water, acetonitrile and TFA and then freeze-dried, in order to be characterized in the form of TFA salts.

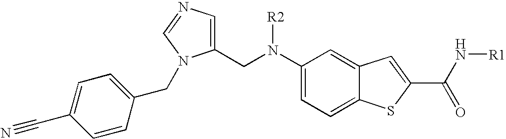

| | | | | Mass | HPLC |
|---|---|---|---|---|---|
| Ex. | R1 | R2 | Compound name | (M + H)⁺ | purity* |
| 186 | 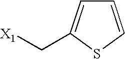 | X₂COEt | N-(Thiophen-2-ylmethyl)-5-{[3-(4-cyano- benzyl)-3H-imidazol-4-ylmeth- yl]propionylamino}benzo[b]thio- phene-2-carboxamide | 540 | 98 |
| 187 | 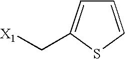 | 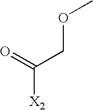 | N-(Thiophen-2-ylmethyl)-5-{[3-(4-cyano- benzyl)-3H-imidazol-4-ylmeth- yl]-(2-methoxyacetyl)-a- mino}benzo[b]thiophene-2-carbox- amide | 556 | 99 |
| 188 | 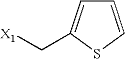 | 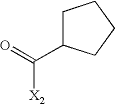 | N-(Thiophen-2-ylmethyl)-5-{[3-(4-cyano- benzyl)-3H-imidazol-4-ylmeth- yl]cyclopentanecarbonyl- amino}benzo[b]thiophene-2-carbox- amide | 580 | 98 |
| 189 | 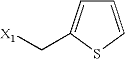 | 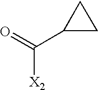 | N-(Thiophen-2-ylmethyl)-5-{[3-(4-cyano- benzyl)-3H-imidazol-4-ylmeth- yl]cyclopropanecarbonyl-a- mino}benzo[b]thiophene-2-carbox- amide | 552 | 98 |
| 190 | 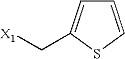 | 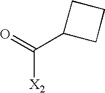 | N-(Thiophen-2-ylmethyl)-5-{[3-(4-cyano- benzyl)-3H-imidazol-4-ylmeth- yl]cyclobutanecarbonyl- amino}benzo[b]thiophene-2-carbo- xamide | 566 | 96 |
| 191 | 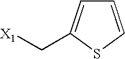 | 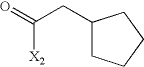 | N-(Thiophen-2-ylmethyl)-5-{[3-(4-cyano- benzyl)-3H-imidazol-4-ylmeth- yl]-(2-cyclopentylacetyl)-a- mino}benzo[b]thiophene-2-carbox- amide | 594 | 96 |
| 192 | X₁nButyl | 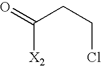 | N-Butyl-5-{(3-chloropropionyl)-[3-(4-cyano- benzyl)-3H-imidazol-4-ylmeth- yl]amino}benzo[b]-thio- phene-2-carboxamide | 534 | 98 |
| 193 | X₁nButyl | X₂COEt | N-Butyl-5-{[3-(4-cyanobenzyl)-3H-imi- | 500 | 99 |
| | | | dazol-4-ylmethyl]propionyl-a- | | |
| | | | mino}benzo[b]thiophene-2-carbox- | | |
| | | | amide | | |
| 194 | X₁nButyl | 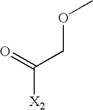 | N-Butyl-5-{[3-(4-cyanobenzyl)-3H-imi- dazol-4-ylmethyl]-(2-meth- oxyacetyl)amino}benzo[b]-thio- phene-2-carboxamide | 516 | 99 |
| 195 | X₁nButyl | 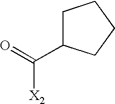 | N-Butyl-5-{[3-(4-cyanobenzyl)-3H-imi- dazol-4-ylmethyl]cyclopentane- carbonylamino}benzo[b]thiophene-2-carbox- amide | 540 | 99 |
| 196 | X₁nButyl | 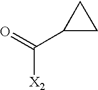 | N-Butyl-5-{[3-(4-cyanobenzyl)-3H-imi- dazol-4-ylmethyl]cyclopropane- carbonylamino}benzo[b]thiophene-2-carbox- amide | 512 | 99 |
| 197 | X₁nButyl | 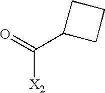 | N-Butyl-5-{[3-(4-cyanobenzyl)-3H-imi- dazol-4-ylmethyl]cyclobutane- carbonylamino}benzo[b]thiophene-2-carbox- amide | 526 | 99 |
| 198 | X₁nButyl | 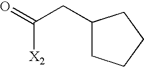 | N-Butyl-5-{[3-(4-cyanobenzyl)-3H-imi- dazol-4-ylmethyl]-(2-cyclo- pentylacetyl)amino}benzo[b]-thio- phene-2-carboxamide | 554 | 94 |
| 199 | 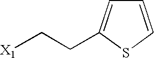 | X₂COnBu | N-(2-Thiophen-2-ylethyl)-5-{[3-(4-cyano- benzyl)-3H-imidazol-4-ylmeth- yl]pentanoylamino}benzo[b]thio- phene-2-carboxamide | 582 | 98 |
| 200 | 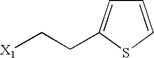 | X₂COnHexyl | N-(2-Thiophen-2-ylethyl)-5-{[3-(4-cyano- benzyl)-3H-imidazol-4-ylmeth- yl]heptanoylamino}benzo[b]thio- phene-2-carboxamide | 610 | 99 |
| 201 | 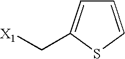 | X₂COnBu | N-(Thiophen-2-ylmethyl)-5-{[3-(4-cyano- benzyl)-3H-imidazol-4-ylmeth- yl]pentanoylamino}benzo[b]thio- phene-2-carboxamide | 568 | 96 |
| 202 | 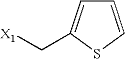 | X₂COnHexyl | N-(Thiophen-2-ylmethyl)-5-{[3-(4-cyano- benzyl)-3H-imidazol-4-ylmeth- yl]heptanoylamino}benzo[b]thio- phene-2-carboxamide | 596 | 98 |
| 203 | X₁nButyl | X₂COnBu | N-Butyl-5-{[3-(4-cyanobenzyl)-3H-imi- | 528 | 99 |
| | | | dazol-4-ylmethyl]pentanoyl-a- | | |
| | | | mino}benzo[b]thiophene-2-carbox- | | |
| | | | amide | | |
| 204 | X₁nButyl | X₂COnHexyl | N-Butyl-5-{[3-(4-cyanobenzyl)-3H-imi- | 556 | 99 |
| | | | dazol-4-ylmethyl]heptanoyl- | | |
| | | | amino}benzo[b]thiophene-2-carbox- | | |
| | | | amide | | |
| 205 | 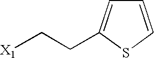 | 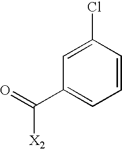 | N-(2-Thiophen-2-ylethyl)-5-{(3-chloro- benzoyl)-[3-(4-cyanobenzyl)-3H-imi- dazol-4-ylmethyl]amino}-ben- zo[b]thiophene-2-carboxamide | 636 | 99 |
| 206 | 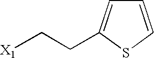 | 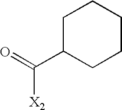 | N-(2-Thiophen-2-ylethyl)-5-{[3-(4-cyano- benzyl)-3H-imidazol-4-ylmeth- yl]cyclohexanecarbonyl- amino}benzo[b]thiophene-2-carbox- amide | 608 | 99 |
| 207 | 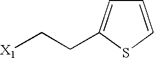 | 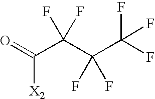 | N-(2-Thiophen-2-ylethyl)-5-{[3-(4-cyano- benzyl)-3H-imidazol-4-ylmeth- yl]-(2,2,3,3,4,4,4-hepta- fluorobutyryl)amino}benzo[b]-thio- phene-2-carboxamide | 694 | 99 |
| 208 | 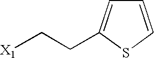 | 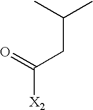 | N-(2-Thiophen-2-ylethyl)-5-{[3-(4-cyano- benzyl)-3H-imidazol-4-ylmeth- yl]-(3-methylbutyryl)-a- mino}benzo[b]thiophene-2-carbox- amide | 582 | 99 |
| 209 | 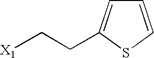 | 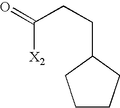 | N-(2-Thiophen-2-ylethyl)-5-{[3-(4-cyano- benzyl)-3H-imidazol-4-ylmeth- yl]-(3-cyclopentyl- propionyl)amino}benzo[b]-thio- phene-2-carboxamide | 622 | 99 |
| 210 | 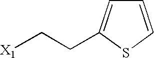 | X₂COnUndecyl | N-(2-Thiophen-2-ylethyl)-5-{[3-(4-cyano- benzyl)-3H-imidazol-4-ylmeth- yl]dodecanoylamino}benzo-[b]thio- phene-2-carboxamide | 680 | 98 |
| 211 | 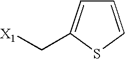 | 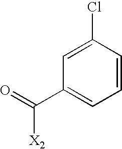 | N-(Thiophen-2-ylmethyl)-5-{(3-chloro- benzoyl)-[3-(4-cyanobenzyl)-3H-imi- dazol-4-ylmethyl]amino}-ben- zo[b]thiophene-2-carboxamide | 622 | 99 |
| 212 | 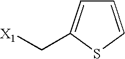 | 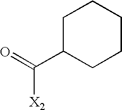 | N-(Thiophen-2-ylmethyl)-5-{[3-(4-cyano- benzyl)-3H-imidazol-4-ylmeth- yl]cyclohexanecarbonyl- amino}benzo[b]thiophene-2-carbox- amide | 594 | 94 |
| 213 | 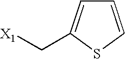 | 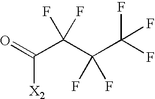 | N-(Thiophen-2-ylmethyl)-5-{[3-(4-cyano- benzyl)-3H-imidazol-4-ylmeth- yl]-(2,2,3,3,4,4,4-hepta- fluorobutyryl)amino}benzo[b]-thio- phene-2-carboxamide | 680 | 97 |
| 214 | 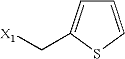 | 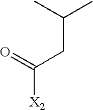 | N-(Thiophen-2-ylmethyl)-5-{[3-(4-cyano- benzyl)-3H-imidazol-4-ylmeth- yl]-(3-methylbutyryl)-a- mino}benzo[b]thiophene-2-carbox- amide | 568 | 98 |
| 215 | 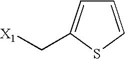 | 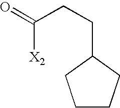 | N-(Thiophen-2-ylmethyl)-5-{[3-(4-cyano- benzyl)-3H-imidazol-4-ylmeth- yl]-(3-cyclopentyl-propio- nyl)amino}benzo[b]-thio- phene-2-carboxamide | 608 | 99 |
| 216 | 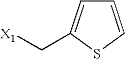 | X₂COnUndecyl | N-(Thiophen-2-ylmethyl)-5-{[3-(4-cyano- benzyl)-3H-imidazol-4-ylmeth- yl]dodecanoylamino}benzo-[b]thio- phene-2-carboxamide | 666 | 97 |
| 217 | X₁nButyl | 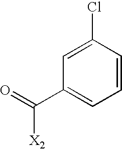 | N-Butyl-5-{(3-chlorobenzoyl)-[3-(4-cyano- benzyl)-3H-imidazol-4-ylmeth- yl]amino}benzo[b]-thio- phene-2-carboxamide | 582 | 97 |
| 218 | X₁nButyl | 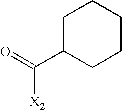 | N-Butyl-5-{[3-(4-cyanobenzyl)-3H-imi- dazol-4-ylmethyl]cyclohexane- carbonylamino}benzo[b]thiophene-2-carbox- amide | 554 | 99 |
| 219 | X₁nButyl | 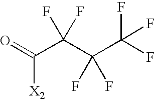 | N-Butyl-5-{[3-(4-cyanobenzyl)-3H-imi- dazol-4-ylmethyl]-(2,2,3,3,4,4,4-hepta- fluorobutyryl)amino}benzo-[b]thio- phene-2-carboxamide | 640 | 99 |
| 220 | X₁nButyl | 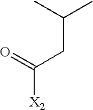 | N-Butyl-5-{[3-(4-cyanobenzyl)-3H-imi- dazol-4-ylmethyl]-(3-methyl- butyryl)amino}benzo[b]-thio- phene-2-carboxamide | 528 | 99 |
| 221 | X₁nButyl | 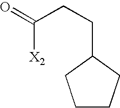 | N-Butyl-5-{[3-(4-cyanobenzyl)-3H-imi- dazol-4-ylmethyl]-(3-cyclo- pentylpropionyl)amino}benzo[b]thio- phene-2-carboxamide | 568 | 99 |
| 222 | X₁nButyl | X₂COnUndecyl | N-Butyl-5-{[3-(4-cyanobenzyl)-3H-imi- | 626 | 99 |
| | | | dazol-4-ylmethyl]dodecanoyl- | | |
| | | | amino}benzo[b]thiophene-2-carbox- | | |
| | | | amide | | |

*HPLC conditions [C18 symmetry, 4.6 × 50 mm, 50 μm; λ = 220 nM; gradient 100% H₂O (+0.05% TFA) to 100% CH₃CN (+0.05% TFA) over 8 minutes]
¹H NMR, DMSO-D₆(ppm).
Example 194: 9.22(s, 1H); 8.77(t, 1H); 8.03(d, 1H); 8.01(s, 1H); 7.9-7.8(m, 3H); 7.57(s, 1H); 7.42(d, 2H); 7.27(dd, 1H); 5.56(s, 2H); 4.90(s, 2H); 3.73(s, 2H); 3.30-3.25(m, H₂O+2H); 3.17(s, 3H); 1.52(quint., 2H); 1.35(sext., 2H); 0.91(t, 3H).
Example 201: 9.45(t, 1H); 8.04(s, 1H); 8.01(d, 1H); 7.80(d, 2H); 7.75(s, 1H); 7.60(s, 1H); 7.41(d, 1H); 7.21(d, 2H); 7.09(d, 1H); 7.05(s, 1H); 6.98(dd, 1H); 6.52(s, 1H); 5.32(s, 2H); 4.82(s, 2H); 4.65(d, 2H); 1.88(t, 2H); 1.34(quint., 2H); 1.07(sext., 2H); 0.71(t, 3H).
Example 202: 9.45(t, 1H); 8.04(s, 1H); 8.01(d, 1H); 7.80(d, 2H); 7.75(s, 1H); 7.59(s, 1H); 7.42(d, 1H); 7.21(d, 2H); 7.09(d, 1H); 7.05(d, 1H); 6.98(dd, 1H); 6.53(s, 1H); 5.32(s, 2H); 4.82(s, 2H); 4.65(d, 2H); 1.87(t, 2H); 1.35-1.00(m, 8H); 0.76(t, 3H).
Example 214: 9.48(t, 1H); 9.18(s, 1H); 8.1-8.0(m, 2H); 7.86(d, 2H); 7.79(d, 1H); 7.50(s, 1H); 7.45-7.40(m, 3H); 7.23(dd, 1H); 7.06(d, 1H); 6.99(dd, 1H); 5.56(s, 2H); 4.93(s, 2H); 4.66(d, 2H); 1.97(sep., 1H); 1.86(d, 2H); 0.74(d, 6H).

### EXAMPLE 223

Ethyl 5-[(3-benzyl-3H-imidazol-4-ylmethyl)amino]benzo[b]thiophene-2-carboxylate

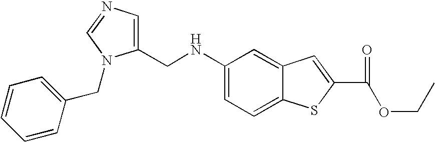

### Example 223A

### 4-(5-Formylimidazol-1-ylmethyl)benzene

Trifluoromethanesulfonic anhydride (0.99 ml; 5.9 mmol) dissolved in DCM (dichloromethane, 22 ml) is cooled to −65° C. under argon. Benzyl alcohol (0.61 ml; 5.9 mmol) dissolved in 9 ml of DCM in the presence of 2,6-ditert-butylpyridine (1.34 ml; 5.9 mmol) is added dropwise over 10 minutes. The reaction mixture is stirred for 15 minutes at −70° C. to complete the formation of the triflate. In a second flask, 1-trityl-1H-imidazole-4-carboxaldehyde (Daminos-Zeghal S. et al., Tetrahedron, 1997, 53(22), 7605-14) (2.0 g; 5.9 mmol) dissolved in DCM (14 ml), under a nitrogen atmosphere, is cooled to −70° C. The triflate solution is then cannulated into this second preparation over 25 minutes. Stirring at low temperature is continued for 2 hours and the cold bath is then removed. Once the reaction mixture has warmed to room temperature, it is neutralized by adding 20 ml of aqueous phosphate buffer solution (1.16 g Na₂HPO₄, 7H₂O; 0.7 g NaH₂PO₄; 20 ml H₂O). The phases are separated and the aqueous phase is extracted 3 times with DCM. The organic phases are combined, dried over magnesium sulfate, filtered and concentrated. The residual oil is then purified by flash chromatography (100% DCM, then 95/5 DCM/acetone and then 95/5 DCM/MeOH) to give the desired product (615 mg; 56%).

¹H NMR, DMSO-d₆ (ppm): 9.71 (s, 1H); 8.26 (s, 1H); 7.93 (s, 1H); 7.6-7.1 (m, 5H); 5.52 (s, 2H).

### EXAMPLE 223

Compound 223 is prepared from derivative 10B (1.5 g; 6.7 mmol) and the aldehyde 223A, according to the conditions described for the preparation of 34, and abiding by the proportions of the various reagents. Amount obtained: 2.19 g (86%). A fraction of this product is taken up in a mixture of water, acetonitrile and TFA, and then freeze-dried in order to be characterized.

HPLC (C₁₈, λ 220 nM), 100% H₂O to 100% CH₃CN (+0.1% TFA) over 8 minutes): purity: 99%.

Mass spectrum (ESI): m/z 392 (MH+).

¹H NMR, DMSO-dr (ppm): 9.2 (s, 1H); 7.89 (s, 1H); 7.72 (d, 1H); 7.64 (s, 1H); 7.5-7.3 (m, 5H); 7.0-6.85 (m, 2H); 6.8-6.0 (br s, 1H NH); 5.55 (s, 2H); 4.33 (q, 2H); 4.29 (s, 2H); 1.32 (t, 3H).

### EXAMPLE 224

### Ethyl 5-[(3-benzyl-3H-imidazol-4-ylmethyl)butylamino]benzo [b]thiophene-2-carboxylate

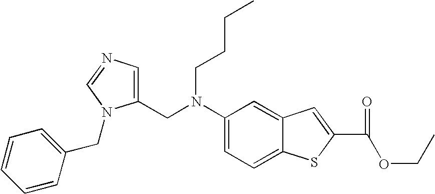

Compound 224 is prepared from the derivative 223 (2.65 g; 6.7 mmol) and n-butyraldehyde, according to the conditions described for the preparation of 34, and abiding by the proportions of the various reagents. Amount obtained: 1.23 g (41%). A fraction of this product is taken up in a mixture of water, acetonitrile and TFA, and then freeze-dried in order to be characterized.

HPLC (C₁₈, λ 220 nM), 100% H₂O to 100% CH₃CN (+0.1% TFA) over 8 minutes): purity: 96%.

Mass spectrum (ESI): m/z 448.(MH+).

¹H NMR, DMSO-d₆ (ppm): 9.21 (s, 1H); 7.91 (s, 1H); 7.76 (d, 1H); 7.55-7.4 (m, 4H); 7.35-7.3 (m, 2H); 7.05 (bs, 1H); 6.91 (dd, 1H); 5.52 (s, 2H); 4.58 (s, 2H); 4.34 (q, 2H); 3.28 (t, 2H); 1.43 (quint., 2H); 1.32 (t, 3H); 1.22 (sext., 2H); 0.85 (t, 3H).

### EXAMPLE 225

{5-[(3-Benzyl-3H-imidazol-4-ylmethyl)butylamino]benzo[b]thiophen-2-yl}methanol

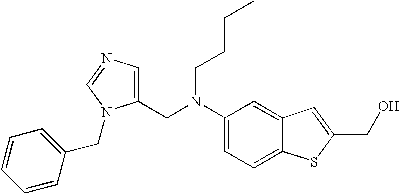

Compound 224 (842 mg; 1.88 mmol) is dissolved under a nitrogen atmosphere in anhydrous THF (25 ml). A solution of LiAlH4 (1M in THF; 3.76 ml; 3.76 mmol) is added dropwise at room temperature. After stirring for 1.5 hours, the reaction mixture is neutralized by successive addition of water (143 el), sodium hydroxide (15% in water; 143 ll) and water (429 el). A precipitate forms. It is filtered off and rinsed with DCM. The filtrate is concentrated to give the desired product (682 mg; 89%). A fraction of this product is taken up in a mixture of water, acetonitrile and TFA, and then freeze-dried in order to be characterized.

HPLC (C₁₈, λ 220 nM), 100% H₂O to 100% CH₂CN (+0.1% TFA) over 8 minutes): purity: 98%.

Mass spectrum (ESI): m/z 406 (MH+).

¹H NMR, DMSO-d₆ (ppm): 7.73 (s, 1H); 7.57 (d, 1H); 7.45-7.3 (m, 3H); 7.09 (d, 2H); 7.00 (s, 1H); 6.90 (d, 1H); 6.71 (dd, 1H); 6.66 (s, 1H); 5.54 (t, 1H, OH); 5.21 (s, 2H); 4.66 (d, 2H); 4.27 (s, 2H); 3.16 (t, 2H); 1.45-1.3 (m, 2H); 1.3-1.15 (m, 2H); 0.81 (t, 3H).

### EXAMPLE 226

N-(3-Benzyl-3H-imidazol-4-ylmethyl)-N-butyl-(2-ethoxymethylbenzol[b]thiophen-5-yl)amine

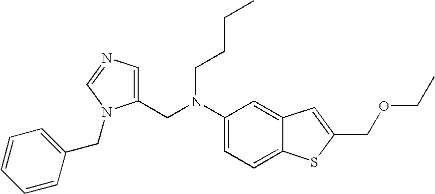

Compound 225 (100 mg; 0.25 mmol) is dissolved under a nitrogen atmosphere in anhydrous DMF (2 ml) in the presence of NaH (60%; 28 mg; 0.49 mmol). The suspension is cooled to 0° C. and ethane bromide (27 μl; 0.37 mmol) is then added. The cold bath is removed. After stirring for 2.5 hours, ethyl acetate and water are added. The phases are separated and the aqueous phase is extracted twice with ethyl acetate. The organic phases are combined, dried over magnesium sulfate, filtered and concentrated. The residual oil is purified by preparative HPLC (Waters Prep 4000) on a Prep Nova-Pak HR C-18 column (Waters; 25×100 mm; 6 μn) using a total gradient of from 100% water (0.1% TFA) to 100% acetonitrile (0.1% TFA) over 15 minutes, to give the desired product in the form of the TFA salt (111 mg; 82%).

HPLC (C₁₈, λ 220 nM), 100% H₂O to 100% CH₃CN (+0.1% TFA) over 8 minutes): purity: 99%.

Mass spectrum (ESI): m/z 434 (MH+).

¹H NMR, DMSO-d₆ (Ppm): 9.21 (s, 1H); 7.62 (d, 1H); 7.5-7.35 (m, 4H); 7.32 (d, 2H); 7.08 (s, 1H); 6.88 (d, 1H); 6.71 (dd, 1H); 5.51 (s, 2H); 4.58 (s, 2H); 4.44 (s, 2H); 3.49 (q, 2H); 3.24 (t, 2H); 1.41 (quint., 2H); 1.23 (sext., 2H); 1.14 (t, 3H); 0.84 (t, 3H).

### EXAMPLE 227

### N-(3-Benzyl-3H-imidazol-4-ylmethyl)-N-butyl-(2-propylaminomethylbenzo[b]thiophen-5-yl)amine

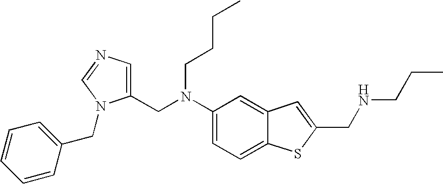

### Example 227A

{5-[(3-Benzyl-3H-imidazol-4-ylmethyl)butylamino]benzo[bl-thiophen-2-yl}methanal. Compound 225 (799 mg; 1.97 mmol) is dissolved in DMSO (10 ml) at room temperature and under a nitrogen atmosphere. Triethylamine (1.1 ml; 7.88 mmol) and sulfur trioxide/pyridine complex (783 mg; 4.92 mmol) are added, and the reaction mixture is then stirred for 5 hours. Ethyl acetate (50 ml) is added and the reaction mixture is washed with 80 ml of water. The aqueous phase is extracted twice with ethyl acetate. The organic phases are combined, dried over magnesium sulfate, filtered and concentrated. Since the residual oil is a mixture of the desired product with compound 225 it is subjected to this oxidizing treatment again, followed by the same washing. The new residual oil is purified by flash chromatography (100% DCM and then 95/5 DCM/MeOH) to give the desired product (703 mg; 88%).

HPLC (C₁₈, λ 220 nM), 100% H₂O to 100% CH₃CN (+0.1% TFA) over 8 minutes): purity: 99%.

Mass spectrum (ESI): m/z 404 (MH+).

¹H NMR, DMSO-d₆ (ppm): 10.09 (s, 1H); 9.22 (s, 1H); 8.13 (s, 1H); 7.81 (d, 1H); 7.5-7.4 (m, 4H); 7.32 (d, 2H); 7.11 (d, 1H); 6.96 (dd, 1H); 5.52 (s, 2H); 4.50 (s, 2H); 3.29 (t, 2H); 1.44 (quint., 2H); 1.24 (sext., 2H); 0.85 (t, 3H).

### EXAMPLE 227

Compound 227 is prepared from the derivative 227A (120 mg; 0.3 mmol) and n-propylamine, according to the conditions described for the preparation of 34, and abiding by the proportions of the various reagents. The residual oil is purified by preparative HPLC (Waters Prep 4000) on a Prep Nova-Pak HR C-18 column (Waters; 25×100 mm; 6 μm) using a total gradient of from 100% water (0.1% TFA) to 100% acetonitrile (0.1% TFA) over 15 minutes, to give the desired product in the form of the TFA salt (47 mg; 46%).

HPLC (C₁₈, λ 220 nM), 100% H₂O to 100% CH₃CN (+0.1% TFA) over 8 minutes): purity: 99%.

Mass spectrum (ESI): m/z 447 (MH+).

¹H NMR, DMSO-dr (ppm): 9.21 (s, 1H); 9.05 (br s, 1H, NH); 7.71 (d, 1H); 7.5-7.3 (m, 7H); 6.94 (d, 1H); 6.78 (dd, 1H); 5.51 (s, 2H); 4.52 (bs, 4H); 3.26 (t, 2H); 2.89 (bs, 2H); 1.63 (sext., 2H); 1.41 (quint., 2H); 1.23 (sext., 2H); 0.90 (t, 3H); 0.84 (t, 3H).

### EXAMPLE 228

N-(3-Benzyl-3H-imidazol-4-ylmethyl)-N-butyl(2-pent-1-enylbenzo[b]thiophen-5-yl)amine

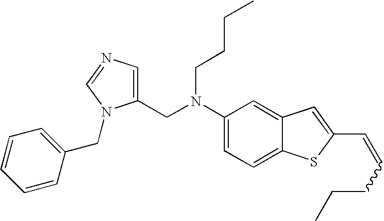

### Example 228A

Butyltriphenylphosphonium iodide. Triphenylphosphine (4.35 g; 17 mmol) is dissolved in toluene (75 ml) under a nitrogen atmosphere, in the presence of 1-iodobutane (1.8 ml; 16 mmol). The reaction mixture is heated overnight at 90° C. and then cooled to 0° C. The desired product precipitates out. It is filtered off and dried (2.55 g; 36%).

¹H NMR, DMSO-d₆ (ppm): 8.0-7.7 (m, 15H); 3.8-3.6 (m, 2H); 1.49 (bs, 4H); 0.89 (t, 3H).

### EXAMPLE 228

Compound 228A (1.1 g; 0.49 mmol) is dissolved, under a nitrogen atmosphere, in 1,4-dioxane (4 ml) and potassium tert-butoxide (1M/THF; 2.5 ml; 2.5 mmol) is then added. A bright orange coloration appears immediately. Compound 227A (200 mg; 0.19 mmol), prediluted in dioxane (4 ml) is then added. The reaction mixture is stirred for 20 minutes at room temperature and then neutralized by adding water. The aqueous phase is extracted three times with DCM. The organic phases are combined, dried over magnesium sulfate, filtered and concentrated. The residual oil is purified by preparative HPLC using a total gradient of from 100% water (0.1% TFA) to 100% acetonitrile (0.1% TFA) over 50 minutes, to give the desired product in the form of the TFA salt (279 mg; 100%).

HPLC (C₁₈, λ 220 nM), 100% H₂O to 100% CH₃CN (+0.1% TFA) over 8 minutes): purity: 99%.

Mass spectrum (ESI): m/z 444 (MH+).

¹H NMR, DMSO-d₆ (ppm): 9.22 (s, 1H); 7.63 (d, 0.7H); 7.55 (d, 0.3H); 7.5-7.3 (m, 6H); 7.09 (s, 0.7H); 6.99 (s, 0.3H); 6.86 (d, 0.7H); 6.81 (d, 0.3H); 6.75-6.65 (m, 2H); 6.06 (dt, 0.3H); 5.70 (dt, 0.7H); 5.51 (s, 2H); 4.45 (s, 1.4H); 4.43 (s, 0.6H); 3.3-3.15 (m, 2H); 2.42 (q, 1.4H); 2.17 (q, 0.6H); 1.6-1.35 (m, 4H); 1.3-1.2 (m, 2H); 0.95 (t, 2.1H); 0.89 (t, 0.9H); 0.82 (t, 3H). Mixture of the cis/trans isomers (70/30).

### EXAMPLE 229

### N-(3-Benzyl-3H-imidazol-4-ylmethyl)-N-butyl-(2-pentylbenzo[b]thiophen-5-yl)amine

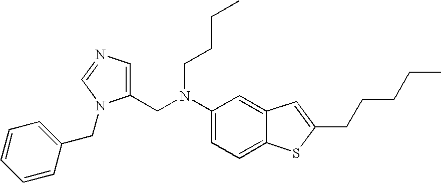

Compount 228 (100 mg; 0.18 mmol) dissolved in methanol (50 ml) is hydrogenated (36 psi) using a Parr hydrogenator and palladium-on-charcoal (10%; 38 mg; 0.04 mmol) for 7 hours. The reaction medium is then degassed by bubbling nitrogen through, filtered through Celite and concentrated. The residual oil is purified by preparative HPLC (Waters Prep 4000) on a Prep Nova-Pak HR C-18 column (Waters; 25×100 mm; 6 μm) using a total gradient of from 100% water (0.1% TFA) to 100% acetonitrile (0.1% TFA) over 15 minutes, to give the desired product in the form of the TFA salt (69 mg; 69%).

HPLC (C₁₈, λ 220 nM), 100% H₂O to 100% CH₃CN (+0.1% TFA) over 8 minutes): purity: 99%.

Mass spectrum (ESI): m/z 446 (MH+).

¹H NMR, DMSO-d₆ (ppm): 9.21 (s, 1H); 7.56 (d, 1H); 7.5-7.4 (m, 3H); 7.39 (s, 1H); 7.31 (d, 2H); 6.87 (s, 1H); 6.82 (d, 1H); 6.66 (dd, 1H); 5.51 (s, 2H); 4.42 (s, 2H); 3.23 (t, 2H); 2.81 (t, 2H); 1.65 (quint., 2H); 1.5-1.15 (m, 8H); 0.95-0.8 (m, 6H).

### EXAMPLE 230

### Ethyl 4-{[3-(4-cyanobenzyl)-3H-imidazol-4-ylmethyl]amino}-3-propylbenzo[b]thiophene-2-carboxylate

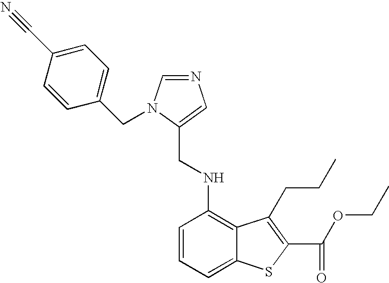

### Example 230A

### 2′-Fluoro-6′-benzylaminobutyrophenone

2′,6′-Difluorobutyrophenone (10 g, 54 mmol) is dissolved in DMF (110 ml) under a nitrogen atmosphere, in the presence of benzylamine (5.9 ml; 54 mmol) and potassium carbonate (11 g; 81 mmol). The mixture is stirred for 18 hours at 140° C. The reaction mixture is then cooled, neutralized by addition of water and extracted three times with ethyl acetate. The organic phases are combined, dried over magnesium sulfate, filtered and concentrated to give the desired product (10.3 g; 68%).

¹H NMR, DMSO-d₆ (ppm): 8.90 (t, 1H); 7.4-7.1 (m, 6H); 6.52 (d, 1H); 6.40 (dd, 1H); 4.45 (d, 2H); 2.85 (t, 2H); 1.62 (sext., 2H); 0.91 (t, 3H).

### Example 230B

### Ethyl 4-benzylamino-3-propylbenzo[b]thiophene-2-carboxylate.

Compound 230B is prepared from compound 230A (11.4 g; 42 mmol) according to the conditions used for the preparation of 1B, and abiding by the proportion of the various reagents. Amount obtained: 11.1 g (74%).

¹H NMR, DMSO-d₆ (ppm): 7.43 (d, 2H); 7.35 (t, 2H); 7.27-7.16 (m, 3H); 6.47 (d, 1H); 5.85 (t, 1H); 4.45 (d, 2H); 4.30 (q, 2H); 3.50 (t, 2H); 1.64 (sext., 2H); 1.31 (t, 3H); 0.89 (t, 3H).

### Example 230C

### Ethyl 4-amino-3-propylbenzo[b]thiophene-2-carboxylate

Compound 230B (3.73 g; 10 mmol), dissolved in a mixture of ethanol and THF (50/50; 50 ml) is hydrogenated (36 psi) using a Parr hydrogenator and palladium hydroxide-on-charcoal (20%; 5.93 g; 8 mmol), for 1.5 hours. The reaction medium is then degassed by bubbling nitrogen through, filtered through Celite and concentrated. The residual oil is coevaporated twice with toluene to give a yellow solid. This solid is purified by flash chromatography (60/40 EDP/DCM to 100% DCM) to give the desired product (1.98 g; 63%).

¹H NMR, DMSO-d₆ (ppm): 7.19 (t, 1H); 7.12 (d, 1H); 6.66 (d, 1H); 5.47 (s, 2H); 4.29 (q, 2H); 3.41 (t, 2H); 1.64 (sext., 2H); 1.31 (t, 3H); 0.96 (t, 3H).

### EXAMPLE 230

Compound 230C (754 mg; 3.2 mmol) is dissolved in 1,2-DCE (28 ml) in the presence of derivative 27A (809 mg; 3.8 mmol) and acetic acid (820 μl; 14 mmol) at room temperature and under a nitrogen atmosphere, for a few minutes, and sodium triacetoxyborohydride (1.03 g; 4.9 mmol) is then added. After stirring for 18 hours, ethyl acetate (50 ml) and saturated aqueous sodium bicarbonate solution (100 ml) are added. The aqueous phase is extracted twice with ethyl acetate. The organic phases are combined, dried over magnesium sulfate, filtered and concentrated. The crude reaction product is then purified by flash chromatography to give the intermediate imine (688 mg). This imine is dissolved in methanol (5 ml) and THF (18 ml) under a nitrogen atmosphere and at room temperature, and sodium borohydride (170 mg) is then added. After stirring for 18 hours, the reaction mixture is concentrated and then taken up in DCM and filtered through Celite. The filtrate is concentrated and the residue is purified by flash chromatography (20/80 acetone/DCM and then 90/10 DCM/MeOH) to give the desired product (790 mg; 66%). A fraction of this product is taken up in a mixture of water, acetonitrile and TFA, and then freeze-dried in order to be characterized.

HPLC (C₁₈, λ220 nM, 100% H₂O to 100% CH₃CN (+0.1% TFA) over 8 minutes): purity: 83%.

Mass spectrum (ESI): m/z 459 (MH+).

¹H NMR, DMSO-d₆ (ppm): 9.23 (s, 1H); 7.76 (d, 2H); 7.68 (s, 1H); 7.36 (d, 2H); 7.26 (t, 1H); 7.21 (d, 1H); 6.46 (d, 1H); 5.73 (s, 2H); 4.46 (s, 2H); 4.29 (q, 2H); 3.30 (t, 2H); 1.54 (sext., 2H); 1.31 (t, 3H); 0.84 (t, 3H).

### EXAMPLES 231 AND 232

Compounds 231 and 232 are prepared in the form of TFA salts from the derivative 230 and from the corresponding acid chlorides, according to the conditions described for the preparation of 231, and abiding by the proportions of the various reagents.

Compound 230 (400 mg; 0.87 mmol) is dissolved in pyridine (23 ml) under a nitrogen atmosphere, and benzoyl chloride (607 μl; 5.2 mmol) is then added. The solution is stirred for 5 hours at 60° C. and then for 18 hours at room temperature. The reaction mixture is coevaporated twice with toluene (2×30 ml). The residue is taken up in water (80 ml) and extracted with dichloromethane (6×100 ml). The organic phases are combined, washed with saturated aqueous NaCl solution, dried over magnesium sulfate, filtered and concentrated. This crude reaction product is then purified by flash chromatography to give product 231 (368 mg; 75%).

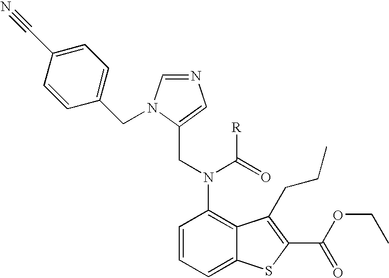

| | | | Mass | HPLC |
|---|---|---|---|---|
| Ex. | R | Compund name | (M + H)⁺ | purity* |
| 231 | 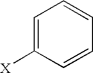 | Ethyl 4-{benzoyl-[3-(4-cyanobenzyl)-3H-imi- dazol-4-ylmethyl]amino}-3-propyl- benzo[b]thiophene-2-carboxylate | 563 | 80 |
| 232 | 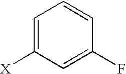 | Ethyl 4-[[3-(4-cyanobenzyl)-3H-imidazol-4-ylmeth- yl]-(3-fluorobenzoyl)amino]-3-propyl- benzo[b]thiophene-2-carboxylate | 581 | 80 |

*HPLC conditions [C18 symmetry, 4.6 × 50 mm, 5 μm; λ = 220 nM; gradient 100% H₂O (+0.05% TFA) to 100% CH₃CN (+0.05% TFA) over 8 minutes]
¹H NMR, DMSO-d₆(ppm):
Example 231; 7.92(d, 1H); 7.81(s, 1H); 7.69(d, 2H); 7.46(t, 1H); 7.35-7.25(m, 1H); 7.20-7.00(m, 6H); 6.83(s, 1H); 6.77(d, 1H); 5.54(d, 1H); 5.42(d, 1H); 5.22(d, 1H); 4.34(q, 2H); 4.20(d, 1H); 3.30-3.10(m, 2H); 1.55-1.35(m, 2H); 1.33(t, 3H); 0.89(t, 3H).
Example 232: 7.95(d, 1H); 7.82(s, 1H); 7.70(d, 2H); 7.33(t, 2H); 7.15-7.00(m, 4H); 6.90-6.79(m, 4H); 5.54(d, 1H); 5.42(d, 1H); 5.26(d, 1H); 4.34(q, 2H); 4.22(d, 1H); 3.22(td, 1H); 3.10(td, 1H); 1.55-1.30(m, 2H); 1.33(t, 3H); 0.91(t, 3H).

### EXAMPLE 233

Ethyl 5-{[3-(4-cyanobenzyl)-3H-imidazol-4-ylmethyl]-(3-methylbutyryl)amino}benzo[b]thiophene-2-carboxylate

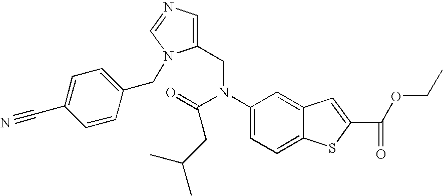

Compound 34 (1.77 g; 4.25 mmol) is dissolved in DCM (70 ml) under a nitrogen atmosphere, in the presence of DIPEA (2.12 ml; 12.2 mmol). Isovaleryl chloride (1.35 ml; 11 mmol) is added dropwise and the reaction mixture is stirred at room temperature for 20 hours. It is then neutralized by adding water. The aqueous phase is extracted twice with DCM. The organic phases are combined, dried over magnesium sulfate, filtered and concentrated. The residue is purified by flash chromatography (90/10 DCM/acetone and then 95/5 DCM/MeOH) to give the desired product (870 mg; 41%).

HPLC (C₁₈, λ 220 nM), 100% H₂O to 100% CH₃CN (+0.1% TFA) over 8 minutes): purity: 97%.

Mass spectrum (ESI): m/z 501 (MH+).

¹H NMR, DMSO-d₆ (ppm): 8.12 (s, 1H); 8.05 (d, 1H); 7.8-7.7 (m, 3H); 7.63 (s, 1H); 7.25-7.1 (m, 3H); 6.56 (s, 1H); 5.31 (s, 2H); 4.83 (s, 2H); 4.36 (q, 2H); 2.0-1.8 (M, 1H); 1.78 (d, 2H); 1.40 (t, 3H); 0.72 (d, 6H).

### EXAMPLE 234

5-[[3-(4-Cyanobenzyl)-3H-imidazol-4-ylmethyl]-(3-methylbutyryl)amino]benzo[b]thiophene-2-carboxylic acid

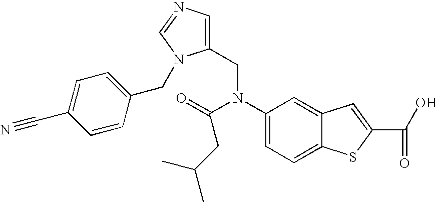

Compound 234 is prepared from the derivative 233 (870 mg; 1.73 mmol), according to the conditions described for the preparation of 1D, and abiding by the proportions of the various reagents, but with heating only at 40° C. for 2 hours. Amount obtained: 668 mg (82%).

HPLC (C₁₈, λ220 nm), 100% H₂O to 100% CH₃CN (+0.1% TFA) over 8 minutes): purity: 94%.

¹H NMR, DMSO-d₆ (ppm): 8.02 (d, 1H); 8.01 (s, 1H); 7.78 (d, 2H); 7.77 (s, 1H); 7.60 (s, 1H); 7.19 (d, 2H); 7.15 (d, 1H); 5.32 (s, 2H); 4.83 (s, 2H); 2.0-1.9 (M, 1H); 1.78 (d, 2H); 0.72 (d, 6H).

### Examples 235 to 247

Compounds 235 to 247 are prepared in the form of TFA salts from the derivative 234 and from the corresponding amines, according to the conditions described for the preparation of 40, and abiding by the proportions of the various reagents. The products are then purified by filtration on silica using CombiFlash Optix 10 (Isco), and using a gradient of methanol in dichloromethane (0 to 10%). They are then taken up in water, acetonitrile and TFA, and then freeze-dried in order to be characterized.

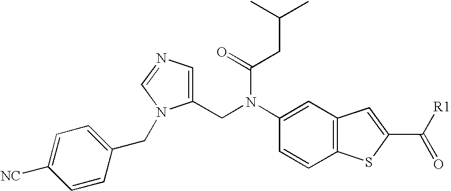

| | | | Mass | HPLC |
|---|---|---|---|---|
| Ex. | R | Compound name | (M + H)⁺ | purity* |
| 235 | 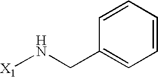 | N-(Benzyl)-5-{[3-(4-cyanobenzyl)-3H-imi- dazol-4-ylmethyl]-(3-methylbutyryl)-a- mino}benzo[b]thiophene-2-carboxamide | 562 | 99 |
| 236 | 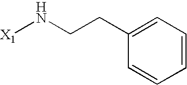 | N-(Phenethyl)-5-{[3-(4-cyanobenzyl)-3H-imi- dazol-4-ylmethyl]-(3-methylbutyryl)-a- mino}benzo[b]thiophene-2-carboxamide | 576 | 95 |
| 237 | 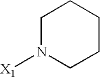 | N-[3-(4-Cyanobenzyl)-3H-imidazol-4-ylmeth- yl]-N-[2-(piperidine-1-carbonyl)-ben- zo[b]thiphen-5-yl]-3-methylbutyramide | 540 | 91 |
| 238 | 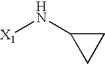 | N-(Cyclopropyl)-5-{[3-(4-cyanobenzyl)-3H-imi- dazol-4-ylmethyl]-(3-methyl- butyryl)amino}benzo[b]thiophene-2-carbox- amide | 512 | 99 |
| 239 | 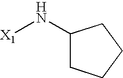 | N-(Cyclopentyl)-5-{[3-(4-cyanobenzyl)-3H-imi- dazol-4-ylmethyl]-(3-methylbutyryl)-a- mino}benzo[b]thiophene-2-carboxamide | 540 | 95 |
| 240 | 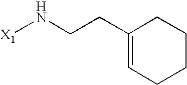 | N-(2-Cyclohex-1-enylethyl)-5-{[3-(4-cyano- benzyl)-3H-imidazol-4-ylmethyl]-(3-meth- ylbutyryl)amino}benzo[b]thiophene-2-carbox- amide | 580 | 96 |
| 241 | 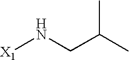 | N-Isobutyl-5-{[3-(4-cyanobenzyl)-3H-imi- dazol-4-ylmethyl]-(3-methylbutyryl)-a- mino}benzo[b]thiophene-2-carboxamide | 528 | 96 |
| 242 | 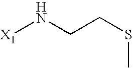 | N-(2-Methylsulfanylethyl)-5-{[3-(4-cyano- benzyl)-3H-imidazol-4-ylmethyl]-(3-methyl- butyryl)amino}benzo[b]thiophene-2-carbox- amide | 546 | 93 |
| 243 | 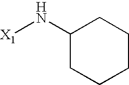 | N-Cyclohexyl-5-{[3-(4-cyanobenzyl)-3H-imi- dazol-4-ylmethyl]-(3-methylbutyryl)-a- mino}benzo[b]thiophene-2-carboxamide | 554 | 99 |
| 244 | 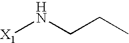 | N-Propyl-5-{[3-(4-cyanobenzyl)-3H-imi- dazol-4-ylmethyl]-(3-methylbutyryl)-a- mino}benzo[b]thiophene-2-carboxamide | 514 | 95 |
| 245 | 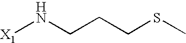 | N-(3-Methylsulfanylpropyl)-5-{[3-(4-cyano- benzyl)-3H-imidazol-4-ylmethyl]-(3-methyl- butyryl)amino}benzo[b]thiophene-2-carbox- amide | 560 | 98 |
| 246 | 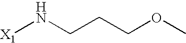 | N-(3-Methoxypropyl)-5-{[3-(4-cyano- benzyl)-3H-imidazol-4-ylmethyl]-(3-methyl- butyryl)amino}benzo[b]thiophene-2-carbox- amide | 544 | 100 |
| 247 | 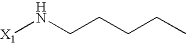 | N-Pentyl-5-{[3-(4-cyanobenzyl)-3H-imi- dazol-4-ylmethyl]-(3-methylbutyryl)-a- mino}benzo[b]thiophene-2-carboxamide | 542 | 99 |

*HPLC conditions [C₁₈ symmetry, 4.6 × 50 mm, 5 μm; λ = 220 nM; gradient 100% H₂O (+0.05% TFA) to 100% CH₃CN (+0.05% TFA) over 8 minutes]
¹H NMR, DMSO-d₆ (ppm):
Example 244: 9.17(s, 1H); 8.79(t, 1H); 8.05-8.00(m, 2H); 7.85(d, 2H); 7.77(d, 1H); 7.49(s, 1H); 7.41(d, 2H); 7.21(dd, 1H); 5.56(s, 2H); 4.93(s, 2H); 3.24(q, 2H); 1.96(sept., 1H); 1.86(d, 2H); 1.55(sext., 2H); 0.91(t, 3H); 0.74(d, 6H).

### EXAMPLE 248

Ethyl 5-{[3-(4-cyanobenzyl)-3H-imidazol-4-ylmethyl]pentanoylamino]benzo[b]thiophene-2-carboxylate

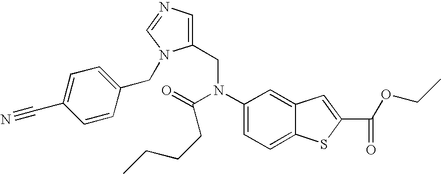

Compound 248 is prepared from the derivative 34 (5.48 g; 13 mmol) and n-pentanoyl chloride, according to the conditions described for the preparation of 233 and abiding by the proportions of the various reagents. Amount obtained: 5.41 g (83%).

HPLC (C₁₈, λ 220 nm), 100% H₂O to 100% CH₃CN (+0.1% TFA) over 8 minutes): purity: 98%.

¹H NMR, DMSO-d₆ (ppm): 8.13 (s, 1H); 8.05 (d, 1H); 7.8-7.75 (m, 3H); 7.64 (s, 1H); 7.3-7.1 (m, 3H); 6.55 (s, 1H); 5.32 (s, 2H); 4.82 (s, 2H); 4.36 (q, 2H); 1.9-1.8 (M, 2H); 1.4-1.25 (m, 5H); 1.06 (sext., 2H); 0.70 (t, 3H).

### EXAMPLE 249

5-{[3-(4-Cyanobenzyl)-3H-imidazol-4-ylmethyl]-pentanoylamino}benzo[b]thiophene-2-carboxylic acid

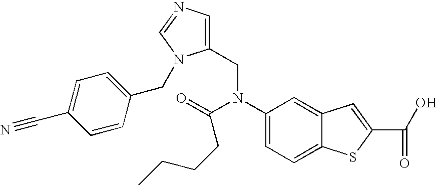

Compound 249 is prepared from the derivative 248 (5.41 g; 11 mmol), according to the conditions described for the preparation of 234, and abiding by the proportions of the various reagents. Amount obtained: 5.1 g (98%).

HPLC (C₁₈, λ 220 nm), 100% H₂O to 100% CH₃CN (+0.1% TFA) over 8 minutes): purity: 98%.

Mass spectrum (ESI): m/z 473 (MH+).

¹H NMR, DMSO-d₆ (ppm): 7.91 (d, 1H); 7.80 (d, 2H); 7.75 (s, 1H); 7.73 (s, 1H); 7.52 (bs, 1H); 7.21 (d, 2H); 7.04 (d, 1H); 6.54 (s, 1H); 5.32 (s, 2H); 4.82 (s, 2H); 1.87 (t, 2H); 1.34 (quint., 2H); 1.07 (sext., 2H); 0.71 (t, 3H).

### Examples 250 to 256

Compounds 250 to 256 are prepared in the form of HCl salts from the derivative 249 and from the corresponding amines, according to the conditions described for the preparation of 40, and abiding by the proportions of the various reagents. The products are then purified by preparative HPLC (Waters Prep 4000) on a Prep Nova-Pak HR C-18 column (Waters; 25×100 mm; 6 μm) using a total gradient of from 100% water (0.1% HCl) to 100% acetonitrile (0.1% HCl) over 15 minutes, and then freeze-dried to give the desired products in the form of HCl salts.

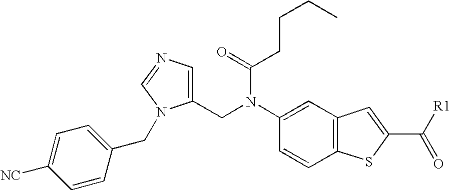

| | | | Mass | HPLC |
|---|---|---|---|---|
| Ex. | R | Compound name | (M + H)⁺ | purity* |
| 250 | 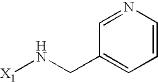 | N-(Pyrid-3-ylmethyl)-5-{[3-(4-cyano- benzyl)-3H-imidazol-4-ylmethyl]pentanoyl- amino}benzo[b]thiophene-2-carboxamide | 563 | 99 |
| 251 | 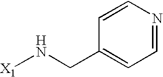 | N-(Pyrid-4-ylmethyl)-5-{[3-(4-cyano- benzyl)-3H-imidazol-4-ylmethyl]pentanoyl- amino}benzo[b]thiophene-2-carboxamide | 563 | 99 |
| 252 | 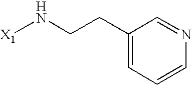 | N-(2-Pyrid-3-ylmethyl)-5-{[3-(4-cyano- benzyl)-3H-imidazol-4-ylmethyl]pentanoyl- amino}benzo[b]thiophene-2-carboxamide | 577 | 99 |
| 253 | 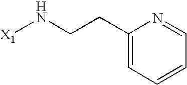 | N-(2-Pyrid-2-ylethyl)-5-{[3-(4-cyano- benzyl)-3H-imidazol-4-ylmethyl]pentanoyl- amino}benzo[b]thiophene-2-carboxamide | 577 | 99 |
| 254 | 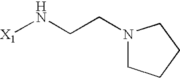 | N-(2-Pyrrolidin-1-ylethyl)-5-{[3-(4-cyano- benzyl)-3H-imidazol-4-ylmethyl]pentanoyl- amino}benzo[b]thiophene-2-carboxamide | 569 | 99 |
| 255 | 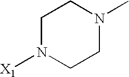 | N-[3-(4-Cyanobenzyl)-3H-imidazol-4-ylmeth- yl]-N-[2-(4-methylpiperazine-1-carbo- nyl)benzo[b]thiophen-5-yl]pentan- amide | 555 | 92 |
| 256 | 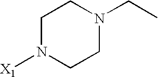 | N-[3-(4-Cyanobenzyl)-3H-imidazol-4-ylmeth- yl]-N-[2-(4-ethylpiperazine-1-carbo- nyl)benzo[b]thiophen-5-ylpentan- amide | 569 | 94 |

*HPLC conditions [C₁₈ symmetry, 4.6 × 50 mm, 5 μm; λ = 220 nm; gradient 100% H₂O (+0.05% TFA) to 100% CH₃CN (+0.05% TFA) over 8 minutes]
¹NMR, DMSO-d₆ (ppm):
Example 252: 9.27(s, 1H); 9.09(t, 1H); 8.84(s, 1H); 8.74(d, 1H); 8.39(d, 1H); 8.07-8.00(m, 2H); 7.90(t, 1H); 7.85(d, 2H); 7.78(s, 1H); 7.53(s, 1H); 7.42(d, 2H); 7.23(d, 1H); 5.58(s, 2H); 4.91(s, 2H); 3.63(t, 2H); 3.08(t, 2H); 1.95(t, 2H); 1.38(quint., 2H); 1.09(sext., 2H); 0.72(t, 3H).

### EXAMPLE 257

N-(Pyrid-3-ylmethyl)-5-{[3-(4-cyanobenzyl)-3H-imidazol-4-ylmethyl]amino}benzo[b]thiophene-2-carboxamide

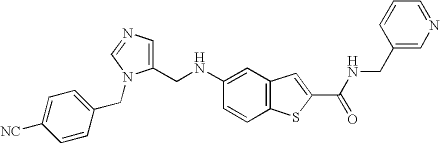

### Example 257A

### 5-Nitrobenzo[b]thiophene-2-carboxylic acid

Compound 257A is prepared from the derivative 10A (6.48 g; 29 mmol), according to the conditions used for the preparation of 1D and abiding by the proportions of the various reagents. Amount obtained: 13.1 g (99%).

HPLC (C₁₈, λ 220 nm), 100% H₂O to 100% CH₃CN (+0.1% TFA) over 8 minutes): purity: 99%.

Mass spectrum (ESI): m/z 221 (M−H—).

¹H NMR, DMSO-d₆ (ppm): 13.88 (bs, 1H); 8.98 (s, 1H); 8.4-8.3 (m, 3H).

### Example 257B

### N-(Pyrid-3-ylmethyl)-5-nitrobenzo[b]thiophene-2-carboxamide

Compound 257B is prepared from the derivative 257A (6.48 g; 29 mmol) and 3-aminomethylpyridine, according to the conditions used for the preparation of 89A, and abiding by the proportions of the various reagents. At the end of the reaction, the medium is concentrated and then taken up in DCM and water. The desired product precipitates out. It is filtered off and dried. Amount obtained: 7.7 g.

Mass spectrum (ESI): m/z 313 (MH+).

¹H NMR, DMSO-d₆ (ppm): 9.60 (t, 1H); 8.92 (s, 1H); 8.67 (s, 1H); 8.51 (d, 1H); 8.33 (d, 1H); 8.31 (s, 1H); 8.25 (dd, 1H); 7.76 (d, 1H); 7.39 (dd, 1H); 4.54 (d, 2H).

### Example 257C

### N-(Pyrid-3-ylmethyl)-5-aminobenzo[b]thiophene-2-carboxamide

Compound 257C is prepared from the derivative 257B (7.9 g; 25 mmol), according to the conditions used for the preparation of 229, and abiding by the proportions of the various reagents. Amount obtained: 6.41 g (90%).

Mass spectrum (ESI): m/z 284 (MH+).

¹H NMR, DMSO-d₆ (ppm): 9.19 (t, 1H); 8.56 (s, 1H); 8.47 (dd, 1H); 7.84 (s, 1H); 7.74 (d, 1H); 7.60 (d, 1H); 7.38 (dd, 1H); 6.98 (d, 1H); 6.81 (dd, 1H); 5.22 (bs, 2H); 4.49 (d, 2H).

### EXAMPLE 257

Compound 257 is prepared from the derivative 257C (3.5 g; 12.3 mmol) and from the aldehyde 27A according to the conditions used for the preparation of 230, and abiding by the proportions of the various reagents. Amount obtained: 5.2 g (72%).

Mass spectrum (ESI): m/z 479 (MH+).

¹H NMR, DMSO-d₆ (ppm): 9.20 (t, 1H); 8.56 (d, 1H); 8.47 (dd, 1H); 7.9-7.7 (m, 5H); 7.62 (d, 1H); 7.37 (dd, 1H); 7.25 (d, 2H); 6.97 (s, 1H); 6.90 (d, 1H); 6.79 (dd, 1H); 6.06 (t, 1H); 5.39 (s, 2H); 4.49 (d, 2H); 4.12 (d, 2H).

### EXAMPLE 258

### N-(2-Pyrid-2-ylethyl)-5-{[3-(4-cyanobenzyl)-3H-imidazol-4-ylmethyl]amino}benzo[b]thiophene-2-carboxamide

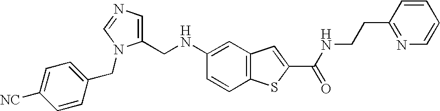

### Example 258A

### N-(2-Pyrid-2-ylethyl)-5-nitrobenzo[b]thiophene-2-carboxamide

Compound 258A is prepared from the derivative 257A (6.4 g; 29 mmol) and 2-(2-aminoethyl)pyridine, according to the conditions used for the preparation of 89A, and abiding by the proportions of the various reagents. At the end of the reaction, the medium is concentrated and then taken up in DCM and water. The desired product precipitates out. It is filtered off and dried. Amount obtained: 8.7 g (68%).

Mass spectrum (ESI): m/z 328 (MH⁺).

¹H NMR, DMSO-d₆ (ppm): 9.10 (t, 1H); 8.90 (s, 1H); 8.51 (d, 1H); 8.31 (d, 1H); 8.3-8.2 (m, 2H); 7.71 (dt, 1H); 7.30 (d, 1H); 7.24 (dd, 1H); 3.66 (q, 2H); 3.04 (t, 2H).

### Example 258B

### N-(2-Pyrid-2-ylethyl)-5-aminobenzo[b]thiophene-2-carboxamide

Compound 258B is prepared from the derivative 258A (8.7 g; 26 mmol), according to the conditions used for the preparation of 229, and abiding by the proportions of the various reagents. Amount obtained: 7.52 g (95%).

Mass spectrum (ESI): m/z 298 (MH⁺).

¹H NMR, DMSO-d₆ (ppm): 8.69 (t, 1H); 8.51 (d, 1H); 7.74 (s, 1H); 7.71 (dt, 1H): 7.58 (d, 1H); 7.28 (d, 1H); 7.23 (dd, 1H); 6.97 (d, 1H); 6.80 (dd, 1H); 5.33 (bs, 2H); 3.60 (q, 2H); 3.00 (t, 2H).

### EXAMPLE 258

Compound 258 is prepared from the derivative 258B (4.0 g; 13.4 mmol) and from the aldehyde 27A according to the conditions used for the preparation of 230 and abiding by the proportions of the various reagents. Amount obtained: 5.38 g.

Mass spectrum (ESI): m/z 493 (MH⁺).

¹H NMR, DMSO-dr (ppm): 8.70 (t, 1H); 8.52 (d, 1H); 7.9-7.65 (m, 5H); 7.60 (d, 1H); 7.35-7.15 (m, 4H); 6.97 (s, 1H); 6.89 (d, 1H); 6.78 (dd, 1H); 6.04 (t, 1H); 5.39 (s, 2H); 4.12 (d, 2H); 3.61 (q, 2H); 3.00 (t, 2H).

### EXAMPLE 259

### 4-(5-{1[2-(4-Methylpiperazine-1-carbonyl)benzo[b]thiophen-5-ylamino]methyl}imidazol-1-ylmethyl)benzonitrile

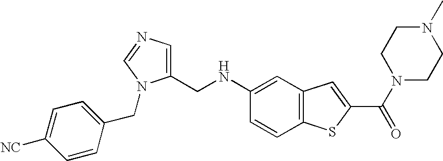

### Example 259A

### (5-Nitrobenzo[b]thiophen-2-yl)-(4-methylpiperazin-1-yl)-methanone

Compound 259A is prepared from the derivative 257A (3.0 g; 13 mmol) and 1-methylpiperazine, according to the conditions used for the preparation of 89A, and abiding by the proportions of the various reagents. At the end of the reaction, the medium is concentrated and then taken up in DCM and water and 20 ml of 1N sodium hydroxide. The aqueous phase is extracted three times with DCM. The organic phases are combined, dried over magnesium sulfate, filtered and concentrated. The crude reaction product is purified by flash chromatography on silica to give the desired compound (3.7 g; 90%).

Mass spectrum (ESI): m/z 306 (MH⁺).

¹H NMR, DMSO-d₆ (ppm): 8.87 (bs, 1H); 8.32 (d, 1H); 8.25 (dd, 1H); 7.98 (s, 1H); 3.66 (bs, 4H); 2.39 (bs, 4H); 2.23 (s, 3H).

### Example 259B

### (5-Aminobenzo[b]thiophen-2-yl)-(4-methylpiperazin-1-yl)-methanone

Compound 259B is prepared from the derivative 259A (3.69 g; 12 mmol), according to the conditions used for the preparation of 229 and abiding by the proportions of the various reagents. Amount obtained: 3.17 g (95%).

¹H NMR, DMSO-dr (ppm): 7.58 (d, 1H); 7.41 (s, 1H); 6.98 (s, 1H); 6.79 (d, 1H); 5.13 (bs, 2H); 3.65 (bs, 4H); 2.37 (bs, 4H); 2.22 (s, 3H).

### EXAMPLE 259

Compound 259 is prepared from the derivative 259B (0.9 g; 3.3 mmol) and from the aldehyde 27A, according to the conditions used for the preparation of 230, and abiding by the proportions of the various reagents. Amount obtained: 1.14 g.

¹H NMR, DMSO-d₆ (ppm): 7.80 (d, 1H); 7.76 (s, 1H); 7.61 (d, 1H); 7.41 (s, 1H); 7.26 (d, 1H); 6.96 (s, 1H); 6.86 (s, 1H); 6.78 (d, 1H); 6.07 (bs, 1H); 5.39 (s, 2H); 4.10 (bs, 2H); 3.64 (bs, 4H); 2.35 (bs, 4H); 2.20 (s, 3H).

### EXAMPLE 260

### N-(2-Pyrrolidin-1-ylethyl)-5-{[3-(4-cyanobenzyl)-3H-imidazol-4-ylmethyl]amino}benzo[b]thiophene-2-carboxamide

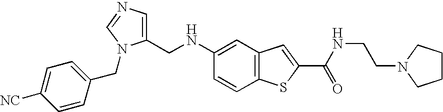

### Example 260A

### N-(2-Pyrrolidin-1-ylethyl)-5-nitrobenzo[b]thiophene-2-carboxamide

Compound 260A is prepared from the derivative 257A (3.2 g; 14 mmol) and from 2-pyrrolidin-1-ylethylamine, according to the conditions used for the preparation of 89A, and abiding by the proportions of the various reagents. At the end of the reaction, the medium is concentrated and then taken up in DCM and water and 20 ml of 1N sodium hydroxide. The aqueous phase is extracted three times with DCM. The organic phases are combined, dried over magnesium sulfate, filtered and concentrated. The crude reaction product is purified by flash chromatography on silica to give the desired compound (3.98 g; 86%).

¹H NMR, DMSO-d₆ (ppm): 8.96 (t, 1H); 8.91 (d, 1H); 8.31 (d, 1H); 8.26 (s, 1H); 8.24 (dd, 1H); 3.41 (q, 2H); 2.60 (t, 2H); 2.50 (bs, 4H+DMSO); 1.69 (bs, 4H).

### Example 260B

### N-(2-Pyrrolidin-1-ylethyl)-5-aminobenzo[b]thiophene-2-carboxamide

Compound 260B is prepared from the derivative 260A (3.98 g; 12 mmol), according to the conditions used for the preparation of 229, and abiding by the proportions of the various reagents. Amount obtained: 2.65 g (73%).

¹H NMR, DMSO-d₆ (ppm): 8.55 (bs, 1H); 7.78 (s, 1H); 7.59 (d, 1H); 6.97 (s, 1H); 6.80 (d, 1H); 5.16 (bs, 2H); 3.38 (q, 2H); 2.60 (t, 2H); 2.50 (bs, 4H+DMSO); 1.69 (bs, 4H).

### EXAMPLE 260

Compound 260 is prepared from the derivative 260B (1.5 g; 5.2 mmol) and from the aldehyde 27A, according to the conditions used for the preparation of 230, and abiding by the proportions of the various reagents. Amount obtained: 1.68 g (57%).

HPLC (XTerra MS, λ 220 nm), 100% H₂O to 100% CH₃CN (+0.1% TFA) over 6 minutes): purity: 86%.

¹H NMR, DMSO-d₆ (ppm): 8.55 (t, 1H); 7.85-7.75 (m, 4H); 7.61 (d, 1H); 7.16 (d, 2H); 6.97 (s, 1H); 6.89 (s, 1H); 6.78 (dd, 1H); 6.04 (t, 1H); 5.39 (s, 2H); 4.11 (d, 2H); 3.4-3.3 (m, 2H); 2.57 (t, 2H); 2.48 (bs, 4H); 1.68 (bs, 4H).

### EXAMPLE 261

### N-(2-Morpholino-4-ylethyl)-5-{1[3-(4-cyanobenzyl)-3H-imidazol-4-ylmethyl]amino}benzo[b]thiophene-2-carboxamide

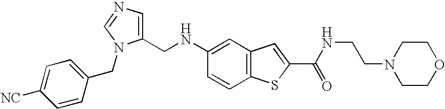

### Example 261A

### N-(2-Morpholino-4-ylethyl)-5-nitrobenzo[b]thiophene-2-carboxamide

Compound 261A is prepared from the derivative 257A (2.6 g; 12 mmol) and from 1-(2-aminoethyl)morpholine, according to the conditions used for the preparation of 89A, and abiding by the proportions of the various reagents. At the end of the reaction, the medium is concentrated and then taken up in DCM and water and 20 ml of 1N sodium hydroxide. The aqueous phase is extracted three times with DCM. The organic phases are combined, dried over magnesium sulfate, filtered and concentrated. The crude reaction product is purified by flash chromatography on silica to give the desired compound (4.76 g).

¹H NMR, DMSO-d₆ (ppm): 8.91 (bs, 2H); 8.31 (d, 1H); 8.26-8.2 (m, 2H); 3.58 (t, 4H); 3.42 (q, 2H); 2.48 (bs, 2H+DMSO); 2.43 (bs, 4H).

### Example 261B

### N-(2-Morpholino-4-ylethyl)-5-aminobenzo[b]thiophene-2-carboxamide

Compound 261B is prepared from the derivative 261A (4.76 g), according to the conditions used for the preparation of 229, and abiding by the proportions of the various reagents. Amount obtained: 3.22 g (90%).

¹H NMR, DMSO-d₆ (ppm): 8.52 (bs, 1H); 7.76 (s, 1H); 7.59 (d, 1H); 6.97 (d, 1H);. 6.80 (dd, 1H); 5.17 (bs, 2H); 3.58 (bs, 4H); 2.37 (q, 2H); 2.55-2.4 (m, 6H+DMSO).

### EXAMPLE 261

Compound 261 is prepared from the derivative 261B (1.00 g; 3.27 mmol) and from the aldehyde 27A, according to the conditions used for the preparation of 230, and abiding by the proportions of the various reagents. Amount obtained: 568 mg.

HPLC (XTerra MS, λ 220 nm), 100% H₂O to 100% CH₃CN (+0.1% TFA) over 6 minutes): purity: 95%.

¹H NMR, DMSO-d₆ (ppm): 8.52 (t, 1H); 7.85-7.75 (m, 4H); 7.60 (d, 1H); 7.25 (d, 2H); 6.97 (s, 1H); 6.89 (s, 1H); 6.78 (dd, 1H); 6.04 (t, 1H); 5.39 (s, 2H); 4.12 (d, 2H); 3.75-3.55 (m, 4H); 3.37 (q, 2H); 2.50-2.40 (m, 6H).

### Examples 262 to 294

Compounds 262 to 264 are prepared in the form of HCl salts from the derivative 127, and from the corresponding sulfonyl chlorides, according to the conditions described for the preparation of 36, and abiding by the proportions of the various reagents. Compounds 265 to 294 are prepared in the form of HCl salts from the derivatives 257, 258, 259 or 260 and from the corresponding sulfonyl chlorides, according to the conditions described for the preparation of 91, and abiding by the proportions of the various reagents.

The products are then purified by filtration on silica using CombiFlash Optix 10 (Isco), and using a gradient of methanol in dichloromethane (0 to 20%)., Finally, they are taken up in a mixture of water, acetonitrile and hydrochloric acid (1N in water), and then freeze-dried in order to be characterized.

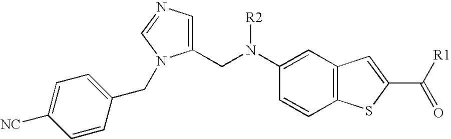

| | | | | Mass | HPLC |
|---|---|---|---|---|---|
| Ex. | R1 | R2 | Compound name | (M + H)⁺ | purity* |
| 262 | 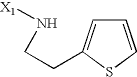 | 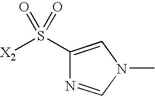 | N-(2-Thiophen-2-ylethyl)-5-{[3-(4-cyano- benzyl)-3H-imidazol-4-ylmethyl]-(1-meth- yl-1H-imidazol-4-sulfonyl)-a- mino}benzo[b]thiophene-2-carboxamide | 642 | 99 |
| 263 | 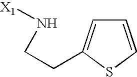 | 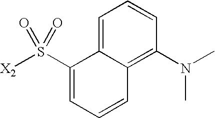 | N-(2-Thiophen-2-ylethyl)-5-{[3-(4-cyano- benzyl)-3H-imidazol-4-ylmethyl]-(5-di- methylaminonaphthalene-1-sulfonyl)-a- mino}benzo[b]thiophene-2-carboxamide | 731 | 96 |
| 264 | 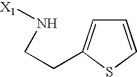 | 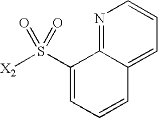 | N-(2-Thiophen-2-ylethyl)-5-{[3-(4-cyano- benzyl)-3H-imidazol-4-ylmethyl]-(quino- line-8-sulfonyl)amino}-ben- zo[b]thiophene-2-carboxamide | 689 | 96 |
| 265 | 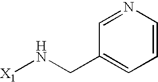 | 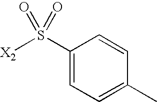 | N-(Pyrid-3-ylmethyl)-5-{[3-(4-cyano- benzyl)-3H-imidazol-4-ylmeth- yl](toluene-4-sulfonyl)amino}-ben- zo[b]thiophene-2-carboxamide | 633 | 99 |
| 266 | 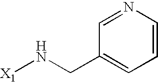 | 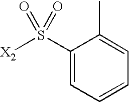 | N-(Pyrid-3-ylmethyl)-5-{[3-(4-cyano- benzyl)-3H-imidazol-4-ylmeth- yl](toluene-2-sulfonyl)amino}-ben- zo[b]thiophene-2-carboxamide | 633 | 92 |
| 267 | 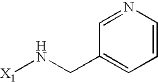 | 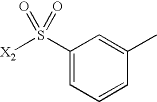 | N-(Pyrid-3-ylmethyl)-5-{[3-(4-cyano- benzyl)-3H-imidazol-4-ylmeth- yl](toluene-3-sulfonyl)amino}-ben- zo[b]thiophene-2-carboxamide | 633 | 95 |
| 268 | 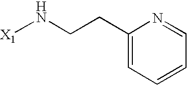 | 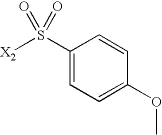 | N-(2-Pyrid-2-ylethyl)-5-{[3-(4-cyano- benzyl)-3H-imidazol-4-ylmethyl]-(4-meth- oxybenzenesulfonyl)amino}benzo-[b]thio- phene-2-carboxamide | 663 | 95 |
| 269 | 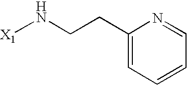 | 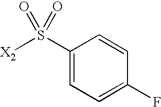 | N-(2-Pyrid-2-ylethyl)-5-{[3-(4-cyano- benzyl)-3H-imidazol-4-ylmethyl]-(4-fluoro- benzenesulfonyl)amino}benzo-[b]thio- phene-2-carboxamide | 651 | 80 |
| 270 | 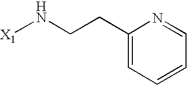 | 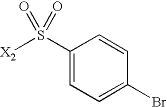 | N-(2-Pyrid-2-ylethyl)-5-{[3-(4-cyano- benzyl)-3H-imidazol-4-ylmethyl]-(4-bromo- benzenesulfonyl)amino}benzo[b]thio- phene-2-carboxamide | 711 713 | 89 |
| 271 | 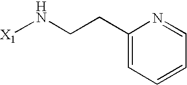 | 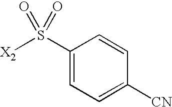 | N-(2-Pyrid-2-ylethyl)-5-{[3-(4-cyano- benzyl)-3H-imidazol-4-ylmethyl]-(4-cyano- benzenesulfonyl)amino}benzo-[b]thio- phene-2-carboxamide | 658 | 85 |
| 272 | 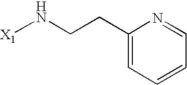 | 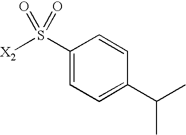 | N-(2-Pyrid-2-ylethyl)-5-{[3-(4-cyano- benzyl)-3H-imidazol-4-ylmethyl]-(4-iso- propylbenzenesulfonyl)amino}benzo-[b]thio- phene-2-carboxamide | 675 | 95 |
| 273 | 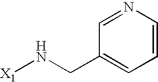 | 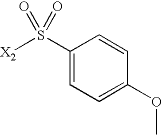 | N-(Pyrid-3-ylmethyl)-5-{[3-(4-cyano- benzyl)-3H-imidazol-4-ylmethyl]-(4-meth- oxybenzenesulfonyl)amino}benzo-[b]thio- phene-2-carboxamide | 649 | 92 |
| 274 | 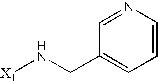 | 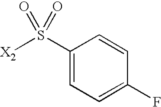 | N-(Pyrid-3-ylmethyl)-5-{[3-(4-cyano- benzyl)-3H-imidazol-4-ylmethyl]-(4-fluoro- benzenesulfonyl)amino}benzo-[b]thio- phene-2-carboxamide | 637 | 93 |
| 275 | 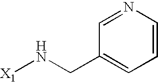 | 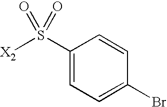 | N-(Pyrid-3-ylmethyl)-5-{[3-(4-cyano- benzyl)-3H-imidazol-4-ylmethyl]-(4-bromo- benzenesulfonyl)amino}benzo-[b]thio- phene-2-carboxamide | 697 699 | 95 |
| 276 | 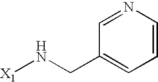 | 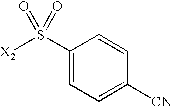 | N-(Pyrid-3-ylmethyl)-5-{[3-(4-cyano- benzyl)-3H-imidazol-4-ylmethyl]-(4-cyano- benzenesulfonyl)amino}benzo-[b]thio- phene-2-carboxamide | 644 | 88 |
| 277 | 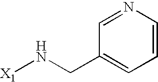 | 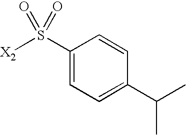 | N-(Pyrid-3-ylmethyl)-5-{[3-(4-cyano- benzyl)-3H-imidazol-4-ylmethyl]-(4-iso- propylbenzenesulfonyl)amino}benzo-[b]thio- phene-2-carboxamide | 661 | 99 |
| 278 | 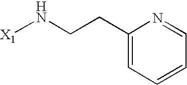 | 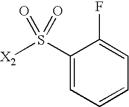 | N-(2-Pyrid-2-ylethyl)-5-{[3-(4-cyano- benzyl)-3H-imidazol-4-ylmethyl]-(2-fluoro- benzenesulfonyl)amino}benzo-[b]thio- phene-2-carboxamide | 651 | 82 |
| 279 | 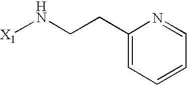 | 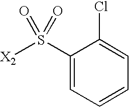 | N-(2-Pyrid-2-ylethyl)-5-{[3-(4-cyano- benzyl)-3H-imidazol-4-ylmethyl]-(2-chloro- benzenesulfonyl)amino}benzo-[b]thio- phene-2-carboxamide | 667 669 | 90 |
| 280 | 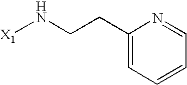 | 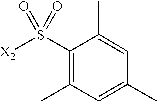 | N-(2-Pyrid-2-ylethyl)-5-{[3-(4-cyano- benzyl)-3H-imidazol-4-ylmethyl]-(2,4,6-tri- methylbenzenesulfonyl)amino}-ben- zo[b]thiophene-2-carboxamide | 675 | 91 |
| 281 | 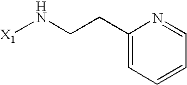 | 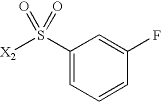 | N-(2-Pyrid-2-ylethyl)-5-{[3-(4-cyano- benzyl)-3H-imidazol-4-ylmethyl]-(3-fluoro- benzenesulfonyl)amino}benzo-[b]thio- phene-2-carboxamide | 651 | 88 |
| 282 | 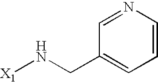 | 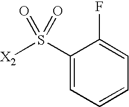 | N-(Pyrid-3-ylmethyl)-5-{[3-(4-cyano- benzyl)-3H-imidazol-4-ylmethyl]-(2-fluoro- benzenesulfonyl)amino}benzo-[b]thio- phene-2-carboxamide | 637 | 88 |
| 283 | 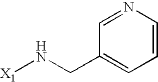 | 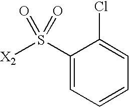 | N-(Pyrid-3-ylmethyl)-5-{[3-(4-cyano- benzyl)-3H-imidazol-4-ylmethyl]-(2-chloro- benzenesulfonyl)amino}benzo-[b]thio- phene-2-carboxamide | 653 655 | 87 |
| 284 | 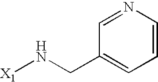 | 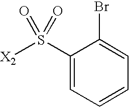 | N-(Pyrid-3-ylmethyl)-5-{[3-(4-cyano- benzyl)-3H-imidazol-4-ylmethyl]-(2-bromo- benzenesulfonyl)amino}benzo-[b]thio- phene-2-carboxamide | 699 701 | 89 |
| 285 | 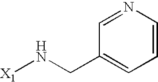 | 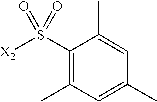 | N-(Pyrid-3-ylmethyl)-5-{[3-(4-cyano- benzyl)-3H-imidazol-4-ylmethyl]-(2,4,6-tri- methylbenzenesulfonyl)amino}benzo-[b]thio- phene-2-carboxamide | 661 | 87 |
| 286 | 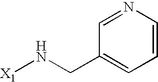 | 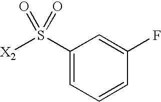 | N-(Pyrid-3-ylmethyl)-5-{[3-(4-cyano- benzyl)-3H-imidazol-4-ylmethyl]-(3-fluoro- benzenesulfonyl)amino}benzo-[b]thio- phene-2-carboxamide | 637 | 96 |
| 287 | 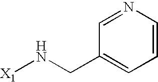 | 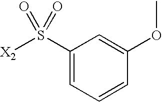 | N-(Pyrid-3-ylmethyl)-5-{[3-(4-cyano- benzyl)-3H-imidazol-4-ylmethyl]-(3-methoxy- benzenesulfonyl)amino}benzo-[b]thio- phene-2-carboxamide | 649 | 99 |
| 288 | 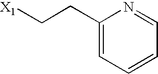 | 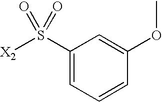 | N-(2-Pyrid-2-ylethyl)-5-{[3-(4-cyano- benzyl)-3H-imidazol-4-ylmethyl]-(3-methoxy- benzenesulfonyl)amino}benzo-[b]thio- phene-2-carboxamide | 663 | 96 |
| 289 | 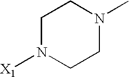 | 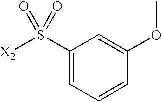 | N-[3-(4-Cyanobenzyl)-3H-imidazol-4-ylmeth- yl]-N-[2-(4-methylpiperazine-4-carbo- nyl)benzo[b]thiophen-5-yl]-3-methoxy- benzenesulfonamide | 641 | 92 |
| 290 | 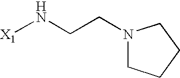 | 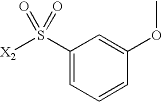 | N-(2-Pyrrolidin-1-ylethyl)-5-{3-methoxy- benzenesulfonyl-[3-(4-cyanobenzyl)-3H-imi- dazol-4-ylmethyl]amino}benzo-[b]thio- phene-2-carboxamide | 655 | 97 |
| 291 | 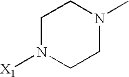 | 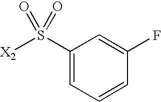 | N-[3-(4-Cyanobenzyl)-3H-imidazol-4-ylmeth- yl]-N-[2-(4-methylpiperazine-4-carbo- nyl)benzo[b]thiophen-5-yl]-3-fluoro- benzenesulfonamide | 629 | 89 |
| 292 | 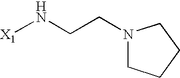 | 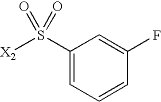 | N-(2-Pyrrolidin-1-ylethyl)-5-{3-fluoro- benzenesulfonyl-[3-(4-cyanobenzyl)-3H-imi- dazol-4-ylmethyl]amino}benzo-[b]thio- phene-2-carboxamide | 647 | 97 |
| 293 | 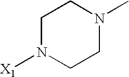 | 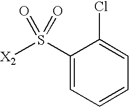 | N-[3-(4-Cyanobenzyl)-3H-imidazol-4-ylmeth- yl]-N-[2-(4-methylpiperazine-4-carbo- nyl)benzo[b]thiophen-5-yl]-2-chloro- benzenesulfonamide | 645 647 | 96 |
| 294 | 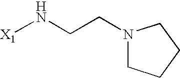 | 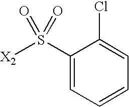 | N-(2-Pyrrolidin-1-ylethyl)-5-{2-chloro- benzenesulfonyl-[3-(4-cyanobenzyl)-3H-imi- dazol-4-ylmethyl]amino}benzo[b]-thio- phene-2-carboxamide | 659 661 | 99 |

*HPLC conditions [C₁₈ XTerra MS, 4.6 × 50 mm, 5 μm; λ = 220 nm; gradient 100% H₂O (+0.05% TFA) to 100% CH₃CN (+0.05% TFA) over 6 minutes]
¹H NMR, DMSO-d₆ (ppm).
Example 262: 9.20(s, 1H); 9.00(t, 1H); 8.05-7.85(m, 5H); 7.69(s, 1H); 7.55(d, 2H); 7.46(d, 2H); 7.34(d, 1H); 7.00-6.90(m, 3H); 5.68(s, 2H); 5.02(s, 2H); 3.80-3.4(m, H₂O+3H); 3.55-3.50(m, 2H); 3.08(t, 2H).
Example 265: 9.77(t, 1H); 9.21(s, 1H); 8.83(s, 1H); 8.74(d, 1H); 8.32(d, 1H); 8.08(s, 1H); 7.93(d, 2H); 7.90(d, 1H); 7.86(dd, 1H); 7.51(d, 1H); 7.50(s, 1H); 7.47(d, 2H); 7.41(AB, 4H); 6.89(dd, 1H); 5.67(s, 2H); 4.89(s, 2H); 4.61(d, 2H); 2.42(s, 3H).
Example 266(mixture of two conformers): 9.80(bt, 1H); 9.22(s, 1H); 8.87(s, 1H); 8.77(d, 1H); 8.40(d, 1H); 8.09(s, 1H); 7.95-7.85(m, 4H); 7.70(d, 1H); 7.60-7.30(m, 6H); 7.10(d, 1H); 7.02(d, 0.6H); 6.89(d, 0.4H); 5.67(s, 0.8H); 5.63(s, 1.2H); 4.97(s, 1.2H); 4.89(s, 0.8H); 4.64(d, 2H); 2.42(s, 1.2H); 2.02(s, 1.8H).
Example 267: 9.78(t, 1H); 9.23(s, 1H); 8.85(s, 1H); 8.76(d, 1H); 8.37(d, 1H); 8.08(s, 1H); 7.96-7.88(m, 4H); 7.57(d, 1H); 7.50-7.40(m, 5H); 7.41(s, 1H); 7.24(d, 1H); 6.89(dd, 1H); 5.67(s, 2H); 4.91(s, 2H); 4.64(d, 2H); 2.37(s, 3H).
Example 272: 9.22(s, 1H); 9.18(t, 1H); 8.78(d, 1H); 8.36(t, 1H): 7.95(s, 1H); 7.94(d, 2H); 7.87(d, 2H); 7.80(bt, 1H); 7.50-7.40(m, 8H); 6.86(d, 1H); 5.67(s, 2H); 4.90(s, 2H); 3.72(q, 2H); 3.29(t, 2H); 3.01(sept., 1H); 1.23(d, 6H).
Example 276: 9.83(t, 1H); 9.24(s, 1H); 8.86(s, 1H); 8.78(d, 1H); 8.39(d, 1H); 8.13(d, 2H); 8.10(s, 1H); 7.95-7.8(m, 3H); 7.71(d, 2H); 7.55(s, 1H); 7.52(s, 1H); 7.46(d, 2H); 6.93(d, 1H); 5.66(s, 2H); 4.98(s, 2H); 4.65(d, 2H).
Example 284: 9.68(t, 1H); 9.20(s, 1H); 8.80(s, 1H); 8.72(d, 1H); 8.27(d, 1H); 8.07(s, 1H); 7.95-7.75(m, 5H); 7.72(d, 1H); 7.68(s, 1H); 7.60-7.2(m, 5H); 7.10(d, 1H); 5.63(s, 2H); 5.17(s, 2H); 4.61(d, 2H).
Example 294: 10.68(bs, 1H); 9.37(t, 1H); 9.24(s, 1H); 8.15(s, 1H); 7.90-7.85(m, 3H); 7.75-7.65(m, 4H); 7.58(s, 1H); 7.45-7.35(m, 3H); 7.09(d, 1H); 5.65(s, 2H); 5.17(s, 2H); 3.64(bs, 4H); 3.34(bd, 2H); 2.95(bs, 2H); 2.09(bs, 2H); 2.05-1.80(m, 2H).

### Examples 295 to 332

Compounds 295 to 332 are prepared in the form of HCl salts from the derivatives 257, 258, 259 or 260 and from the corresponding acid chlorides, according to the conditions described for the preparation of 91, and abiding by the proportions of the various reagents. The products are then purified by filtration on silica using CombiFlash Optix 10 (Isco), and using a gradient of methanol in dichloromethane (0 to 20%). Finally, they are taken up in a mixture of water, acetonitrile and hydrochloric acid (1N in water), and then freeze-dried in order to be characterized.

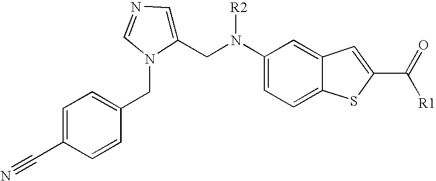

| | | | | Mass | HPLC |
|---|---|---|---|---|---|
| Ex. | R1 | R2 | Compound name | (M + H)⁺ | purity* |
| 295 | 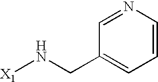 | X₂COnPr | N-(Pyrid-3-ylmethyl)-5-{butyryl-[3-(4- cyanobenzyl)-3H-imidazol-4-ylmethyl]- amino}benzo[b]thiophene-2-carboxamide | 549 | 98 |
| 296 | 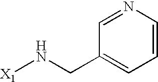 | 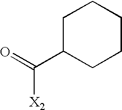 | N-(Pyrid-3-ylmethyl)-5-{[3-(4-cyano- benzyl)-3H-imidazol-4-ylmethyl]cyclo- hexanecarbonylamino}benzo[b]thiophene- 2-carboxamide | 589 | 99 |
| 297 | 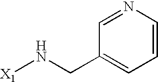 | 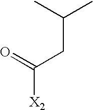 | N-(Pyrid-3-ylmethyl)-5-{[3-(4-cyano- benzyl)-3H-imidazol-4-ylmethyl]-(3- methylbutyryl)amino}benzo[b]thiophene-2- carboxamide | 563 | 93 |
| 298 | 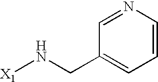 | X₂COnPentyl | N-(Pyrid-3-ylmethyl)-5-{[3-(4-cyano- benzyl)-3H-imidazol-4-ylmethyl]- hexanoylamino}benzo[b]thiophene-2- carboxamide | 577 | 99 |
| 299 | 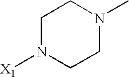 | X₂COnPr | N-[3-(4-Cyanobenzyl)-3H-imidazol-4- ylmethyl]-N-[2-(4-methylpiperazine-1- carbonyl)benzo[b]thiophen-5-yl]butyramide | 541 | 96 |
| 300 | 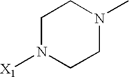 | 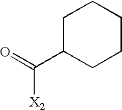 | N-[3-(4-Cyanobenzyl)-3H-imidazol-4- ylmethyl]-N-[2-(4-methylpiperazine-1- carbonyl)benzo[b]thiophen-5- yl]cyclohexanecarboxamide | 581 | 96 |
| 301 | 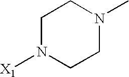 | 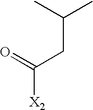 | N-[3-(4-Cyanobenzyl)-3H-imidazol-4- ylmethyl]-3-methyl-N-[2-(4-methyl- piperazine-1-carbonyl)benzo[b]thiophen-5- yl]butyramide | 555 | 95 |
| 302 | 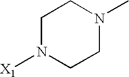 | X₂COnPentyl | N-[3-(4-Cyanobenzyl)-3H-imidazol-4- ylmethyl]-N-[2-(4-methylpiperazine-1- carbonyl)benzo[b]thiophen-5-yl]hexanamide | 569 | 97 |
| 303 | 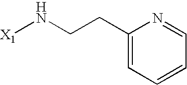 | 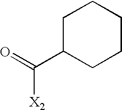 | N-(2-Pyrid-2-ylethyl)-5-{[3-(4-cyano- benzyl)-3H-imidazol-4-ylmethyl]cyclo- hexanecarbonylamino}benzo[b]thiophene- 2-carboxamide | 603 | 94 |
| 304 | 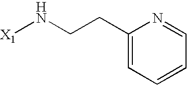 | 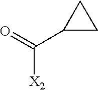 | N-(2-Pyrid-2-ylethyl)-5-{[3-(4-cyano- benzyl)-3H-imidazol-4-ylmethyl]cyclo- propanecarbonylamino}benzo[b]thiophene- 2-carboxamide | 561 | 98 |
| 305 | 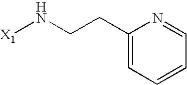 | 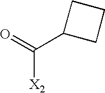 | N-(2-Pyrid-2-ylethyl)-5-{[3-(4-cyano- benzyl)-3H-imidazol-4-ylmethyl]cyclo- butanecarbonylamino}benzo[b]thiophene-2- carboxamide | 575 | 99 |
| 306 | 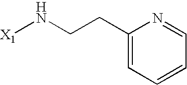 | X₂COnPr | N-(2-Pyrid-2-ylethyl)-5-{[3-(4-cyano- benzyl)-3H-imidazol-4-ylmethyl]- butyrylamino}benzo[b]thiophene-2- carboxamide | 563 | 99 |
| 307 | 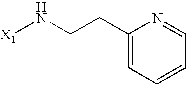 | 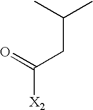 | N-(2-Pyrid-2-ylethyl)-5-{[3-(4-cyano- benzyl)-3H-imidazol-4-ylmethyl]-(3- methylbutyryl)amino}benzo[b]thiophene-2- carboxamide | 577 | 99 |
| 308 | 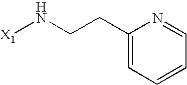 | 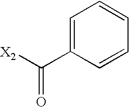 | N-(2-Pyrid-2-ylethyl)-5-{benzoyl-[3-(4- cyanobenzyl)-3H-imidazol-4-ylmethyl]- amino}benzo[b]thiophene-2-carboxamide | 597 | 94 |
| 309 | 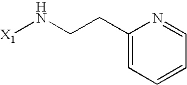 | 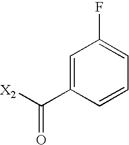 | N-(2-Pyrid-2-ylethyl)-5-{[3-(4-cyano- benzyl)-3H-imidazol-4-ylmethyl]-(3- fluorobenzoyl)amino}benzo[b]thiophene-2- carboxamide | 615 | 88 |
| 310 | 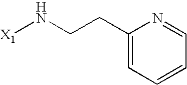 | 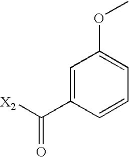 | N-(2-Pyrid-2-ylethyl)-5-{[3-(4-cyano- benzyl)-3H-imidazol-4-ylmethyl]-(3- methoxybenzoyl)amino}benzo[b]thiophene- 2-carboxamide | 627 | 96 |
| 311 | 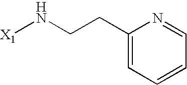 | 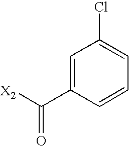 | N-(2-Pyrid-2-ylethyl)-5-{(3-chlorobenzoyl)- [3-(4-cyanobenzyl)-3H-imidazol-4- ylmethyl]amino}benzo[b]thiophene-2- carboxamide | 631 633 | 95 |
| 312 | 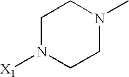 | 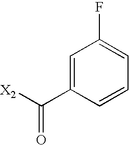 | N-[3-(4-Cyanobenzyl)-3H-imidazol-4- ylmethyl]-3-methyl-N-[2-(4-methyl- piperazine-1-carbonyl)benzo[b]thiophen-5- yl]-3-fluorobenzamide | 593 | 90 |
| 313 | 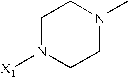 | 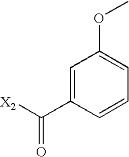 | N-[3-(4-Cyanobenzyl)-3H-imidazol-4- ylmethyl]-3-methyl-N-[2-(4-methyl- piperazine-1-carbonyl)benzo[b]thiophen-5- yl]-3-methoxybenzamide | 605 | 87 |
| 314 | 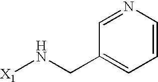 | 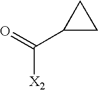 | N-(Pyrid-3-ylmethyl)-5-{[3-(4-cyano- benzyl)-3H-imidazol-4-ylmethyl]cyclo- propanecarbonylamino}benzo[b]thiophene- 2-carboxamide | 547 | 99 |
| 315 | 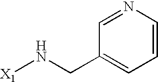 | 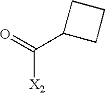 | N-(Pyrid-3-ylmethyl)-5-{[3-(4-cyano- benzyl)-3H-imidazol-4-ylmethyl]cyclo- butanecarbonylamino}benzo[b]thiophene-2- carboxamide | 561 | 99 |
| 316 | 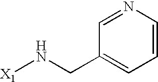 | 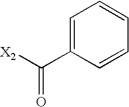 | N-(Pyrid-3-ylmethyl)-5-{benzoyl-[3-(4- cyanobenzyl)-3H-imidazol-4-ylmethyl]- amino}benzo[b]thiophene-2-carboxamide | 583 | 92 |
| 317 | 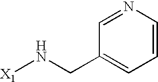 | 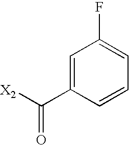 | N-(Pyrid-3-ylmethyl)-5-{[3-(4-cyano- benzyl)-3H-imidazol-4-ylmethyl]-3- fluorobenzoylamino}benzo[b]thiophene-2- carboxamide | 601 | 97 |
| 318 | 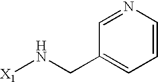 | 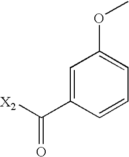 | N-(Pyrid-3-ylmethyl)-5-{[3-(4-cyano- benzyl)-3H-imidazol-4-ylmethyl]-3- methoxybenzoylamino}benzo[b]thiophene- 2-carboxamide | 613 | 94 |
| 319 | 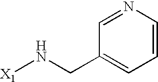 | 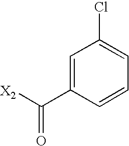 | N-(Pyrid-3-ylmethyl)-5-{[3-(4-cyano- benzyl)-3H-imidazol-4-ylmethyl]-3-chloro- benzoylamino}benzo[b]thiophene-2- carboxamide | 617 619 | 96 |
| 320 | 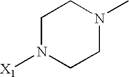 | 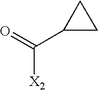 | N-[3-(4-Cyanobenzyl)-3H-imidazol-4- ylmethyl]-N-[2-(4-methylpiperazine-1- carbonyl)benzo[b]thiophen-5-yl]cyclo- propanecarboxamide | 539 | 95 |
| 321 | 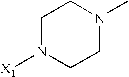 | 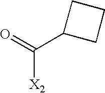 | N-[3-(4-Cyanobenzyl)-3H-imidazol-4- ylmethyl]-N-[2-(4-methylpiperazine-1- carbonyl)benzo[b]thiophen-5-yl]cyclo- butanecarboxamide | 553 | 99 |
| 322 | 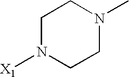 | 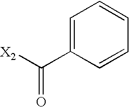 | N[3-(4-Cyanobenzyl)-3H-imidazol-4- ylmethyl]-3-methyl-N-[2-(4-methyl- piperazine-1-carbonyl)benzo[b]thiophen-5- yl]benzamide | 575 | 91 |
| 323 | 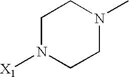 | 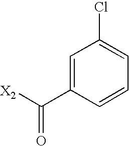 | N-[3-(4-Cyanobenzyl)-3H-imidazol-4- ylmethyl]-3-methyl-N-[2-(4-methyl- piperazine-1-carbonyl)benzo[b]thiophen-5- yl]-3-chlorobenzamide | 611 | 89 |
| 324 | 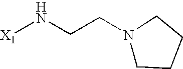 | 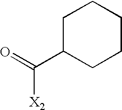 | N-(2-Pyrrolidin-1-ylethyl)-5-{[3-(4-cyano- benzyl)-3H-imidazol-4-ylmethyl]cyclo- hexanecarbonylamino}benzo[b]thiophene- 2-carboxamide | 595 | 97 |
| 325 | 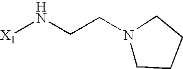 | 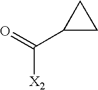 | N-(2-Pyrrolidin-1-ylethyl)-5-{[3-(4-cyano- benzyl)-3H-imidazol-4-ylmethyl]cyclo- propanecarbonylamino}benzo[b]thiophene- 2-carboxamide | 553 | 91 |
| 326 | 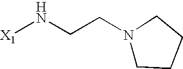 | 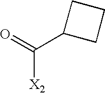 | N-(2-Pyrrolidin-1-ylethyl)-5-{[3-(4-cyano- benzyl)-3H-imidazol-4-ylmethyl]cyclo- butanecarbonylamino}benzo[b]thiophene-2- carboxamide | 567 | 95 |
| 327 | 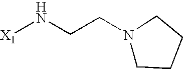 | X₂COnPr | N-(2-Pyrrolidin-1-ylethyl)-5-{[3-(4-cyano- benzyl)-3H-imidazol-4-ylmethyl]- butanoylamino}benzo[b]thiophene-2- carboxamide | 555 | 95 |
| 328 | 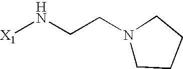 | 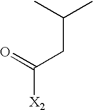 | N-(2-Pyrrolidin-1-ylethyl)-5-{[3-(4-cyano- benzyl)-3H-imidazol-4-ylmethyl]-3- methylbutyrylamino}benzo[b]thiophene-2- carboxamide | 569 | 94 |
| 329 | 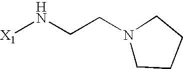 | 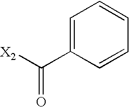 | N-(2-Pyrrolidin-1-ylethyl)-5-{benzoyl-[3- (4-cyanobenzyl)-3H-imidazol-4-ylmethyl]- amino}benzo[b]thiophene-2-carboxamide | 589 | 91 |
| 330 | 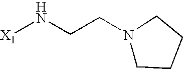 | 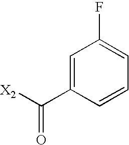 | N-(2-Pyrrolidin-1-ylethyl)-5-{[3-(4-cyano- benzyl)-3H-imidazol-4-ylmethyl]-3- fluorobenzoylamino}benzo[b]thiophene-2- carboxamide | 607 | 93 |
| 331 | 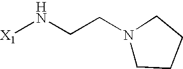 | 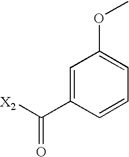 | N-(2-Pyrrolidin-1-ylethyl)-5-{[3-(4-cyano- benzyl)-3H-imidazol-4-ylmethyl]-3- methoxybenzoylamino}benzo[b]thiophene- 2-carboxamide | 619 | 94 |
| 332 | 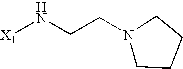 | 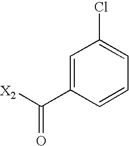 | N-(2-Pyrrolidin-1-ylethyl)-5-{[3-(4-cyano- benzyl)-3H-imidazol-4-ylmethyl]-3- chlorobenzoylamino}benzo[b]thiophene-2- carboxamide | 624 | 91 |

*HPLC conditions [C₁₈ XTerra MS, 4.6 × 50 mm, 5 μm; λ = 220 nM; gradient 100% H₂O(+0.05% TFA) to 100% CH₃CN(+0.05% TFA) over 6 minutes] ¹H NMR, DMSO-d₆(ppm).
Example 298: 9.90(t, 1H); 9.31(s, 1H); 8.90(s, 1H); 8.80(d, 1H); 8.46(d, 1H); 8.23(s, 1H); 8.05(d, 1H); 7.97(t, 1H); 7.84(d, 2H); 7.80(s, 1H); 7.55(s, 1H); 7.41(d, 2H); 7.26(d, 1H); 5.59(s, 2H); 4.92(s, 2H); 4.58(d, 2H); 1.94(t, 2H); 1.40(quint., 2H); 1.20-1.00(m, 4H); 0.76(t, 3H).
Example 304: 9.30(s, 1H); 9.20(t, 1H): 8.82(d, 1H); 8.45(t, 1H); 8.08(s, 1H); 8.03(d, 1H); 7.95(d, 1H); 7.89(t, 1H); 8.85(d, 2H); 7.79(s, 1H); 7.51(s, 1H); 7.40(d, 2H); 7.26(d, 1H); 5.58(s, 2H); 4.96(s, 2H); 3.75(q, 2H); 3.34(t, 2H); 1.90(bs, 1H); 0.79(bs, 2H); 0.60(bs, 2H).
Example 306: 9.27(s, 1H); 9.03(t, 1H); 8.84(s, 1H); 8.74(d, 1H); 8.39(d, 1H); 8.10-8.00(m, 2H); 7.90(t, 1H); 7.85(d, 2H); 7.78(s, 1H); 7.53(s, 1H); 7.42(d, 2H); 7.23(d, 1H); 5.58(s, 2H); 4.91(s, 2H); 3.8-3.2(m, H₂O + 2H); 3.08(t, 2H); 1.95(t, 2H); 1.38(quint., 2H); 1.09(sext., 2H); 0.72(t, 3H).
Example 314: 9.88(t, 1H); 9.31(s, 1H); 8.88(s, 1H); 8.79(d, 1H); 8.43(d, 1H); 8.23(s, 1H); 8.06(d, 1H); 7.94(dd, 1H); 7.84(d, 2H); 7.81(s, 1H); 7.51(s, 1H); 7.39(d, 2H); 7.29(dd, 1H); 5.58(s, 2H); 4.98(s, 2H); 4.66(d, 2H); 1.2(bs, 1H); 0.80(bs, 2H); 0.61(q, 2H).
Example 322: 11.56(bs, 1H); 9.30(s, 1H); 7.90-7.80(m, 3H); 7.68(s, 1H); 7.62(s, 1H); 7.60(s, 1H); 7.42(d, 2H); 7.35-7.15(m, 6H); 5.67(s, 2H); 5.13(s, 2H); 4.35(bs, 2H); 3.70(bs, H₂O + 2H); 3.43(bs, 2H); 3.10(bs, 2H); 2.77(s, 3H).
Example 327: 10.64(bs, 1H); 9.35(t, 1H); 9.25(s, 1H); 8.22(s, 1H); 8.04(d, 1H); 7.85(d, 2H); 7.78(s, 1H); 7.54(s, 1H); 7.41(d, 2H); 7.26(dd, 1H); 5.57(s, 2H); 4.92(s, 2H); 3.63(bt, 4H); 3.35(bd, 2H); 3.04(bs, 2H); 2.05-1.85(m, 6H); 1.43(sext., 2H); 0.73(t, 3H).
Example 331: 10.62(bs, 1H); 9.32(t, 1H); 9.28(s, 1H); 8.10(s, 1H); 7.9-7.8(m, 3H); 7.70(s, 1H); 7.64(s, 1H); 7.40(d, 2H); 7.19(d, 1H); 7.06(t, 1H); 6.78(bs, 3H); 5.66(s, 2H); 5.13(s, 2H); 3.65-3.5(m, 7H); 3.33(bd, 2H); 3.02(bs, 2H); 2.00(bs, 2H); 1.95-1.85(m, 2H).

### Examples 333 to 340

Compounds 333 to 340 are prepared in the form of HCl salts, from the derivatives 257, 258, 259 or 260 and from the corresponding aldehydes, according to the conditions described for the preparation of 170, and abiding by the proportions of the various reagents. The products are then purified by filtration on silica using CombiFlash Optix 10 (Isco), and using a gradient of methanol in dichloromethane (0 to 20%). Finally, they are taken up in a mixture of water, acetonitrile and hydrochloric acid (1N in water), and then freeze-dried in order to be characterized.

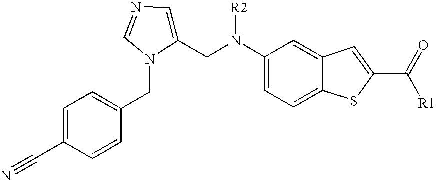

| | | | | Mass | HPLC |
|---|---|---|---|---|---|
| Ex. | R1 | R2 | Compound name | (M + H)⁺ | purity* |
| 333 | 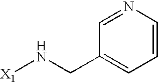 | X₂nPr | N-(Pyrid-3-ylmethyl)-5-{[3-(4-cyano- benzyl)-3H-imidazol-4-ylmethyl]- propylamino}benzo[b]thiophene-2- carboxamide | 521 | 98 |
| 334 | 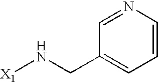 | X₂nBu | N-(Pyrid-3-ylmethyl)-5-{butyl-[3-(4- cyanobenzyl)-3H-imidazol-4-ylmethyl]- amino}benzo[b]thiophene-2-carboxamide | 535 | 95 |
| 335 | 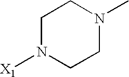 | X₂nPr | 4-[5-({[2-(4-Methylpiperazine-1- carbonyl)-benzo[b]thiophen-5-yl]- propylamino}methyl)imidazol-1- ylmethyl]benzonitrile | 513 | 85 |
| 336 | 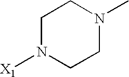 | X₂nBu | 4-[5-({Butyl-[2-(4-methylpiperazine-1- carbonyl)benzo[b]thiophen-5-yl]amino}- methyl)imidazol-1-ylmethyl]benzonitrile | 527 | 90 |
| 337 | 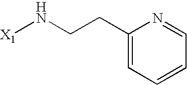 | X₂nPr | N-(2-Pyrid-2-ylethyl)-5-{[3-(4-cyano- benzyl)-3H-imidazol-4-ylmethyl]- propylamino}benzo[b]thiophene-2- carboxamide | 535 | 96 |
| 338 | 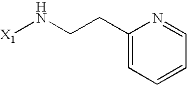 | X₂nBu | N-(2-Pyrid-2-ylethyl)-5-{[3-(4- cyanobenzyl)-3H-imidazol-4-ylmethyl]- butylamino}benzo[b]thiophene-2- carboxamide | 549 | 96 |
| 339 | 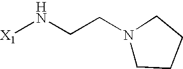 | X₂nPr | N-(2-Pyrrolidin-1-ylethyl)-5-{[3-(4-cyano- benzyl)-3H-imidazol-4-ylmethyl]propyl- amino}benzo[b]thiophene-2-carboxamide | 527 | 96 |
| 340 | 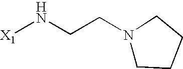 | X₂nBu | N-(2-Pyrrolidin-1-ylethyl)-5-{butyl-[3-(4- cyanobenzyl)-3H-imidazol-4-ylmethyl]- amino}benzo[b]thiophene-2-carboxamide | 541 | 90 |

*HPLC conditions [C₁₈ XTerra MS, 4.6 × 50 mm, 5 μm; λ = 220 nm; gradient 100% H₂O(+0.05% TFA) to 100% CH₃CN(+0.05% TFA) over 6 minutes] ¹H NMR, DMSO-d₆(ppm).
Example 335: 8.01(s, 1H); 7.81(d, 2H); 7.70(d, 1H); 7.53(s, 1H); 7.22(d, 2H); 7.01(d, 1H); 6.9-6.8(m, 2H); 5.39(s, 2H); 4.35(s, 2H); 3.90(bs, 4H); 3.12(bs, 6H); 2.69(s, 3H); 1.41(sext., 2H); 0.78(t, 3H).
Example 336: 9.32(s, 1H); 7.88(d, 2H); 7.74(d, 1H); 7.58(s, 1H); 7.50(s, 1H); 7.44(d, 2H); 7.06(bs, 1H); 6.85(bd, 1H); 5.64(s, 2H); 4.47(s, 2H); 4.42(bd, 2H); 3.54(bs, 2H); 3.45(bd, 2H); 3.22(bt, 2); 3.11(qe, 2H); 2.78(d, 3H); 1.38(quint., 2H), 1.22(sext., 2H); 0.83(t, 3H).
Example 337: 9.30(s, 1H); 9.03(bs, 1H); 8.82(d, 1H); 8.47(t, 1H); 7.95(d, 1H); 7.93-7.85(m, 4H); 7.69(d, 1H); 7.48(s, 1H); 7.43(d, 2H); 7.00(bs, 1H); 6.83(d, 1H); 5.63(s, 2H); 4.48(s, 2H); 3.73(bd, 2H); 3.34(t, 2H); 3.19(t, 2H); 1.43(q, 2H); 0.80(t, 3H).

### EXAMPLE 341

{5-[(1-Methyl-1H-benzoimidazol-2-ylmethyl)amino]benzo[b]thiophen-2-yl}-(4-methylpiperazin-1-yl)methanone

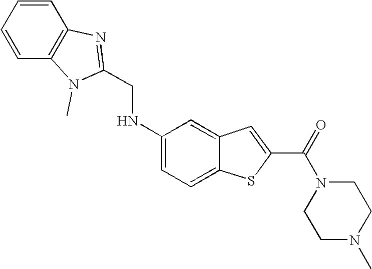

Compound 341 is prepared, in the form of the HCl salt, from the derivative 259B (60 mg; 0.22 mmol) and from 1-methyl-2-formylbenzimidazole, according to the conditions described for the preparation of 170, and abiding by the proportions of the various reagents. The residue is purified by preparative HPLC (Waters Prep 4000) on a Prep Nova-Pak HR C-18 column (Waters; 25×100 mm; 6 μm) using a total gradient of from 100% water (0.1% HCl) to 100% acetonitrile (0.1% HCl) over 15 minutes, to give the desired product 170 in the form of hydrochloride. Amount obtained: 56 mg (58%).

HPLC (C₁₈ XTerra, % 220 nm, 100% H₂O to 100% CH₃CN (+0.1% TFA) over 8 minutes): purity: 97%.

Mass spectrum (ESI): m/z 420 (MH⁺)

¹HNMR, DMSO-dr (ppm): 11.59 (bs, 1H); 8.01 (d, 1H); 7.79 (d, 1H); 7.76 (d, 1H); 7.7-7.55 (m, 2H); 7.56 (s, 1H); 7.17 (d, 1H); 7.08 (dd, 1H); 5.00 (s, 2H); 4.37 (bd, 2H); 4.09 (s, 3H); 3.53 (bs, 2H); 3.41 (bd, 2H); 3.08 (q, 2H); 2.75 (bs, 3H).

### EXAMPLE 342

N-(Pyrid-3-ylmethyl)-5-[(1-methyl-1H-benzoimidazol-2-ylmethyl)amino]benzo[b]thiophene-2-carboxamide

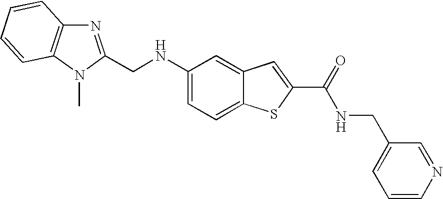

Compound 342 is prepared, in the form of the HCl salt, from the derivative 257C (60 mg; 0.21 mmol) and from 1-methyl-2-formylbenzimidazole, according to the conditions described for the preparation of 170, and abiding by the proportions of the various reagents. The residue is purified by preparative HPLC (Waters Prep 4000) on a Prep Nova-Pak HR C-18 column (Waters; 25×100 mm; 6 μm) using a total gradient of from 100% water (0.1% HCl) to 100% acetonitrile (0.1% HCl) over 15 minutes, to give the desired product 170 in the form of hydrochloride. Amount obtained: 10 mg (9%).

HPLC (C₁₈ XTerra, λ 220 nm, 100% H₂O to 100% CH₃CN (+0.1% TFA) over 8 minutes): purity: 97%.

Mass spectrum (ESI): m/z 428 (MH⁺)

¹H NMR, DMSO-d₆ (ppm): 9.23 (t, 1H); 8.56 (bs, 1H); 8.47 (d, 1H); 7.88 (s, 1H); 7.73 (d, 1H); 7.67 (d, 1H); 7.60 (d, 1H); 7.51 (d, 1H); 7.36 (dd, 1H); 7.25-7.15 (m, 3H); 7.02 (dd, 1H); 6.46 (t, 1H); 4.61 (d, 2H); 4.48 (d, 2H); 3.85 (t, 3H).

### EXAMPLE 343

N-[2-(4-Methylpiperazine-1-carbonyl)benzo[b]thiophen-5-yl]-2-pyrid-3-ylacetamide

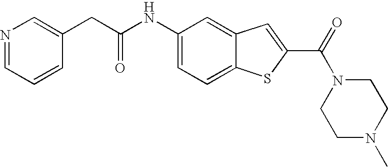

Compound 343 is prepared from derivative 259B (64 mg; 0.23 mmol) and from 2-pyrid-3-ylacetic acid, according to the conditions described for the preparation of 40, and abiding by the proportions of the various reagents. Amount obtained: 35 mg (42%).

HPLC (C₁₈ XTerra, λ 220 nm, 100% H₂O to 100% CH₃CN (+0.1% TFA) over 8 minutes): purity: 99%.

Mass spectrum (ESI): m/z 395 (MH⁺)

¹HNMR, DMSO-d₆ (ppm): 11.08 (bs, 1H); 10.79 (s, 1H); 8.91 (s, 1H); 8.82 (d, 1H); 8.48 (d, 1H); 8.37 (s, 1H); 8.0-7.95 (m, 2H); 7.80 (s, 1H); 7.61 (d, 1H); 4.5-4.35 (m, 2H); 4.05 (s, 2H); 4.1-3.3 (m, H₂O+4H); 3.2-3.05 (m, 2H); 2.79 (s, 3H).

### EXAMPLE 344

N-(Pyrid-4-yl)-5-[(1-methyl-1H-benzoimidazol-2-ylmethyl)amino]benzo[b]thiophene-2-carboxamide

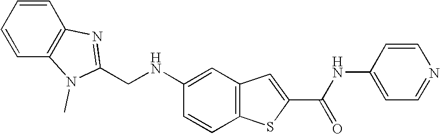

### Example 344A

N-(Pyrid-4-yl)-5-nitrobenzo[b]thiophene-2-carboxamide. Compound 344A is prepared from derivative 257A (2.5 g; 11 mmol) and from 4-aminopyridine (1.37 g; 14 mmol), according to the conditions used for the preparation of 89A, and abiding by the proportions of the various reagents, using DMF as solvent, and heating at 45° C. for 24 hours. At the end of the reaction, the medium is concentrated and then taken up in DCM and water and 20 ml of 1N sodium hydroxide. The aqueous phase is extracted three times with DCM. The organic phases are combined, dried over magnesium sulfate, filtered and concentrated. The crude reaction product is purified by flash chromatography on silica to give the desired compound (1.56 g; 46%).

¹H NMR, DMSO-d₆ (ppm): 11.08 (s, 1H); 9.00 (s, 1H); 8.59 (s, 1H); 8.53 (d, 2H); 8.38 (d, 1H); 8.31 (d, 1H); 7.78 (d, 2H).

### Example 344B

N-(Pyrid-4-yl)-5-aminobenzo[b]thiophene-2-carboxamide. The hydrochloride of compound 344B is prepared from the hydrochloride of derivative 344A (630 mg), according to the conditions used for the preparation of 229, and abiding by the proportions of the various reagents and using water and methanol as solvents. Amount obtained: 496 mg (86%).

¹H NMR, DMSO-d₆ (ppm): 11.07 (s, 1H); 8.60 (d, 2H); 8.21 (s, 1H); 7.99 (d, 2H); 7.69 (d, 1H); 7.09 (s, 1H); 6.91 (dd, 1H).

### EXAMPLE 344

Compound 344 is prepared from the derivative 344B (780 mg) and 1-methyl-2-formylbenzimidazole, according to the conditions used for the preparation of 230 and abiding by the proportions of the various reagents. Amount obtained: 613 mg (51%).

¹H NMR, DMSO-d₆ (ppm): 10.65 (s, 1H); 8.48 (d, 2H); 8.18 (s, 1H); 7.80-7.72 (m, 3H); 7.61 (d, 1H); 7.52 (d, 1H); 7.25-7.15 (m, 3H); 7.08 (dd, 1H); 6.56 (t, 1H); 4.64 (d, 2H); 3.86 (s, 3H).

### EXAMPLE 345

{5-[(1-Methyl-1H-benzoimidazol-2-ylmethyl)amino]benzo[b]thiophen-2-yl}-(4-ethylpiperazin-1-yl)methanone

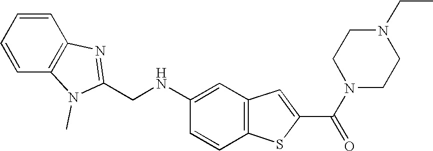

### Example 345A

### (5-Nitrobenzo[b]thiophen-2-yl)-(4-ethylpiperazin-1-yl)-methanone

Compound 345A is prepared from derivative 257A (1.6 g; 7.2 mmol) and from 1-ethylpiperazine, according to the conditions used for the preparation of 89A, and abiding by the proportions of the various reagents. At the end of the reaction, the medium is concentrated, and then taken up in DCM and water and 20 ml of 1N sodium hydroxide. The aqueous phase is extracted three times with DCM. The organic phases are combined, dried over magnesium sulfate, filtered and concentrated. The crude reaction product is purified by flash chromatography on silica to give the desired compound (2.2 g; 96%).

¹H NMR, DMSO-dr (ppm): 8.87 (d, 1H); 8.32 (d, 1H); 8.25 (dd, 1H); 7.98 (s, 1H); 3.68 (bs, 4H); 2.44 (bs, 4H); 2.38 (q, 2H); 1.02 (s, 3H).

### Example 345B

### b 5-Aminobenzo[b]thiophen-2-yl)-(4-ethylpiperazin-1-yl)-methanone

Compound 345B is prepared from derivative 345A (2.2 g; 6.8 mmol), according to the conditions used for the preparation of 229, and abiding by the proportions of the various reagents. Amount obtained: 1.93 g (97%).

¹H NMR, DMSO-d₆ (ppm): 7.58 (d, 1H); 7.41 (s, 1H); 6.98 (d, 1H); 6.79 (dd, 1H); 5.19 (bs, 2H); 3.65 (bs, 4H); 2.45-2.35 (m, 6H); 1.01 (t, 3H).

### EXAMPLE 345

Compound 345 is prepared from the derivative 345B (1.0 g) and from 1-methyl-2-formylbenzimidazole, according to the conditions described for the preparation of 170, and abiding by the proportions of the various reagents. Amount obtained: 1.25 g (83%).

HPLC (C₁₈ XTerra, λ 220 nm, 100% H₂O to 100% CH₃CN (+0.1% TFA) over 8 minutes): purity: 99%.

¹H NMR, DMSO-d₆ (ppm): 7.66 (d, 1H); 7.60 (d, 1H); 7.52 (d, 1H); 7.45 (s, 1H); 7.25-7.10 (m, 3H); 7.02 (dd, 1H); 6.42 (t, 1H); 4.59 (d, 2H); 3.84 (s, 3H); 3.64 (bs, 4H); 2.40-2.30 (m, 6H); 1.01 (t, 3H).

### Examples 346 to 348

Compounds 346 to 349 are prepared in the form of HCl salts from the derivatives 260B, 258B or 261B and from 1-methyl-2-formylbenzimidazole, according to the conditions described for the preparation of 344, and abiding by the proportions of the various reagents. The products are then purified by flash chromatography on silica, and then taken up in a mixture of water, acetonitrile and hydrochloric acid (1N in water), and finally freeze-dried in order to be characterized.

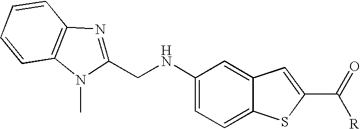

| | | | Mass | Purity |
|---|---|---|---|---|
| Ex. | R | Compound name | (M + H)⁺ | HPLC* |
| 346 | 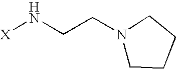 | N-(2-Pyrrolidin-1-ylethyl)-5-[(1-methyl-1H- benzoimidazol-2-ylmethyl)amino]benzo[b]- thiophene-2-carboxamide | 434 | 99 |
| 347 | 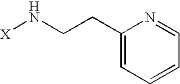 | N-(2-Pyrid-2-ylethyl)-5-[(1-methyl-1H- benzoimidazol-2-ylmethyl)amino]benzo[b]- thiophene-2-carboxamide | 441 | 98 |
| 348 | 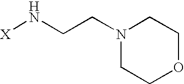 | N-(2-Morpholino-4-ylethyl)-5-[(1-methyl- 1H-benzoimidazol-2-ylmethyl)amino]- benzo[b]thiophene-2-carboxamide | 450 | 96 |

*HPLC conditions [C₁₈ XTerra MS, 4.6 × 50 mm, 5 μm; λ = 220 nm; gradient 100% H₂O(+0.05% TFA) to 100% CH₃CN(+0.05% TFA) over 6 minutes] ¹H NMR, DMSO-d₆(ppm).
Example 346: 8.56(t, 1H); 7.82(s, 1H); 7.66(d, 1H); 7.60(d, 1H); 7.52(d, 1H); 7.4-7.15(m, 3H); 7.01(dd, 1H); 6.44(t, 1H); 4.60(d, 2H); 3.85(s, 3H); 3.36(t, 2H); 2.56(t, 2H); 2.54-5.49(m, 4H + DMSO); 1.67(bs, 4H).
Example 347: 8.92(t, 1H); 8.76(d, 1H); 8.34(t, 1H); 7.97(d, 1H); 7.85-7.70(m, 6H); 7.65-7.50(m, 2H); 7.12(s, 1H); 7.03(d, 1H); 4.97(s, 2H); 4.07(s, 3H); 3.69(d, 2H); 3.85(s, 3H); 3.60(q, 2H); 3.00(t, 2H).
Example 348: 8.54(bs, 1H); 7.81(s, 1H); 7.66(d, 1H); 7.60(d, 1H); 7.51(d, 1H); 7.3-7.15(m, 3H); 7.01(d, 1H); 6.44(bs, 1H); 4.61(bs, 2H); 3.85(s, 3H); 3.56(bs, 4H); 3.36(bs, 2H); 2.50-5.2(m, 6H + DMSO).

### Examples 349 to 376

Compounds 349 to 376 are prepared in the form of HCl salts from the derivatives 341, 342, 344, 345, 346, 347 or 348 and from the corresponding acid chlorides, according to the conditions described for the preparation of 91, and abiding by the proportions of the various reagents. The products are then purified by filtration on silica using CombiFlash Optix 10 (Isco), and using a gradient of methanol in dichloromethane (0 to 20%). Finally, they are taken up in water, acetonitrile and hydrochloric acid (1N in water), and then freeze-dried in order to be characterized.

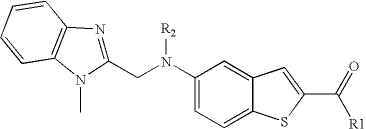

| | | | | Mass | HPLC |
|---|---|---|---|---|---|
| Ex. | R1 | R2 | Compound name | (M + H)⁺ | purity* |
| 349 | 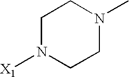 | X₂COnPr | N-(1-Methyl-1H-benzoimidazol-2-ylmethyl)- N-[2-(4-methylpiperazine-1- carbonyl)benzo[b]thiophen-5-yl]butyramide | 490 | 97 |
| 350 | 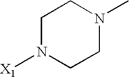 | 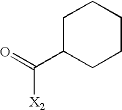 | N-(1-Methyl-1H-benzoimidazol-2-ylmethyl)- N-[2-(4-methylpiperazine-1- carbonyl)benzo[b]thiophen-5-yl]cyclo- hexanecarboxamide | 530 | 97 |
| 351 | 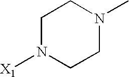 | 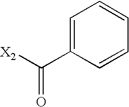 | N-(1-Methyl-1H-benzoimidazol-2-ylmethyl)- N-[2-(4-methylpiperazine-1- carbonyl)benzo[b]thiophen-5-yl]benzamide | 524 | 95 |
| 352 | 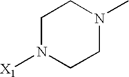 | 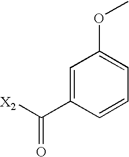 | N-(1-Methyl-1H-benzoimidazol-2-ylmethyl)- N-[2-(4-methylpiperazine-1- carbonyl)benzo[b]thiophen-5-yl]-3- methoxybenzamide | 554 | 84 |
| 353 | 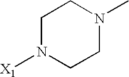 | X₂COnPr | N-(Pyrid-3-ylmethyl)-5-[butyryl-(1-methyl- 1H-benzoimidazol-2-ylmethyl)amino]- benzo[b]thiophene-2-carboxamide | 498 | 94 |
| 354 | 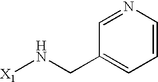 | 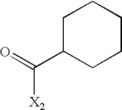 | N-(Pyrid-3-ylmethyl)-5-[(1-methyl-1H- benzoimidazol-2-ylmethyl)cyclohexane- carbonylamino]benzo[b]thiophene-2- carboxamide | 538 | 89 |
| 355 | 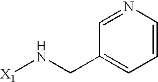 | 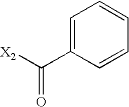 | N-(Pyrid-3-ylmethyl)-5-[benzoyl-(1-methyl- 1H-benzoimidazol-2-ylmethyl)amino]- benzo[b]thiophene-2-carboxamide | 532 | 94 |
| 356 | 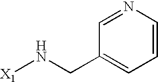 | 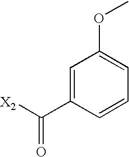 | N-(Pyrid-3-ylmethyl)-5-[(1-methyl-1H- benzoimidazol-2-ylmethyl)-(3-methoxy- benzoyl)amino]benzo[b]thiophene-2- carboxamide | 562 | 95 |
| 357 | 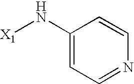 | X₂COnPr | N-(Pyrid-4-yl)-5-[butyryl-(1-methyl-1H- benzoimidazol-2-ylmethyl)amino]- benzo[b]thiophene-2-carboxamide | 484 | 88 |
| 358 | 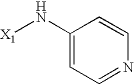 | 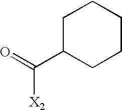 | N-(Pyrid-4-yl)-5-[(1-methyl-1H- benzoimidazol-2-ylmethyl)cyclohexane- carbonylamino]benzo[b]thiophene-2- carboxamide | 524 | 92 |
| 359 | 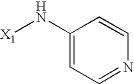 | 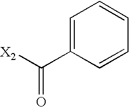 | N-(Pyrid-4-yl)-5-[benzoyl-(1-methyl-1H- benzoimidazol-2-ylmethyl)amino]- benzo[b]thiophene-2-carboxamide | 518 | 94 |
| 360 | 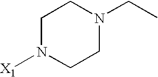 | X₂COnPr | N-(1-Methyl-1H-benzoimidazol-2-ylmethyl)- N-[2-(4-ethylpiperazine-1- carbonyl)benzo[b]thiophen-5-yl]butyramide | 95 | 504 |
| 361 | 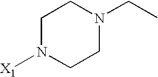 | 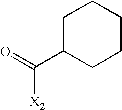 | N-(1-Methyl-1H-benzoimidazol-2-ylmethyl)- [2-(4-ethylpiperazine-1- carbonyl)benzo[b]thiophen-5- yl]cyclohexanecarboxamide | 91 | 544 |
| 362 | 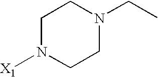 | 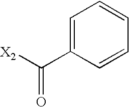 | N-(1-Methyl-1H-benzoimidazol-2-ylmethyl)- N-[2-(4-ethylpiperazine-1- carbonyl)benzo[b]thiophen-5-yl]benzamide | 93 | 538 |
| 363 | 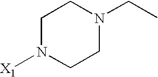 | 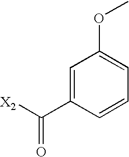 | N-(1-Methyl-1H-benzoimidazol-2-ylmethyl)- N-[2-(4-ethylpiperazine-1- carbonyl)benzo[b]thiophen-5-yl]-3- methoxybenzamide | 93 | 568 |
| 364 | 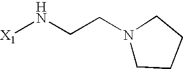 | X₂COnPr | N-(2-Pyrrolidin-1-ylethyl)-5-[(1-methyl-1H- benzoimidazol-2-ylmethyl)butanoylamino]- benzo[b]thiophene-2-carboxamide | 504 | 90 |
| 365 | 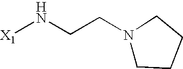 | 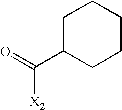 | N-(2-Pyrrolidin-1-ylethyl)-5-[(1-methyl-1H- benzoimidazol-2-ylmethyl)cyclohexane- carbonylamino]benzo[b]thiophene-2- carboxamide | 544 | 95 |
| 366 | 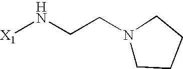 | 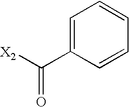 | N-(2-Pyrrolidin-1-ylethyl)-5-[benzoyl-(1- methyl-1H-benzoimidazol-2-ylmethyl)- amino]benzo[b]thiophene-2-carboxamide | 538 | 92 |
| 367 | 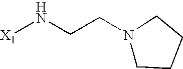 | 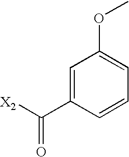 | N-(2-Pyrrolidin-1-ylethyl)-5-[(1-methyl-1H- benzoimidazol-2-ylmethyl)-(3-methoxy- benzoyl)amino]benzo[b]thiophene-2- carboxamide | 568 | 87 |
| 368 | 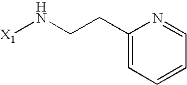 | X₂COnPr | N-(2-Pyrid-2-ylethyl)-5-[butyryl-(1-methyl- 1H-benzoimidazol-2-ylmethyl)amino]- benzo[b]thiophene-2-carboxamide | 512 | 96 |
| 369 | 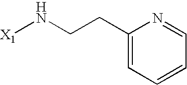 | 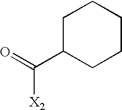 | N-(2-Pyrid-2-ylethyl)-5-[(1-methyl-1H- benzoimidazol-2-ylmethyl)cyclohexane- carbonylamino]benzo[b]thiophene-2- carboxamide | 552 | 98 |
| 370 | 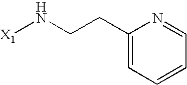 | 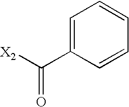 | N-(2-Pyrid-2-ylethyl)-5-[benzoyl-(1-methyl- 1H-benzoimidazol-2-ylmethyl)amino]- benzo[b]thiophene-2-carboxamide | 546 | 97 |
| 371 | 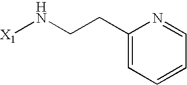 | 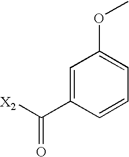 | N-(2-Pyrid-2-ylethyl)-5-[(1-methyl-1H- benzoimidazol-2-ylmethyl)-(3-methoxy- benzoyl)amino]benzo[b]thiophene-2- carboxamide | 576 | 98 |
| 372 | 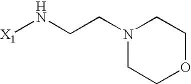 | X₂COnPr | N-(2-Morpholino-4-ylethyl)-5-[(1-methyl- 1H-benzoimidazol-2-ylmethyl)butanoyl- amino]benzo[b]thiophene-2-carboxamide | 520 | 98 |
| 373 | 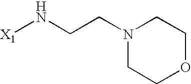 | 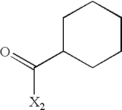 | N-(2-Morpholino-4-ylethyl)-5-[(1-methyl- 1H-benzoimidazol-2-ylmethyl)cyclohexane- carbonylamino]benzo[b]thiophene-2- carboxamide | 560 | 97 |
| 374 | 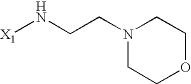 | 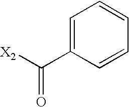 | N-(2-Morpholino-4-ylethyl)-5-[benzoyl-(1- methyl-1H-benzoimidazol-2-ylmethyl)- amino]benzo[b]thiophene-2-carboxamide | 554 | 94 |
| 375 | 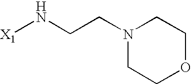 | 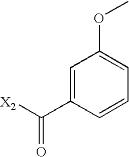 | N-(2-Morpholino-4-ylethyl)-5-[(1-methyl- 1H-benzoimidazol-2-ylmethyl)-(3-methoxy- benzoyl)amino]benzo[b]thiophene-2- carboxamide | 584 | 95 |

*HPLC conditions [C₁₈ XTerra MS, 4.6 × 50 mm, 5 μm; λ = 220 nm; gradient 100% H₂O(+0.05% TFA) to 100% CH₃CN(+0.05% TFA) over 6 minutes] ¹H NMR, DMSO-d₆(ppm).
Example 351: 11.19(bs, 1H); 8.00(s, 1H); 7.95(d, 1H); 7.88(bd, 1H); 7.80(d, 1H); 7.71(s, 1H); 7.55-7.45(m, 3H); 7.44(d, 2H); 7.30-7.05(m, 3H); 5.60(s, 2H); 4.55-4.3(m, 2H); 4.00(s, 3H); 3.9-3.3(m, H₂O + 4H); 3.15-3.05(m, 2H); 2.77(bs, 3H).
Example 356: 9.72(t, 1H); 8.85(s, 1H); 8.76(d, 1H); 8.38(d, 1H); 8.13(d, 1H); 8.00-7.80(m, 4H); 7.60-7.53(m, 3H); 7.12(t, 1H); 7.07(s, 1H); 6.97(d, 1H); 6.82(dd, 1H); 5.64(s, 2H); 4.62(d, 2H); 3.95(s, 3H); 3.60(s, 3H).
Example 358: 11.88(bs, 1H); 10.85(bs, 1H); 8.50(d, 2H); 8.35(s, 1H); 8.09(d, 1H); 8.05(s, 1H); 7.75(d, 2H); 7.53-7.46(m, 3H); 7.22(t, 1H); 7.14(t, 1H); 5.20(s, 2H); 3.79(s, 3H); 2.2-2.15(m, 1H); 1.8-0.8(m, 10H).
Example 365: 10.70(bs, 1H); 9.38(t, 1H); 8.29(bs, 2H); 8.12(d, 1H); 7.89(d, 1H); 7.79(d, 1H); 7.65-7.50(m, 3H); 5.41(s, 2H); 3.95(s, 3H); 3.65(bs, H₂O + 4H); 3.35(bd, 2H): 3.05-3.00(m, 2H); 2.22(bt, 1H); 1.95-0.70(m, 14H).
Example 372: 11.04(s, 1H); 9.39(t, 1H); 8.29(s, 1H); 8.25(s, 1H); 8.13(d, 1H); 7.93(d, 1H); 7.83(d, 1H); 7.65-7.50(m, 3H); 5.46(s, 2H); 3.98(bd, 2H); 3.96(s, 3H); 3.84(bt, 2H); 3.75-3.70(m, 2H); 3.55(bd, 2H); 3.35-3.30(m, 2H); 3.13(qe., 2H); 2.16(t, 2H); 2.51(sext., 2H); 0.78(t, 3H).

### Examples 377 to 383

Compounds 377 to 383 are prepared in the form of HCl salts from the derivatives 341, 342, 344, 345, 346, 347 or 348 and from n-propionaldehyde, according to the conditions described for the preparation of 170, and abiding by the portions of the various reagents. The products are then purified by filtration on silica using CombiFlash Optix 10 (Isco), and using a gradient of methanol in dichloromethane (0 to 20%). Finally, they are taken up in water, acetonitrile and hydrochloric acid (1N in water), and then freeze-dried in order to be characterized.

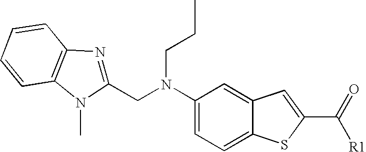

| | | | Mass | Purity |
|---|---|---|---|---|
| Ex. | R₁ | Compound name | (M + H)⁺ | HPLC* |
| 377 | 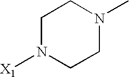 | {5-[(1-Methyl-1H-benzoimidazol-2- ylmethyl)propylamino]- benzo[b]thiophen-2-yl}-(4- methylpiperazin-1-yl)methanone | 462 | 99 |
| 378 | 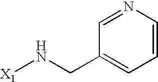 | N-(Pyrid-3-ylmethyl)-5-[(1-methyl-1H-benzo- imidazol-2-ylmethyl)propylamino]- benzo[b]thiophene-2-carboxamide | 470 | 97 |
| 379 | 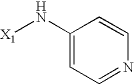 | N-(Pyrid-4-yl)-5-[(1-methyl-1H-benzoimidazol-2- ylmethyl)propylamino]benzo[b]thiophene-2- carboxamide | 456 | 92 |
| 380 | 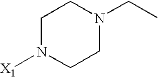 | {5-[(1-Methyl-1H-benzoimidazol-2- ylmethyl)propylamino]benzo[b]thiophen-2-yl}-(4- ethylpiperazin-1-yl)methanone | 476 | 99 |
| 381 | 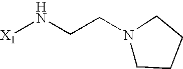 | N-(2-Pyrrolidin-1-ylethyl)-5-[(1-methyl-1H-benzo- imidazol-2-ylmethyl)propylamino]- benzo[b]thiophene-2-carboxamide | 476 | 98 |
| 382 | 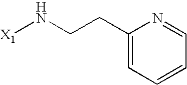 | N-(2-Pyrid-2-ylethyl)-5-[(1-methyl-1H-benzo- imidazol-2-ylmethyl)propylamino]benzo[b]- thiophene-2-carboxamide | 484 | 95 |
| 383 | 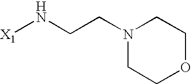 | N-(2-Morpholino-4-ylethyl)-5-[(1-methyl-1H- benzoimidazol-2-ylmethyl)propylamino]- benzo[b]thiophene-2- carboxamide | 492 | 97 |

*HPLC conditions [C₁₈ XTerra MS, 4.6 × 50 mm, 5 μm; λ = 220 nm; gradient 100% H₂O(+0.05% TFA) to 100% CH₃CN(+0.05% TFA) over 6 minutes] ¹H NMR, DMSO-d₆(ppm).
Example 378: 9.63(t, 1H); 8.84(s, 1H); 8.76(d, 1H); 8.37(d, 1H); 8.05-7.95(m, 2H); 7.90(t, 1H); 7.84(d, 1H); 7.72(d, 1H); 7.61(t, 1H); 7.55(t, 1H); 7.30(s, 1H); 7.17(d, 1H); 5.23(s, 2H); 6.61(d, 2H); 4.04(s, 3H); 3.52(t, 2H); 1.68(qe, 2H); 0.94(t, 3H).
Example 381: 10.82(bs, 1H); 9.22(t, 1H); 8.04(s, 1H); 7.99(d, 1H); 7.83(d, 1H); 7.72(d, 1H); 7.61(t, 1H); 7.55(t, 1H); 7.29(d, 1H); 7.16(dd, 1H); 5.23(s, 2H); 4.11(s, 3H); 3.9-3.5(m, H₂O + 4H); 3.51(t, 2H); 3.33(bd, 2H); 3.01(bs, 2H); 2.00(bs, 2H); 1.90-1.85(m, 2H); 1.68(sext., 2H); 0.94(t, 3H).
Example 383: 9.25(t, 1H); 8.05(s, 1H); 8.01(d, 1H); 7.83(d, 1H); 7.73(d, 1H); 7.63(t, 1H); 7.56(t, 1H); 7.29(s, 1H); 7.16(dd, 1H); 5.24(s, 2H); 4.05(s, 3H); 4.00-3.95(m, 2H); 3.90-3.80(m, 2H); 3.69(bd, 2H); 3.55-3.45(m, 4H); 3.30(bd, 2H); 3.11(qe, 2H); 1.68(sext., 2H); 0.94(t, 3H).

### Examples 384 to 390

Compounds 384 and 388 are prepared in the form of HCl salts from the derivatives 341 or 346 and from benzenesulfonyl chloride, according to the conditions described for the preparation of 91, and abiding by the proportions of the various reagents. Compounds 385, 386, 387, 389 and 390 are prepared in the form of HCl salts from the derivatives 342, 344, 345, 347 or 348 and from benzenesulfonyl chloride, according to the conditions described for the preparation of 36, and abiding by the proportions of the various reagents.

The products are then purified by filtration on silica using CombiFlash Optix 10 (Isco), and using a gradient of methanol in dichloromethane (0 to 20%). Finally, they are taken up in water, acetonitrile and hydrochloric acid (1N in water), and then freeze-dried in order to be characterized.

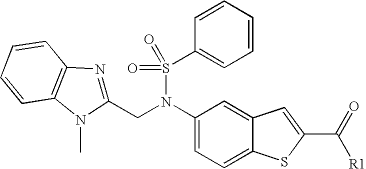

| | | | Mass | Purity |
|---|---|---|---|---|
| Ex. | R₁ | Compound name | (M + H)⁺ | HPLC* |
| 384 | 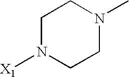 | {5-[Benzenesulfonyl-(1-methyl-1H- benzoimidazol-2-ylmethyl)amino]- benzo[b]thiophen-2-yl}-(4- methylpiperazin-1-yl)methanone | 560 | 90 |
| 385 | 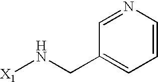 | N-(Pyrid-3-ylmethyl)-5-[benzenesulfonyl-(1- methyl-1H-benzoimidazol-2-ylmethyl)amino]- benzo[b]thiophene-2-carboxamide | 568 | 96 |
| 386 | 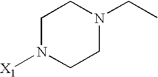 | {5-[Benzenesulfonyl-(1-methyl-1H- benzoimidazol-2-ylmethyl)amino]- benzo[b]thiophen-2-yl}-(4-ethyl- piperazin-1-yl)methanone | 574 | 94 |
| 387 | 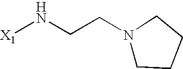 | N-(Pyrrolidin-1-ylethyl)-5-[benzenesulfonyl-(1- methyl-1H-benzoimidazol-2-ylmethyl)amino]- benzo[b]thiophene-2-carboxamide | 574 | 91 |
| 388 | 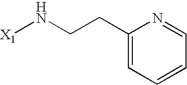 | N-(2-Pyrid-2-ylethyl)-5-[benzenesulfonyl-(1- methyl-1H-benzoimidazol-2-ylmethyl)amino]- benzo[b]thiophene-2-carboxamide | 582 | 98 |
| 389 | 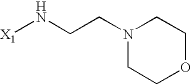 | N-(2-Morpholino-4-ylethyl)-5-[benzenesulfonyl- (1-methyl-1H-benzoimidazol-2-ylmethyl)amino]- benzo[b]thiophene-2-carboxamide | 590 | 99 |

*HPLC conditions [C₁₈ XTerra MS, 4.6 × 50 mm, 5 μm; λ = 220 nm; gradient 100% H₂O(+0.05% TFA) to 100% CH₃CN(+0.05% TFA) over 6 minutes] ¹H NMR, DMSO-d₆(ppm).
Example 384: 11.34(bs, 1H); 8.00(d, 1H); 7.95-7.60(m, 9H); 7.55-7.45(m, 2H); 7.22(d, 1H); 5.46(s, 2H); 4.50-4.25(m, 2H); 4.03(s, 3H); 3.60-3.30(m, 4H); 3.20-3.00(m, 2H); 2.77(s, 3H).
Example 388: 9.15(t, 1H); 8.80(d, 1H); 8.43(t, 1H); 8.03(s, 1H); 8.00-7.60(m, 11H); 7.58-7.45(m, 2H); 7.25(d, 1H); 5.49(s, 2H); 4.03(s, 3H); 3.75-3.70(m, 2H); 3.31(t, 2H).
Example 389: 12.03(bs, 1H); 9.38(t, 1H); 8.21(s, 1H); 7.99(d, 1H); 7.88-7.63(m, 8H); 7.55-7.48(m, 2H); 7.28(dd, 1H); 5.50(s, 2H); 4.03(s, 3H); 3.97(bd, 2H); 3.83(bt, 2H); 3.75-3.65(m, 2H); 3.53(bd, 2H); 3.25-3.00(m, 2H); 3.32(bs, 2H).

### EXAMPLE 391

### N-(Pyrid-4-yl)-5-{[3-(4-cyanobenzyl)-3H-imidazol-4-ylmethyl]amino}benzo[b]thiophene-2-carboxamide

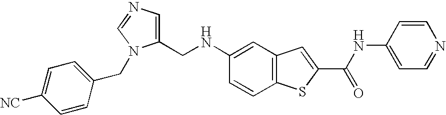

Compound 391 is prepared from derivative 88 according to the method described for the preparation of 344A, in the presence of an excess of 4-aminopyridine.

### Examples 392 to 395

Compounds 392 to 395 are prepared in the form of HCl salts from the derivative 261 and from the corresponding acid chlorides, according to the conditions described for the preparation of 91, and abiding by the proportions of the various reagents. The products are then purified by filtration on silica using CombiFlash Optix 10 (Isco), and using a gradient of methanol in dichloromethane (0 to 20%). Finally, they are taken up in water, acetonitrile and hydrochloric acid (1N in water), and then freeze-dried in order to be characterized.

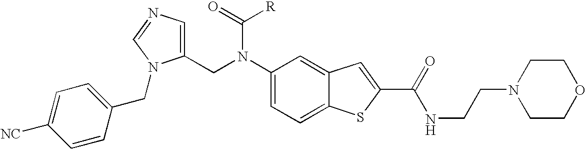

| | | | Mass | Purity |
|---|---|---|---|---|
| Ex. | R | Compound name | (M + H)⁺ | HPLC* |
| 392 | 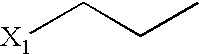 | N-(2-Morpholino-4-ylethyl)-5-{[3-(4- cyanobenzyl)-3H-imidazol-4- ylmethyl]butanoylamino}benzo[b]thiophene-2- carboxamide | 571 | 99 |
| 393 | 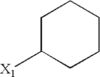 | N-(2-Morpholino-4-ylethyl)-5-{[3-(4- cyanobenzyl)-3H-imidazol-4- ylmethyl]cyclohexanecarbonylamino}- benzo[b]thiophene-2-carboxamide | 611 | 97 |
| 394 | 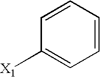 | N-(2-Morpholino-4-ylethyl)-5-{benzoyl-[3-(4- cyanobenzyl)-3H-imidazol-4- ylmethyl]amino}benzo[b]thiophene-2-carboxamide | 605 | 98 |
| 395 | 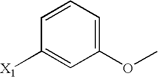 | N-(2-Morpholino-4-ylethyl)-5-{[3-(4- cyanobenzyl)-3H-imidazol-4-ylmethyl]-3- methoxybenzoylamino}benzo[b]thiophene-2- carboxamide | 635 | 97 |

*HPLC conditions [C₁₈ XTerra MS, 4.6 × 50 mm, 5 μm; λ = 220 nm; gradient 100% H₂O(+0.05% TFA) to 100% CH₃CN(+0.05% TFA) over 6 minutes] ¹H NMR, DMSO-d₆(ppm).
Example 395: 11.06(bs, 1H); 9.38(t, 1H); 9.31(s, 1H); 8.10(s, 1H); 7.90-7.80(m, 3H); 7.72(s, 1H); 7.65(s, 1H); 7.41(d, 2H); 7.19(d, 1H); 7.06(t, 1H); 6.79(bs, 3H); 5.66(s, 2H); 5.13(s, 2H); 4.0-3.95(m, 2H); 3.88-3.81(m, 2H); 3.71-3.68(m, 2H); 3.57(s, 3H); 3.57-3.52(m, 2H); 3.32(bs, 2H); 3.20-3.10(m, 2H).

### Examples 396 to 400

Compounds 396 to 400 are prepared in the form of HCl salts from derivative 261 or derivative 391 and from the corresponding acid chlorides, according to the conditions described for the preparation of 91, and abiding by the proportions of the various reagents. The products are then purified by filtration on silica using CombiFlash Optix 10 (Isco), and using a gradient of methanol in dichloromethane (0 to 20%). Finally, they are taken up in water, acetonitrile and hydrochloric acid (1N in water), and then freeze-dried in order to be characterized.

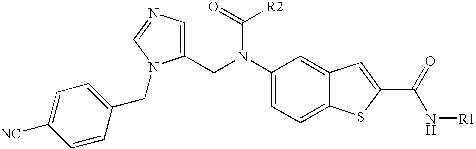

| Ex. | R₁ | R₂ | Compound name |
|---|---|---|---|
| 396 | 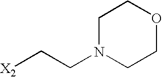 | 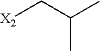 | N-(2-Morpholino-4-ylethyl)-5-{[3-(4-cyanobenzyl)- 3H-imidazol-4-ylmethyl]-3-methylbutanoyl- amino}-benzo[b]thiophene-2-carboxamide |
| 397 | 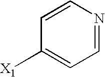 | 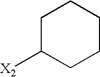 | N-(Pyrid-4-yl)-5-{[3-(4-cyanobenzyl)-3H-imidazol- 4-ylmethyl]cyclohexanecarbonylamino}benzo[b]- thiophene-2-carboxamide |
| 398 | 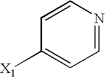 | 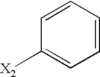 | N-(Pyrid-4-yl)-5-{benzoyl-[3-(4-cyanobenzyl)-3H- imidazol-4-ylmethyl]amino}benzo[b]- thiophene-2-carboxamide |
| 399 | 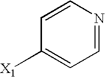 | 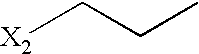 | N-(Pyrid-4-yl)-5-{[3-(4-cyanobenzyl)-3H-imidazol- 4-ylmethyl]butanoylamino}benzo[b]- thiophene-2-carboxamide |
| 400 | 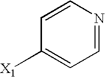 | 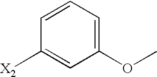 | N-(Pyrid-4-yl)-5-{[3-(4-cyanobenzyl)-3H-imidazol- 4-ylmethyl]-3-methoxybenzoylamino}benzo[b]- thiophene-2-carboxamide |

Example 396: HPLC(XTerra MS, λ = 220 nm, 100% H₂O to 100% CH₃CN(+0.1% TFA) over 6 minutes): purity 93%. Mass spectrum(ESI): m/z 585(MH+).
¹H NMR, DMSO-d₆(ppm): 9.43(bs, 1H); 9.29(s, 1H); 8.25(s, 1H); 8.04(d, 1H); 7.85(d, 2H); 7.77(s, 1H); 7.54(s, 1H); 7.42(d, 2H); 7.25(d, 1H); 5.58(s, 2H); 4.94(s, 2H); 4.00-3.96(m, 2H); 3.90-3.86(m, 2H); 3.74-3.72(m, 2H); 3.58-3.54(m, 2H); 3.34(bs, 2H); 3.15-3.12(m, 2H); 2.05-1.92(m, 1H); 1.87(bs, 2H); 0.75(d, 6H).

### Examples 401 to 403

Compound 401 is prepared in the form of HCl salts from derivative 261 and from benzenesulfonyl chloride, according to the conditions described for the preparation of 36, and abiding by the proportions of the various reagents. Compound 402 is prepared in the form of HCl salts from derivative 261 and from benzaldehyde, according to the conditions described for the preparation of 159A, and abiding by the proportions of the various reagents. Compound 403 is prepared in the form of HCl salts from derivative 261 and from propionaldehyde, according to the conditions described for the preparation of 170, and abiding by the proportions of the various reagents.

The products are then purified by filtration on silica using CombiFlash Optix 10 (Isco), and using a gradient of methanol in dichloromethane (0 to 20%).

Finally, they are taken up in water, acetonitrile and hydrochloric acid (1N in water), and then freeze-dried in order to be characterized.

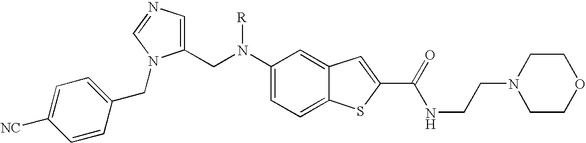

| | | | Mass | Purity |
|---|---|---|---|---|
| Ex. | R | Compound name | (M + H)⁺ | HPLC* |
| 401 | 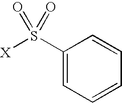 | N-(2-Morpholino-4-ylethyl)-5-{benzenesulfonyl- [3-(4-cyanobenzyl)-3H-imidazol-4-ylmethyl]- amino}benzo[b]thiophene-2-carboxamide | 641 | 99 |
| 402 | 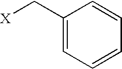 | N-(2-Morpholino-4-ylethyl)-5-{benzyl-[3-(4- cyanobenzyl)-3H-imidazol-4-ylmethyl]amino}- benzo[b]thiophene-2-carboxamide | 591 | 93 |
| 403 | 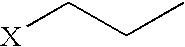 | N-(2-Morpholino-4-ylethyl)-5-{[3-(4- cyanobenzyl)-3H-imidazol-4-ylmethyl]- propylamino}benzo[b]thiophene-2-carboxamide | 543 | 99 |

*HPLC conditions [C₁₈ XTerra MS, 4.6 × 50 mm, 5 μm; λ = 220 nm; gradient 100% H₂O(+0.05% TFA) to 100% CH₃CN(+0.05% TFA) over 6 minutes] ¹H NMR, DMSO-d₆(ppm).
Example 401: 11.03(bs, 1H); 9.38(t, 1H); 9.21(s, 1H); 8.10(s, 1H); 7.94(d, 2H); 7.89(d, 1H); 7.77(t, 1H); 7.64-7.59(m, 2H); 7.54-7.46(m, 6H); 6.88(dd, 1H); 5.67(s, 2H); 4.93(s, 2H); 4.00-3.96(m, 2H); 3.88-3.81(m, 2H); 3.72-3.70(m, 2H); 3.57-3.53(m, 2H); 3.33(bs, 2H); 3.15-3.10(m, 2H).
Example 403: 11.25(bs, 1H); 9.44-9.21(m, 2H); 8.04(s, 1H); 7.91(d, 2H); 7.73(d, 1H); 7.48(s, 1H); 7.44(d, 2H); 6.99(bs, 1H); 6.87(d, 1H); 5.66(s, 2H); 4.49(s, 2H); 3.99-3.96(m, 2H); 3.91-3.84(m, 2H); 3.72-3.70(m, 2H); 3.57-3.53(m, 2H); 3.34-3.32(m, 2H); 3.20(t, 2H); 3.15-3.11(m, 2H); 1.44(sext., 2H); 0.81(t, 3H).

### Examples 404 to 406

Compound 404 is prepared in the form of HCl salts from derivative 391 and from benzenesulfonyl chloride, according to the conditions described for the preparation of 36, and abiding by the proportions of the various reagents.

Compounds 405 and 406 are prepared in the form of HCl salts from derivative 391 or from derivative 259B and from the corresponding aldehydes, according to the conditions described for the preparation of 170, and abiding by the proportions of the various reagents.

The products are then purified by filtration on silica using CombiFlash Optix 10 (Isco), and using a gradient of methanol in dichloromethane (0 to 20%). Finally, they are taken up in water, acetonitrile and hydrochloric acid (1N in water), and then freeze-dried in order to be characterized.

| Ex. | Structures | Compound name |
|---|---|---|
| 404 | 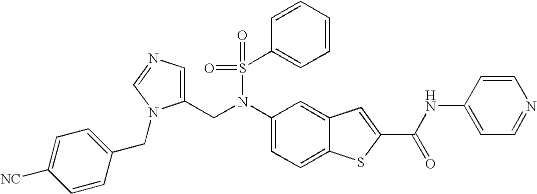 | N-(Pyrid-4-yl)-5-{benzenesulfonyl-[3- (4-cyanobenzyl)-3H-imidazol-4-ylmethyl]- amino}benzo[b]thiophene-2-carboxamide |
| 405 | 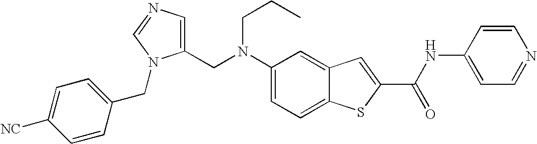 | N-(Pyrid-4-yl)-5-{[3-(4-cyanobenzyl)-3H- imidazol-4-ylmethyl]propylamino}- benzo[b]thiophene-2-carboxamide |
| 406 | 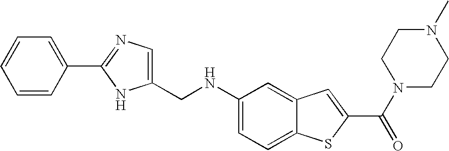 | {5-[(2-Phenyl-3H-imidazol-4- ylmethyl)amino]benzo[b]thiophen-2-yl}- (4-methylpiperazin-1-yl)methanone |

^{°}HPLC(XTerra MS, λ = 220 nm, 100% H₂O to 100% CH₃CN(+0.1% TFA) over 6 minutes): purity 91%. Mass spectrum(ESI): m/z 432(MH+).

### EXAMPLES 407 AND 408

Compounds 407 and 408 are prepared from derivative 249 according to the method described for the preparation of 344A in the presence of two equivalents of aminopyridine.

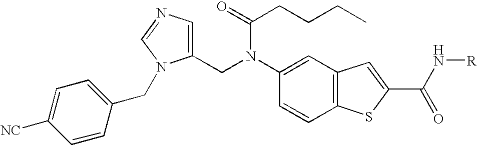

| | | | Mass | Purity |
|---|---|---|---|---|
| Ex. | R | Compound name | (M + H)⁺ | HPLC* |
| 407 | 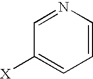 | N-(Pyrid-3-yl)-5-{[3-(4-cyanobenzyl)-3H-imidazol-4-ylmeth- yl]pentanoylamino}benzo[b]thiophene-2-carbox- amide | 549 | 99 |
| 408 | 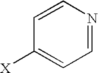 | N-(Pyrid-4-yl)-5-{[3-(4-cyanobenzyl)-3H-imidazol-4-ylmeth- yl]pentanoylamino}benzo[b]thiophene-2-carbox- amide | 549 | 95 |

¹H NMR, DMSO-d₆(ppm): 12.38(s, 1H); 9.28(s, 1H); 9.20(s, 1H); 8.78(d, 2H); 8.50(d, 2H); 8.12(d, 1H); 7.88(s, 1H); 7.84(d, 2H); 7.58(s, 1H); 7.40(d, 2H); 7.36(dd, 1H); 5.58(s, 2H); 4.94(s, 2H); 1.98(t, 2H); 1.40(quint., 2H); 1.12(sext., 2H); 0.73(t, 3H).

### EXAMPLE 409

4-(5-{[2-(4-Methylpiperazine-1-ylmethyl)benzo[b]thiophen-5-ylamino]methyl}imidazol-1-ylmethyl)benzonitrile

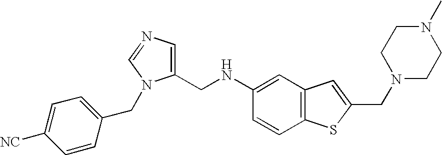

### Example 409A

### 2-(4-Methylpiperazin-1-ylmethyl)benzo[b]thiophen-5-ylamine

Compound 259B (300 mg; 1.0 mmol) is dissolved in anhydrous THF (3 ml) and lithium aluminum hydride (1M solution in THF; 4.35 ml; 4.3 mmol) is added slowly under a nitrogen atmosphere. The reaction mixture is stirred at 70° C. until the reaction is complete (5 hours). It is then cooled to room temperature and then neutralized (warning: very violent reaction) by successive additions of water (165 μl), of sodium hydroxide solution (15% in water; 165 μl) and of water (495 μl). The resulting suspension is filtered and the precipitate is washed with DCM. The filtrate is concentrated to give a pale yellow solid (336 mg; purity 84%). It is used in the rest of the operations without any other form of purification.

¹H NMR, DMSO-d₆ (ppm): 7.45 (d, 1H); 6.99 (s, 1H); 6.85 (s, 1H); 6.63 (d, 1H); 4.99 (s, 2H); 3.66 (s, 2H); 2.50-2.30 (m, 8H); 2.15 (s, 3H).

### EXAMPLE 409

Compound 409 (336 mg; 84%) is prepared from derivative 409A and from derivative 27A, according to the conditions described for the preparation of 170, and abiding by the proportions of the various reagents. Amount obtained: 388 mg (78%).

¹H NMR, DMSO-d₆ (ppm): 7.80 (d, 2H); 7.75 (s, 1H); 7.47 (d, 1H); 7.26 (d, 2H); 6.99 (s, 1H); 6.93 (s, 1H); 6.73 (s, 1H); 6.62 (d, 1H); 5.87 (bs, 1H); 5.38 (s, 2H); 4.07 (s, 2H); 3.67 (s, 2H); 2.55-2.20 (m, 8H); 2.14 (s, 3H).

### EXAMPLE 410

N-[3-(4-Cyanobenzyl)-3H-imidazol-4-ylmethyl]-N-[2-(4-methylpiperazin-1-ylmethyl)benzo[b]thiophen-5-yl]pentanamide

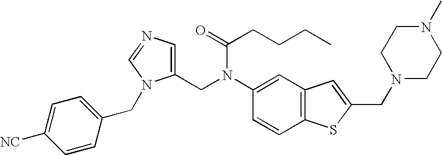

Compound 410 is prepared from derivative 409 (388 mg) and from pentanoyl chloride, according to the conditions described for the preparation of 91, and abiding by the proportions of the various reagents. The crude reaction product is purified by flash chromatography (90/9/1 DCMIMeOH/NH₄OH) to give the desired product (284 mg; 62%).

HPLC (C₁₈ XTerra, λ 220 nm), 100% H₂O to 100% CH₃CN (+0.1% TFA) over 8 minutes): purity: 98%.

¹H NMR, DMSO-d₆ (ppm): 7.86 (d, 1H); 7.77 (d, 2H); 7.74 (s, 1H); 7.38 (s, 1H); 7.23 (s, 1H); 7.19 (d, 2H); 6.95 (d, 1H); 6.53 (s, 1H); 5.32 (s, 2H); 4.81 (s, 2H); 3.76 (s, 2H); 2.6-2.2 (m, 8H); 2.16 (s, 3H); 1.87 (t, 2H); 1.36 (quint., 2H); 1.15-1.03 (m, 2H); 0.71 (s, 3H).

### EXAMPLE 411

N-[3-(4-Cyanobenzyl)-3H-imidazol-4-ylmethyl]-N-[2-(4-methylpiperazin-1-ylmethyl)benzo[b]thiophen-5-yl]pentanethioamide

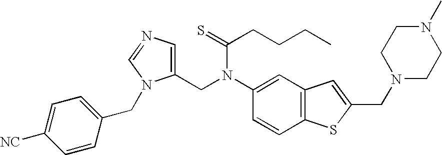

Compound 410 (215 mg; 0.4 mmol) is dissolved in toluene (3 ml) under a nitrogen atmosphere, and Lawesson's reagent (96 mg; 0.24 mmol) is added. The reaction medium is then heated at 115° C. for 3 hours. Pyridine (3 ml) is added and heating is continued for 18 hours. The mixture is then coevaporated twice with toluene and the residual oil is purified by preparative HPLC (Waters Prep 4000), on a LiChroprep RP-18 column (Merck; 50×150 mm; 15-25/μm) using a total gradient of from 100% water (0.1% HCl) to 100% acetonitrile (0.1% HCl) over 25 minutes, and then freeze-dried to give product 411 in the form of HCl salt (30 mg).

¹H NMR, DMSO-d₆ (ppm): 9.28 (s, 1H); 8.02 (d, 1H); 7.77-7.75 (m, 3H); 7.68 (s, 1H); 7.33 (d, 2H); 7.23 (d, 1H); 6.99 (d, 1H); 5.7-5.3 (m, 4H); 4.44-4.1 (m, 2H); 3.6-2.7 (m, 8H); 2.77 (s, 3H); 2.33 (t, 2H); 1.56 (quint., 2H); 1.06 (sext., 2H); 0.67 (s, 3H).

### EXAMPLES 412 to 418

Compounds 412 to 417 are prepared, in the form of HCl salts, from the derivatives 341, 346 or 348 and from the corresponding acid chlorides, according to the conditions described for the preparation of 91, and abiding by the proportions of the various reagents. Compound 418 is prepared in the form of the HCl salt from derivative 341 and from benzaldehyde, according to the conditions described for the preparation of 170, and abiding by the proportions of the various reagents.

The products are then purified by filtration on silica using CombiFlash Optix 10 (Isco), and using a gradient of methanol in dichloromethane (0 to 20%). Finally, they are taken up in water, acetonitrile and hydrochloric acid (1N in water), and then freeze-dried in order to be characterized.

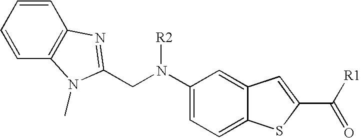

| Ex. | R₁ | R₂ | Compound Name |
|---|---|---|---|
| 412 | 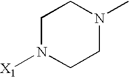 | 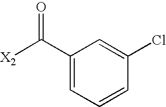 | N-(1-Methyl-1H-benzoimidazol-2-ylmethyl)-N-[2-(4-methyl- piperazine-1-carbo- nyl)benzo[b]thiophen-5-yl]-3-chloro- benzamide |
| 413 | 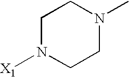 | 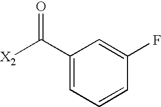 | N-(1-Methyl-1H-benzoimidazol-2-ylmethyl)-N-[2-(4-methyl- piperazine-1-carbo- nyl)benzo[b]thiophen-5-yl]-3-fluoro- benzamide |
| 414 | 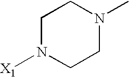 | 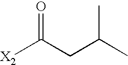 | N-(1-Methyl-1H-benzoimidazol-2-ylmethyl)-N-[2-(4-methyl- piperazine-1-carbo- nyl)benzo[b]thiophen-5-yl]-3-methyl- butanamide |
| 415 | 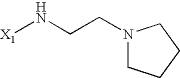 | 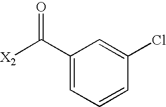 | N-(2-Pyrrolidin-1-ylethyl)-5-[(1-methyl-1H-benzo- imidazol-2-ylmethyl)-3-chloro- benzoylamino]benzo[b]thiophene-2-carbox- amide |
| 416 | 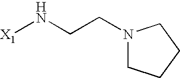 | 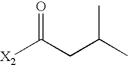 | N-(2-Pyrrolidin-1-ylethyl)-5-[(1-methyl-1H-benzo- imidazol-2-ylmethyl)-3-methyl- pentanoylamino]benzo[b]thiophene-2-carbox- amide |
| 417 | 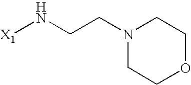 | 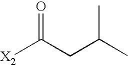 | N-(2-Morpholino-4-ylethyl)-5-[(1-methyl-1H-benzo- imidazol-2-ylmethyl)-3-methyl- pentanoylamino]benzo[b]thiophene-2-carbox- amide |
| 418 | 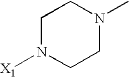 | 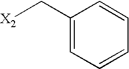 | {5-[(1-Methyl-1H-benzoimidazol-2-ylmeth- yl)benzylamino]benzo[b]thiophen-2-yl}-(4-methyl- piperazin-1-yl)methanone |

*HPLC (XTerra MS, λ = 220 nm, 100% H₂O to 100% CH₃CN (+0.1% TFA) over 6 minutes): purity 91%. Mass spectrum (ESI): m/z 561 (MH+).

A) Evaluation of the inhibition of protein farnesyl transferase

Principle:

The farnesylation of the dansylated peptide GCVLS, catalyzed with the enzyme protein farnesyl transferase, results in a change in the emission spectrum of the dansyl group, and especially an increase in the emission at 505 nm when the molecule is excited at 340 nm. When measured using the spectrofluorimeter, this emission is proportional to the activity of the enzyme (Pompliano et al., J. Am. Chem. Soc. 1992; 114: 7945-7946).

Materials

Reaction buffer:

55 mM TRIS/HCl pH 7.5; 5.5 mM DTT; 5.5 mM MgCl₂; 110 μM ZnCl₂, 0.22% B-octyl-B D-glucopyrannoside.

Substrates:

Farnesyl pyrophosphate (FPP), (Sigma)

Dansylated peptide dansyl-GCVLS (Neosystem/Strasbourg, France)

Enzyme:

The protein farnesyl transferase is partially purified from bovine brain by ion-exchange chromatography on Q-sepharose (Pharmacia) (Moores et al., J. Biol. Chem. 1991, 266: 14603-14610, Reiss et al., Cell 1990, 62: 81-88).

Method

The reaction mixture containing 2 μM of FPP, 2 μM of dansyl GCVLS with or without (zero) the amount of enzyme giving an intensity of 100 on the spectrofluorimeter after incubation for 10 minutes at 37° C., is prepared on ice.

In an Eppendorf tube, 360 μl of reaction mixture are mixed with 40 μl of 10× concentrated test product or of solvent, and incubated for 10 minutes at 37° C. The reaction is quenched on ice and the fluorescence intensity is measured (excitation at 340 nm, 4 nm slit, emission at 505 nm, 10 nm slit). The tests are performed in duplicate. The results are expressed as a percentage of inhibition. Under these conditions, the derivatives of the present invention were identified as powerful inhibitors of protein farnesyl transferase (IC₅₀<10 μM).

Adaptation of the method to a 96-wellformat:

The procedure is similar to that above, except that the measurements are performed in a “Black Fluorotrack 200” 96-well device (Greiner, Poitiers, France) and the readings are performed using a “Spectrametrix Gemini” 96-well fluorimeter (Molecular Devices, Sunnyvale, Calif., USA)

B) Evaluation of the inhibition of geranyl geraryl transferase protein I:

Materials

Reaction buffer:

55 mM TRIS/HCl pH 7.5; 5.5 mM DTT; 5.5 mM MgCl₂; 110 μM ZnCl₂, 0.22% N-octyl-B D-glucopyrannoside.

Substrates:

³H-geranylgeranyl pyrophosphate (GGPP), 66 μM, 15 CI/mmol, (Isotopchim)

Rho-GST recombinant protein

Enzyme:

GGPT I is partially purified from bovine brain by ion-exchange chromatography on Q-sepharose (Pharmacia); elution at 0.23 and 0.4 M NaCl, respectively.

(Moores et al., J. Biol. Chem. 1991, 266: 14603-14610; Reiss et al., Cell 1990, 62: 81-88).

Method

The reaction mixture containing 0.2 μM of ³H-GGPP, 1 μM of RhoA-GST with or without (zero) 5 μl of GGPT/test, is prepared on ice.

In an Eppendorf tube, 45 μl of reaction mixture are mixed with 5 μl of 10× concentrated test product or of solvent, and incubated for 45 minutes at 37° C. A 45 μl aliquot is placed on a phosphocellulose P81 filter (Whatman, Maidstone, UK) numbered, washed with 50% ethanol, phosphoric acid (0.5%) and counted by scintillation.

The tests are performed in duplicate. The results are expressed as a percentage of inhibition.

Adaptation of the method to a 96-wellformat

The procedure is similar to that above, except that the measurements are performed in 96-well plates (Nunc, France) and the reactions are then passed through a 96-well “Unifilter” (Whatman, Maidstone, UK) containing a phosphocellulose P81 buffer using a “Filtermate 196” system (Packard, France).

After washing with 50% ethanol and phosphoric acid (0.5%), the filters are counted by scintillation on a “Packard Topcount” instrument.

The tests are performed in triplicate. The results are expressed as a percentage of inhibition.

The derivatives of the present invention are inhibitors of enzymes that catalyze the prenylation of proteins and more particularly of PFTase. They are distinguished from the closest derivatives of the prior art not only by their novel chemical structure, but also by their biological activity and more particularly by their efficacy in inhibiting PFTase.

C) Results:

The compounds of the present invention described in the above examples were tested to determine their inhibitory activity on PFTase according to the above method. They were found to inhibit PFTase with an IC₅₀ value<1 μM.

The few examples that follow, chosen from the compounds of the present invention, illustrate the entirely unexpected capacity of these compounds to exert powerful inhibition on PFTase either selectively relative to PGGTase or in an equivalent manner:

| Example | IC₅₀ PFTase (nM) | IC₅₀ PGGTase (nM) |
|---|---|---|
| 28 | 4 | 10 |
| 30 | 6 | 10000 |
| 33 | 6 | 70 |
| 34 | 10 | 10000 |
| 40 | 2 | — |
| 46 | 1 | — |
| 55 | 3 | 10000 |
| 57 | 3 | 10000 |
| 91 | 2 | — |
| 92 | 2 | — |
| 97 | 1 | — |
| 100 | 6 | — |
| 101 | 5 | — |
| 113 | 8 | — |
| 164 | 8 | 20 |
| 165 | 10 | 60 |
| 167 | 400 | 7 |

Pharmaceutical compositions containing, as active ingredients, a compound of general formula (I) or a physiologically acceptable salt of a compound of general formula (I) combined with one or more therapeutic agents such as, for example, anticancer agents such as, for example, cytotoxic anticancer agents such as navelbine, taxol, taxotere, 5-fluorouracil, methotrexate, doxorubicin, camptothecin, gemcitabine, etoposide, cisplatin or BCNU, or hormonal anticancer agents, for instance tamoxifen or medroxyprogesterone, should also be considered as forming part of the present invention. Alternatively, in combination with an inhibitor of the biosynthesis of farnesyl and geranylgeranyl pyrophosphates, such as an HMG-CoA reductase inhibitor, for instance lovastatin, simvastatin, pravastatin, fluvastatin, atorvastatin or cerivastatin. Treatment with radiation (X-rays or gamma rays), which may be delivered using an external source or by implanting minute internal radioactive sources, may also be combined with the administration of an inhibitor of protein farnesyl transferase belonging to the present invention. These treatments may be used for the treatment or prevention of cancers such as cancer of the lungs, of the pancreas, of the skin, of the head, of the neck, of the uterus, of the ovaries, anal cancer, cancer of the stomach, of the colon, of the breast, of the esophagus, of the small intestine, of the thyroid gland, of the prostate, of the kidney, of the bladder, acute or chronic leukemias, or alternatively a combination of 2 or more of these cancers. These treatments may also be used for the treatment or prevention of restenosis or atherosclerosis, infections associated with PFTase such as delta hepatitis, or benign proliferative disorders.

A subject of the present invention is also pharmaceutical compositions containing as active principle a compound of general formula (I) or a pharmaceutically acceptable salt thereof, mixed or combined with a suitable excipient. These compositions may be, for example, in the form of solid or liquid compositions, emulsions, lotions or creams.

Solid compositions for oral administration that may be used include tablets, pills, powders (gelatin capsules or wafer capsules) or granules. In these compositions, the active principle according to the invention is mixed with one or more inert diluents such as starch, cellulose, sucrose, lactose or silica, under a stream of argon. These compositions may also comprise substances other than diluents, for example one or more lubricants such as magnesium stearate or talc, a colorant, a coating (dragees) or a varnish.

Liquid compositions for oral administration that may be used include pharmaceutically acceptable solutions, suspensions, emulsions, syrups and elixirs containing inert diluents such as water, ethanol, glycerol, plant oils or liquid paraffin. These compositions may comprise substances other than diluents, for example wetting, sweetening, thickening, flavoring or stabilizing products.

The sterile compositions for parenteral administration may preferably be aqueous or nonaqueous solutions, suspensions or emulsions. Solvents or vehicles that may be used include water, propylene glycol, a polyethylene glycol, plant oils, in particular olive oil, and injectable organic esters, for example ethyl oleate, or other suitable organic solvents. These compositions may also contain adjuvants, in particular wetting agents, isotonic agents, emulsifiers, dispersants and stabilizers. The sterilization may be performed in several ways, for example by aseptic filtration, by incorporating sterilizing agents into the composition, by irradiation or by heating. They may also be prepared in the form of sterile solid compositions that may be dissolved at the time of use in sterile water or any other injectable sterile medium.

The compositions for rectal administration are suppositories or rectal capsules containing, in addition to the active product, excipients such as cocoa butter, semisynthetic glycerides or polyethylene glycols.

The compositions for topical administration may be, for example, creams, lotions, eyedrops, mouth washes, nasal drops or aerosols.

The doses depend on the desired effect, the duration of the treatment and the administration route used; they are generally between 0.001 g and 1 g (preferably between 0.005 g and 0.75 g) per day, preferably orally, for an adult, with unit doses ranging from 0.1 mg to 500 mg of active substance.

In general, the doctor will determine the appropriate dosage as a function of the age and weight and all the other personal factors of the individual to be treated.

1. A compound corresponding to the general formula (I): 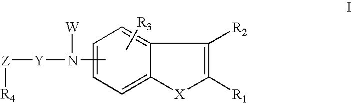
in which:
W represents:
hydrogen, SO₂R₅, CO(CH₂)ₙR₅, (CH₂)ₙR₆, CS(CH₂)ₙR₅
X represents:
S or NH
Y represents:
(CH₂)ₚ, CO, (CH₂)ₚCO, CH═CH—CO
when Y═CO, (CH₂)ₚCO or CH═CH—CO, then W represents only hydrogen or (CH₂)ₙR₆.
When Y═CO, then X represents only S.
Z represents:
imidazole, benzimidazole, isoxazole, tetrazole, oxadiazole, thiadiazole, pyridine, quinazoline, quinoxaline, quinoline, thiophene, these heterocycles possibly being unsubstituted or substituted with one or more groups chosen from C₁-C₁₅ alkyl, halogen, OMe, CN, NO₂, OH, CF₃, OCF₃, OCH₂Ph, SMe, COOMe, COOEt, COOH, CONHOH, SO₂NH2, CONH₂.
When Z=pyridine, then X represents only S.
R₁ represents:
COOR₆, CONR₆R₇, CO—NH—CH(R₆)—COOR₇, CH₂NR₆R₇, CH₂OR₆, (CH₂)ₚR₆, CH═CHR₆.
R₂ represents:
a) hydrogen,
b) C₁-C₁₀ alkyl, cycloalkyl, C₃-C₃₀ alkenyl, C₃-C₂₀ alkynyl
c) a phenyl, which is unsubstituted or substituted with one or more residues chosen from C₁-C₆ alkyl, halogen, phenyl, naphthyl, NO₂, CN, CF₃, OR₆, SR₆, NR₆R₇, COOR₆, CONR₆R₇, COR₆.
R₃ represents:
hydrogen, C₁-C₆ alkyl, halogen, OMe, CN, NO₂, OH, CF₃, OCF₃, OCH₂Ph, SMe, COOMe, COOEt, COOH, CONHOH, SO₂NH2, CONH₂.
R₄ represents:
a) hydrogen,
b) C₁-C₆ alkyl, which is unsubstituted or substituted with one or more residues chosen from aryl, cyanophenyl, nitrophenyl, aminophenyl, methoxyphenyl, hydroxyphenyl, heterocycle, halogen, CN, NO₂, OR₂, SR₂, NR₂R₃, COOR₂;
c) an aryl,
d) a heterocycle.
R₅ represents:
a) a phenyl or naphthyl, which is unsubstituted or substituted with one or more residues chosen from C₁-C₆ alkyl, halogen, phenyl, naphthyl, NO₂, CN, CF₃, OR₆, SR₆, NR₆R₇, COOR₆, CONR₆R₇, COR₆;
b) C₁-C₁₅ alkyl, C₃-C₃₀ alkenyl or C₃-C₂₀ alkynyl, which is unsubstituted or substituted with one or more residues chosen from halogen, COOMe, COOH, OR₂, CF₃, CN, SR₂; a cycloalkyl, which is unsubstituted or substituted with a halogen, OR₂, CF₃, CN, SR₂; an alkylcycloalkyl, which is unsubstituted or substituted with a halogen, OR₂, CF₃, CN, SR₂;
c) a heterocycle,
d) NR₆R₇.
R₆ and R₇, which may be identical or different, represent:
a) hydrogen; C₁-C₁₅ alkyl, C₃-C₃₀ alkenyl or C₃-C₂₀ alkynyl, which is unsubstituted or substituted with one or more residues chosen from halogen, COOMe, COOH, OR₂, CF₃, CN, SR₂; a cycloalkyl, which is unsubstituted or substituted with a halogen, OR₂, CF₃, CN, SR₂; an alkylcycloalkyl, which is unsubstituted or substituted with a halogen, OMe, OH, CF₃, CN or SMe,
b) a heterocycle or an alkylheterocycle,
c) an aryl, an alkylaryl or an alkyldiaryl,
d) R₆ and R₇, when they are adjacent, taken together, may form a 4- to 6-membered ring with the nitrogen atom to which they are attached, which may contain one or more hetero atoms chosen from N, S and O and which may be unsubstituted or substituted with one or more groups chosen from C₁-C₁₅ alkyl, aryl and alkylaryl.
n represents:
0 to 10
p represents:
1 to 6
said compounds of formula I possibly being in any stereoisomeric form thereof, including any optical isomer thereof, and also racemic mixtures thereof, and the therapeutically acceptable salts and solvates thereof.

2. The compound as claimed in claim 1, characterized in that R₂, R₃ and R₄ represent a hydrogen and Y represents a methylene (CH₂).

3. The compound as claimed in claim 1, characterized in that Z represents an imidazolyl or pyridyl residue.

4. The compound as claimed in claim 1, characterized in that Z represents an imidazolyl residue and R₄ represents a methyl or benzyl group, which is unsubstituted or substituted with a nitrile, nitro or methoxy group in position 4.

5. The compound as claimed in claim 1, characterized in that X represents a sulfur atom.

6. The compound as claimed in claim 1, characterized in that X represents an NH and R₂ represents a phenyl.

7. The compound as claimed in claim 1, characterized in that R₁ represents CONR₆R₇.

8. The compound as claimed in claim 1, characterized in that W represents SO₂R₅.

9. The compound as claimed in claim 1, characterized in that W represents CO(CH₂)ₙR₅.

10. The compound as claimed in claim 1, characterized in that W represents (CH₂)ₙR₆.

11. A compound as claimed in claim 1, selected from:
(2S)-2-[(5-{2-[3-(4-cyanobenzyl)-3H-imidazol-4-yl]acetylamino}benzo[b]thiophene-2-carbonyl)amino]-4-methylpentanoic acid
(2S)-2-[(5-{[3-(4-cyanobenzyl)-3H-imidazol-4-ylmethyl]amino}benzo[b]thiophene-2-carbonyl)amino]-4-methylpentanoic acid
(2S)-2-[(5-{benzenesulfonyl-[3-(4-cyanobenzyl)-3H-imidazol-4-ylmethyl]amino}benzo[b]thiophene-2-carbonyl)amino]-4-methylpentanoic acid
(2S)-2-[(5-{2-[3-(4-benzenesulfonyl-[3-(4-cyanobenzyl)-3H-imidazol-4-ylmethyl]amino}benzo[b]thiophene-2-carbonyl)amino]-4-methylsulfanyl)butyric acid
(2S)-2-[(4-{benzenesulfonyl-[3-(4-cyanobenzyl)-3H-imidazol-4-ylmethyl]amino}benzo[b]thiophene-2-carbonyl)amino]-4-(methylsulfanyl)butyric acid
(2S)-2-[(4-{benzenesulfonyl-[3-(4-cyanobenzyl)-3H-imidazol-4-ylmethyl]amino}benzo[b]thiophene-2-carbonyl)amino]-4-methylpentanoic acid
(2S)-2-[(4-{(2-chlorobenzenesulfonyl)-[3-(4-cyanobenzyl)-3H-imidazol-4-ylmethyl]amino}benzo[b]thiophene-2-carbonyl)amino]-4-methylpentanoic acid
(2S)-2-[(5-{benzenesulfonyl-[3-(4-cyanobenzyl)-3H-imidazol-4-ylmethyl]amino}benzo[b]thiophene-2-carbonyl)amino]-4-methylpentanoic acid
ethyl 5-{[3-(4-cyanobenzyl)-3H-imidazol-4-ylmethyl]amino}benzo[b]thiophene-2-carboxylate
ethyl 4-{[3-(4-cyanobenzyl)-3H-imidazol-4-ylmethyl]amino}benzo[b]thiophene-2-carboxylate
methyl 5-{(benzenesulfonyl)-[3-(4-cyanobenzyl)-3H-imidazol-4-ylmethyl]-amino}benzo[b]thiophene-2-carboxylate
methyl 4-{(benzenesulfonyl)-[3-(4-cyanobenzyl)-3H-imidazol-4-ylmethyl]-amino}benzo[b]thiophene-2-carboxylate
methyl (2S)-2-[(5-{benzenesulfonyl-[3-(4-cyanobenzyl)-3H-imidazol-4-ylmethyl]-amino}benzo[b]thiophen-2-carbonyl)amino]-4-(methylsulfanyl)butyrate methyl
(2S)-2-[(4-{benzenesulfonyl-[3-(4-cyanobenzyl)-3H-imidazol-4-ylmethyl]amino}benzo[b]thiophen-2-carbonyl)amino]-4-(methylsulfanyl)butyrate
N-(2-thiophen-2-ylethyl)-4-{benzenesulfonyl-[3-(4-cyanobenzyl)-3H-imidazol-4-ylmethyl]amino}benzo[b]thiophene-2-carboxamide
N-(2-methylsulfanylethyl)-4-{benzenesulfonyl-[3-(4-cyanobenzyl)-3H-imidazol-4-ylmethyl]amino}benzo[b]thiophene-2-carboxamide
N-[3-(4-cyanobenzyl)-3H-imidazol-4-ylmethyl]-N-[2-thiomorpholine-4-carbonyl)benzo[b]thiophen-4-yl]benzenesulfonamide
N-(thiophen-2-ylmethyl)-5-{benzenesulfonyl-[3-(4-cyanobenzyl)-3H-imidazol-4-ylmethyl]amino}benzo[b]thiophene-2-carboxamide
N-(2-thiophen-2-ylethyl)-5-{benzenesulfonyl-[3-(4-cyanobenzyl)-3H-imidazol-4-ylmethyl]amino}benzo[b]thiophene-2-carboxamide
N-(2-methylsulfanylethyl)-5-{benzenesulfonyl-[3-(4-cyanobenzyl)-3H-imidazol-4-ylmethyl]amino}benzo[b]thiophene-2-carboxamide
N-[3-(4-cyanobenzyl)-3H-imidazol-4-ylmethyl]-N-[2-(thiomorpholine-4-carbonyl)benzo[b]thiophen-5-yl]benzenesulfonamide
methyl (2S)-2-[(4-{benzenesulfonyl-[3-(4-cyanobenzyl)-3H-imidazol-4-ylmethyl]-amino}benzo[b]thiophen-2-carbonyl)amino]-4-methylpentanoate
methyl (2S)-2-[(5-{benzenesulfonyl-[3-(4-cyanobenzyl)-3H-imidazol-4-ylmethyl]-amino}benzo[b]thiophen-2-carbonyl)amino]-4-methylpentanoate
methyl (2S)-2-[(5-{benzenesulfonyl-[3-(4-cyanobenzyl)-3H-imidazol-4-ylmethyl]-amino}benzo[b]thiophen-2-carbonyl)amino]acetate
N-cyclopentyl-4-{benzenesulfonyl-[3-(4-cyanobenzyl)-3H-imidazol-4-ylmethyl]amino}benzo[b]thiophene-2-carboxamide
N-cyclopentyl-5-{benzenesulfonyl-[3-(4-cyanobenzyl)-3H-imidazol-4-ylmethyl]amino}benzo[b]thiophene-2-carboxamide
N-butyl-4-{benzenesulfonyl-[3-(4-cyanobenzyl)-3H-imidazol-4-ylmethyl]amino}benzo[b]thiophene-2-carboxamide
N-butyl-5-{benzenesulfonyl-[3-(4-cyanobenzyl)-3H-imidazol-4-ylmethyl]amino}benzo[b]thiophene-2-carboxamide
N-[3-methylsulfanyl)propyl]-4-{benzenesulfonyl-[3-(4-cyanobenzyl)-3H-imidazol-4-ylmethyl]amino} benzo[b]thiophene-2-carboxamide
N-[3-(methylsulfanyl)propyl]-5-{benzenesulfonyl-[3-(4-cyanobenzyl)-3H-imidazol-4-ylmethyl]amino}benzo[b]thiophene-2-carboxamide
N-[3-(isopropoxy)propyl]-4-{benzenesulfonyl-[3-(4-cyanobenzyl)-3H-imidazol-4-ylmethyl]amino}benzo[b]thiophene-2-carboxamide
N-[3-(isopropoxy)propyl]-5-{benzenesulfonyl-[3-(4-cyanobenzyl)-3H-imidazol-4-ylmethyl]amino}benzo[b]thiophene-2-carboxamide
N-[3,7-dimethylocta-2,6-dienyl]-4-{benzenesulfonyl-[3-(4-cyanobenzyl)-3H-imidazol-4-ylmethyl]amino}benzo[b]thiophene-2-carboxamide
N-[3,7-dimethylocta-2,6-dienyl]-5-{benzenesulfonyl-[3-(4-cyanobenzyl)-3H-imidazol-4-ylmethyl]amino}benzo[b]thiophene-2-carboxamide
N-cyclohexyl-5-{benzenesulfonyl-[3-(4-cyanobenzyl)-3H-imidazol-4-ylmethyl]amino}benzo[b]thiophene-2-carboxamide
N-(2-methylpropyl)-5-{benzenesulfonyl-[3-(4-cyanobenzyl)-3H-imidazol-4-ylmethyl]amino}benzo[b]thiophene-2-carboxamide
N-methyl-S-{benzenesulfonyl-[3-(4-cyanobenzyl)-3H-imidazol-4-ylmethyl]amino}benzo[b]thiophene-2-carboxamide
N-ethyl-5-{benzenesulfonyl-[3-(4-cyanobenzyl)-3H-imidazol-4-ylmethyl]amino}benzo[b]thiophene-2-carboxamide
N,N-diethyl-5-{benzenesulfonyl-[3-(4-cyanobenzyl)-3H-imidazol-4-ylmethyl]amino}benzo[b]thiophene-2-carboxamide
N-(2,2-dimethylpropyl)-5-{benzenesulfonyl-[3-(4-cyanobenzyl)-3H-imidazol-4-ylmethyl]amino}benzo[b]thiophene-2-carboxamide
N-propyl-5-{benzenesulfonyl-[3-(4-cyanobenzyl)-3H-imidazol-4-ylmethyl]amino} benzo[b]thiophene-2-carboxamide
N-allyl-5-{benzenesulfonyl-[3-(4-cyanobenzyl)-3H-imidazol-4-ylmethyl]amino}benzo[b]thiophene-2-carboxamide
N-(2-methoxyethyl)-5-{benzenesulfonyl-[3-(4-cyanobenzyl)-3H-imidazol-4-ylmethyl]amino}benzo[b]thiophene-2-carboxamide
N-cyclopropyl-5-{benzenesulfonyl-[3-(4-cyanobenzyl)-3H-imidazol-4-ylmethyl]amino} benzo[b]thiophene-2-carboxamide
N-(2-pyrrolidin-1-ylethyl)-5-{benzenesulfonyl-[3-(4-cyanobenzyl)-3H-imidazol-4-ylmethyl]amino}benzo[b]thiophene-2-carboxamide
N-(pyrid-2-ylmethyl)-5-{benzenesulfonyl-[3-(4-cyanobenzyl)-3H-imidazol-4-ylmethyl]amino} benzo[b]thiophene-2-carboxamide
N-pyrid-3-ylmethyl)-5-benzenesulfonyl-[3-(4-cyanobenzyl)-3H-imidazol-4-ylmethyl]amino}benzo[b]thiophene-2-carboxamide
N-(pyrid-4-ylmethyl)-5-(benzenesulfonyl-[3-(4-cyanobenzyl)-3H-imidazol-4-ylmethyl]amino}benzo[b]thiophene-2-carboxamide
N-(pyrid-2-ylethyl)-5-benzenesulfonyl-[3-(4-cyanobenzyl)-3H-imidazol-4-ylmethyl]amino}benzo[b]thiophene-2-carboxamide
N-(3-oxo-2,3-dihydroisoxazol-5-ylmethyl)-5-{benzenesulfonyl-[3-(4-cyanobenzyl)-3H-imidazol-4-ylmethyl]amino}benzo[b]thiophene-2-carboxamide
N-[3-(4-cyanobenzyl)-3H-imidazol-4-ylmethyl]-N-[2-(4-methylpiperazine-4-carbonyl]benzo[b]thiophen-5-yl]benzenesulfonamide
N-[3-(4-cyanobenzyl)-3H-imidazol-4-ylmethyl]-N-[2-(4-benzylpiperazine-4-carbonyl)benzo[b]thiophen-5-yl]benzenesulfonamide
N-[3-(4-cyanobenzyl)-3H-imidazol-4-ylmethyl]—N {2-[4-(4-fluorophenyl)piperazine-4-carbonyl]benzo[b]thiophen-5-yl}benzenesulfonamide
N-[3-(4-cyanobenzyl)-3H-imidazol-4-ylmethyl]-N-{2-[4-(2-cyanophenyl)piperazine-4-carbonyl]benzo[b]thiophen-5-yl} benzenesulfonamide
N-(benzyl-5-{benzenesulfonyl-[3-(4-cyanobenzyl)-3H-imidazol-4-ylmethyl]amino}benzo[b]thiophene-2-carboxamide
N-(2-phenylethyl)-5-{benzenesulfonyl-[3-(4-cyanobenzyl)-3H-imidazol-4-ylmethyl]amino}benzo[b]thiophene-2-carboxamide
4-{[3-(4-cyanobenzyl)-3H-imidazol-4-ylmethyl]amino}benzo[b]thiophen-2-carboxylic acid
4-(5-{[2-(thiomorpholine-4-carbonyl)benzo[b]thiophen-5-ylamino]methyl} imidazol-1-ylmethyl)benzonitrile
4-(5-{[2-(thiomorpholine-4-carbonyl)benzo[b]thiophen-4-ylamino]methyl} imidazol-1-ylmethyl)benzonitrile
N-[3-(4-cyanobenzyl)-3H-imidazol-4-ylmethyl]-N-[2-(thiomorpholine-4-carbonyl)benzo[b]thiophen-5-yl]thiophene-2-carboxamide
N-[3-(4-cyanobenzyl)-3H-imidazol-4-ylmethyl]-N-[2-(thiomorpholine-4-carbonyl)benzo[b]thiophen-5-yl]butyramide
N-[3-(4-cyanobenzyl)-3H-imidazol-4-ylmethyl]-N-[2-(thiomorpholine-4-carbonyl)benzo[b]thiophen-5-yl]benzamide
N-[3-(4-cyanobenzyl)-3H-imidazol-4-ylmethyl]-N-[2-(thiomorpholine-4-carbonyl)benzo[b]thiophen-5-yl]-2-chlorobenzamide
N-[3-(4-cyanobenzyl)-3H-imidazol-4-ylmethyl]-N-[2-(thiomorpholine-4-carbonyl)benzo[b]thiophen-5-yl]-3-chlorobenzamide
N-[3-(4-cyanobenzyl)-3H-imidazol-4-ylmethyl]-N-[2-(thiomorpholine-4-carbonyl)benzo[b]thiophen-5-yl]-4-chlorobenzamide
N-[3-(4-cyanobenzyl)-3H-imidazol-4-ylmethyl]-N-[2-(thiomorpholine-4-carbonyl)benzo[b]thiophen-5-yl]-3-fluorobenzamide
N-[3-(4-cyanobenzyl)-3H-imidazol-4-ylmethyl]-N-[2-(thiomorpholine-4-carbonyl)benzo[b]thiophen-5-yl]-2-trifluoromethylbenzamide
N-[3-(4-cyanobenzyl)-3H-imidazol-4-ylmethyl]-N-[2-(thiomorpholine-4-carbonyl)benzo[b]thiophen-5-yl]-4-cyanobenzamide
N-[3-(4-cyanobenzyl)-3H-imidazol-4-ylmethyl]-N-[2-(thiomorpholine-4-carbonyl)benzo[b]thiophen-5-yl]-2-phenylacetamide
N-[3-(4-cyanobenzyl)-3H-imidazol-4-ylmethyl]-N-[2-(thiomorpholine-4-carbonyl)benzo[b]thiophen-5-yl]cyclohexanecarboxamide
N-[3-(4-cyanobenzyl)-3H-imidazol-4-ylmethyl]-N-[2-(thiomorpholine-4-carbonyl)benzo[b]thiophen-4-yl]-3-chlorobenzamide
1-[3-(4-cyanobenzyl)-3H-imidazol-4-yl]-3-[2-(methylsulfanyl)phenyl]-1-[2-(thiomorpholine-4-carbonyl)benzo[b]thiophen-5-yl]urea
4-[5-({(2-phenylethyl)[2-(thiomorpholine-4-carbonyl)benzo[b]thiophen-5-yl]amino}methyl)imidazol-1-ylmethyl]benzonitrile
4-[5-({ethyl[2-(thiomorpholine-4-carbonyl)benzo[b]thiophen-5-yl]amino} methyl)imidazol-1-ylmethyl]benzonitrile
4-[5-({propyl[2-(thiomorpholine-4-carbonyl)benzo[b]thiophen-5-yl]amino} methyl)imidazol-1-ylmethyl]benzonitrile
4-[5-({butyl[2-(thiomorpholine-4-carbonyl)benzo[b]thiophen-5-yl]amino}methyl)imidazol-1-ylmethyl]benzonitrile
4-[5-({(2-phenylethyl)[2-(thiomorpholine-4-carbonyl)benzo[b]thiophen-4-yl]amino}methyl)imidazol-1-ylmethyl]benzonitrile
4-[5-({propyl[2-(thiomorpholine-4-carbonyl)benzo[b]thiophen-4-yl]amino}methyl)imidazol-1-ylmethyl]benzonitrile
4-[5-({butyl[2-(thiomorpholine-4-carbonyl)benzo[b]thiophen-4-yl]amino} methyl)imidazol-1-ylmethyl]benzonitrile
4-[5-({cyclohexylmethyl[2-(thiomorpholine-4-carbonyl)benzo[b]thiophen-4-yl]amino} methyl)imidazol-1-ylmethyl]benzonitrile
ethyl 3-butyl-7-{[3-(4-cyanobenzyl)-3H-imidazol-4-ylmethyl]amino}benzo[b]-thiophene-2-carboxylate
3-butyl-7-{[3-(4-cyanobenzyl)-3H-imidazol-4-ylmethyl]amino} benzo[b]-thiophene-2-carboxylic acid
4-(5-{[3-butyl-2-(thiomorpholine-4-carbonyl)benzo[b]thiophen-7-ylamino]methyl} imidazol-1-ylmethyl)benzonitrile
4-(5-{[3-butyl-2-(piperidine-1-carbonyl)benzo[b]thiophen-7-ylamino]methyl} imidazol-1-ylmethyl)benzonitrile
N-butyl-3-butyl-7-{[3-(4-cyanobenzyl)-3H-imidazol-4-ylmethyl]amino} benzo[b]-thiophene-2-carboxamide
N-(2-thiophen-2-ylethyl)-5-{[3-(4-cyanobenzyl)-3H-imidazol-4-ylmethyl]amino}benzo[b]thiophene-2-carboxamide
N-(2-thiophen-2-ylethyl)-5-{[pyrid-4-ylmethyl]amino}benzo[b]thiophene-2-carboxamide
N-(2-thiophen-2-ylethyl)-5-{[pyrid-3-ylmethyl]amino}benzo[b]thiophene-2-carboxamide
N-(2-thiophen-2-ylethyl)-5-{[3-methyl-3H-imidazol-4-ylmethyl]amino}benzo[b]-thiophene-2-carboxamide
N-(2-thiophen-2-ylethyl)-5-{[3-benzyl-3H-imidazol-4-ylmethyl]amino} benzo[b]-thiophene-2-carboxamide
N-(2-thiophen-2-ylethyl)-5-{[thiophen-2-ylmethyl]amino} benzo[b]thiophene-2-carboxamide
N-(2-thiophen-2-ylethyl)-5-{[thiophen-3-ylmethyl]amino} benzo[b]thiophene-2-carboxamide
N-(2-thiophen-2-ylethyl)-5-{[quinolein-3-ylmethyl]amino}benzo[b]thiophene-2-carboxamide
N-(thiophen-2-ylmethyl)-5-{[3-(4-cyanobenzyl)-3H-imidazol-4-ylmethyl]amino}benzo[b]thiophene-2-carboxamide
N-butyl-5-{[3-(4-cyanobenzyl)-3H-imidazol-4-ylmethyl]amino}benzo[b]-thiophene-2-carboxamide
4-[5-({2-[2-thiophen-2-ylethylamino)methyl]benzo[b]thiophen-5-ylamino} methyl)imidazol-1-ylmethyl]benzonitrile
N-(2-thiophen-2-ylethyl)-5-{butyryl-[3-(4-cyanobenzyl)-3H-imidazol-4-ylmethyl]amino}benzo[b]thiophene-2-carboxamide
N-(2-thiophen-2-ylethyl)-5-{hexanoyl-[3-(4-cyanobenzyl)-3H-imidazol-4-ylmethyl]amino}benzo[b]thiophene-2-carboxamide
N-(2-thiophen-2-ylethyl)-5-{benzoyl-[3-(4-cyanobenzyl)-3H-imidazol-4-ylmethyl]amino}benzo[b]thiophene-2-carboxamide
N-(2-thiophen-2-ylethyl)-5-{(3-fluorobenzoyl)-[3-(4-cyanobenzyl)-3H-imidazol-4-ylmethyl]amino}benzo[b]thiophene-2-carboxamide
N-(2-thiophen-2-ylethyl)-5-{(3-methoxybenzoyl)-[3-(4-cyanobenzyl)-3H-imidazol-4-ylmethyl]amino} benzo[b]thiophene-2-carboxamide
N-(2-thiophen-2-ylethyl)-5-{(4-fluorobenzoyl)-[3-(4-cyanobenzyl)-3H-imidazol-4-ylmethyl]amino}benzo[b]thiophene-2-carboxamide
N-(2-thiophen-2-ylmethyl)-5-{butyryl-[3-(4-cyanobenzyl)-3H-imidazol-4-ylmethyl]amino}benzo[b]thiophene-2-carboxamide
N-(2-thiophen-2-ylmethyl)-5-{hexanoyl-[3-(4-cyanobenzyl)-3H-imidazol-4-ylmethyl]amino}benzo[b]thiophene-2-carboxamide
N-(2-thiophen-2-ylmethyl)-5-{benzoyl-[3-(4-cyanobenzyl)-3H-imidazol-4-ylmethyl]amino}benzo[b]thiophene-2-carboxamide
N-(2-thiophen-2-ylmethyl)-5-{(3-fluorobenzoyl)-[3-(4-cyanobenzyl)-3H-imidazol-4-ylmethyl]amino}benzo[b]thiophene-2-carboxamide
N-(2-thiophen-2-ylmethyl)-5-{(3-methoxybenzoyl)-[3-(4-cyanobenzyl)-3H-imidazol-4-ylmethyl]aminio}benzo[b]thiophene-2-carboxamide
N-(2-thiophen-2-ylmethyl)-5-{(4-fluorobenzoyl)-[3-(4-cyanobenzyl)-3H-imidazol-4-ylmethyl]amino}benzo[b]thiophene-2-carboxamide
N-butyl-5-{butyryl-[3-(4-cyanobenzyl)-3H-imidazol-4-ylmethyl]amino}-benzo[b]thiophene-2-carboxamide
N-butyl-5-{hexanoyl-[3-(4-cyanobenzyl)-3H-imidazol-4-ylmethyl]amino}-benzo[b]thiophene-2-carboxamide
N-butyl-5-{benzoyl-[3-(4-cyanobenzyl)-3H-imidazol-4-ylmethyl]amino}-benzo[b]thiophene-2-carboxamide
N-butyl-5-{(3-fluorobenzoyl)-[3-(4-cyanobenzyl)-3H-imidazol-4-ylmethyl]amino}benzo[b]thiophene-2-carboxamide
N-butyl-5-{(3-methoxybenzoyl)-[3-(4-cyanobenzyl)-3H-imidazol-4-ylmethyl]amino} benzo[b]thiophene-2-carboxamide
N-butyl-5-{(4-fluorobenzoyl)-[3-(4-cyanobenzyl)-3H-imidazol-4-ylmethyl]amino}benzo[b]thiophene-2-carboxamide
N,N-dimethyl-5-{benzenesulfonyl-[3-(4-cyanobenzyl)-3H-imidazol-4-ylmethyl]amino}benzo[b]thiophene-2-carboxamide
N-[3-(4-cyanobenzyl)-3H-imidazol-4-ylmethyl]-N-[3-phenyl-2-(piperidine-1-carbonyl)-1H-indol-5-yl]benzamide
N-[3-(4-cyanobenzyl)-3H-imidazol-4-ylmethyl]-N-[3-phenyl-2-(thiomorpholine-4-carbonyl)-1H-indol-5-yl]benzamide
N-(isobutyl)-5-{benzoyl-[3-(4-cyanobenzyl)-3H-imidazol-4-ylmethyl]amino)}-3-phenyl-1H-indole-2-carboxamide
cyclohexanecarboxylic acid [3-(4-cyanobenzyl)-3H-imidazol-4-ylmethyl]-[3-phenyl-2-(piperidine-1-carbonyl)-1H-indol-5-yl]amide
cyclohexanecarboxylic acid [3-(4-cyanobenzyl)-3H-imidazol-4-ylmethyl]-[3-phenyl-2-(thiomorpholine-4-carbonyl)-1H-indol-5-yl]amide
N-(isobutyl)-5-{[3-(4-cyanobenzyl)-3H-imidazol-4-ylmethyl]cyclohexanecarbonylamino}-3-phenyl-1H-indole-2-carboxamide
N-(2-thiophen-2-ylethyl)-5-{[3-(4-cyanobenzyl)-3H-imidazol-4-ylmethyl]pentanoylamino}benzo[b]thiophene-2-carboxamide
N-(thiophen-2-ylmethyl)-5-{[3-(4-cyanobenzyl)-3H-imidazol-4-ylmethyl]pentanoylamino}benzo[b]thiophene-2-carboxamide
N-(thiophen-2-ylmethyl)-5-{[3-(4-cyanobenzyl)-3H-imidazol-4-ylmethyl]heptanoylamino}benzo[b]thiophene-2-carboxamide
N-(thiophen-2-ylmethyl)-5-{[3-(4-cyanobenzyl)-3H-imidazol-4-ylmethyl]cyclohexanecarbonylamino}benzo[b]thiophene-2-carboxamide
N-(thiophen-2-ylmethyl)-5-{[3-(4-cyanobenzyl)-3H-imidazol-4-ylmethyl]-(3-methylbutyryl)amino}benzo[b]thiophene-2-carboxamide
N-butyl-5-{[3-(4-cyanobenzyl)-3H-imidazol-4-ylmethyl]cyclohexanecarbonyl-amino}benzo[b]thiophene-2-carboxamide
N-butyl-5-{[3-(4-cyanobenzyl)-3H-imidazol-4-ylmethyl]-(3-methylbutyryl)amino}benzo[b]thiophene-2-carboxamide
N-(2-thiophen-2-ylethyl)-5-{[3-(4-cyanobenzyl)-3H-imidazol-4-ylmethyl]-heptanoylamino}benzo[b]thiophene-2-carboxamide
N-butyl-5-{[3-(4-cyanobenzyl)-3H-imidazol-4-ylmethyl]heptanoylamino}-benzo[b]thiophene-2-carboxamide
N-(thiophen-2-ylmethyl)-5-{[3-(4-cyanobenzyl)-3H-imidazol-4-ylmethyl]-(2,2,3,3,4,4,4-heptafluorobutyryl)amino}benzo[b]thiophene-2-carboxamide
N-(thiophen-2-ylmethyl)-5-{[3-(4-cyanobenzyl)-3H-imidazol-4-ylmethyl]-(3-cyclopentylpropionyl)amino}benzo[b]thiophene-2-carboxamide
N-butyl-5-{[3-(4-cyanobenzyl)-3H-imidazol-4-ylmethyl]-(3-cyclopentylpropionyl)amino}benzo[b]thiophene-2-carboxamide
N-(pyrid-3-ylmethyl)-5-{butyryl-[3-(4-cyanobenzyl)-3H-imidazol-4-ylmethyl]amino}benzo[b]thiophene-2-carboxamide
N-(pyrid-3-ylmethyl)-5-{[3-(4-cyanobenzyl)-3H-imidazol-4-ylmethyl]cyclohexanecarbonylamino}benzo[b]thiophene-2-carboxamide
N-(pyrid-3-ylmethyl)-5-{[3-(4-cyanobenzyl)-3H-imidazol-4-ylmethyl]-(3-methylbutyryl)amino}benzo[b]thiophene-2-carboxamide
N-(pyrid-3-ylmethyl)-5-{[3-(4-cyanobenzyl)-3H-imidazol-4-ylmethyl]pentanoylamino}benzo[b]thiophene-2-carboxamide
N-(pyrid-3-ylmethyl)-5-{[3-(4-cyanobenzyl)-3H-imidazol-4-ylmethyl]propylamino}benzo[b]thiophene-2-carboxamide
N-(pyrid-3-ylmethyl)-5-{butyl-[3-(4-cyanobenzyl)-3H-imidazol-4-ylmethyl]amino}benzo[b]thiophene-2-carboxamide
N-(2-pyrid-2-ylethyl)-5-{[3-(4-cyanobenzyl)-3H-imidazol-4-ylmethyl]-(3-fluorobenzenesulfonyl)amino}benzo[b]thiophene-2-carboxamide
N-(pyrid-3-ylmethyl)-5-{[3-(4-cyanobenzyl)-3H-imidazol-4-ylmethyl]-(3-fluorobenzenesulfonyl)amino}benzo[b]thiophene-2-carboxamide
N-(pyrid-3-ylmethyl)-5-{[3-(4-cyanobenzyl)-3H-imidazol-4-ylmethyl]-(3-methoxybenzenesulfonyl)amino}benzo[b]thiophene-2-carboxamide
N-(2-pyrid-2-ethyl)-5-{[3-(4-cyanobenzyl)-3H-imidazol-4-ylmethyl]-(3-methoxybenzenesulfonyl)amino)benzo[b]thiophene-2-carboxamide
N-[3-(4-cyanobenzyl)-3H-imidazol-4-ylmethyl]-N-[2-(4-methylpiperazin-4-carbonyl)benzo[b]thiophen-5-yl]-3-methoxybenzenesulfonamide
N-(2-pyrrolidin-1-ylethyl)-5-{3-methoxybenzenesulfonyl-[3-(4-cyanobenzyl)-3H-imidazol-4-ylmethyl]amino}benzo[b]thiophene-2-carboxamide
N-[3-(4-cyanobenzyl)-3H-imidazol-4-ylmethyl]-N-[2-(4-methylpiperazin-4-carbonyl]benzo[b]thiophen-5-yl]-3-fluorobenzenesulfonamide
N-(2-pyrrolidin-1-ylethyl)-5-{3-fluorobenzenesulfonyl-[3-(4-cyanobenzyl)-3H-imidazol-4-ylmethyl]amino}benzo[b]thiophene-2-carboxamide
N-(pyrid-3-ylmethyl)-5-{butyryl-[3-(4-cyanobenzyl)-3H-imidazol-4-ylmethyl]amino}benzo[b]thiophene-2-carboxamide
N-(pyrid-3-ylmethyl)-5-{[3-(4-cyanobenzyl)-3H-imidazol-4-ylmethyl]cyclohexanecarbonylamino}benzo[b]thiophene-2-carboxamide
N-(pyrid-3-ylmethyl)-5-{[3-(4-cyanobenzyl)-3H-imidazol-4-ylmethyl]-(3-methylbutyryl)amino}benzo[b]thiophene-2-carboxamide
N-(pyrid-3-ylmethyl)-5-{[3-(4-cyanobenzyl)-3H-imidazol-4-ylmethyl]hexanoylamino}benzo[b]thiophene-2-carboxamide
N-[3-(4-cyanobenzyl)-3H-imidazol-4-ylmethyl]-N-[2-(4-methylpiperazine-1-carbonyl)benzo[b]thiophen-5-yl]cyclohexanecarboxamide
N-[3-(4-cyanobenzyl)-3H-imidazol-4-ylmethyl]-3-methyl-N-[2-(4-methylpiperazine-1-carbonyl)benzo[b]thiophen-5-yl]butyramide
N-[3-(4-cyanobenzyl)-3H-imidazol-4-ylmethyl]-N-[2-(4-methylpiperazine-1-carbonyl]benzo[b]thiophen-5-yl]hexanamide
N-(2-pyrid-2-ylethyl)-5-{[3-(4-cyanobenzyl)-3H-imidazol-4-ylmethyl]cyclohexanecarbonylamino}benzo[b]thiophene-2-carboxamide
N-(2-pyrid-2-ylethyl)-5-{[3-(4-cyanobenzyl)-3H-imidazol-4-ylmethyl]-(3-methylbutyryl)amino}benzo[b]thiophene-2-carboxamide
N-(2-pyrid-2-ylethyl)-5-{benzoyl-[3-(4-cyanobenzyl)-3H-imidazol-4-ylmethyl]amino}benzo[b]thiophene-2-carboxamide
N-(2-pyrid-2-ylethyl)-5-{[3-(4-cyanobenzyl)-3H-imidazol-4-ylmethyl]-(3-fluorobenzoyl)amino}benzo[b]thiophene-2-carboxamide
N-(2-pyrid-2-ylethyl)-5-{[3-(4-cyanobenzyl)-3H-imidazol-4-ylmethyl]-(3-methoxybenzoyl)amino}benzo[b]thiophene-2-carboxamide
N-(2-pyrid-2-ylethyl)-5-{(3-chlorobenzoyl)-[3-(4-cyanobenzyl)-3H-imidazol-4-ylmethyl]amino}benzo[b]thiophene-2-carboxamide
N-[3-(4-cyanobenzyl)-3H-imidazol-4-ylmethyl]-3-methyl-N-[2-(4-methylpiperazine-1-carbonyl)benzo[b]thiophen-5-yl]-3-fluorobenzamide
N-[3-(4-cyanobenzyl)-3H-imidazol-4-ylmethyl]-3-methyl-N-[2-(4-methylpiperazine-1-carbonyl)benzo[b]thiophen-5-yl]-3-methoxybenzamide
N-(pyrid-3-ylmethyl)-5-{benzoyl-[3-(4-cyanobenzyl)-3H-imidazol-4-ylmethyl]amino}benzo[b]thiophene-2-carboxamide
N-(pyrid-3-ylmethyl)-5-{[3-(4-cyanobenzyl)-3H-imidazol-4-ylmethyl]-3-fluorobenzoylamino}benzo[b]thiophene-2-carboxamide
N-(pyrid-3-ylmethyl)-5-{[3-(4-cyanobenzyl)-3H-imidazol-4-ylmethyl]-3-methoxybenzoylamino}benzo[b]thiophene-2-carboxamide
N-(pyrid-3-ylmethyl)-5-{[3-(4-cyanobenzyl)-3H-imidazol-4-ylmethyl]-3-chlorobenzoylamino}benzo[b]thiophene-2-carboxamide
N-[3-(4-cyanobenzyl)-3H-imidazol-4-ylmethyl]-3-methyl-N-[2-(4-methylpiperazine-1-carbonyl)benzo[b]thiophen-5-yl]benzamide
N-[3-(4-cyanobenzyl)-3H-imidazol-4-ylmethyl]-3-methyl-N-[2-(4-methylpiperazine-1-carbonyl)benzo[b]thiophen-5-yl]-3-chlorobenzamide
N-(2-pyrrolidin-1-ylethyl)-5-{[3-(4-cyanobenzyl)-3H-imidazol-4-ylmethyl]cyclohexanecarbonylamino}benzo[b]thiophene-2-carboxamide
4-[5-({[2-(4-methylpiperazine-1-carbonyl)benzo[b]thiophen-5-yl]propylamino} methyl)imidazol-1-ylmethyl]benzonitrile
4-[5-({butyl-[2-(4-methylpiperazine-1-carbonyl)benzo[b]thiophen-5-yl]amino} methyl)imidzol-1-ylmethyl]benzonitrile
N-(2-pyrid-2-ylethyl)-5-{[3-(4-cyanobenzyl)-3H-imidazol-4-ylmethyl]propylamino}benzo[b]thiophene-2-carboxamide
N-(2-pyrid-2-ylethyl)-5-{[3-(4-cyanobenzyl)-3H-imidazol-4-ylmethyl]butylamino}benzo[b]thiophene-2-carboxamide
N-(1-methyl-1H-benzoimidazol-2-ylmethyl)-N-[2-(4-methylpiperazine-1-carbonyl)benzo[b]thiophen-5-yl]butyramide
N-(1-methyl-1H-benzoimidazol-2-ylmethyl)-N-[2-(4-methylpiperazine-1-carbonyl)benzo[b]thiophen-5-yl]cyclohexanecarboxamide
N-(1-methyl-1H-benzoimidazol-2-ylmethyl)-N-[2-(4-methylpiperazine-1-carbonyl)benzo[b]thiophen-5-yl]benzamide
N-(1-methyl-1H-benzoimidazol-2-ylmethyl)-N-[2-(4-methylpiperazine-1-carbonyl)benzo[b]thiophen-5-yl]-3-methoxybenzamide
N-(1-methyl-1H-benzoimidazol-2-ylmethyl)-N-[2-(4-ethylpiperazine-1-carbonyl)benzo[b]thiophen-5-yl]butyramide
N-(1-methyl-1H-benzoimidazol-2-ylmethyl)-[2-(4-ethylpiperazine-1-carbonyl)benzo[b]thiophen-5-yl]cyclohexanecarboxamide
N-(1-methyl-1H-benzoimidazol-2-ylmethyl)-N-[2-(4-ethylpiperazine-1-carbonyl)benzo[b]thiophen-5-yl]benzamide
N-(1-methyl-1H-benzoimidazol-2-ylmethyl)-N-[2-(4-ethylpiperazine-1-carbonyl)benzo[b]thiophen-5-yl]-3-methoxybenzamide
{5-[(1-methyl-1H-benzoimidazol-2-ylmethyl)propylamino]benzo[b]thiophen-2-yl}-(4-methylpiperazin-1-yl)methanone
{5-[(1-methyl-1H-benzoimidazol-2-ylmethyl)propylamino]benzo[b]thiophen-2-yl}-(4-ethylpiperazin-1-yl)methanone
{5-[benzenesulfonyl-(1-methyl-1H-benzoimidazol-2-ylmethyl)amino]benzo[b]thiophen-2-yl}-(4-methylpiperazin-1-yl)methanone
{5-[benzenesulfonyl-(1-methyl-1H-benzoimidazol-2-ylmethyl)amino]benzo[b]thiophen-2-yl}-(4-ethylpiperazin-1-yl)methanone and also the therapeutically acceptable salts and solvates thereof.

12. A process for preparing the compounds of general formula (Ia) as claimed in one of claims 1 to 11, characterized in that an intermediate of general formula (V) 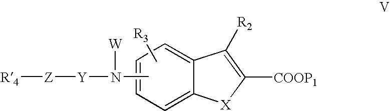
in which R₂, R₃, R′₄, W, X, Y and Z are defined as above, and either P₁ represents a protecting group or the species COOP, may represent an ester, which will be removed just before the condensation to give the free carboxylic acid, is condensed with an intermediate of formula R₆R₇NH in which R₆ and R₇ are defined as above. These steps will then be followed by a conversion of R′₄ into R₄.

13. A process for preparing the compounds of general formula (Ia) as claimed in one of claims 1 to 11, characterized in that an intermediate of general formula (VIII) 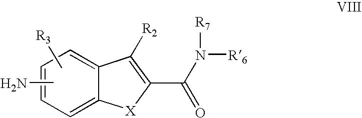
in which R₂, R₃, R′₆, R₇ and X are defined as above, is condensed with an intermediate of general formula R′₄-Z-Y-L₁, in which Z, Y and R′₄ are defined as above and L₁ represents either a leaving group or a hydroxyl. Or, finally, the portion Y-L₁ represents (CH₂)ₙ₋₁—CHO and the reaction with the amine (VIII) amounts to a reductive amination reaction. This step leads to an intermediate of general formula (IX): 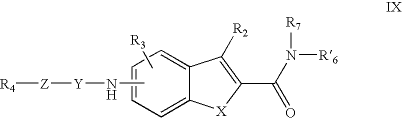
which may either directly give the compounds of general formula (Ia) in which W is a hydrogen, or may be treated with a reagent of general formula W-L₂ in which W is defined as above and L₂ represents either a leaving group or a hydroxyl. The species W-L₂ may also represent either an isocyanate or an isothiocyanate or an aldehyde. These steps will then be followed by a conversion of R′₆ into R₆.

14. A process for preparing the compounds of general formula (Ib) as claimed in one of claims 1 to 11, characterized in that an intermediate of general formula (XI) 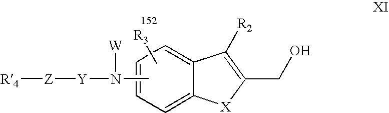
in which R₂, R₃, R′₄, Z, Y, W and X are defined as above, is condensed with an intermediate of general formula R′₆-L₃ in which R′₆ is defined as above and L₃ represents either a leaving group or an alcohol, followed by a conversion of R′₄ into R₄ and R′₆ into R₆.

15. A process for preparing the compounds of general formula (Ib) as claimed in one of claims 1 to 11, characterized in that an intermediate of general formula (XV) 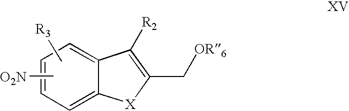
in which R₂, R₃ and X are defined as above and R′₆ corresponds either to R′₆ or to a precursor of R′₆, is converted via reduction of the nitro function to an amine, followed by successive condensation with an intermediate of general formula R₄-Z-Y-_{L1}, in which Z, Y, R₄ and L₁ are defined as above, and then with an intermediate of general formula W-L₂ in which W and L₂ are defined as above, followed by a conversion of R′₆ into R₆.

16. A process for preparing the compounds of general formula (Ic) as claimed in one of claims 1 to 11, characterized in that an intermediate of general formula (XVII) 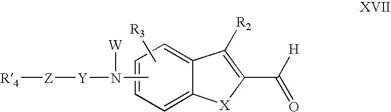
in which R₂, R₃, W, Z, Y, R′₄ and X are defined as above, is converted via reaction with a phosphonium salt of general formula Ph₃PCH₂R₆⁺X⁻in which R₆ is defined as above, followed by a conversion of R′₄ into R₄.

17. A process for preparing the compounds of general formula (Id) as claimed in one of claims 1 to 11, characterized in that an intermediate of general formula (XVII) 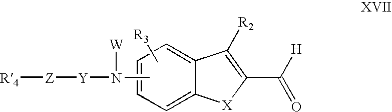
in which R₂, R₃, W, Z, Y, R′₄ and X are defined as above, is converted via reaction with a phosphonium salt of general formula Ph₃PCH₂R₆⁺X⁻in which R₆is defined as above, followed by a reduction of the double bond formed, and then a conversion of R′₄ into R₄.

18. A pharmaceutical composition containing, as active ingredient, at least one compound as claimed in one of claims 1 to 11 in combination with an acceptable pharmaceutical vehicle, as a medicinal product.

19. A pharmaceutical composition containing, as active ingredient, at least one compound as claimed in one of claims 1 to 11 in combination with an acceptable pharmaceutical vehicle, for the curative or preventive treatment of disorders associated with farnesylation and/or geranylgeranylation of proteins.

20. A pharmaceutical composition containing, as active ingredient, at least one compound as claimed in one of claims 1 to 11 in combination with an acceptable pharmaceutical vehicle, for the treatment or prevention of cancers such as cancer of the lung, of the pancreas, of the skin, of the head, of the neck, of the uterus, of the ovaries, anal cancer, cancer of the stomach, of the colon, of the breast, of the esophagus, of the small intestine, of the thyroid gland, of the prostate, of the kidney, of the bladder, acute or chronic leukemias, or alternatively a combination of 2 or more of these cancers.

21. A pharmaceutical composition containing, as active ingredient, at least one compound as claimed in one of claims 1 to 11 in combination with an acceptable pharmaceutical vehicle and administered in combination with an anticancer agent such as, for example, cytotoxic anticancer agents such as navelbine, vinflunine, taxol, taxotere, 5-fluorouracil, methotrexate, doxorubicin, camptothecin, gemcitabine, etoposide, cisplatin or BCNU, or hormonal anticancer agents, for instance tamoxifen or medroxyprogesterone, for the treatment or prevention of cancers.

22. A pharmaceutical composition containing, as active ingredient, at least one compound as claimed in one of claims 1 to 11 in combination with an acceptable pharmaceutical vehicle and administered in combination with an inhibitor of the biosynthesis of farnesyl and geranylgeranyl pyrophosphates, such as an HMG-CoA reductase inhibitor, for instance lovastatin, simvastatin, pravastatin, fluvastatin, atorvastatin or cerivastatin.

23. A pharmaceutical composition containing, as active ingredient, at least one compound as claimed in one of claims 1 to 11 in combination with an acceptable pharmaceutical vehicle and administered in combination with a treatment with radiation (X-rays or gamma rays) for the treatment or prevention of cancers.

24. A pharmaceutical composition containing, as active ingredient, at least one compound as claimed in one of claims 1 to 11 in combination with an acceptable pharmaceutical vehicle, for the treatment or prevention of restenosis or atherosclerosis.

25. A pharmaceutical composition containing, as active ingredient, at least one compound as claimed in one of claims 1 to 11 in combination with an acceptable pharmaceutical vehicle, for the treatment or prevention of infections associated with PFTase such as delta hepatitis.

26. A pharmaceutical composition containing, as active ingredient, at least one compound as claimed in one of claims 1 to 11 in combination with an acceptable pharmaceutical vehicle, for the treatment or prevention of benign proliferative disorders.
